(19) United States
(12) Patent Application Publication
(10) Pub. No.: US 2023/0183319 A1
(43) Pub. Date: Jun. 15, 2023

(54) VARIANT ACTRIIB PROTEINS AND USES THEREOF

(71) Applicant: Acceleron Pharma Inc., Cambridge, MA (US)

(72) Inventors: Ravindra Kumar, Cambridge, MA (US); Roselyne Castonguay, Cambridge, MA (US); Brantley Herrin, Cambridge, MA (US); Rose Maria Silva Garcia Grenha, Cambridge, MA (US)

(21) Appl. No.: 17/797,238

(22) PCT Filed: Feb. 3, 2021
(86) PCT No.: PCT/US2021/016434

§ 371 Date: Aug. 3, 2022

Related U.S. Application Data
(60) Provisional application No. 62/969,635, filed on Feb. 3, 2020.
(60) Provisional application No. 63/074,742, filed on Sep. 4, 2020.

(51) Int. Cl. C07K 14/71; A61K 45/06; A61K 38/17; A61P 13/12; A61P 29/00

(52) U.S. Cl. CPC C07K 14/71; A61K 38/179; A61K 45/06; A61P 13/12; A61P 29/00; C07K 2319/30

(57) ABSTRACT

In certain aspects, the present invention provides novel ActRIIB variants (in a homomultimeric or heteromultimeric form), as well as compositions and methods for using those variants to treat an indication associated with undesired activity of one or more TGFβ-superfamily ligands. The present invention also provides methods of screening compounds that modulate activity of an ActRIIB protein and/or an ActRIIB ligand. The compositions and methods provided herein are useful in treating diseases associated with abnormal activity of an ActRIIB protein and/or an ActRIIB ligand.

### CROSS-REFERENCE TO RELATED APPLICATIONS

This application claims the benefit of and priority to U.S. Provisional Application No. 62/969,635, filed Feb. 3, 2020 and U.S. Provisional Application No. 63/074,742, filed Sep. 4, 2020. The foregoing applications are incorporated herein by reference in their entirety.

### BACKGROUND OF THE INVENTION

The transforming growth factor-beta (TGF-beta) superfamily contains a variety of growth factors that share common sequence elements and structural motifs. These proteins are known to exert biological effects on a large variety of cell types in both vertebrates and invertebrates. Members of the superfamily perform important functions during embryonic development in pattern formation and tissue specification and can influence a variety of differentiation processes, including adipogenesis, myogenesis, chondrogenesis, cardiogenesis, hematopoiesis, neurogenesis, and epithelial cell differentiation. The family is divided into two general branches: the BMP/GDF and the TGF-beta/Activin/BMP10 branches, whose members have diverse, often complementary effects. By manipulating the activity of a member of the TGF-beta family, it is often possible to cause significant physiological changes in an organism. For example, the Piedmontese and Belgian Blue cattle breeds carry a loss-of-function mutation in the GDF8 (also called myostatin) gene that causes a marked increase in muscle mass. Grobet et al., Nat Genet. 1997, 17(1):71-4. Furthermore, in humans, inactive alleles of GDF8 are associated with increased muscle mass and, reportedly, exceptional strength. Schuelke et al., N Engl J Med 2004, 350:2682-8.

Changes in red blood cell levels, renal, pulmonary, cardiac, bone, cartilage and other tissues may be achieved by agonizing or antagonizing signaling that is mediated by an appropriate TGF-beta family member. Thus, there is a need for agents that function as potent regulators of TGF-beta signaling.

### SUMMARY OF THE INVENTION

In certain aspects, the present disclosure provides polypeptide, particularly variant ActRIIB polypeptides, variant ActRIIB homomultimer proteins, and variant ActRIIB heteromultimer proteins. In particular, the disclosure provides variant ActRIIB polypeptides with reduced binding affinity to BMP9 while retaining binding affinity to one or more of activin B, activin A, GDF11, GDF8, and BMP10. Accordingly, these variant ActRIIB polypeptides may be more useful than an unmodified ActRIIB polypeptide in certain applications where such selective antagonism is advantageous. Examples include therapeutic applications where it is desirable to retain antagonisms of one or more of activin A, activin B, GDF8, GDF11, and BMP10, while reducing antagonism of BMP9.

In part, the disclosure provides polypeptides, particularly variant ActRIIB polypeptides, variant ActRIIB homomultimer proteins, and variant ActRIIB heteromultimer proteins, that can be used to treat renal diseases or conditions (e.g., Alport syndrome, focal segmental glomerulosclerosis (FSGS), polycystic kidney disease, chronic kidney disease). Positive effects were observed for a variant ActRIIB polypeptide comprising a K mutation at position F82 in the UUO and Col4a3 (-/-) Alport syndrome models. The disclosure establishes that antagonists of the ActRII (e.g., ActRIIA and ActRIIB) signaling pathways may be used to reduce the severity of a renal disease or condition (e.g., Alport syndrome, focal segmental glomerulosclerosis (FSGS), polycystic kidney disease, chronic kidney disease), and that desirable therapeutic agents may be selected on the basis of ActRII signaling antagonist activity. Therefore, in some embodiments, the disclosure provides methods for using polypeptides, particularly variant ActRIIB polypeptides, variant ActRIIB homomultimer proteins, and variant ActRIIB heteromultimer proteins, for treating renal diseases or conditions including but not limited to Alport syndrome, focal segmental glomerulosclerosis (FSGS), polycystic kidney disease, and chronic kidney disease, including, for example, a variant ActRIIB polypeptide (e.g., a variant ActRIIB homomultimer protein or a variant ActRIIB heteromultimer protein) that inhibits one or more ActRIIA or ActRIIB ligands [e.g., activin A, activin B, GDF11, GDF8, GDF3, BMP6, BMP5, and BMP10].

In certain aspects, the present disclosure relates to a polypeptide comprising an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20 -29 (e.g., amino acid residues 20, 21, 22, 23, 24, 25, 26, 27, 28, or 29) of SEQ ID NO: 2 and ends at any one of amino acids 109 -134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises one or more amino acid substitutions at a position of SEQ ID NO: 2 selected from the group consisting of: N35, E50, E52, K55, L57, Y60, G68, K74, W78, L79, F82, N83, and E94. In some embodiments, the polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to amino acids 25-131 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to amino acids 20-134 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 53. In some embodiments, the polypeptide is a fusion protein further comprising a first polypeptide domain and one or more heterologous polypeptide domains. In some embodiments, the polypeptide is an ActRIIB-Fc fusion protein. In some embodiments, the fusion protein further comprises a linker domain positioned between the first polypeptide domain and the one or more heterologous domains or Fc domain. In some embodiments, the linker domain is selected from: TGGG, TGGGG, SGGGG, GGGGS, GGG, GGGG, SGGG, and GGGGS. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 5. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 12. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 519. In some embodiments, the polypeptide comprises one or more amino acid substitution with respect to the amino acid sequence of SEQ ID NO: 2 selected from the group consisting of: L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G. In some embodiments, the polypeptide comprises one or more amino acid substitution with respect to the amino acid sequence of SEQ ID NO: 2 selected from the group consisting of: L38N, E50L, E52D, E52N, E52Y, K55A, K55E, L57E, L57I, L57R, L57T, L57V, Y60D, G68R, K74E, W78Y, L79E, L79F, L79H, L79R, L79S, L79T, L79W, F82D, F82E, F82I, F82K, F82L, F82S, F82T, F82Y, N83R, E94K, and V99G. In some embodiments, the polypeptide comprises one or more amino acid substitution with respect to the amino acid sequence of SEQ ID NO: 2 selected from the group consisting of: A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132. In some embodiments, the polypeptide comprises an L substitution at the position corresponding to E50 of SEQ ID NO: 2. In some embodiments, the polypeptide comprises an N substitution at the position corresponding to L38 of SEQ ID NO: 2. In some embodiments, the polypeptide comprises a G substitution at the position corresponding to V99 of SEQ ID NO: 2. In some embodiments, the polypeptide comprises an R substitution at the position corresponding to N83 of SEQ ID NO: 2. In some embodiments, the polypeptide comprises an T substitution at the position corresponding to F82 of SEQ ID NO: 2. In some embodiments, the polypeptide comprises an H substitution at the position corresponding to L79 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 522. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 524. In some embodiments, the polypeptide comprises a K substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 276. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 278. In some embodiments, the polypeptide comprises an I substitution at the position corresponding to F82 of SEQ ID NO: 2 and an R substitution at the position corresponding to N83. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 279. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 332. In some embodiments, the polypeptide comprises a K substitution at the position corresponding to F82 of SEQ ID NO: 2 and an R substitution at the position corresponding to N83. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 333. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 335. In some embodiments, the polypeptide comprises a T substitution at the position corresponding to F82 of SEQ ID NO: 2 and an R substitution at the position corresponding to N83. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 336. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 338. In some embodiments, the polypeptide comprises a T substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 339. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 341. In some embodiments, the polypeptide comprises an H substitution at the position corresponding to L79 of SEQ ID NO: 2 and an I substitution at the position corresponding to F82. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 342. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 344. In some embodiments, the polypeptide comprises an H substitution at the position corresponding to L79 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 345. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 347. In some embodiments, the polypeptide comprises an H substitution at the position corresponding to L79 of SEQ ID NO: 2 and an K substitution at the position corresponding to F82. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 348. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 350. In some embodiments, the polypeptide comprises an L substitution at the position corresponding to E50 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 351. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 353. In some embodiments, the polypeptide comprises an N substitution at the position corresponding to L38 of SEQ ID NO: 2 and an R substitution at the position corresponding to L79. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 354. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 356. In some embodiments, the polypeptide comprises an G substitution at the position corresponding to V99 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 366. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 368. In some embodiments, the polypeptide comprises an E substitution at the position corresponding to N35 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 369. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 371. In some embodiments, the polypeptide comprises an N substitution at the position corresponding to E52 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 372. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 374. In some embodiments, the polypeptide comprises a D substitution at the position corresponding to Y60 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 375. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 377. In some embodiments, the polypeptide comprises an R substitution at the position corresponding to G68 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 378. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 380. In some embodiments, the polypeptide comprises an E substitution at the position corresponding to K74 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 381. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 383. In some embodiments, the polypeptide comprises an Y substitution at the position corresponding to W78 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 384. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 386. In some embodiments, the polypeptide comprises an A substitution at the position corresponding to L79 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 387. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 389. In some embodiments, the polypeptide comprises a K substitution at the position corresponding to L79 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 390. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 392. In some embodiments, the polypeptide comprises an S substitution at the position corresponding to L79 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 393. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 395. In some embodiments, the polypeptide comprises an W substitution at the position corresponding to L79 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 396. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 398. In some embodiments, the polypeptide comprises a D substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 399. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 401. In some embodiments, the polypeptide comprises an E substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 402. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 404. In some embodiments, the polypeptide comprises a L substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 405. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 407. In some embodiments, the polypeptide comprises a S substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 408. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 410. In some embodiments, the polypeptide comprises a Y substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 411. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 413. In some embodiments, the polypeptide comprises a K substitution at the position corresponding to E94 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 414. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 416. In some embodiments, the polypeptide comprises a D substitution at the position corresponding to E52 of SEQ ID NO: 2 and a D substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 417. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 419. In some embodiments, the polypeptide comprises a D substitution at the position corresponding to E52 of SEQ ID NO: 2 and a T substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 420. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 422. In some embodiments, the polypeptide comprises an R substitution at the position corresponding to L57 of SEQ ID NO: 2 and a D substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 423. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 425. In some embodiments, the polypeptide comprises an R substitution at the position corresponding to L57 of SEQ ID NO: 2 and an S substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 426. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 428. In some embodiments, the polypeptide comprises an R substitution at the position corresponding to L57 of SEQ ID NO: 2 and a T substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 429. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 431. In some embodiments, the polypeptide comprises an F substitution at the position corresponding to L79 of SEQ ID NO: 2 and a D substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 432. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 434. In some embodiments, the polypeptide comprises an F substitution at the position corresponding to L79 of SEQ ID NO: 2 and a T substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 435. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 437. In some embodiments, the polypeptide comprises a D substitution at the position corresponding to F82 of SEQ ID NO: 2 and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 438. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 440. In some embodiments, the polypeptide comprises an E substitution at the position corresponding to F82 of SEQ ID NO: 2 and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 441. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 443. In some embodiments, the polypeptide comprises an S substitution at the position corresponding to F82 of SEQ ID NO: 2 and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 444. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 446. In some embodiments, the polypeptide comprises an W substitution at the position corresponding to F82 of SEQ ID NO: 2 and an A substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 447. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 449. In some embodiments, the polypeptide comprises an I substitution at the position corresponding to F82 of SEQ ID NO: 2 and a K substitution at the position corresponding to E94 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 450. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 452. In some embodiments, the polypeptide comprises an L substitution at the position corresponding to E50 of SEQ ID NO: 2, a D substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 453. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 455. In some embodiments, the polypeptide comprises a D substitution at the position corresponding to E52 of SEQ ID NO: 2, a D substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 456. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 458. In some embodiments, the polypeptide comprises a D substitution at the position corresponding to E52 of SEQ ID NO: 2, an E substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 459. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 461. In some embodiments, the polypeptide comprises a D substitution at the position corresponding to E52 of SEQ ID NO: 2, a T substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 462. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 464. In some embodiments, the polypeptide comprises an N substitution at the position corresponding to E52 of SEQ ID NO: 2, an I substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 465. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 467. In some embodiments, the polypeptide comprises an N substitution at the position corresponding to E52 of SEQ ID NO: 2, a Y substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 468. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 470. In some embodiments, the polypeptide comprises a Y substitution at the position corresponding to E52 of SEQ ID NO: 2, a D substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 471. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 473. In some embodiments, the polypeptide comprises an E substitution at the position corresponding to L57 of SEQ ID NO: 2, an E substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 474. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 476. In some embodiments, the polypeptide comprises an I substitution at the position corresponding to L57 of SEQ ID NO: 2, a D substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 477. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 479. In some embodiments, the polypeptide comprises an I substitution at the position corresponding to L57 of SEQ ID NO: 2, an E substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 480. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 482. In some embodiments, the polypeptide comprises an R substitution at the position corresponding to L57 of SEQ ID NO: 2, an D substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 483. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 485. In some embodiments, the polypeptide comprises an R substitution at the position corresponding to L57 of SEQ ID NO: 2, an E substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 486. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 488. In some embodiments, the polypeptide comprises an R substitution at the position corresponding to L57 of SEQ ID NO: 2, an L substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 489. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 491. In some embodiments, the polypeptide comprises a T substitution at the position corresponding to L57 of SEQ ID NO: 2, a Y substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 492. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 494. In some embodiments, the polypeptide comprises a V substitution at the position corresponding to L57 of SEQ ID NO: 2, a D substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 495. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 497. In some embodiments, the polypeptide comprises a V substitution at the position corresponding to L57 of SEQ ID NO: 2, a Y substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 498. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 500. In some embodiments, the polypeptide comprises an R substitution at the position corresponding to G68 of SEQ ID NO: 2, a Y substitution at the position corresponding to W78 of SEQ ID NO: 2, and a Y substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 501. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 503. In some embodiments, the polypeptide comprises an R substitution at the position corresponding to G68 of SEQ ID NO: 2, an S substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 504. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 506. In some embodiments, the polypeptide comprises an N substitution at the position corresponding to E52 of SEQ ID NO: 2, an R substitution at the position corresponding to G68 of SEQ ID NO: 2, an Y substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 507. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 509. In some embodiments, the polypeptide comprises an R substitution at the position corresponding to G68 of SEQ ID NO: 2, an E substitution at the position corresponding to L79 of SEQ ID NO: 2, a T substitution at the position corresponding to F82 of SEQ ID NO: 2, and an R substitution at the position corresponding to N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 510. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 512. In some embodiments, the polypeptide comprises an R substitution at the position corresponding to G68 of SEQ ID NO: 2, an E substitution at the position corresponding to L79 of SEQ ID NO: 2, a Y substitution at the position corresponding to F82 of SEQ ID NO: 2, and an R substitution at the position correspondingto N83 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 513. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 515. In some embodiments, the polypeptide comprises an R substitution at the position corresponding to G68 of SEQ ID NO: 2, a T substitution at the position corresponding to L79 of SEQ ID NO: 2, a T substitution at the position corresponding to F82 of SEQ ID NO: 2, and an R substitution at the position corresponding to N83 of SEQ ID NO: 2.

In some embodiments of the present disclosure, the polypeptide displays a significant reduction in activin A binding, a significant reduction in GDF11 binding, no detected BMP10 binding, a minor reduction in BMP6 binding, and a significant reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays a significant reduction in activin A binding, near-WT levels of GDF11 binding, near-WT levels of BMP10 binding, and a slight increase in BMP6 binding, and a modest reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays a significant reduction in activin A binding, near-WT levels of GDF11 binding, a minor reduction in BMP10 binding, near-WT levels of BMP6 binding, and a minor reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays a modest reduction in activin A binding, a significant reduction in GDF11 binding, no detectable BMP10 binding, a minor reduction in BMP6 binding, and a significant reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays modest reduction in activin A binding, a significant reduction in GDF11 binding, no detectable BMP10 binding, a minor reduction in BMP6 binding, and a significant reduction in BMP9 binding, compared to binding profiles of an unmodified ActRIIB-Fc protein. In some embodiments, the polypeptide displays a modest reduction in activin B binding, compared to binding profiles of an unmodified ActRIIB-Fc protein.

In some embodiments of the present disclosure, the polypeptide displays modest reduction in activin A binding, a significant reduction in GDF11 binding, no detectable BMP10 binding, a significant reduction in BMP6 binding, and a significant reduction in BMP9 binding, compared to binding profiles of an unmodified ActRIIB-Fc protein. In some embodiments, the polypeptide displays modest reduction in activin B binding, compared to binding profiles of an unmodified ActRIIB-Fc protein.

In some embodiments of the present disclosure, the polypeptide displays a modest reduction in activin A binding, a significant reduction in GDF11 binding, no detectable BMP10 binding, a minor reduction in BMP6 binding, and a significant reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays a modest reduction in activin A binding, a significant reduction in GDF11 binding, minimal BMP10 binding, a minor reduction in BMP6 binding, and a significant reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays a modest reduction in activin A binding, a significant reduction in GDF11 binding, minimal BMP10 binding, a minor reduction in BMP6 binding, and a significant reduction in BMP9 binding, compared to binding profiles of an unmodified ActRIIB-Fc protein. In some embodiments, the polypeptide displays a modest reduction in activin B binding, compared to binding profiles of an unmodified ActRIIB-Fc protein.

In some embodiments of the present disclosure, the polypeptide displays a modest reduction in activin A binding, a significant reduction in GDF11 binding, minimal BMP10 binding, a modest reduction in BMP6 binding, and a significant reduction in BMP9 binding, compared to binding profiles of an unmodified ActRIIB-Fc protein. In some embodiments, the polypeptide displays a modest reduction in activin A binding, compared to binding profiles of an unmodified ActRIIB-Fc protein.

In some embodiments of the present disclosure, the polypeptide displays a modest reduction in activin A binding, a significant reduction in GDF11 binding, a modest reduction in BMP10 binding, a minor reduction in BMP6 binding, and a significant reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays a modest reduction in activin A binding, a significant reduction in GDF11 binding, a modest reduction in BMP10 binding, minimal BMP6 binding, and a significant reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays a modest reduction in activin A binding, a significant reduction in GDF11 binding, a modest reduction in BMP10 binding, minimal BMP6 binding, and a modest reduction in BMP9 binding, compared to binding profiles of an unmodified ActRIIB-Fc protein. In some embodiments, the polypeptide displays a modest reduction in activin B binding, compared to binding profiles of an unmodified ActRIIB-Fc protein.

In some embodiments of the present disclosure, the polypeptide displays a modest reduction in activin A binding, a modest reduction in GDF11 binding, no detectable BMP10 binding, a minor reduction in BMP6 binding, and a significant reduction in BMP9 binding, compared to binding profiles of an unmodified ActRIIB-Fc protein. In some embodiments, the polypeptide displays a minor reduction in activin B binding, compared to binding profiles of an unmodified ActRIIB-Fc protein.

In some embodiments of the present disclosure, the polypeptide displays a modest reduction in activin A binding, near-WT levels of GDF11 binding, near-WT levels of BMP10 binding, a slight increase in BMP6 binding, and near-WT levels of BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays a modest reduction in activin A binding, near-WT levels of GDF11 binding, near-WT levels of BMP10 binding, a slight increase in BMP6 binding, and near-WT levels of BMP9 binding, compared to binding profiles of an unmodified ActRIIB-Fc protein. In some embodiments, the polypeptide displays a modest reduction in activin B binding, compared to binding profiles of an unmodified ActRIIB-Fc protein.

In some embodiments of the present disclosure, the polypeptide displays a minor reduction in activin A binding, a modest reduction in GDF11 binding, a modest reduction in BMP10 binding, a significant reduction in BMP6 binding, and s significant reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays near-WT levels of activin A binding, near-WT levels of GDF11 binding, a modest reduction in BMP10 binding, a modest reduction in BMP6 binding, and a modest reduction in BMP9 binding, compared to binding profiles of an unmodified ActRIIB-Fc protein. In some embodiments, the polypeptide displays a minor reduction in activin B binding, compared to binding profiles of an unmodified ActRIIB-Fc protein.

In some embodiments of the present disclosure, the polypeptide displays near-WT levels of activin A binding, near-WT levels of GDF11 binding, near-WT levels of BMP10 binding, a minimal binding to BMP6, and a minor reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays near-WT levels of activin A binding, near-WT levels of GDF11 binding, near-WT levels of BMP10 binding, near-WT levels of BMP6 binding, and a minor reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays near-WT levels of activin A binding, near-WT levels of GDF11 binding, near-WT levels of BMP10 binding, near-WT levels of BMP6 binding, and near-WT levels of BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays near-WT levels of activin A binding, near-WT levels of GDF11 binding, near-WT levels of BMP10 binding, near-WT levels of BMP6 binding, and a modest reduction in BMP9 binding, compared to binding profiles of an unmodified ActRIIB-Fc protein. In some embodiments, the polypeptide displays a minor reduction in activin B binding, compared to binding profiles of an unmodified ActRIIB-Fc protein.

In some embodiments of the present disclosure, the polypeptide displays near-WT levels of activin A binding, near-WT levels of GDF11 binding, a minor reduction in BMP10 binding, near-WT levels of BMP6 binding, and a modest reduction in BMP9 binding, compared to binding profiles of an unmodified ActRIIB-Fc protein. In some embodiments, the polypeptide displays a minor reduction in activin B binding, compared to binding profiles of an unmodified ActRIIB-Fc protein.

In some embodiments of the present disclosure, the polypeptide displays near-WT levels of activin A binding, near-WT levels of GDF11 binding, a minor reduction in BMP10 binding, near-WT levels of BMP6 binding, and a modest reduction in BMP9 binding, compared to binding profiles of an unmodified ActRIIB-Fc protein. In some embodiments, the polypeptide displays near-WT levels of activin B binding, compared to binding profiles of an unmodified ActRIIB-Fc protein.

In some embodiments of the present disclosure, the polypeptide displays near-WT levels of activin A binding, near-WT levels of GDF11 binding, near-WT levels of BMP10 binding, a minor reduction in BMP6 binding, and a modest reduction in BMP9 binding, compared to binding profiles of an unmodified ActRIIB-Fc protein. In some embodiments, the polypeptide displays a minor reduction in activin B binding, compared to binding profiles of an unmodified ActRIIB-Fc protein.

In some embodiments of the present disclosure, the polypeptide is a homodimer protein.

In certain aspects, the present disclosure relates to a heteromultimer protein comprising a variant ActRIIB polypeptide and an ALK4 polypeptide, wherein the variant ActRIIB polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2 and one or more amino acid substitutions at a position of SEQ ID NO: 2 selected from the group consisting of: L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G. In certain aspects, the present disclosure relates to a heteromultimer protein comprising a variant ActRIIB polypeptide and an ALK7 polypeptide, wherein the variant ActRIIB polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2 and one or more amino acid substitutions at a position of SEQ ID NO: 2 selected from the group consisting of: L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G. In certain aspects, the present disclosure relates to a heteromultimer protein comprising a variant ActRIIB polypeptide and an ALK4 polypeptide, wherein the variant ActRIIB polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2 and one or more amino acid substitutions at a position of SEQ ID NO: 2 selected from the group consisting of: L38N, E50L, E52D, E52N, E52Y, K55A, K55E, L57E, L57I, L57R, L57T, L57V, Y60D, G68R, K74E, W78Y, L79E, L79F, L79H, L79R, L79S, L79T, L79W, F82D, F82E, F82I, F82K, F82L, F82S, F82T, F82Y, N83R, E94K, and V99G. In certain aspects, the present disclosure relates to a heteromultimer protein comprising a variant ActRIIB polypeptide and an ALK7 polypeptide, wherein the variant ActRIIB polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2 and one or more amino acid substitutions at a position of SEQ ID NO: 2 selected from the group consisting of: L38N, E50L, E52D, E52N, E52Y, K55A, K55E, L57E, L57I, L57R, L57T, L57V, Y60D, G68R, K74E, W78Y,L79E, L79F, L79H, L79R, L79S, L79T, L79W, F82D, F82E, F82I, F82K, F82L, F82S,F82T, F82Y, N83R, E94K, and V99G.

In certain aspects, the present disclosure relates to a heteromultimer protein comprising a variant ActRIIB polypeptide and an ALK4 polypeptide, wherein the variant ActRIIB polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2 and one or more amino acid substitutions at a position of SEQ ID NO: 2 selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.

In certain aspects, the present disclosure relates to a heteromultimer protein comprising a variant ActRIIB polypeptide and an ALK7 polypeptide, wherein the variant ActRIIB polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2 and one or more amino acid substitutions at a position of SEQ ID NO: 2 selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.56. The heteromultimer of any one of claims 52-55, wherein the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2.

In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 25-131 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 20-134 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 53. In some embodiments, the variant ActRIIB polypeptide comprises one or more amino acid substitution with respect to the amino acid sequence of SEQ ID NO: 2 selected from the group consisting of: L38N, E50L, E52D, E52N, E52Y, L57E, L57I, L57R, L57T, L57V, Y60D, G68R, K74E, W78Y, L79E, L79F, L79H, L79R, L79S, L79T, L79W, F82D, F82E, F82I, F82K, F82L, F82S, F82T, F82Y, N83R, E94K, and V99G. In some embodiments, the variant ActRIIB polypeptide comprises one or more amino acid substitution with respect to the amino acid sequence of SEQ ID NO: 2 selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.

In some embodiments, the ALK4 polypeptide comprises an amino acid sequence that is at least 75%, 80%, 85%, 90%, 91%, 92%, 93%, 94%, 95%, 96%, 97%, 98%, 99%, or 100% identical to an amino acid sequence selected from the group consisting of SEQ ID NOs: 84, 85, 86, 87, 88, 89, 92, 93, 247, and 249. In some embodiments, the ALK7 polypeptide comprises an amino acid sequence that is at least 75%, 80%, 85%, 90%, 91%, 92%, 93%, 94%, 95%, 96%, 97%, 98%, 99%, or 100% identical to an amino acid sequence selected from the group consisting of SEQ ID NOs: 120, 121, 122, 123, 124, 125, 126, 127, 128, 129, 130, 133, and 134. In some embodiments, the variant ActRIIB polypeptide is a fusion protein comprising an ActRIIB polypeptide domain and one or more heterologous domains. In some embodiments, the ALK4 polypeptide is a fusion protein comprising an ALK4 polypeptide domain and one or more heterologous domains. In some embodiments, the ALK7 polypeptide is a fusion protein comprising an ALK7 polypeptide domain and one or more heterologous domains. In some embodiments, the variant ActRIIB polypeptide is an ActRIIB-Fc fusion protein. In some embodiments, the ALK4 polypeptide is an ALK4-Fc fusion protein. In some embodiments, the ALK7 polypeptide is an ALK7-Fc fusion protein. In some embodiments, the variant ActRIIB-Fc fusion protein further comprises a linker domain positioned between the ActRIIB polypeptide domain and the one or more heterologous domains or Fc domain. In some embodiments, the ALK4-Fc fusion protein further comprises a linker domain positioned between the ALK4 polypeptide domain and the one or more heterologous domains or Fc domain. In some embodiments, the ALK7-Fc fusion protein further comprises a linker domain positioned between the ALK7 polypeptide domain and the one or more heterologous domains or Fc domain. In some embodiments, the linker domain is selected from: TGGG, TGGGG, SGGGG, GGGGS, GGG, GGGG, SGGG, and GGGGS. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13; (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14; (c) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15; (d) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16; and (e) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13; (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14; (c) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15; (d) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16; and (e) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26. In some embodiments, the heteromultimer comprises an Fc domain selected from: (a) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27; and (b) The variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26. In some embodiments, the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK4- Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29. In some embodiments, the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29. In some embodiments, the variant ActRIIB-Fc fusion protein Fc domain comprises a cysteine at amino acid position 132, glutamic acid at amino acid position 138, a tryptophan at amino acid position 144, and a aspartic acid at amino acid position 217, and wherein the ALK4-Fc fusion protein Fc domain comprises a cysteine at amino acid position 127, a serine at amino acid position 144, an alanine at position 146 an arginine at amino acid position 162, an arginine at amino acid position 179, and a valine at amino acid position 185. In some embodiments, the variant ActRIIB-Fc fusion protein Fc domain comprises a cysteine at amino acid position 132, glutamic acid at amino acid position 138, a tryptophan at amino acid position 144, and a aspartic acid at amino acid position 217, and wherein the ALK7-Fc fusion protein Fc domain comprises a cysteine at amino acid position 127, a serine at amino acid position 144, an alanine at position 146 an arginine at amino acid position 162, an arginine at amino acid position 179, and a valine at amino acid position 185. In some embodiments, the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29. In some embodiments, the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29. In some embodiments, the ALK4-Fc fusion protein Fc domain comprises a cysteine at amino acid position 132, glutamic acid at amino acid position 138, a tryptophan at amino acid position 144, and a aspartic acid at amino acid position 217, and wherein the variant ActRIIB-Fc fusion protein Fc domain comprises a cysteine at amino acid position 127, a serine at amino acid position 144, an alanine at position 146 an arginine at amino acid position 162, an arginine at amino acid position 179, and a valine at amino acid position 185. In some embodiments, the ALK7-Fc fusion protein Fc domain comprises a cysteine at amino acid position 132, glutamic acid at amino acid position 138, a tryptophan at amino acid position 144, and a aspartic acid at amino acid position 217, and wherein the variant ActRIIB-Fc fusion protein Fc domain comprises a cysteine at amino acid position 127, a serine at amino acid position 144, an alanine at position 146 an arginine at amino acid position 162, an arginine at amino acid position 179, and a valine at amino acid position 185. In some embodiments, the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23. In some embodiments, the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23. In some embodiments, the variant ActRIIB-Fc fusion protein Fc domain comprises a cysteine at amino acid position 132, a tryptophan at amino acid position 144, and a arginine at amino acid position 435, and wherein the ALK4-Fc fusion protein Fc domain comprises cysteine at amino acid position 127, a serine at amino acid position 144, an alanine at amino acid position 146, and a valine at amino acid position 185. In some embodiments, the variant ActRIIB-Fc fusion protein Fc domain comprises a cysteine at amino acid position 132, a tryptophan at amino acid position 144, and a arginine at amino acid position 435, and wherein the ALK7-Fc fusion protein Fc domain comprises cysteine at amino acid position 127, a serine at amino acid position 144, an alanine at amino acid position 146, and a valine at amino acid position 185. In some embodiments, the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23. In some embodiments, the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23. In some embodiments, the ALK4-Fc fusion protein Fc domain comprises a cysteine at amino acid position 132, a tryptophan at amino acid position 144, and a arginine at amino acid position 435, and wherein the variant ActRIIB-Fc fusion protein Fc domain comprises cysteine at amino acid position 127, a serine at amino acid position 144, an alanine at amino acid position 146, and a valine at amino acid position 185. In some embodiments, the ALK7-Fc fusion protein Fc domain comprises a cysteine at amino acid position 132, a tryptophan at amino acid position 144, and a arginine at amino acid position 435, and wherein the variant ActRIIB-Fc fusion protein Fc domain comprises cysteine at amino acid position 127, a serine at amino acid position 144, an alanine at amino acid position 146, and a valine at amino acid position 185. In some embodiments, the heteromultimer is a heterodimer.

In certain aspects, the disclosure relates to isolated and/or recombinant nucleic acids comprising a coding sequence for one or more of the ALK4 polypeptide(s) described herein. For example, in some embodiments, the disclosure relates to an isolated and/or recombinant nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 221, 222, 223, and 224. In some embodiments, the disclosure relates to isolated and/or recombinant nucleic acids comprising a coding sequence for one or more of the ALK7 polypeptide(s) described herein. For example, in some embodiments, the disclosure relates to an isolated and/or recombinant nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID NOs: 233, 234, 235, 236, 237, 238, 239, 240, and 255. In some embodiments, the disclosure relates to isolated and/or recombinant nucleic acids comprising a coding sequence for one or more of the polypeptide(s) described herein. For example, in some embodiments, the disclosure relates to an isolated and/or recombinant nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 10, 32, 35, 38, 41, 44, 47, 277, 331, 334, 337, 340, 343, 346, 349, 352, 355, 367, 370, 373, 376, 379, 382, 385, 388, 391, 394, 397, 400, 403, 406, 409, 412, 415, 418, 421, 424, 427, 430, 433, 436, 439, 442, 445, 448, 451, 454, 457, 460, 463, 466, 469, 472, 475, 478, 481, 484, 487, 490, 493, 496, 499, 502, 505, 508, 511, 514, 517, 521, and 523. In some embodiments, an isolated and/or recombinant polynucleotide sequence of the disclosure comprises a coding sequence for a heteromultimer described herein (e.g., an ActRIIB-Fc:ALK4-Fc heteromultimer or an ActRIIB-Fc:ALK7-Fc heteromultimer). In some embodiments, an isolated and/or recombinant polynucleotide sequence of the disclosure comprises a promoter sequence operably linked to the coding sequence described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 10, 32, 35, 38, 41, 44, 47, 277, 331, 334, 337, 340, 343, 346, 349, 352, 355, 367, 370, 373, 376, 379, 382, 385, 388, 391, 394, 397, 400, 403, 406, 409, 412, 415, 418, 421, 424, 427, 430, 433, 436, 439, 442, 445, 448, 451, 454, 457, 460, 463, 466, 469, 472, 475, 478, 481, 484, 487, 490, 493, 496, 499, 502, 505, 508, 511, 514, 517, 521, and 523). In some embodiments, the disclosure relates to a vector comprising an isolated nucleic acid described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 10, 32, 35, 38, 41, 44, 47, 277, 331, 334, 337, 340, 343, 346, 349, 352, 355, 367, 370, 373, 376, 379, 382, 385, 388, 391, 394, 397, 400, 403, 406, 409, 412, 415, 418, 421, 424, 427, 430, 433, 436, 439, 442, 445, 448, 451, 454, 457, 460, 463, 466, 469, 472, 475, 478, 481, 484, 487, 490, 493, 496, 499, 502, 505, 508, 511, 514, 517, 521, and 523). In some embodiments, the disclosure relates to a cell comprising a recombinant polynucleotide sequence or a vector described herein.

In certain aspects, the disclosure relates to methods of making a polypeptide comprising culturing a cell under conditions suitable for expression of a polypeptide, wherein the cell comprises a nucleic acid comprising a coding sequence for the polypeptide described herein. Such a method may include expressing any of the nucleic acids) disclosed herein in a suitable cell (e.g., a CHO cell or COS cell). In some embodiments, the disclosure relates to methods of making a heteromultimer comprising a variant ActRIIB polypeptide and an ALK4 polypeptide comprising culturing a cell under conditions suitable for expression of a variant ActRIIB polypeptide and the ALK4 polypeptide, wherein the cell comprises a first nucleic acid comprising a coding sequence for a variant ActRIIB polypeptide described herein and a second nucleic acid comprising a coding sequence for an ALK4 polypeptide described herein. In some embodiments, the disclosure relates to methods of making a heteromultimer comprising a variant ActRIIB polypeptide and an ALK7 polypeptide comprising culturing a cell under conditions suitable for expression of a variant ActRIIB polypeptide and the ALK7 polypeptide, wherein the cell comprises a first nucleic acid comprising a coding sequence for the variant ActRIIB polypeptide described herein and a second nucleic acid comprising a coding sequence for an ALK7 polypeptide described herein. In some embodiments, the method comprises a further step of recovering the heteromultimer. In some embodiments, the disclosure relates to methods of making a heteromultimer comprising a variant ActRIIB polypeptide and an ALK4 polypeptide comprising: (a) culturing a first cell under conditions suitable for expression of a variant ActRIIB polypeptide, wherein the cell comprises a nucleic acid comprising a coding sequence for a variant ActRIIB polypeptide; (b) recovering the variant ActRIIB polypeptide; (c) culturing a second cell under conditions suitable for expression of an ALK4 polypeptide, wherein the cell comprises a nucleic acid comprising a coding sequence for a ALK4 polypeptide; (d) recovering the ALK4 polypeptide; (e) combining the recovered variant ActRIIB polypeptide and the ALK4 polypeptide under conditions suitable for heteromultimer formation. In some embodiments, the disclosure relates to methods of making a heteromultimer comprising a variant ActRIIB polypeptide and an ALK7 polypeptide comprising: (a) culturing a first cell under conditions suitable for expression of a variant ActRIIB polypeptide, wherein the cell comprises a nucleic acid comprising a coding sequence for a variant ActRIIB polypeptide; (b) recovering the variant ActRIIB polypeptide; (c) culturing a second cell under conditions suitable for expression of an ALK7 polypeptide, wherein the cell comprises a nucleic acid comprising a coding sequence for a ALK7 polypeptide; (d) recovering the ALK7 polypeptide; (e) combining the recovered variant ActRIIB polypeptide and the ALK7 polypeptide under conditions suitable for heteromultimer formation. In some embodiments, the method further comprises recovering the expressed heteromultimer polypeptide.

In some embodiments, the disclosure relates to methods for increasing red blood cell levels or hemoglobin levels in a patient, comprising administering a patient in need thereof a polypeptide or heteromultimer described herein. In some embodiments, the disclosure relates to methods for treating anemia or a disorder associated with anemia in a patient, comprising administering a patient in need thereof a polypeptide or heteromultimer described herein. In some embodiments, the anemia or disorder associated with anemia is selected from the group consisting of MDS, thalassemia, and myelofibrosis. In some embodiments, the disclosure relates to methods for increasing muscle mass and/or muscle strength in a patient, comprising administering a patient in need thereof a polypeptide or heteromultimer described herein. In some embodiments, the disclosure relates to methods for treating a muscle-related disorder in a patient, comprising administering a patient in need thereof a polypeptide or heteromultimer described herein. In some embodiments, the muscle-related disorder is associated with undesirably low muscle growth and/or muscle weakness. In some embodiments, the muscle-related disorder is selected from the group consisting of Duchenne muscular dystrophy (DMD), Becker muscular dystrophy (BMD), Charcot-Marie-Tooth disease (CMT), facioscapulohumeral muscular dystrophy (FSH or FSHD), Amyotrophic Lateral Sclerosis (ALS), and spinal muscular atrophy (SMA).

In some embodiments, the disclosure relates to methods for treating pulmonary arterial hypertension in a patient, comprising administering a patient in need thereof a polypeptide or heteromultimer described herein. In some embodiments, the disclosure relates to methods for treating pulmonary hypertension in a patient, comprising administering a patient in need thereof a polypeptide or heteromultimer described herein. In some embodiments, the disclosure relates to methods for treating interstitial lung disease in a patient, comprising administering a patient in need thereof a polypeptide or heteromultimer described herein. In some embodiments, the disclosure relates to methods for treating kidney-associated disease in a patient, comprising administering a patient in need thereof a polypeptide or heteromultimer described herein. In some embodiments, the kidney-associated disease is selected from the group consisting of Alport syndrome, focal segmental glomerulosclerosis, polycystic kidney disease, or chronic kidney disease. In some embodiments, the disclosure relates to methods for treating a bone related disorder in a patient, comprising administering a patient in need thereof a polypeptide or heteromultimer described herein. In some embodiments, the bone related disorder is selected from the group consisting of osteoporosis, hyperparathyroidism, Cushing’s disease, thyrotoxicosis, chronic diarrheal state or malabsorption, renal tubular acidosis, anorexia nervosa, or fibrodysplasia ossificans progressiva (FOP).

In some embodiments, polypeptides of the present disclosure bind to one or more ligands selected from the group consisting of activin A, activin B, GDF11, GDF8, GDF3, BMP5, BMP6, and BMP10. In some embodiments, polypeptides of the present disclosure bind to activin A, GDF8, GDF11, and BMP10. In some embodiments, polypeptides of the present disclosure inhibit activity of one or more ligands in a cell-based assay.

In some embodiments, heteromultimer proteins of the present disclosure bind to one or more ligands selected from the group consisting of activin A, activin B, GDF11, GDF8, GDF3, BMP5, BMP6, and BMP10. In some embodiments, heteromultimer proteins of the present disclosure bind to activin A, GDF8, GDF11, and BMP10. In some embodiments, heteromultimer proteins of the present disclosure inhibit activity of one or more ligands in a cell-based assay.

In certain aspects, the disclosure relates to methods of treating a renal disease or condition comprising administering a polypeptide to a subject in need thereof, wherein the polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises one or more amino acid substitutions at a position of SEQ ID NO: 2 selected from the group consisting of: N35, E50, E52, K55, L57, Y60, G68, K74, W78, L79, F82, N83, E94.

In some embodiments, any of the polypeptides, including heteromultimers thereof, described herein, may be used in accordance with methods of the present disclosure. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 31. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 33. In some embodiments, the polypeptide comprises an A substitution at the position corresponding to K55 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 34. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 36. In some embodiments, the polypeptide comprises an E substitution at the position corresponding to K55 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 40. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 42. In some embodiments, the polypeptide comprises a K substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 37. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 39. In some embodiments, the polypeptide comprises an I substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 43. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 45. In some embodiments, the polypeptide comprises an E substitution at the position corresponding to L79 of SEQ ID NO: 2. In some embodiments, the polypeptide comprises a polypeptide of the present disclosure (e.g., an amino acid sequence corresponding to any one of SEQ ID NOs: 366, 368, 369, 371, 372, 374, 375, 377, 378, 380, 381, 383, 384, 386, 387, 389, 390, 392, 393, 395, 396, 398, 399, 401, 402, 404, 405, 407, 408, 410, 411, 413, 414, 416, 417, 419, 420, 422, 423, 425, 426, 428, 429, 431, 432, 434, 435, 437, 438, 440, 441, 443, 444, 446, 447, 449, 450, 452, 453, 455, 456, 458, 459, 461, 462, 464, 465, 467, 468, 470, 471, 473, 474, 476, 477, 479, 480, 482, 483, 485, 486, 488, 489, 491, 492, 494, 495, 497, 498, 500, 501, 503, 504, 506, 507, 509, 510, 512, 513, 515, 522, and 524)

In some embodiments of the present disclosure, the renal disease or condition is Alport syndrome. In some embodiments of the present disclosure, the renal disease or condition is focal segmental glomerulosclerosis (FSGS). In some embodiments of the present disclosure, the renal disease or condition is polycystic kidney disease. In some embodiments, of the present disclosure the renal disease or condition is autosomal dominant polycystic kidney disease (ADPKD). In some embodiments of the present disclosure, the renal disease or condition is autosomal recessive polycystic kidney disease (ARPKD). In some embodiments of the present disclosure, the renal disease or condition is chronic kidney disease (CKD). In some embodiments, the subject has a decline in kidney function. In some embodiments, the method slows kidney function decline.

In some embodiments, methods of the present disclosure further comprise administering to the subject an additional active agent and/or supportive therapy for treating a renal disease or condition. In some embodiments, the additional active agent and/or supportive therapy for treating a renal disease or condition is selected from the group consisting of: an angiotensin receptor blocker (ARB) (e.g., losartan, irbesartan, olmesartan, candesartan, valsartan, fimasartan, azilsartan, salprisartan, and telmisartan), an angiotensin-converting enzyme (ACE) inhibitor (e.g., benazepril, captopril, enalapril, lisinopril, perindopril, ramipril, trandolapril, and zofenopril), a glucocorticoid (e.g., beclomethasone, betamethasone, budesonide, cortisone, dexamethasone, hydrocortisone, methylprednisolone, prednisolone, methylprednisone, prednisone, and triamcinolone), a calcineurin inhibitor (e.g., cyclosporine, tacrolimus), cyclophosphamide, chlorambucil, a janus kinase inhibitor (e.g., tofacitinib), an mTOR inhibitor (e.g., sirolimus, everolimus), an IMDH inhibitor (e.g., azathioprine, leflunomide, mycophenolate), a biologic (e.g., abatacept, adalimumab, anakinra, basiliximab, certolizumab, daclizumab, etanercept, fresolimumab, golimumab, infliximab, ixekizumab, natalizumab, rituximab, secukinumab, tocilizumab, ustekinumab, vedolizumab), a statin (e.g., benazepril, valsartan, fluvastatin, pravastatin), bardoxolone methyl, Achtar gel, tolvaptan, abatacept in combination with sparsentan, aliskiren, allopurinol, ANG-3070, atorvastatin, bleselumab, bosutinib, CCX140-B, CXA-10, D6-25-hydroxyvitamin D3, dapagliflozin, dexamethasone in combination with MMF, emodin, FG-3019, FK506, FK-506 and MMF, FT-011, galactose, GC1008, GFB-887, isotretinoin, lademirsen, lanreotide, levamisole, lixivaptan, losmapimod, metformin, mizorbine, N-acetylmannosamine, octreotide, paricalcitol, PF-06730512, pioglitazone, propagermanium, propagermanium and irbesartan, rapamune, rapamycin, RE-021 (e.g., sparsentan), RG012, rosiglitazone (e.g., Avandia), saquinivir, SAR339375, somatostatin, spironolactone, tesevatinib (KD019), tetracosactin, tripterygium wilfordii (TW), valproic acid, VAR-200, venglustat (GZ402671), verinurad, voclosporin, VX-417, kidney dialysis, kidney transplant, mesenchymal stem cell therapy, bone marrow stem cells, lipoprotein removal, a Liposorber LA-15 device, plasmapheresis, plasma exchange, and a change in diet (e.g., dietary sodium intake). In some embodiments, the additional active agent and/or supportive therapy for treating a renal disease or condition is an angiotensin receptor blocker (ARB) selected from the group consisting of losartan, irbesartan, olmesartan, candesartan, valsartan, fimasartan, azilsartan, salprisartan, and telmisartan. In some embodiments, the additional active agent and/or supportive therapy for treating a renal disease or condition is an angiotensin-converting enzyme (ACE) inhibitor selected from the group consisting of benazepril, captopril, enalapril, lisinopril, perindopril, ramipril, trandolapril, and zofenopril. In some embodiments, the additional active agent and/or supportive therapy for treating a renal disease or condition is a combination of an ARB and an ACE inhibitor.

In some embodiments of the present disclosure, the subject has proteinuria. In some embodiments, the subject has albuminuria. In some embodiments, the subject has moderate albuminuria. In some embodiments, the subject has severe albuminuria. In some embodiments, the method reduces severity, occurrence and/or duration of one or more of albuminuria, proteinuria, microalbuminuria, and macroalbuminuria in a subject in need thereof.

In some embodiments of the present disclosure, the subject has an albumin-creatinine ratio (ACR) of between about 30 and about 300 mg albumin per 24 hours of urine collection. In some embodiments, the subject has an ACR of between about 30 and about 300 mg albumin/g of creatinine. In some embodiments, the subject has an albumin-creatinine ratio (ACR) of above about 300 mg albumin/24 hours. In some embodiments, the subject has an ACR of above about 300 mg albumin/g of creatinine. In some embodiments, the subject has Stage A1 albuminuria. In some embodiments, the subject has Stage A2 albuminuria. In some embodiments, the subject has Stage A3 albuminuria. In some embodiments of the present disclosure, the method reduces severity, occurrence and/or duration of Stage A1 albuminuria. In some embodiments, the method reduces severity, occurrence and/or duration of Stage A2 albuminuria. In some embodiments, the method reduces severity, occurrence and/or duration of Stage A3 albuminuria. In some embodiments, the method delays or prevents a subject with Stage A1 albuminuria from progressing to Stage A2 albuminuria. In some embodiments, the method delays or prevents a subject with Stage A2 from progressing to Stage A3 albuminuria. In some embodiments, methods of the present disclosure delay and/or prevent worsening of albuminuria stage progression in a subject in need thereof. In some embodiments of the present disclosure, methods of the present disclosure improve albuminuria classification in a subject by one or more stages. In some embodiments, methods of the present disclosure reduce an ACR of the subject. In some embodiments, methods of the present disclosure reduce the subject’s ACR by between about 0.1 and about 100.0 mg albumin/g creatinine (e.g., by between about 0.1 and about 2.5 mg albumin/g , between about 2.5 and about 3.5 mg albumin/g creatinine, between about 3.5 and about 5.0 mg albumin/g creatinine, between about 5.0 and about 7.5 mg albumin/g creatinine, between about 7.5 and about 10.0 mg albumin/g creatinine, between about 10.0 and about 15.0 mg albumin/g creatinine, between about 15.0 and about 20.0 mg albumin/g creatinine, between about 20.0 and about 25.0 mg albumin/g creatinine, between about 30.0 and about 35.0 mg albumin/g creatinine, between about 40.0 and about 45.0 mg albumin/g creatinine, between about 45.0 and about 50.0 mg albumin/g creatinine, between about 50.0 and about 60.0 mg albumin/g creatinine, between about 60.0 and about 70.0 mg albumin/g creatinine, between about 70.0 and about 80.0 mg albumin/g creatinine, between about 80.0 and about 90.0 mg albumin/g creatinine, between about 90.0 and about 100.0 mg albumin/g creatinine). In some embodiments, methods of the present disclosure reduce the subject’s ACR by at least 2.5% (e.g., 5%, 10%, 15%, 20%, 25%, 30%, 40%, 50%, 60%, 70%. 80%, 90%, 95%, or 99%) compared to a baseline measurement.

In some embodiments, methods of the present disclosure reduce a urinary protein-creatinine ratio (UPCR) of the subject. In some embodiments, methods of the present disclosure reduce the subject’s UPCR between about 0.1 and about 100.0 mg urinary protein/mg creatinine (e.g., by between about 0.1 and about 2.5 mg urinary protein/mg creatinine, between about 2.5 and about 3.5 mg urinary protein/mg creatinine, between about 3.5 and about 5.0 mg urinary protein/mg creatinine, between about 5.0 and about 7.5 mg urinary protein/mg creatinine, between about 7.5 and about 10.0 mg urinary protein/mg creatinine, between about 10.0 and about 15.0 mg urinary protein/mg creatinine, between about 15.0 and about 20.0 mg urinary protein/mg creatinine, between about 20.0 and about 25.0 mg urinary protein/mg creatinine, between about 30.0 and about 35.0 mg urinary protein/mg creatinine, between about 40.0 and about 45.0 mg urinary protein/mg creatinine, between about 45.0 and about 50.0 mg urinary protein/mg creatinine, between about 50.0 and about 60.0 mg urinary protein/mg creatinine, between about 60.0 and about 70.0 mg urinary protein/mg creatinine, between about 70.0 and about 80.0 mg urinary protein/mg creatinine, between about 80.0 and about 90.0 mg urinary protein/mg creatinine, between about 90.0 and about 100.0 mg urinary protein/mg creatinine). In some embodiments, methods of the present disclosure reduce the subject’s UPCR by at least 2.5% (e.g., 5%, 10%, 15%, 20%, 25%, 30%, 40%, 50%, 60%, 70%. 80%, 90%, 95%, or 99%) compared to a baseline measurement.

In some embodiments, methods of the present disclosure increase the subject’s estimated glomerular filtration rate (eGFR) and/or glomerular filtration rate (GFR). In some embodiments, the eGFR is measured using serum creatinine, age, ethnicity, and gender variables. In some embodiments, the eGFR is measured using one or more of Cockcroft-Gault formula, Modification of Diet in Renal Disease (MDRD) formula, CKD-EPI formula, Mayo quadratic formula, and Schwartz formula. In some embodiments, the eGFR and/or GFR is increased by at least 2.5% (e.g., 5%, 10%, 15%, 20%, 25%, 30%, 40%, 50%, 60%, 70%. 80%, 90%, 95%, or 99%) compared to a baseline measurement. In some embodiments, the GFR is increased by about 1 mL/min/1.73 m² (e.g., 3, 5, 7, 9, 10, 15, 20, 25, 30, 35, 40, 45, 50, 55, 60, 65, 70, 75, 80, 85, 90, 95, or 100 mL/min/1.73 m²) compared to a baseline measurement.

In some embodiments, the renal disease or condition is evaluated in stages of chronic kidney disease (CKD). In some embodiments, the subject has stage one chronic kidney disease (CKD). In some embodiments, the subject has stage two chronic kidney disease (CKD). In some embodiments, the subject has stage three chronic kidney disease (CKD). In some embodiments, the subject has stage four chronic kidney disease (CKD). In some embodiments, the subject has stage five chronic kidney disease (CKD). In some embodiments, methods of the present disclosure reduce severity, occurrence and/or duration of Stage 1 CKD. In some embodiments, methods of the present disclosure reduce severity, occurrence and/or duration of Stage 2 CKD. In some embodiments, methods of the present disclosure reduce severity, occurrence and/or duration of Stage 3 CKD. In some embodiments, methods of the present disclosure reduce severity, occurrence and/or duration of Stage 3a CKD. In some embodiments, methods of the present disclosure reduce severity, occurrence and/or duration of Stage 3b CKD. In some embodiments, methods of the present disclosure reduce severity, occurrence and/or duration of Stage 4 CKD. In some embodiments, methods of the present disclosure reduce severity, occurrence and/or duration of Stage 5 CKD. In some embodiments, methods of the present disclosure prevent or delay a subject with Stage 1 CKD from progressing to Stage 2 CKD. In some embodiments, methods of the present disclosure prevent or delay a subject with Stage 2 CKD from progressing to Stage 3 CKD. In some embodiments, methods of the present disclosure prevent or delay a subject with Stage 2 CKD from progressing to Stage 3a CKD. In some embodiments, methods of the present disclosure prevent or delay a subject with Stage 3a CKD from progressing to Stage 3b CKD. In some embodiments, methods of the present disclosure prevent or delay a subject with Stage 3 CKD from progressing to Stage 4 CKD. In some embodiments, methods of the present disclosure prevent or delay a subject with Stage 3b CKD from progressing to Stage 4 CKD. In some embodiments, methods of the present disclosure prevent or delay a subject with Stage 4 CKD from progressing to Stage 5 CKD. In some embodiments, methods of the present disclosure delay and/or prevent worsening of CKD stage progression in a subject in need thereof. In some embodiments, methods of the present disclosure improve renal damage CKD classification in a subject by one or more stages. In some embodiments, methods of the present disclosure reduce total kidney volume in a subject. In some embodiments, the total kidney volume is reduced by at least 2.5% (e.g., 5%, 10%, 15%, 20%, 25%, 30%, 40%, 50%, 60%, 70%. 80%, 90%, 95%, or 99%) compared to a baseline measurement. In some embodiments, methods of the present disclosure reduce the subject’s blood urea nitrogen (BUN). In some embodiments, the BUN is reduced by at least 2.5% (e.g., 5%, 10%, 15%, 20%, 25%, 30%, 40%, 50%, 60%, 70%. 80%, 90%, 95%, or 99%) compared to a baseline measurement. In some embodiments, methods of the present disclosure prevent or delay clinical worsening of a renal disease or condition. In some embodiments, methods of the present disclosure reduce risk of hospitalization for one or more complications associated with a renal disease or condition. In some embodiments, the polypeptide is administered by subcutaneous injection. In some embodiments, the heteromultimer protein is administered by subcutaneous injection.

In certain aspects, the disclosure relates to methods of treating a renal disease or condition comprising administering a polypeptide to a subject in need thereof, wherein the polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises a K substitution at the position corresponding to F82 of SEQ ID NO: 2 . In some embodiments, the polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to amino acids 25-131 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to amino acids 20-134 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 53. In some embodiments, the polypeptide is a fusion protein further comprising a first polypeptide domain and one or more heterologous polypeptide domains. In some embodiments, the polypeptide is an ActRIIB-Fc fusion protein. In some embodiments, the fusion protein further comprises a linker domain positioned between the first polypeptide domain and the one or more heterologous domains or Fc domain. In some embodiments, the linker domain is selected from: TGGG, TGGGG, SGGGG, GGGGS, GGG, GGGG, SGGG, and GGGGS. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 5. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 12. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 40. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 42. In some embodiments, the polypeptide binds to one or more ligands selected from the group consisting of activin A, activin B, GDF11, GDF8, GDF3, BMP5, BMP6, and BMP10. In some embodiments, the polypeptide binds to activin A, GDF8, GDF11, and BMP10. In some embodiments, the polypeptide inhibits the activity of one or more ligands in a cell-based assay.

In some embodiments of the present disclosure, the renal disease or condition is Alport syndrome. In some embodiments of the present disclosure, the renal disease or condition is focal segmental glomerulosclerosis (FSGS). In some embodiments, the subject has a decline in kidney function. In some embodiments, the method slows kidney function decline.

In some embodiments, methods of the present disclosure further comprise administering to the subject an additional active agent and/or supportive therapy for treating a renal disease or condition. In some embodiments, the additional active agent and/or supportive therapy for treating a renal disease or condition is selected from the group consisting of: an angiotensin receptor blocker (ARB) (e.g., losartan, irbesartan, olmesartan, candesartan, valsartan, fimasartan, azilsartan, salprisartan, and telmisartan), an angiotensin-converting enzyme (ACE) inhibitor (e.g., benazepril, captopril, enalapril, lisinopril, perindopril, ramipril, trandolapril, and zofenopril), a glucocorticoid (e.g., beclomethasone, betamethasone, budesonide, cortisone, dexamethasone, hydrocortisone, methylprednisolone, prednisolone, methylprednisone, prednisone, and triamcinolone), a calcineurin inhibitor (e.g., cyclosporine, tacrolimus), cyclophosphamide, chlorambucil, a janus kinase inhibitor (e.g., tofacitinib), an mTOR inhibitor (e.g., sirolimus, everolimus), an IMDH inhibitor (e.g., azathioprine, leflunomide, mycophenolate), a biologic (e.g., abatacept, adalimumab, anakinra, basiliximab, certolizumab, daclizumab, etanercept, fresolimumab, golimumab, infliximab, ixekizumab, natalizumab, rituximab, secukinumab, tocilizumab, ustekinumab, vedolizumab), a statin (e.g., benazepril, valsartan, fluvastatin, pravastatin), bardoxolone methyl, Achtar gel, tolvaptan, abatacept in combination with sparsentan, aliskiren, allopurinol, ANG-3070. atorvastatin, bleselumab, bosutinib, CCX140-B, CXA-10, D6-25-hydroxyvitamin D3, dapagliflozin, dexamethasone in combination with MMF, emodin, FG-3019, FK506, FK-506 and MMF, FT-011, galactose, GC1008, GFB-887, isotretinoin, lademirsen, lanreotide, levamisole, lixivaptan, losmapimod, metformin, mizorbine, N-acetylmannosamine, octreotide, paricalcitol, PF-06730512, pioglitazone, propagermanium, propagermanium and irbesartan, rapamune, rapamycin, RE-021 (e.g., sparsentan), RG012, rosiglitazone (e.g., Avandia), saquinivir, SAR339375, somatostatin, spironolactone, tesevatinib (KD019), tetracosactin, tripterygium wilfordii (TW), valproic acid, VAR-200. venglustat (GZ402671), verinurad, voclosporin, VX-147, kidney dialysis, kidney transplant, mesenchymal stem cell therapy, bone marrow stem cells, lipoprotein removal, a Liposorber LA-15 device, plasmapheresis, plasma exchange, and a change in diet (e.g., dietary sodium intake). In some embodiments, the additional active agent and/or supportive therapy for treating a renal disease or condition is an angiotensin receptor blocker (ARB) selected from the group consisting of losartan, irbesartan, olmesartan, candesartan, valsartan, fimasartan, azilsartan, salprisartan, and telmisartan. In some embodiments, the additional active agent and/or supportive therapy for treating a renal disease or condition is an angiotensin-converting enzyme (ACE) inhibitor selected from the group consisting of benazepril, captopril, enalapril, lisinopril, perindopril, ramipril, trandolapril, and zofenopril. In some embodiments, the additional active agent and/or supportive therapy for treating a renal disease or condition is a combination of an ARB and an ACE inhibitor.

In some embodiments of the present disclosure, the subject has proteinuria. In some embodiments of the present disclosure, the subject has albuminuria. In some embodiments, methods of the present disclosure reduce albumin creatinine ratio (ACR) of the subject. In some embodiments, methods of the present disclosure reduce the subject’s ACR by between about 0.1 and about 100.0 mg albumin/g creatinine (e.g., by between about 0.1 and about 2.5 mg albumin/g , between about 2.5 and about 3.5 mg albumin/g creatinine, between about 3.5 and about 5.0 mg albumin/g creatinine, between about 5.0 and about 7.5 mg albumin/g creatinine, between about 7.5 and about 10.0 mg albumin/g creatinine, between about 10.0 and about 15.0 mg albumin/g creatinine, between about 15.0 and about 20.0 mg albumin/g creatinine, between about 20.0 and about 25.0 mg albumin/g creatinine, between about 30.0 and about 35.0 mg albumin/g creatinine, between about 40.0 and about 45.0 mg albumin/g creatinine, between about 45.0 and about 50.0 mg albumin/g creatinine, between about 50.0 and about 60.0 mg albumin/g creatinine, between about 60.0 and about 70.0 mg albumin/g creatinine, between about 70.0 and about 80.0 mg albumin/g creatinine, between about 80.0 and about 90.0 mg albumin/g creatinine, between about 90.0 and about 100.0 mg albumin/g creatinine). In some embodiments, methods of the present disclosure reduce the subject’s ACR by at least 2.5% (e.g., 5%, 10%, 15%, 20%, 25%, 30%, 40%, 50%, 60%, 70%. 80%, 90%, 95%, or 99%) compared to a baseline measurement. In some embodiments, methods of the present disclosure reduce a urinary protein-creatinine ratio (UPCR) of the subject. In some embodiments, methods of the present disclosure reduce the subject’s UPCR between about 0.1 and about 100.0 mg urinary protein/mg creatinine (e.g., by between about 0.1 and about 2.5 mg urinary protein/mg creatinine, between about 2.5 and about 3.5 mg urinary protein/mg creatinine, between about 3.5 and about 5.0 mg urinary protein/mg creatinine, between about 5.0 and about 7.5 mg urinary protein/mg creatinine, between about 7.5 and about 10.0 mg urinary protein/mg creatinine, between about 10.0 and about 15.0 mg urinary protein/mg creatinine, between about 15.0 and about 20.0 mg urinary protein/mg creatinine, between about 20.0 and about 25.0 mg urinary protein/mg creatinine, between about 30.0 and about 35.0 mg urinary protein/mg creatinine, between about 40.0 and about 45.0 mg urinary protein/mg creatinine, between about 45.0 and about 50.0 mg urinary protein/mg creatinine, between about 50.0 and about 60.0 mg urinary protein/mg creatinine, between about 60.0 and about 70.0 mg urinary protein/mg creatinine, between about 70.0 and about 80.0 mg urinary protein/mg creatinine, between about 80.0 and about 90.0 mg urinary protein/mg creatinine, between about 90.0 and about 100.0 mg urinary protein/mg creatinine). In some embodiments, methods of the present disclosure reduce the subject’s UPCR by at least 2.5% (e.g., 5%, 10%, 15%, 20%, 25%, 30%, 40%, 50%, 60%, 70%. 80%, 90%, 95%, or 99%) compared to a baseline measurement. In some embodiments, methods of the present disclosure increase the subject’s estimated glomerular filtration rate (eGFR) and/or glomerular filtration rate (GFR). In some embodiments, the eGFR is measured using serum creatinine, age, ethnicity, and gender variables. In some embodiments, the eGFR is measured using one or more of Cockcroft-Gault formula, Modification of Diet in Renal Disease (MDRD) formula, CKD-EPI formula, Mayo quadratic formula, and Schwartz formula. In some embodiments, the eGFR and/or GFR is increased by at least 2.5% (e.g., 5%, 10%, 15%, 20%, 25%, 30%, 40%, 50%, 60%, 70%. 80%, 90%, 95%, or 99%) compared to a baseline measurement. In some embodiments, the GFR is increased by about 1 mL/min/1.73 m² (e.g., 3, 5, 7, 9, 10, 15, 20, 25, 30, 35, 40, 45, 50, 55, 60, 65, 70, 75, 80, 85, 90, 95, or 100 mL/min/1.73 m²) compared to a baseline measurement.

In some embodiments of the present disclosure, the renal disease or condition is evaluated in stages of chronic kidney disease (CKD). In some embodiments, methods of the present disclosure prevent or delay a subject with Stage 1 CKD from progressing to Stage 2 CKD. In some embodiments, methods of the present disclosure prevent or delay a subject with Stage 2 CKD from progressing to Stage 3 CKD. In some embodiments, methods of the present disclosure prevent or delay a subject with Stage 2 CKD from progressing to Stage 3a CKD. In some embodiments, methods of the present disclosure prevent or delay a subject with Stage 3a CKD from progressing to Stage 3b CKD. In some embodiments, methods of the present disclosure prevent or delay a subject with Stage 3 CKD from progressing to Stage 4 CKD. In some embodiments, methods of the present disclosure prevent or delay a subject with Stage 3b CKD from progressing to Stage 4 CKD. In some embodiments, methods of the present disclosure prevent or delay a subject with Stage 4 CKD from progressing to Stage 5 CKD.

In some embodiments, methods of the present disclosure reduce total kidney volume in a subject. In some embodiments, the total kidney volume is reduced by at least 2.5% (e.g., 5%, 10%, 15%, 20%, 25%, 30%, 40%, 50%, 60%, 70%. 80%, 90%, 95%, or 99%) compared to a baseline measurement.

In some embodiments, methods of the present disclosure reduce the subject’s blood urea nitrogen (BUN). In some embodiments, the BUN is reduced by at least 2.5% (e.g., 5%, 10%, 15%, 20%, 25%, 30%, 40%, 50%, 60%, 70%. 80%, 90%, 95%, or 99%) compared to a baseline measurement.

In some embodiments, methods of the present disclosure prevent or delay clinical worsening of a renal disease or condition. In some embodiments, methods of the present disclosure reduce risk of hospitalization for one or more complications associated with a renal disease or condition. In some embodiments of the present disclosure, the polypeptide is administered by subcutaneous injection.

### BRIEF DESCRIPTION OF THE DRAWINGS

FIG. 1A and FIG. 1B show schematic examples of heteromeric protein complexes comprising a variant ActRIIB polypeptide (indicated as “X”) and either an ALK4 polypeptide (indicated as “Y”) or an ALK7 polypeptide (indicated as “Y”). In the illustrated embodiments, the variant ActRIIB polypeptide is part of a fusion polypeptide that comprises a first member of an interaction pair (“Ci”), and either an ALK4 polypeptide or an ALK7 polypeptide is part of a fusion polypeptide that comprises a second member of an interaction pair (“C₂”). Suitable interaction pairs include, for example, heavy chain and/or light chain immunoglobulin interaction pairs, truncations, and variants thereof such as those described herein [e.g., Spiess et al (2015) Molecular Immunology 67(2A): 95-106]. In each fusion polypeptide, a linker may be positioned between the variant ActRIIB polypeptide, ALK4 polypeptide, or ALK7 polypeptide and the corresponding member of the interaction pair. The first and second members of the interaction pair may be unguided, meaning that the members of the pair may associate with each other or self-associate without substantial preference, and they may have the same or different amino acid sequences. See FIG. 1A. Alternatively, the interaction pair may be a guided (asymmetric) pair, meaning that the members of the pair associate preferentially with each other rather than self-associate. See FIG. 1B.

FIG. 2 shows a multiple sequence alignment of various vertebrate ActRIIB precursor proteins without their intracellular domains (SEQ ID NOs: 358-363), human ActRIIA precursor protein without its intracellular domain (SEQ ID NO: 364), and a consensus ActRII precursor protein (SEQ ID NO: 365). Upper case letters in the consensus sequence indicate positions that are conserved. Lower case letters in the consensus sequence indicate an amino acid residue that is the predominant form, but not universal at that position.

FIG. 3 shows multiple sequence alignment of Fc domains from human IgG isotypes using Clustal 2.1. Hinge regions are indicated by dotted underline. Double underline indicates examples of positions engineered in IgG1 (SEQ ID NO: 13) Fc to promote asymmetric chain pairing and the corresponding positions with respect to other isotypes IgG4 (SEQ ID NO: 17), IgG2 (SEQ ID NO: 14), and IgG3 (SEQ ID NO: 15).

FIG. 4 shows the amino acid sequence of human ActRIIB precursor protein (SEQ ID NO: 2); NCBI Reference Sequence NP_001097.2). The signal peptide is underlined, the extracellular domain is in bold (also referred to as SEQ ID NO: 1), and the potential N-linked glycosylation sites are boxed. SEQ ID NO: 2 is used as the wild-type reference sequence for human ActRIIB in this disclosure, and the numbering for the variants described herein are based on the numbering in SEQ ID NO: 2

FIG. 5 shows the amino acid sequence of a human ActRIIB extracellular domain polypeptide (SEQ ID NO: 1) in which numbering is based on the native human ActRIIB precursor sequence (see SEQ ID NO: 2).

FIG. 6 shows a nucleic acid sequence encoding human ActRIIB precursor protein. SEQ ID NO: 4 consists of nucleotides 434-1972 of NCBI Reference Sequence NM _001106.4.

FIG. 7 shows a nucleic acid sequence (SEQ ID NO: 3) encoding a human ActRIIB(20-134) extracellular domain polypeptide.

FIG. 8A and FIG. 8B show values for ligand binding kinetics of homodimeric Fc-fusion proteins comprising variant or unmodified ActRIIB domains, as determined by surface plasmon resonance at 37° C. Amino acid numbering is based on SEQ ID NO: 2. ND# indicates that the value is not detectable over concentration range tested. Transient* indicates that the value is indeterminate due to transient nature of interaction. Control sample is ActRIIB-G1Fc (SEQ ID NO: 5).

FIG. 9 shows values for ligand binding kinetics of homodimeric Fc-fusion proteins comprising variant or unmodified ActRIIB domains, as determined by surface plasmon resonance at 37° C. Amino acid numbering is based on SEQ ID NO: 2. ND# indicates that the value is not detectable over concentration range tested. Transient binding* indicates that the value is indeterminate due to transient nature of interaction. Control sample is ActRIIB-G1Fc (SEQ ID NO: 5).

FIG. 10 shows values for ligand binding kinetics of homodimeric Fc-fusion proteins comprising variant or unmodified ActRIIB domains, as determined by surface plasmon resonance at 25° C. ND# indicates that the value is not detectable over concentration range tested. Amino acid numbering is based on SEQ ID NO: 2.

FIG. 11 shows therapeutic effect of variant ActRIIB F82K mFc fusion (“ActRIIB (F82K)-mFc”) in a UUO model. Sixteen mice underwent left unilateral ureteral ligation twice at the level of the lower pole of kidney, and after 3 days, they were randomized into two groups: i) “UUO/PBS” (eight mice were injected subcutaneously with vehicle control, phosphate buffered saline (PBS), at days 3, 7, 10, and 14 after surgery) and ii) “UUO/F82K” (eight mice were injected subcutaneously with variant ActRIIB F82K mFc fusion at a dose of 10 mg/kg at days 3, 7, 10, and 14 after surgery). The “CTRL” is the contralateral kidney that did not undergo the unilateral ureteral obstruction procedure. FIGS. 11A-11E show gene expression analysis of fibrotic gene markers (Fibronectin, PAI-I, Col-I, Col-III, a-SMA, respectively), FIGS. 11F-11G show gene expression analysis of inflammatory gene markers (MCP-1, TNFa, respectively), FIG. 11H shows gene expression analysis of a kidney injury marker (NGAL), and FIGS. 11I-11J show gene expression analysis of TGFβ ligands(Tgfb 1 and Activin A, respectively). Relative to “UUO/PBS” treated mice, “UUO/F82K” treated mice demonstrated significantly lower expression of fibrotic and inflammatory genes, reduced upregulation of TGFβ 1 and activin A, and reduced kidney injury gene expression. Statistical significance (p value) is depicted as * p<0.05, **p<0.01, ***p<0.001, and ****p<0.0001 for comparison between “CTRL” and sample “UUO/PBS”. Statistical significance (p value) is depicted as # p<0.05, ##p<0.01, ###p<0.001, and ####p<0.0001 for comparison between “UUO/PBS” and sample “UUO/F82K”. “B.D.L.” means that the measurement value was below the limit of detection, and no statistics were calculated for a value in comparison to a “B.D.L.” value.

FIG. 12 shows therapeutic effect of variant ActRIIB F82K mFc fusion (“ActRIIB (F82K)-mFc”) in a Col4a3 (-/-) Alport syndrome model. Fourteen Col4a3-/- mice were randomized into two groups: i) “Col4a3-Veh” (eight mice injected subcutaneously with vehicle control, phosphate buffered saline (PBS), twice a week) and ii) “Col4a3-F82K” (six mice injected subcutaneously with variant ActRIIB F82K mFc fusion (“ActRIIB (F82K)-mFc”) at a dose of 10 mg/kg twice a week. “WT” mice, which are non-treated Col4a3+/+ mice, were also analyzed along with the above at day 49 (7 weeks). Relative to Col4a3-Veh mice, treatment of mice with ActRIIB (F82K)-mFc (“Col4a3-F82K”) significantly reduced albuminuria (depicted as an albumin-creatinine ratio (ACR)) by about 38.9% (p<0.05) at 7 weeks, and by about 45.1% (p<0.001) at 9 weeks (FIG. 12A). To evaluate the therapeutic benefits of ActRIIB (F82K)-mFc in the presence of angiotensin-converting enzyme inhibitor (ACEi) in Alport model, ActRIIB (F82K)-mFc was further assessed in Col4a3-/- mice treated with Ramipril. Thirty-one Col4a3-/- mice were fed with Ramipril (10 mg/kg/day) in drinking water throughout the study, and randomized into two groups: i) sixteen mice were injected subcutaneously with vehicle control, phosphate buffered saline (PBS), twice a week (Col4a3-ACEi/Veh″), and ii) fifteen mice were injected subcutaneously with ActRIIB (F82K)-mFc at a dose of 10 mg/kg twice a week (“Col4a3-ACEi/F82K”). “WT” mice, which are non-treated Col4a3+/+ mice, were also analyzed along with the above at day 49 (7 weeks) to measure albumin to creatinine ratio (ACR, FIG. 12B). Albuminuria was significantly increased from 4 weeks to 17 weeks in Col4a3-/- mice treated with Ramipril, but treatment of these mice with ActRIIB (F82K)-mFc significantly reduced albuminuria by 76.7%% (p<0.05) at 12 weeks, 59% (p<0.05) at 15 weeks, and 86% (p<0.001) at 17 weeks respectively in Col4a3-/-mice, which was associated with decreased BUN in Col4a3-/- mice (FIG. 12C). ActRIIB (F82K)-mFc significantly increased life span in Col4a3-/- mice treated with Ramipril (p<0.05), with a median survival time of 141 days in the mice treated with ActRIIB (F82K)-mFc (“Col4a3-ACEi/F82K”) and 119 days in the cohort treated with PBS (“Col4a3-ACEi/Veh”) (FIG. 12D). Statistical significance (p value) is depicted as * p<0.05, **p<0.01, ***p<0.001, and ****p<0.0001.

FIG. 13 shows therapeutic effect of variants ActRIIB-(K55A)-mFc, ActRIIB (K55E)-mFc, and ActRIIB (F82I)-mFc in a UUO model. Thirty-two mice underwent left unilateral ureteral ligation twice at the level of the lower pole of kidney, and after 3 days, they were randomized into four groups: i) eight mice were injected subcutaneously with vehicle control, phosphate buffered saline (PBS), at day 3, day 7, day 10, and day 14 after surgery (“UUO/PBS”), ii) eight mice were injected subcutaneously with ActRIIB (K55A)-mFc at a dose of 10 mg/kg at day 3, day 7, day 10, and day 14 after surgery, iii) eight mice were injected subcutaneously with ActRIIB (K55E)-mFc at a dose of 10 mg/kg at day 3, day 7, day 10, and day 14 after surgery, and iv) eight mice were injected subcutaneously with ActRIIB (F82I)-mFc at a dose of 10 mg/kg at day 3, day 7, day 10, and day 14 after surgery. The “CTRL” is the contralateral kidney that did not undergo the unilateral ureteral obstruction procedure. FIGS. 13A-13E show gene expression analysis of fibrotic gene markers (Fibronectin, PAI-I, Col-I, Col-III, a-SMA, respectively), FIGS. 13F-13G show gene expression analysis of inflammatory gene markers (MCP-1, TNFa, respectively), FIG. 13H shows gene expression analysis of a kidney injury marker (NGAL), and FIGS. 13I-13J show gene expression analysis of TGFβ ligands(Tgfb1 and Activin A, respectively). Relative to “UUO/PBS” treated mice, treatment of mice with ActRIIB (K55A)-mFc significantly suppressed the expression of fibrotic and inflammatory genes, inhibited the upregulation of TGFβ 1 and Activin A, and reduced kidney injury. Relative to “UUO/PBS” treated mice, treatment of mice with ActRIIB (K55E)-mFc significantly suppressed the expression of PAI-1 and a-SMA, but not Col1a1, Col3a1, and Fibronectin. In addition, treatment of mice with ActRIIB (K55E)-mFc did not significantly suppress inflammatory gene expression, nor reduce kidney injury. Relative to “UUO/PBS” treated mice, treatment of mice with ActRIIB (F82I)-mFc significantly suppressed the expression of PAI-1 and a-SMA, but not Col1a1, Col3a1, and Fibronectin. In addition, treatment of mice with ActRIIB (F82I)-mFc significantly inhibited the upregulation of TGFβ 1 and Activin A, and reduced kidney injury. However, ActRIIB (F82I)-mFc did not significantly suppress inflammatory gene expression. Statistical significance (p value) is depicted as * p<0.05, **p<0.01, ***p<0.001, and ****p<0.0001 for comparison between “CTRL” and sample “UUO/PBS”. Statistical significance (p value) is depicted as # p<0.05, ##p<0.01, ###p<0.001, and ####p<0.0001 for comparison between “UUO/PBS” and samples “UUO/K55A”, “UUO/K55E”, or “UUO/F82I”. “B.D.L.” means that the measurement value was below the limit of detection, and no statistics were calculated for a value in comparison to a “B.D.L.” value.

FIG. 14 shows activity of variant ActRIIB-Fc proteins from an A204 cell-based assay to screen variant ActRIIB-Fc proteins for inhibitory effects on cell signaling by activin A, activin B, GDF8, GDF11, BMP6, BMP9, and BMP10. Potencies of homodimeric Fc fusion proteins incorporating amino acid substitutions in the human ActRIIB extracellular domain were compared with that of an Fc fusion protein comprising unmodified human ActRIIB extracellular domain (SEQ ID NO: 519). ND means not detectable, a slash (-) means not tested and/or not calculated, and * means the value was extrapolated, wherein the IC50 for the curve that does not plateau is estimated by extending the curve out according to its slope to reach the positive and negative controls within one additional concentration at the highest or lowest concentration.

FIG. 15A and FIG. 15B show values for ligand binding kinetics of homodimeric Fc-fusion proteins comprising variant or unmodified ActRIIB domains, as determined by surface plasmon resonance at 37° C. Amino acid numbering is based on SEQ ID NO: 2. N.B. means no binding (less than 5 Resonance Units (RU) in signal), M.B. means minimal binding (less than 10 RU in signal), and transient binding indicates that the value is indeterminate due to transient nature of interaction. Control sample is ActRIIB-G1Fc (SEQ ID NO: 519).

FIG. 16A and FIG. 16B show values for ligand binding kinetics of homodimeric Fc-fusion proteins comprising variant or unmodified ActRIIB domains, as determined by surface plasmon resonance at 37° C. Amino acid numbering is based on SEQ ID NO: 2. N.B. means no binding (less than 5 Resonance Units (RU) in signal), M.B. means minimal binding (less than 10 RU in signal), and transient binding indicates that the value is indeterminate due to transient nature of interaction. Control sample is ActRIIB-G1Fc (SEQ ID NO: 519).

### DETAILED DESCRIPTION

### 1. Overview

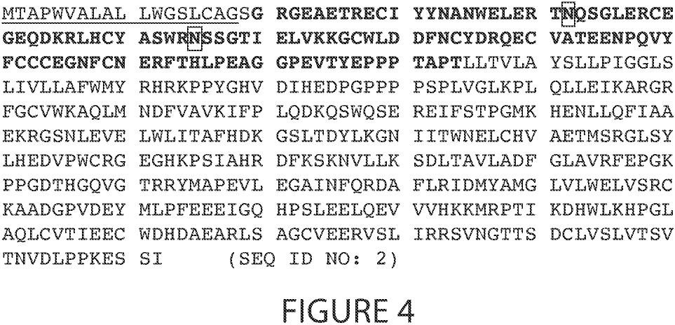

In certain aspects, the present disclosure relates to ActRIIB polypeptides. As used herein, the term “ActRIIB” refers to a family of activin receptor type IIB (ActRIIB) proteins and ActRIIB-related proteins, derived from any species. Members of the ActRIIB family are generally all transmembrane proteins, composed of a ligand-binding extracellular domain with cysteine-rich region, a transmembrane domain, and a cytoplasmic domain with predicted serine/threonine kinase specificity. The amino acid sequence of human ActRIIB precursor protein is shown in FIG. 4 (SEQ ID NO: 2). Examples of variant ActRIIB polypeptides are provided throughout the present disclosure as well as in International Pat. Application Publication Nos. WO 2006/012627, WO 2008/097541, WO 2010/151426, WO 2011/020045, WO 2018/009624, and WO 2018/067874 which are incorporated herein by reference in their entirety.

The term “ActRIIB polypeptide” is used to refer to polypeptides comprising any naturally occurring polypeptide of an ActRIIB family member as well as any variants thereof (including mutants, fragments, fusions, and peptidomimetic forms) that retain a useful activity. For example, ActRIIB polypeptides include polypeptides derived from the sequence of any known ActRIIB having a sequence at least about 80% identical to the sequence of an ActRIIB polypeptide, and preferably at least 85%, 90%, 91%, 92%, 93%, 94%, 95%, 96%, 97%, 98%, 99% or greater identity.

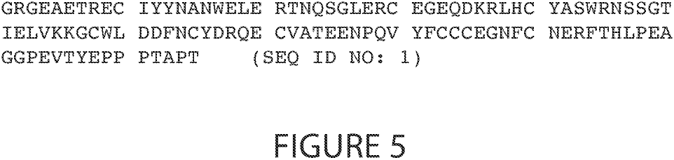

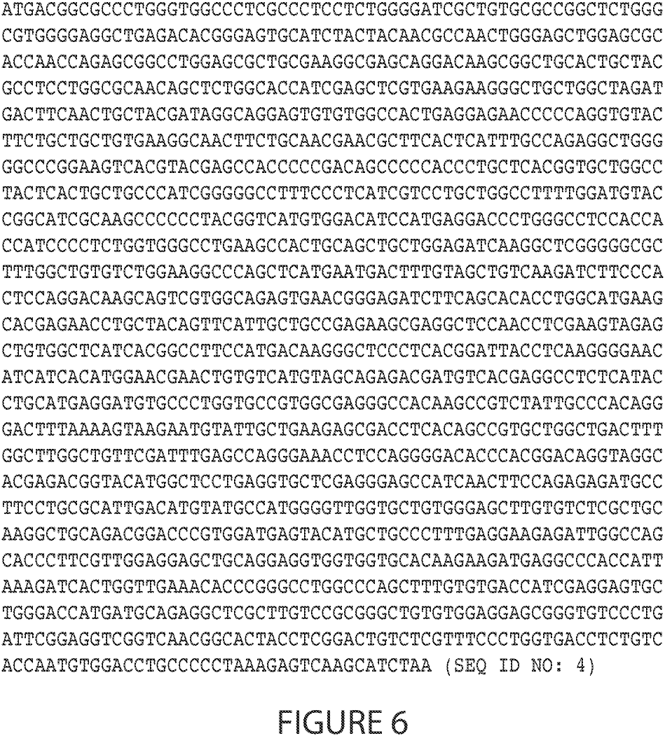

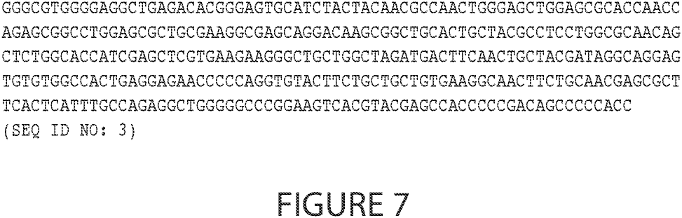

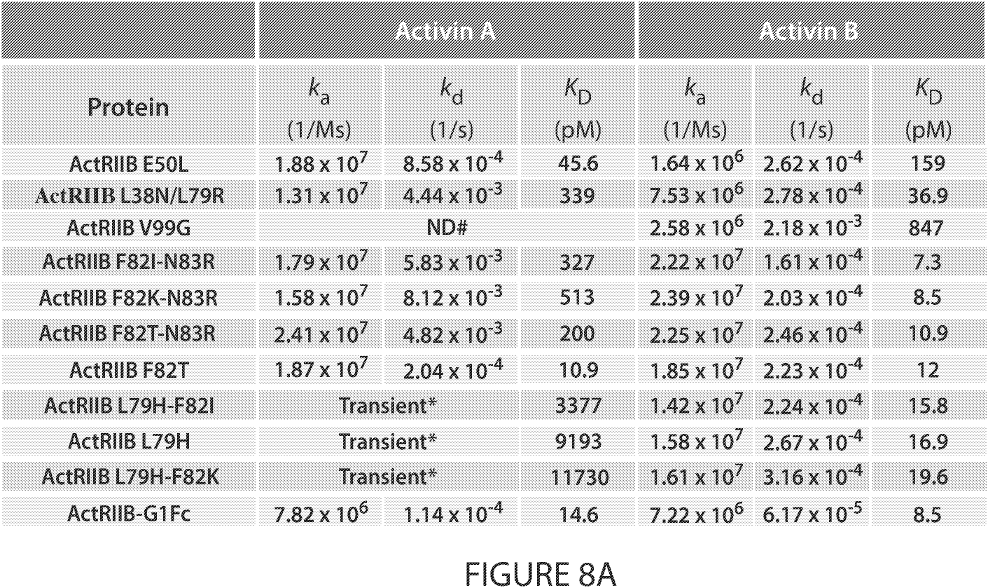

In a specific embodiment, the disclosure relates to soluble ActRIIB polypeptides. As described herein, the term “soluble ActRIIB polypeptide” generally refers to polypeptides comprising an extracellular domain of an ActRIIB protein. The term “soluble ActRIIB polypeptide,” as used herein, includes any naturally occurring extracellular domain of an ActRIIB protein as well as any variants thereof (including mutants, fragments and peptidomimetic forms) that retain a useful activity. For example, the extracellular domain of an ActRIIB protein binds to a ligand and is generally soluble. Examples of soluble ActRIIB polypeptides include an ActRIIB extracellular domain (SEQ ID NO: 1) shown in FIG. 5 as well as SEQ ID NO: 53. This truncated ActRIIB extracellular domain (SEQ ID NO: 53) is denoted ActRIIB(25-131) based on numbering in SEQ ID NO: 2. Other examples of soluble ActRIIB polypeptides comprise a signal sequence in addition to the extracellular domain of an ActRIIB protein (see Example 1). The signal sequence can be a native signal sequence of an ActRIIB, or a signal sequence from another protein, such as a tissue plasminogen activator (TPA) signal sequence or a honey bee melatin signal sequence.

TGF-β signals are mediated by heteromeric complexes of type I and type II serine/ threonine kinase receptors, which phosphorylate and activate downstream Smad proteins upon ligand stimulation (Massague, 2000, Nat. Rev. Mol. Cell Biol. 1:169-178). These type I and type II receptors are all transmembrane proteins, composed of a ligand-binding extracellular domain with cysteine-rich region, a transmembrane domain, and a cytoplasmic domain with predicted serine/threonine specificity. Type I receptors are essential for signaling, and type II receptors are required for binding ligands. Type I and type II activin receptors form a stable complex after ligand binding, resulting in phosphorylation of type I receptors by type II receptors.

Two related type II receptors, ActRIIA and ActRIIB, have been identified as the type II receptors for activins (Mathews and Vale, 1991, Cell 65:973-982; Attisano et al., 1992, Cell 68: 97-108). Besides activins, ActRIIA and ActRIIB can biochemically interact with several other TGF-β family proteins, including BMP7, Nodal, GDF8, and GDF11 (Yamashita et al., 1995, J. Cell Biol. 130:217-226; Lee and McPherron, 2001, Proc. Natl. Acad. Sci. 98:9306-9311; Yeo and Whitman, 2001, Mol. Cell 7: 949-957; Oh et al., 2002, Genes Dev. 16:2749-54). Applicants have found that soluble ActRIIA-Fc fusion proteins and ActRIIB-Fc fusion proteins have substantially different effects in vivo, with ActRIIA-Fc having primary effects on bone and ActRIIB-Fc having primary effects on skeletal muscle.

In certain embodiments, the present disclosure relates to antagonizing a ligand of ActRIIB receptors (also referred to as an ActRIIB ligand) with a subject ActRIIB polypeptide (e.g., a variant ActRIIB polypeptide). In some embodiments, the variant ActRIIB polypeptide is a member of a homomultimer (e.g., homodimer). In some embodiments, the variant ActRIIB polypeptide is a member of a heteromultimer (e.g., a heterodimer). In some embodiments, the variant ActRIIB polypeptide heteromultimerizes with any of the other soluble receptors disclosed herein. In some embodiments, the variant ActRIIB polypeptide is fused to any of the proteins disclosed herein (any of the soluble receptors disclosed herein). Thus, compositions and methods of the present disclosure are useful for treating disorders associated with abnormal activity of one or more ligands of ActRIIB receptors. Exemplary ligands of ActRIIB receptors include some TGF-β family members, such as activin A, activin B, GDF3, GDF8, GDF11, BMP6, BMP9 and BMP10. In some embodiments, any of the heteromultimers disclosed herein have a different binding profile as compared to any of the ActRIIB homomultimers (e.g., homodimers) disclosed herein.

Activins are dimeric polypeptide growth factors and belong to the TGF-beta superfamily. There are three activins (A, B, and AB) that are homo/heterodimers of two closely related β subunits (β_{A}β_{A}, β_{B}β_{B}, and β_{A}β_{B}). In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos (DePaolo et al., 1991, Proc SocEp Biol Med. 198:500-512; Dyson et al., 1997, Curr Biol. 7:81-84; Woodruff, 1998, Biochem Pharmacol. 55:953-963). Moreover, erythroid differentiation factor (EDF) isolated from the stimulated human monocytic leukemic cells was found to be identical to activin A (Murata et al., 1988, PNAS, 85:2434). It was suggested that activin A acts as a natural regulator of erythropoiesis in the bone marrow. In several tissues, activin signaling is antagonized by its related heterodimer, inhibin. For example, during the release of follicle-stimulating hormone (FSH) from the pituitary, activin promotes FSH secretion and synthesis, while inhibin prevents FSH secretion and synthesis. Other proteins that may regulate activin bioactivity and/or bind to activin include follistatin (FS), follistatin-related protein (FSRP), α₂-macroglobulin, Cerberus, and endoglin.

Growth and differentiation factor-8 (GDF8) is also known as myostatin. GDF8 is a negative regulator of skeletal muscle mass. GDF8 is highly expressed in the developing and adult skeletal muscle. The GDF8 null mutation in transgenic mice is characterized by a marked hypertrophy and hyperplasia of the skeletal muscle (McPherron et al., Nature, 1997, 387:83-90). Similar increases in skeletal muscle mass are evident in naturally occurring mutations of GDF8 in cattle (Ashmore et al., 1974, Growth, 38:501-507; Swatland and Kieffer, J. Anim. Sci., 1994, 38:752-757; McPherron and Lee, Proc. Natl. Acad. Sci. USA, 1997, 94:12457-12461; and Kambadur et al., Genome Res., 1997, 7:910-915) and, strikingly, in humans (Schuelke et al., N Engl J Med 2004;350:2682-8). Studies have also shown that muscle wasting associated with HIV-infection in humans is accompanied by increases in GDF8 protein expression (Gonzalez-Cadavid et al., PNAS, 1998, 95:14938-43). In addition, GDF8 can modulate the production of muscle-specific enzymes (e.g., creatine kinase) and modulate myoblast cell proliferation (WO 00/43781). The GDF8 propeptide can noncovalently bind to the mature GDF8 domain dimer, inactivating its biological activity (Miyazono et al. (1988) J. Biol. Chem., 263: 6407-6415; Wakefield et al. (1988) J. Biol. Chem., 263; 7646-7654; and Brown et al. (1990) Growth Factors, 3: 35-43). Other proteins which bind to GDF8 or structurally related proteins and inhibit their biological activity include follistatin, and potentially, follistatin-related proteins (Gamer et al. (1999) Dev. Biol., 208: 222-232).

Growth and differentiation factor-11 (GDF11), also known as BMP11, is a secreted protein (McPherron et al., 1999, Nat. Genet. 22: 260-264). GDF11 is expressed in the tail bud, limb bud, maxillary and mandibular arches, and dorsal root ganglia during mouse development (Nakashima et al., 1999, Mech. Dev. 80: 185-189). GDF11 plays a unique role in patterning both mesodermal and neural tissues (Gamer et al., 1999, Dev Biol., 208:222-32). GDF11 was shown to be a negative regulator of chondrogenesis and myogenesis in developing chick limb (Gamer et al., 2001, Dev Biol. 229:407-20). The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium (Wu et al., 2003, Neuron. 37:197-207). Hence, GDF11 may have in vitro and in vivo applications in the treatment of diseases such as muscle diseases and neurodegenerative diseases (e.g., amyotrophic lateral sclerosis and spinal muscular atrophy).

In certain aspects, the present disclosure relates to the use of certain ActRIIB polypeptides (e.g., soluble ActRIIB polypeptides) to antagonize the signaling of ActRIIB ligands generally, in any process associated with ActRIIB activity. Optionally, ActRIIB polypeptides of the disclosure may antagonize one or more ligands of ActRIIB receptors, such as activin A, activin B, GDF8, GDF11, or BMP10, and may therefore be useful in the treatment of additional disorders. In particular, the disclosure provides variant ActRIIB polypeptides with reduced binding affinity to BMP9 while retaining binding affinity to one or more of activin A, activin B , GDF8, GDF11, and BMP10. Accordingly, these variant ActRIIB polypeptides may be more useful than an unmodified ActRIIB polypeptide in certain applications where such selective antagonism is advantageous. Examples include therapeutic applications where it is desirable to retain antagonisms of one or more of activin A, activin B, GDF8, GDF11, and BMP10, while reducing antagonism of BMP9. In some embodiments, any of the ActRIIB polypeptides disclosed herein may be combined with any of the other polypeptides disclosed herein. In some embodiments, the ActRIIB polypeptide is a member of a heteromultimer (e.g., a heterodimer) with any of the proteins disclosed herein.

Therefore, the present disclosure contemplates using ActRIIB polypeptides, and variants thereof, in treating or preventing diseases or conditions that are associated with abnormal activity of an ActRIIB or an ActRIIB ligand. ActRIIB ligands are involved in the regulation of many critical biological processes. Due to their key functions in these processes, they may be desirable targets for therapeutic intervention. For example, ActRIIB polypeptides (e.g., variant ActRIIB polypeptides) may be used to treat human or animal disorders or conditions. Example of such disorders or conditions include, but are not limited to, pulmonary disorders (e.g., pulmonary hypertension, interstitial lung disease, idiopathic pulmonary fibrosis), renal diseases or conditions (e.g., Alport syndrome, focal segmental glomerulosclerosis (FSGS), polycystic kidney disease, chronic kidney disease) , peripheral neuropathy, Charcot-Marie-Tooth disease, anemia (e.g., anemia associated with myelodysplastic syndrome, thalassemia or myelofibrosis), metabolic disorders such as type 2 diabetes, impaired glucose tolerance, metabolic syndrome (e.g., syndrome X), and insulin resistance induced by trauma (e.g., burns or nitrogen imbalance); adipose tissue disorders (e.g., obesity); muscle and neuromuscular disorders such as muscular dystrophy (including Duchenne muscular dystrophy); amyotrophic lateral sclerosis (ALS); spinal muscular atrophy (SMA); muscle atrophy; organ atrophy; frailty; carpal tunnel syndrome; congestive obstructive pulmonary disease; and sarcopenia, cachexia and other muscle wasting syndromes. Other examples include osteoporosis, especially in the elderly and/or postmenopausal women; glucocorticoid-induced osteoporosis; osteopenia; osteoarthritis; and osteoporosis-related fractures. Yet further examples include low bone mass due to chronic glucocorticoid therapy, premature gonadal failure, androgen suppression, vitamin D deficiency, secondary hyperparathyroidism, nutritional deficiencies, and anorexia nervosa. These disorders and conditions are discussed below under “Exemplary Therapeutic Uses.”

The terms used in this specification generally have their ordinary meanings in the art, within the context of this disclosure and in the specific context where each term is used. Certain terms are discussed below or elsewhere in the specification, to provide additional guidance to the practitioner in describing the compositions and methods of the disclosure and how to make and use them. The scope or meaning of any use of a term will be apparent from the specific context in which the term is used.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values.

Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The methods of the disclosure may include steps of comparing sequences to each other, including an unmodified (wild-type) sequence to one or more mutants (sequence variants). Such comparisons typically comprise alignments of polymer sequences, e.g., using sequence alignment programs and/or algorithms that are well known in the art (for example, BLAST, FASTA and MEGALIGN, to name a few). The skilled artisan can readily appreciate that, in such alignments, where a mutation contains a residue insertion or deletion, the sequence alignment will introduce a “gap” (typically represented by a dash, or “A”) in the polymer sequence not containing the inserted or deleted residue.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

The term “sequence similarity,” in all its grammatical forms, refers to the degree of identity or correspondence between nucleic acid or amino acid sequences that may or may not share a common evolutionary origin.

However, in common usage and in the instant application, the term “homologous,” when modified with an adverb such as “highly,” may refer to sequence similarity and may or may not relate to a common evolutionary origin.

“Agonize”, in all its grammatical forms, refers to the process of activating a protein and/or gene (e.g., by activating or amplifying that protein’s gene expression or by inducing an inactive protein to enter an active state) or increasing a protein’s and/or gene’s activity.

“Antagonize”, in all its grammatical forms, refers to the process of inhibiting a protein and/or gene (e.g., by inhibiting or decreasing that protein’s gene expression or by inducing an active protein to enter an inactive state) or decreasing a protein’s and/or gene’s activity.

The terms “about” and “approximately” as used in connection with a numerical value throughout the specification and the claims denotes an interval of accuracy, familiar and acceptable to a person skilled in the art. In general, such interval of accuracy is ± 10%, Alternatively, and particularly in biological systems, the terms “about” and “approximately” may mean values that are within an order of magnitude, preferably ≤ 5 -fold and more preferably ≤ 2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### 2. ActRIIB Polypeptides

In certain specific embodiments, the present disclosure contemplates making mutations in the extracellular domain (also referred to as ligand-binding domain) of an ActRIIB polypeptide such that the variant (or mutant) ActRIIB polypeptide has altered ligand-binding activities (e.g., binding affinity or binding selectivity). In certain cases, such variant ActRIIB polypeptides have altered (elevated or reduced) binding affinity for a specific ligand. In other cases, the variant ActRIIB polypeptides have altered binding selectivity for their ligands. For example, the disclosure provides a number of variant ActRIIB polypeptides that have reduced binding affinity to BMP9, compared to a non-modified ActRIIB polypeptide, but retain binding affinity for one or more of activin A, activin B, GDF8, GDF11, and BMP10. Optionally, the variant ActRIIB polypeptides have similar or the same biological activities of their corresponding wild-type ActRIIB polypeptides. For example, a variant ActRIIB polypeptide of the disclosure may bind to and inhibit function of an ActRIIB ligand (e.g., activin A, activin B, GDF8, GDF11 or BMP10). In some embodiments, a variant ActRIIB polypeptide of the disclosure treats human or animal disorders or conditions such as pulmonary disorders (e.g., pulmonary hypertension, interstitial lung disease, idiopathic pulmonary fibrosis), renal diseases or conditions (e.g., Alport syndrome, focal segmental glomerulosclerosis (FSGS), polycystic kidney disease, chronic kidney disease), peripheral neuropathy, Charcot-Marie-Tooth disease, and anemia (e.g., anemia associated with myelodysplastic syndrome, thalassemia or myelofibrosis). Examples of ActRIIB polypeptides include human ActRIIB precursor polypeptide (SEQ ID NO: 2), and soluble human ActRIIB polypeptides (e.g., SEQ ID NOs: 1, 5, 6, 12, 276, 278, 279, 332, 333, 335, 336, 338, 339, 341, 342, 344, 345, 347, 348, 350, 351, 353, 354, 356, 357, 366, 368, 369, 371, 372, 374, 375, 377, 378, 380, 381, 383, 384, 386, 387, 389, 390, 392, 393, 395, 396, 398, 399, 401, 402, 404, 405, 407, 408, 410, 411, 413, 414, 416, 417, 419, 420, 422, 423, 425, 426, 428, 429, 431, 432, 434, 435, 437, 438, 440, 441, 443, 444, 446, 447, 449, 450, 452, 453, 455, 456, 458, 459, 461, 462, 464, 465, 467, 468, 470, 471, 473, 474, 476, 477, 479, 480, 482, 483, 485, 486, 488, 489, 491, 492, 494, 495, 497, 498, 500, 501, 503, 504, 506, 507, 509, 510, 512, 513, 515, 516, 518, 519, 520, 522, and 524). In some embodiments, the variant ActRIIB polypeptide is a member of a homomultimer (e.g., homodimer). In some embodiments, the variant ActRIIB polypeptide is a member of a heteromultimer (e.g., a heterodimer). In some embodiments, any of the variant ActRIIB polypeptides may be combined (e.g., heteromultimerized with and/or fused to) with any of proteins disclosed herein.

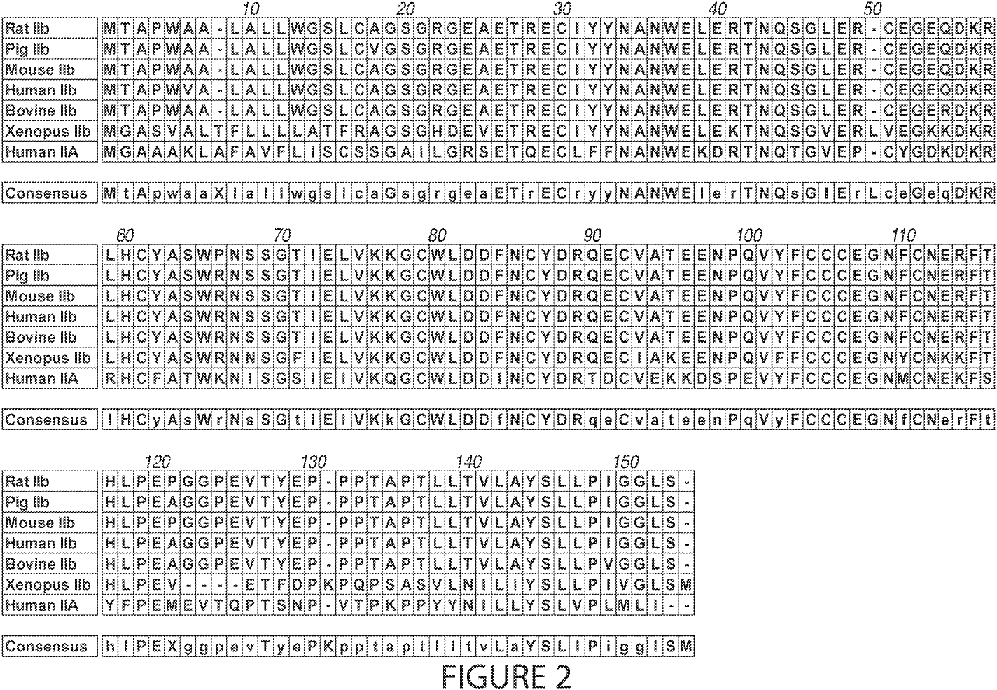

ActRIIB is well-conserved across nearly all vertebrates, with large stretches of the extracellular domain conserved completely. See FIG. 2. Many of the ligands that bind to ActRIIB are also highly conserved. Accordingly, comparisons of ActRIIB sequences from various vertebrate organisms provide insights into residues that may be altered. Therefore, an active, human ActRIIB variant may include one or more amino acids at corresponding positions from the sequence of another vertebrate ActRIIB, or may include a residue that is similar to that in the human or other vertebrate sequence.

The disclosure identifies functionally active portions and variants of ActRIIB. Applicant has previously ascertained that an Fc fusion protein having the sequence disclosed by Hilden et al. (Blood. 1994 Apr 15;83(8):2163-70), which has an alanine at the position corresponding to amino acid 64 of SEQ ID NO: 2 (A64), has a relatively low affinity for activin and GDF11. By contrast, the same Fc fusion protein with an arginine at position 64 (R64) has an affinity for activin and GDF-11 in the low nanomolar to high picomolar range. Therefore, a sequence with an R64 (SEQ ID NO: 2) is used as the wild-type reference sequence for human ActRIIB in this disclosure, and the numbering for the variants described herein are based on the numbering in SEQ ID NO: 2. Additionally, one of skill in the art can make any of the ActRIIB variants described herein in the A64 background.

A processed extracellular ActRIIB polypeptide sequence is shown in SEQ ID NO: 1 (see, e.g., FIG. 5). In some embodiments, a processed ActRIIB polypeptide may be produced with an “SGR...” sequence at the N-terminus. In some embodiments, a processed ActRIIB polypeptide may be produced with a “GRG...” sequence at the N-terminus. For example, it is expected that some constructs, if expressed with a TPA leader, will lack the N-terminal serine. Accordingly, mature ActRIIB sequences described herein may begin with either an N-terminal serine or an N-terminal glycine (lacking the N-terminal serine).

Attisano et al. (Cell. 1992 Jan 10;68(1):97-108) showed that a deletion of the proline knot at the C-terminus of the extracellular domain of ActRIIB reduced the affinity of the receptor for activin. Data disclosed in WO2008097541 show that an ActRIIB-Fc fusion protein containing amino acids 20-119 of SEQ ID NO: 2, “ActRIIB(20-119)-Fc” has reduced binding to GDF11 and activin relative to an ActRIIB(20-134)-Fc, which includes the proline knot region and the complete juxtamembrane domain. However, an ActRIIB(20-129)-Fc protein retains similar but somewhat reduced activity relative to the wild type, even though the proline knot region is disrupted. Thus, ActRIIB extracellular domains that stop at amino acid 134, 133, 132, 131, 130 and 129 are all expected to be active, but constructs stopping at 134 or 133 may be most active. Similarly, mutations at any of residues 129-134 are not expected to alter ligand binding affinity by large margins. In support of this, mutations of P129 and P130 do not substantially decrease ligand binding. Therefore, an ActRIIB-Fc fusion protein may end as early as amino acid 109 (the final cysteine), however, forms ending at or between 109 and 119 are expected to have reduced ligand binding. Amino acid 119 is poorly conserved and so is readily altered or truncated. Forms ending at 128 or later retain ligand binding activity. Forms ending at or between 119 and 127 will have an intermediate binding ability. Any of these forms may be desirable to use, depending on the clinical or experimental setting.

At the N-terminus of ActRIIB, it is expected that a protein beginning at amino acid 29 or before will retain ligand binding activity. Amino acid 29 represents the initial cysteine. An alanine-to-asparagine mutation at position 24 introduces an N-linked glycosylation sequence without substantially affecting ligand binding. This confirms that mutations in the region between the signal cleavage peptide and the cysteine cross-linked region, corresponding to amino acids 20-29, are well tolerated. In particular, constructs beginning at position 20, 21, 22, 23 and 24 will retain activity, and constructs beginning at positions 25, 26, 27, 28 and 29 are also expected to retain activity. Data shown in WO2008097541 demonstrate that, surprisingly, a construct beginning at 22, 23, 24 or 25 will have the most activity.

Taken together, an active portion of ActRIIB comprises amino acids 29-109 of SEQ ID NO: 2, and constructs may, for example, begin at a residue corresponding to amino acids 20-29 and end at a position corresponding to amino acids 109-134. Other examples include constructs that begin at a position from 20-29 or 21-29 and end at a position from 119-134, 119-133 or 129-134, 129-133. Other examples include constructs that begin at a position from 20-24 (or 21-24, or 22-25) and end at a position from 109-134 (or 109-133), 119-134 (or 119-133) or 129-134 (or 129-133). Variants within these ranges are also contemplated, particularly those having at least 80%, 85%, 90%, 91%, 92%, 93%, 94%, 95%, 96%, 97%, 98%, or 99% identity to the corresponding portion of SEQ ID NO: 1.

In certain embodiments, a variant ActRIIB polypeptide has an amino acid sequence that is at least 75% identical to an amino acid sequence selected from SEQ ID NOs: 1, 2, and 53. In certain cases, the variant ActRIIB polypeptide has an amino acid sequence at least 80%, 85%, 90%, 91%, 92%, 93%, 94%, 95%, 96%, 97%, 98%, 99%, or 100% identical to an amino acid sequence selected from SEQ ID NOs: 1, 2, and 53. In certain cases, the variant ActRIIB polypeptide has an amino acid sequence at least 80%, 85%, 90%, 91%, 92%, 93%, 94%, 95%, 96%, 97%, 98%, 99%, or 100% identical to SEQ ID NO: 1. In certain cases, the variant ActRIIB polypeptide has an amino acid sequence at least 80%, 85%, 90%, 91%, 92%, 93%, 94%, 95%, 96%, 97%, 98%, 99%, or 100% identical to SEQ ID NO: 2. In certain cases, the variant ActRIIB polypeptide has an amino acid sequence at least 80%, 85%, 90%, 91%, 92%, 93%, 94%, 95%, 96%, 97%, 98%, 99%, or 100% identical to SEQ ID NO: 53.

In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of any one of SEQ ID NOs: 1, 2, 5, 6, 12, 31, 33, 34, 36, 37, 39, 40, 42, 43, 45, 46, 48, 49, 50, 51, 52, 53, 276, 278, 279, 332, 333, 335, 336, 338, 339, 341, 342, 344, 345, 347, 348, 350, 351, 353, 354, 356, 357, 366, 368, 369, 371, 372, 374, 375, 377, 378, 380, 381, 383, 384, 386, 387, 389, 390, 392, 393, 395, 396, 398, 399, 401, 402, 404, 405, 407, 408, 410, 411, 413, 414, 416, 417, 419, 420, 422, 423, 425, 426, 428, 429, 431, 432, 434, 435, 437, 438, 440, 441, 443, 444, 446, 447, 449, 450, 452, 453, 455, 456, 458, 459, 461, 462, 464, 465, 467, 468, 470, 471, 473, 474, 476, 477, 479, 480, 482, 483, 485, 486, 488, 489, 491, 492, 494, 495, 497, 498, 500, 501, 503, 504, 506, 507, 509, 510, 512, 513, 515, 516, 518, 519, 520, 522, and 524. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 1. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 2. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 5. The amino acid sequence of SEQ ID NO: 5 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 6. The amino acid sequence of SEQ ID NO: 6 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 519. The amino acid sequence of SEQ ID NO: 519 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 520. The amino acid sequence of SEQ ID NO: 520 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 12. The amino acid sequence of SEQ ID NO: 12 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 31. The amino acid sequence of SEQ ID NO: 31 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 33. The amino acid sequence of SEQ ID NO: 33 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 34. The amino acid sequence of SEQ ID NO: 34 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 36. The amino acid sequence of SEQ ID NO: 36 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 37. The amino acid sequence of SEQ ID NO: 37 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 39. The amino acid sequence of SEQ ID NO: 39 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 40. The amino acid sequence of SEQ ID NO: 40 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 42. The amino acid sequence of SEQ ID NO: 42 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 43. The amino acid sequence of SEQ ID NO: 43 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 45. The amino acid sequence of SEQ ID NO: 45 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 46. The amino acid sequence of SEQ ID NO: 46 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 48. The amino acid sequence of SEQ ID NO: 48 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 49. The amino acid sequence of SEQ ID NO: 49 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 50. The amino acid sequence of SEQ ID NO: 50 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 51. The amino acid sequence of SEQ ID NO: 51 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 52. The amino acid sequence of SEQ ID NO: 52 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 53. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 276. The amino acid sequence of SEQ ID NO: 276 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 278. The amino acid sequence of SEQ ID NO: 278 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 279. The amino acid sequence of SEQ ID NO: 279 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 332. The amino acid sequence of SEQ ID NO: 332 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 333. The amino acid sequence of SEQ ID NO: 333 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 335. The amino acid sequence of SEQ ID NO: 335 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 336. The amino acid sequence of SEQ ID NO: 336 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 338. The amino acid sequence of SEQ ID NO: 338 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 339. The amino acid sequence of SEQ ID NO: 339 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 341. The amino acid sequence of SEQ ID NO: 341 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 342. The amino acid sequence of SEQ ID NO: 342 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 344. The amino acid sequence of SEQ ID NO: 344 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 345. The amino acid sequence of SEQ ID NO: 345 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 347. The amino acid sequence of SEQ ID NO: 347 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 348. The amino acid sequence of SEQ ID NO: 348 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 350. The amino acid sequence of SEQ ID NO: 350 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 351. The amino acid sequence of SEQ ID NO: 351 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 353. The amino acid sequence of SEQ ID NO: 353 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 354. The amino acid sequence of SEQ ID NO: 354 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 356. The amino acid sequence of SEQ ID NO: 356 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 357. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 366. The amino acid sequence of SEQ ID NO: 366 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 368. The amino acid sequence of SEQ ID NO: 368 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 369. The amino acid sequence of SEQ ID NO: 369 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 371. The amino acid sequence of SEQ ID NO: 371 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 372. The amino acid sequence of SEQ ID NO: 372 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 374. The amino acid sequence of SEQ ID NO: 374 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 375. The amino acid sequence of SEQ ID NO: 375 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 377. The amino acid sequence of SEQ ID NO: 377 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 378. The amino acid sequence of SEQ ID NO: 378 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 380. The amino acid sequence of SEQ ID NO: 380 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 381. The amino acid sequence of SEQ ID NO: 381 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 383. The amino acid sequence of SEQ ID NO: 383 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 384. The amino acid sequence of SEQ ID NO: 384 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 386. The amino acid sequence of SEQ ID NO: 386 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 387. The amino acid sequence of SEQ ID NO: 387 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 389. The amino acid sequence of SEQ ID NO: 389 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 390. The amino acid sequence of SEQ ID NO: 390 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 392. The amino acid sequence of SEQ ID NO: 392 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 393. The amino acid sequence of SEQ ID NO: 393 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 395. The amino acid sequence of SEQ ID NO: 395 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 396. The amino acid sequence of SEQ ID NO: 396 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 398. The amino acid sequence of SEQ ID NO: 398 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 399. The amino acid sequence of SEQ ID NO: 399 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 401. The amino acid sequence of SEQ ID NO: 401 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 402. The amino acid sequence of SEQ ID NO: 402 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 404. The amino acid sequence of SEQ ID NO: 404 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 405. The amino acid sequence of SEQ ID NO: 405 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 407. The amino acid sequence of SEQ ID NO: 407 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 408. The amino acid sequence of SEQ ID NO: 408 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 410. The amino acid sequence of SEQ ID NO: 410 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 411. The amino acid sequence of SEQ ID NO: 411 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 413. The amino acid sequence of SEQ ID NO: 413 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 414. The amino acid sequence of SEQ ID NO: 414 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 416. The amino acid sequence of SEQ ID NO: 416 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 417. The amino acid sequence of SEQ ID NO: 417 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 419. The amino acid sequence of SEQ ID NO: 419 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 420. The amino acid sequence of SEQ ID NO: 420 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 422. The amino acid sequence of SEQ ID NO: 422 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 423. The amino acid sequence of SEQ ID NO: 423 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 425. The amino acid sequence of SEQ ID NO: 425 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 426. The amino acid sequence of SEQ ID NO: 426 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 428. The amino acid sequence of SEQ ID NO: 428 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 429. The amino acid sequence of SEQ ID NO: 429 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 431. The amino acid sequence of SEQ ID NO: 431 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 432. The amino acid sequence of SEQ ID NO: 432 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 434. The amino acid sequence of SEQ ID NO: 434 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 435. The amino acid sequence of SEQ ID NO: 435 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 437. The amino acid sequence of SEQ ID NO: 437 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 438. The amino acid sequence of SEQ ID NO: 438 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 440. The amino acid sequence of SEQ ID NO: 440 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 441. The amino acid sequence of SEQ ID NO: 441 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 443. The amino acid sequence of SEQ ID NO: 443 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 444. The amino acid sequence of SEQ ID NO: 444 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 446. The amino acid sequence of SEQ ID NO: 446 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 447. The amino acid sequence of SEQ ID NO: 447 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 449. The amino acid sequence of SEQ ID NO: 449 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 450. The amino acid sequence of SEQ ID NO: 450 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 452. The amino acid sequence of SEQ ID NO: 452 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 453. The amino acid sequence of SEQ ID NO: 453 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 455. The amino acid sequence of SEQ ID NO: 455 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 456. The amino acid sequence of SEQ ID NO: 456 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 458. The amino acid sequence of SEQ ID NO: 458 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 459. The amino acid sequence of SEQ ID NO: 459 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 461. The amino acid sequence of SEQ ID NO: 461 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 462. The amino acid sequence of SEQ ID NO: 462 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 464. The amino acid sequence of SEQ ID NO: 464 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 465. The amino acid sequence of SEQ ID NO: 465 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 467. The amino acid sequence of SEQ ID NO: 467 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 468. The amino acid sequence of SEQ ID NO: 468 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 470. The amino acid sequence of SEQ ID NO: 470 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 471. The amino acid sequence of SEQ ID NO: 471 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 473. The amino acid sequence of SEQ ID NO: 473 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 474. The amino acid sequence of SEQ ID NO: 474 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 476. The amino acid sequence of SEQ ID NO: 476 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 477. The amino acid sequence of SEQ ID NO: 477 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 479. The amino acid sequence of SEQ ID NO: 479 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 480. The amino acid sequence of SEQ ID NO: 480 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 482. The amino acid sequence of SEQ ID NO: 482 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 483. The amino acid sequence of SEQ ID NO: 483 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 485. The amino acid sequence of SEQ ID NO: 485 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 486. The amino acid sequence of SEQ ID NO: 486 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 488. The amino acid sequence of SEQ ID NO: 488 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 489. The amino acid sequence of SEQ ID NO: 489 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 491. The amino acid sequence of SEQ ID NO: 491 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 492. The amino acid sequence of SEQ ID NO: 492 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 494. The amino acid sequence of SEQ ID NO: 494 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 495. The amino acid sequence of SEQ ID NO: 495 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 497. The amino acid sequence of SEQ ID NO: 497 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 498. The amino acid sequence of SEQ ID NO: 498 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 500. The amino acid sequence of SEQ ID NO: 500 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 501. The amino acid sequence of SEQ ID NO: 501 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 503. The amino acid sequence of SEQ ID NO: 503 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 504. The amino acid sequence of SEQ ID NO: 504 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 506. The amino acid sequence of SEQ ID NO: 506 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 507. The amino acid sequence of SEQ ID NO: 507 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 509. The amino acid sequence of SEQ ID NO: 509 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 510. The amino acid sequence of SEQ ID NO: 510 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 512. The amino acid sequence of SEQ ID NO: 512 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 513. The amino acid sequence of SEQ ID NO: 513 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 515. The amino acid sequence of SEQ ID NO: 515 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 516. The amino acid sequence of SEQ ID NO: 516 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 518. The amino acid sequence of SEQ ID NO: 518 may optionally be provided with the lysine removed from the C-terminus. The G1Fc region may also comprise a mutation to an alanine at position 234 or a mutation to an alanine at position 235, or a combination thereof. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 522. The amino acid sequence of SEQ ID NO: 522 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 524. The amino acid sequence of SEQ ID NO: 524 may optionally be provided with the lysine removed from the C-terminus.

In certain aspects, the disclosure relates to variant ActRIIB polypeptides comprising an amino acid sequence that is at least 70%, 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises one or more amino acid substitutions at a position of SEQ ID NO: 2 selected from the group consisting of:A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132, as well as heteromultimer complexes comprising one or more such variant ActRIIB polypeptides. In certain aspects, the disclosure relates to variant ActRIIB polypeptides comprising an amino acid sequence that is at least 70%, 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises one or more amino acid substitutions at a position of SEQ ID NO: 2 selected from the group consisting of: N35, E50, E52, K55, L57, Y60, G68, K74, W78, L79, F82, N83, E94, as well as heteromultimer complexes comprising one or more such variant ActRIIB polypeptides. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 25-131 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 20-134 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 53. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 12. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 5. In some embodiments, the polypeptide comprises an amino acid substitution at the amino acid position corresponding to A24 of SEQ ID NO: 2. For example, in some embodiments, the substitution is A24N. In some embodiments, the polypeptide comprises an amino acid substitution at the amino acid position corresponding to S26 of SEQ ID NO: 2. For example, in some embodiments, the substitution is S26T. In some embodiments, the polypeptide comprises an amino acid substitution at the amino acid position corresponding to N35 of SEQ ID NO: 2. For example, in some embodiments, the substitution is N35E. In some embodiments, the polypeptide comprises an amino acid substitution at the amino acid position corresponding to E37 of SEQ ID NO: 2. For example, in some embodiments, the substitution is E37A. In some embodiments, the substitution is E37D. In some embodiments, the polypeptide comprises an amino acid substitution at the amino acid position corresponding to L38 of SEQ ID NO: 2. For example, in some embodiments, the substitution is L38N. In some embodiments, the polypeptide comprises an amino acid substitution at the amino acid position corresponding to R40 of SEQ ID NO: 2. For example, in some embodiments, the substitution is R40A. In some embodiments, the substitution is R40K. In some embodiments, the polypeptide comprises an amino acid substitution at the amino acid position corresponding to S44 of SEQ ID NO: 2. For example, in some embodiments, the substitution is S44T. In some embodiments, the polypeptide comprises an amino acid substitution at the amino acid position corresponding to L46 of SEQ ID NO: 2. For example, in some embodiments, the substitution is L46A. For example, in some embodiments, the substitution is L46I. For example, in some embodiments, the substitution is L46F. For example, in some embodiments, the substitution is L46V. In some embodiments, the polypeptide comprises an amino acid substitution at the amino acid position corresponding to E50 of SEQ ID NO: 2. For example, in some embodiments, the substitution is E50K. In some embodiments, the substitution is E50L. In some embodiments, the substitution is E50P. In some embodiments, the polypeptide comprises an amino acid substitution at the amino acid position corresponding to E52 of SEQ ID NO: 2. For example, in some embodiments, the substitution is E52A. In some embodiments, the substitution is E52D. In some embodiments, the substitution is E52G. In some embodiments, the substitution is E52H. In some embodiments, the substitution is E52K. In some embodiments, the substitution is E52N. In some embodiments, the substitution is E52P. In some embodiments, the substitution is E52R. In some embodiments, the substitution is E52S. In some embodiments, the substitution is E52T. In some embodiments, the substitution is E52Y. In some embodiments, the polypeptide comprises an amino acid substitution at the amino acid position corresponding to Q53 of SEQ ID NO: 2. For example, in some embodiments, the substitution is Q53R. For example, in some embodiments, the substitution is Q53K. For example, in some embodiments, the substitution is Q53N. For example, in some embodiments, the substitution is Q53H. In some embodiments, the polypeptide comprises an amino acid substitution at the amino acid position corresponding to D54 of SEQ ID NO: 2. For example, in some embodiments, the substitution is D54A. In some embodiments, the polypeptide comprises an amino acid substitution at the amino acid position corresponding to K55 of SEQ ID NO: 2. For example, in some embodiments, the substitution is K55A. In some embodiments, the substitution is K55E. In some embodiments, the substitution is K55D. In some embodiments, the substitution is K55R. In some embodiments, the polypeptide comprises an amino acid substitution at the amino acid position corresponding to R56 of SEQ ID NO: 2. For example, in some embodiments, the substitution is R56A. In some embodiments, the polypeptide comprises an amino acid substitution at the amino acid position corresponding to L57 of SEQ ID NO: 2. For example, in some embodiments, the substitution is L57R. In some embodiments, the substitution is L57E. In some embodiments, the substitution is L57I. In some embodiments, the substitution is L57T. In some embodiments, the substitution is L57V. In some embodiments, the polypeptide comprises an amino acid substitution at the amino acid position corresponding to Y60 of SEQ ID NO: 2. For example, in some embodiments, the substitution is Y60F. In some embodiments, the substitution is Y60D. In some embodiments, the substitution is Y60K. In some embodiments, the substitution is Y60P. In some embodiments, the polypeptide comprises an amino acid substitution at the amino acid position corresponding to R64 of SEQ ID NO: 2. For example, in some embodiments, the substitution is R64K. In some embodiments, the substitution is R64N. In some embodiments, the substitution is R64A. In some embodiments, the substitution is R64H. In some embodiments, the polypeptide comprises an amino acid substitution at the amino acid position corresponding to N65 of SEQ ID NO: 2. For example, in some embodiments, the substitution is N65A. In some embodiments, the polypeptide comprises an amino acid substitution at the amino acid position corresponding to S67 of SEQ ID NO: 2. For example, in some embodiments, the substitution is S67N. In some embodiments, the substitution is S67T. In some embodiments, the polypeptide comprises an amino acid substitution at the amino acid position corresponding to G68 of SEQ ID NO: 2. For example, in some embodiments, the substitution is G68R. In some embodiments, the polypeptide comprises an amino acid substitution at the amino acid position corresponding to K74 of SEQ ID NO: 2. For example, in some embodiments, the substitution is K74A. In some embodiments, the substitution is K74E. In some embodiments, the substitution is K74F. In some embodiments, the substitution is K74I. In some embodiments, the substitution is K74Y. In some embodiments, the substitution is K74R. In some embodiments, the polypeptide comprises an amino acid substitution at the amino acid position corresponding to W78 of SEQ ID NO: 2. For example, in some embodiments, the substitution is W78A. In some embodiments, the substitution is W78Y. In some embodiments, the polypeptide comprises an amino acid substitution at the amino acid position corresponding to L79 of SEQ ID NO: 2. For example, in some embodiments, the substitution is L79D. In some embodiments, the substitution does not comprise an acidic amino acid at the position corresponding to L79 of SEQ ID NO: 2. In some embodiments, the substitution does not comprise an aspartic acid (D) at the position corresponding to L79 of SEQ ID NO: 2. In some embodiments, the substitution is L79A. In some embodiments, the substitution is L79E. In some embodiments, the substitution is L79F. In some embodiments, the substitution is L79H. In some embodiments, the substitution is L79K. In some embodiments, the substitution is L79P. In some embodiments, the substitution is L79R. In some embodiments, the substitution is L79S. In some embodiments, the substitution is L79T. In some embodiments, the substitution is L79W. In some embodiments, the polypeptide comprises an amino acid substitution at the amino acid position corresponding to D80 of SEQ ID NO: 2. For example, in some embodiments, the substitution is D80A. In some embodiments, the substitution is D80F. In some embodiments, the substitution is D80K. In some embodiments, the substitution is D80G. In some embodiments, the substitution is D80M. In some embodiments, the substitution is D80I. In some embodiments, the substitution is D80N. In some embodiments, the substitution is D80R. In some embodiments, the polypeptide comprises an amino acid substitution at the amino acid position corresponding to F82 of SEQ ID NO: 2. For example, in some embodiments, the substitution is F82I. In some embodiments, the substitution is F82K. In some embodiments, the substitution is F82A. In some embodiments, the substitution is F82W. In some embodiments, the substitution is F82D. In some embodiments, the substitution is F82Y. In some embodiments, the substitution is F82E. In some embodiments, the substitution is F82L. In some embodiments, the substitution is F82T. In some embodiments, the substitution is F82S. In some embodiments, the polypeptide comprises an amino acid substitution at the amino acid position corresponding to N83 of SEQ ID NO: 2. For example, in some embodiments, the substitution is N83A. In some embodiments, the substitution is N83R. In some embodiments, the polypeptide comprises an amino acid substitution at the amino acid position corresponding to T93 of SEQ ID NO: 2. For example, in some embodiments, the substitution is T93D. In some embodiments, the substitution is T93E. In some embodiments, the substitution is T93H. In some embodiments, the substitution is T93G. In some embodiments, the substitution is T93K. In some embodiments, the substitution is T93P. In some embodiments, the substitution is T93R. In some embodiments, the substitution is T93S. In some embodiments, the substitution is T93Y. In some embodiments, the polypeptide comprises an amino acid substitution at the amino acid position corresponding to E94 of SEQ ID NO: 2. For example, in some embodiments, the substitution is E94K. In some embodiments, the polypeptide comprises an amino acid substitution at the amino acid position corresponding to Q98 of SEQ ID NO: 2. For example, in some embodiments, the substitution is Q98D. In some embodiments, the substitution is Q98E. In some embodiments, the substitution is Q98K. In some embodiments, the substitution is Q98R. In some embodiments, the polypeptide comprises an amino acid substitution at the amino acid position corresponding to V99 of SEQ ID NO: 2. For example, in some embodiments, the substitution is V99E. In some embodiments, the substitution is V99G. In some embodiments, the substitution is V99K. In some embodiments, the polypeptide comprises an amino acid substitution at the amino acid position corresponding to E105 of SEQ ID NO: 2. For example, in some embodiments, the substitution is E105N. In some embodiments, the polypeptide comprises an amino acid substitution at the amino acid position corresponding to E106 of SEQ ID NO: 2. For example, in some embodiments, the substitution is E106N. In some embodiments, the polypeptide comprises an amino acid substitution at the amino acid position corresponding to F108 of SEQ ID NO: 2. For example, in some embodiments, the substitution is F108I. In some embodiments, the substitution is F108L. In some embodiments, the substitution is F108V. In some embodiments, the substitution is F108Y. In some embodiments, the polypeptide comprises an amino acid substitution at the amino acid position corresponding to E111 of SEQ ID NO: 2. For example, in some embodiments, the substitution is E111K. In some embodiments, the substitution is E111D. In some embodiments, the substitution is E111R. In some embodiments, the substitution is E111H. In some embodiments, the substitution is E111Q. In some embodiments, the substitution is E111N. In some embodiments, the polypeptide comprises an amino acid substitution at the amino acid position corresponding to R112 of SEQ ID NO: 2. For example, in some embodiments, the substitution is R112H. In some embodiments, the substitution is R112K. In some embodiments, the substitution is R112N. In some embodiments, the substitution is R112S. In some embodiments, the substitution is R112T. In some embodiments, the polypeptide comprises an amino acid substitution at the amino acid position corresponding to A119 of SEQ ID NO: 2. For example, in some embodiments, the substitution is A119P. In some embodiments, the substitution is A119Y. In some embodiments, the polypeptide comprises an amino acid substitution at the amino acid position corresponding to G120 of SEQ ID NO: 2. For example, in some embodiments, the substitution is G120N. In some embodiments, the polypeptide comprises an amino acid substitution at the amino acid position corresponding to E123 of SEQ ID NO: 2. For example, in some embodiments, the substitution is E123N. In some embodiments, the polypeptide comprises an amino acid substitution at the amino acid position corresponding to P129 of SEQ ID NO: 2. For example, in some embodiments, the substitution is P129S. In some embodiments, the substitution is P129N. In some embodiments, the polypeptide comprises an amino acid substitution at the amino acid position corresponding to P130 of SEQ ID NO: 2. For example, in some embodiments, the substitution is P130A. In some embodiments, the substitution is P130R. In some embodiments, the polypeptide comprises an amino acid substitution at the amino acid position corresponding to A132 of SEQ ID NO: 2. For example, in some embodiments, the substitution is A132N.

In some embodiments, any of the variant ActRIIB polypeptides disclosed herein comprises a substitution at a position of SEQ ID NO: 2 selected from the group consisting of: A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132. In some embodiments, any of the variant ActRIIB polypeptides disclosed herein comprises a substitution at a position of SEQ ID NO: 2 selected from the group consisting of: N35, E50, E52, K55, L57, Y60, G68, K74, W78, L79, F82, N83, E94. In some embodiments, the variant ActRIIB polypeptide comprises a substitution at position A24 with respect to SEQ ID NO: 2. In some embodiments, the variant ActRIIB polypeptide comprises a substitution at position S26 with respect to SEQ ID NO: 2. In some embodiments, the variant ActRIIB polypeptide comprises a substitution at position N35 with respect to SEQ ID NO: 2. In some embodiments, the variant ActRIIB polypeptide comprises a substitution at position E37 with respect to SEQ ID NO: 2. In some embodiments, the variant ActRIIB polypeptide comprises a substitution at position L38 with respect to SEQ ID NO: 2. In some embodiments, the variant ActRIIB polypeptide comprises a substitution at position R40 with respect to SEQ ID NO: 2. In some embodiments, the variant ActRIIB polypeptide comprises a substitution at position S44 with respect to SEQ ID NO: 2. In some embodiments, the variant ActRIIB polypeptide comprises a substitution at position D54 with respect to SEQ ID NO: 2. In some embodiments, the variant ActRIIB polypeptide comprises a substitution at position K55 with respect to SEQ ID NO: 2. In some embodiments, the variant ActRIIB polypeptide comprises a substitution at position L46 with respect to SEQ ID NO: 2. In some embodiments, the variant ActRIIB polypeptide comprises a substitution at position E50 with respect to SEQ ID NO: 2. In some embodiments, the variant ActRIIB polypeptide comprises a substitution at position E52 with respect to SEQ ID NO: 2. In some embodiments, the variant ActRIIB polypeptide comprises a substitution at position Q53 with respect to SEQ ID NO: 2. In some embodiments, the variant ActRIIB polypeptide comprises a substitution at position R56 with respect to SEQ ID NO: 2. In some embodiments, the variant ActRIIB polypeptide comprises a substitution at position L57 with respect to SEQ ID NO: 2. In some embodiments, the variant ActRIIB polypeptide comprises a substitution at position Y60 with respect to SEQ ID NO: 2. In some embodiments, the variant ActRIIB polypeptide comprises a substitution at position R64 with respect to SEQ ID NO: 2. In some embodiments, the variant ActRIIB polypeptide comprises a substitution at position N65 with respect to SEQ ID NO: 2. In some embodiments, the variant ActRIIB polypeptide comprises a substitution at position S67 with respect to SEQ ID NO: 2. In some embodiments, the variant ActRIIB polypeptide comprises a substitution at position G68 with respect to SEQ ID NO: 2. In some embodiments, the variant ActRIIB polypeptide comprises a substitution at position K74 with respect to SEQ ID NO: 2. In some embodiments, the variant ActRIIB polypeptide comprises a substitution at position W78 with respect to SEQ ID NO: 2. In some embodiments, the variant ActRIIB polypeptide comprises a substitution at position L79 with respect to SEQ ID NO: 2. In some embodiments, the variant ActRIIB polypeptide comprises a substitution at position D80 with respect to SEQ ID NO: 2. In some embodiments, the variant ActRIIB polypeptide comprises a substitution at position F82 with respect to SEQ ID NO: 2. In some embodiments, the variant ActRIIB polypeptide comprises a substitution at position N83 with respect to SEQ ID NO: 2. In some embodiments, the variant ActRIIB polypeptide comprises a substitution at position T93 with respect to SEQ ID NO: 2. In some embodiments, the variant ActRIIB polypeptide comprises a substitution at position E94 with respect to SEQ ID NO: 2. In some embodiments, the variant ActRIIB polypeptide comprises a substitution at position Q98 with respect to SEQ ID NO: 2. In some embodiments, the variant ActRIIB polypeptide comprises a substitution at position V99 with respect to SEQ ID NO: 2. In some embodiments, the variant ActRIIB polypeptide comprises a substitution at position E105 with respect to SEQ ID NO: 2. In some embodiments, the variant ActRIIB polypeptide comprises a substitution at position E106 with respect to SEQ ID NO: 2. In some embodiments, the variant ActRIIB polypeptide comprises a substitution at position F108 with respect to SEQ ID NO: 2. In some embodiments, the variant ActRIIB polypeptide comprises a substitution at position E111 with respect to SEQ ID NO: 2. In some embodiments, the variant ActRIIB polypeptide comprises a substitution at position R112 with respect to SEQ ID NO: 2. In some embodiments, the variant ActRIIB polypeptide comprises a substitution at position A119 with respect to SEQ ID NO: 2. In some embodiments, the variant ActRIIB polypeptide comprises a substitution at position G120 with respect to SEQ ID NO: 2. In some embodiments, the variant ActRIIB polypeptide comprises a substitution at position E123 with respect to SEQ ID NO: 2. In some embodiments, the variant ActRIIB polypeptide comprises a substitution at position P129 with respect to SEQ ID NO: 2. In some embodiments, the variant ActRIIB polypeptide comprises a substitution at position P130 with respect to SEQ ID NO: 2. In some embodiments, the variant ActRIIB polypeptide comprises a substitution at position A132 with respect to SEQ ID NO: 2.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 31. In some embodiments, the variant ActRIIB polypeptide comprises an alanine at the position corresponding to K55 of SEQ ID NO: 2. In some embodiments, the amino acid sequence of SEQ ID NO: 31 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 33. In some embodiments, the variant ActRIIB polypeptide comprises an alanine at the position corresponding to K55 of SEQ ID NO: 2. In some embodiments, the amino acid sequence of SEQ ID NO: 33 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 34. In some embodiments, the variant ActRIIB polypeptide comprises a glutamic acid at the position corresponding to K55 of SEQ ID NO: 2. In some embodiments, the amino acid sequence of SEQ ID NO: 34 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 36. In some embodiments, the variant ActRIIB polypeptide comprises a glutamic acid at the position corresponding to K55 of SEQ ID NO: 2. In some embodiments, the amino acid sequence of SEQ ID NO: 36 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 37. In some embodiments, the variant ActRIIB polypeptide comprises an isoleucine at the position corresponding to F82 of SEQ ID NO: 2. In some embodiments, the amino acid sequence of SEQ ID NO: 37 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 39. In some embodiments, the variant ActRIIB polypeptide comprises an isoleucine at the position corresponding to F82 of SEQ ID NO: 2. In some embodiments, the amino acid sequence of SEQ ID NO: 39 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 40. In some embodiments, the variant ActRIIB polypeptide comprises a lysine at the position corresponding to F82 of SEQ ID NO: 2. In some embodiments, the amino acid sequence of SEQ ID NO: 40 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 42. In some embodiments, the variant ActRIIB polypeptide comprises a lysine at the position corresponding to F82 of SEQ ID NO: 2. In some embodiments, the amino acid sequence of SEQ ID NO: 42 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 43. In some embodiments, the variant ActRIIB polypeptide comprises a glutamic acid at the position corresponding to L79 of SEQ ID NO: 2. In some embodiments, the amino acid sequence of SEQ ID NO: 43 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 45. In some embodiments, the variant ActRIIB polypeptide comprises a glutamic acid at the position corresponding to L79 of SEQ ID NO: 2. In some embodiments, the amino acid sequence of SEQ ID NO: 45 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 336. In some embodiments, the variant ActRIIB polypeptide comprises a threonine at the position corresponding to F82 of SEQ ID NO: 2. In some embodiments, the amino acid sequence of SEQ ID NO: 336 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 338. In some embodiments, the variant ActRIIB polypeptide comprises a threonine at the position corresponding to F82 of SEQ ID NO: 2. In some embodiments, the amino acid sequence of SEQ ID NO: 338 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 342. In some embodiments, the variant ActRIIB polypeptide comprises a histidine at the position corresponding to L79 of SEQ ID NO: 2. In some embodiments, the amino acid sequence of SEQ ID NO: 342 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 344. In some embodiments, the variant ActRIIB polypeptide comprises a histidine at the position corresponding to L79 of SEQ ID NO: 2. In some embodiments, the amino acid sequence of SEQ ID NO: 344 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 348. In some embodiments, the variant ActRIIB polypeptide comprises a leucine at the position corresponding to E50 of SEQ ID NO: 2. In some embodiments, the amino acid sequence of SEQ ID NO: 348 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 350. In some embodiments, the variant ActRIIB polypeptide comprises a leucine at the position corresponding to E50 of SEQ ID NO: 2. In some embodiments, the amino acid sequence of SEQ ID NO: 350 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 354. In some embodiments, the variant ActRIIB polypeptide comprises a glycine at the position corresponding to V99 of SEQ ID NO: 2. In some embodiments, the amino acid sequence of SEQ ID NO: 354 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 356. In some embodiments, the variant ActRIIB polypeptide comprises a glycine at the position corresponding to V99 of SEQ ID NO: 2. In some embodiments, the amino acid sequence of SEQ ID NO: 356 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 366. In some embodiments, the variant ActRIIB polypeptide comprises a glutamic acid at the position corresponding to N35 of SEQ ID NO: 2. In some embodiments, the amino acid sequence of SEQ ID NO: 366 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 368. In some embodiments, the variant ActRIIB polypeptide comprises a glutamic acid at the position corresponding to N35 of SEQ ID NO: 2. In some embodiments, the amino acid sequence of SEQ ID NO: 368 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 369. In some embodiments, the variant ActRIIB polypeptide comprises an asparagine at the position corresponding to E52 of SEQ ID NO: 2. In some embodiments, the amino acid sequence of SEQ ID NO: 369 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 371. In some embodiments, the variant ActRIIB polypeptide comprises an asparagine at the position corresponding to E52 of SEQ ID NO: 2. In some embodiments, the amino acid sequence of SEQ ID NO: 371 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 372. In some embodiments, the variant ActRIIB polypeptide comprises an aspartic acid at the position corresponding to Y60 of SEQ ID NO: 2. In some embodiments, the amino acid sequence of SEQ ID NO: 372 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 374. In some embodiments, the variant ActRIIB polypeptide comprises an aspartic acid at the position corresponding to Y60 of SEQ ID NO: 2. In some embodiments, the amino acid sequence of SEQ ID NO: 374 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 375. In some embodiments, the variant ActRIIB polypeptide comprises an arginine at the position corresponding to G68 of SEQ ID NO: 2. In some embodiments, the amino acid sequence of SEQ ID NO: 375 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 377. In some embodiments, the variant ActRIIB polypeptide comprises an arginine at the position corresponding to G68 of SEQ ID NO: 2. In some embodiments, the amino acid sequence of SEQ ID NO: 377 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 378. In some embodiments, the variant ActRIIB polypeptide comprises a glutamic acid at the position corresponding to K74 of SEQ ID NO: 2. In some embodiments, the amino acid sequence of SEQ ID NO: 378 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 380. In some embodiments, the variant ActRIIB polypeptide comprises a glutamic acid at the position corresponding to K74 of SEQ ID NO: 2. In some embodiments, the amino acid sequence of SEQ ID NO: 380 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 381. In some embodiments, the variant ActRIIB polypeptide comprises a tyrosine at the position corresponding to W78 of SEQ ID NO: 2. In some embodiments, the amino acid sequence of SEQ ID NO: 381 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 383. In some embodiments, the variant ActRIIB polypeptide comprises a tyrosine at the position corresponding to W78 of SEQ ID NO: 2. In some embodiments, the amino acid sequence of SEQ ID NO: 383 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 384. In some embodiments, the variant ActRIIB polypeptide comprises an alanine at the position corresponding to L79 of SEQ ID NO: 2. In some embodiments, the amino acid sequence of SEQ ID NO: 384 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 386. In some embodiments, the variant ActRIIB polypeptide comprises an alanine at the position corresponding to L79 of SEQ ID NO: 2. In some embodiments, the amino acid sequence of SEQ ID NO: 386 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 387. In some embodiments, the variant ActRIIB polypeptide comprises a lysine at the position corresponding to L79 of SEQ ID NO: 2. In some embodiments, the amino acid sequence of SEQ ID NO: 387 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 389. In some embodiments, the variant ActRIIB polypeptide comprises a lysine at the position corresponding to L79 of SEQ ID NO: 2. In some embodiments, the amino acid sequence of SEQ ID NO: 389 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 390. In some embodiments, the variant ActRIIB polypeptide comprises a serine at the position corresponding to L79 of SEQ ID NO: 2. In some embodiments, the amino acid sequence of SEQ ID NO: 390 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 392. In some embodiments, the variant ActRIIB polypeptide comprises a serine at the position corresponding to L79 of SEQ ID NO: 2. In some embodiments, the amino acid sequence of SEQ ID NO: 392 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 393. In some embodiments, the variant ActRIIB polypeptide comprises a tryptophan at the position corresponding to L79 of SEQ ID NO: 2. In some embodiments, the amino acid sequence of SEQ ID NO: 393 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 395. In some embodiments, the variant ActRIIB polypeptide comprises a tryptophan at the position corresponding to L79 of SEQ ID NO: 2. In some embodiments, the amino acid sequence of SEQ ID NO: 395 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 396. In some embodiments, the variant ActRIIB polypeptide comprises an aspartic acid at the position corresponding to F82 of SEQ ID NO: 2. In some embodiments, the amino acid sequence of SEQ ID NO: 396 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 398. In some embodiments, the variant ActRIIB polypeptide comprises an aspartic acid at the position corresponding to F82 of SEQ ID NO: 2. In some embodiments, the amino acid sequence of SEQ ID NO: 398 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 399. In some embodiments, the variant ActRIIB polypeptide comprises a glutamic acid at the position corresponding to F82 of SEQ ID NO: 2. In some embodiments, the amino acid sequence of SEQ ID NO: 399 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 401. In some embodiments, the variant ActRIIB polypeptide comprises a glutamic acid at the position corresponding to F82 of SEQ ID NO: 2. In some embodiments, the amino acid sequence of SEQ ID NO: 401 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 402. In some embodiments, the variant ActRIIB polypeptide comprises a leucine at the position corresponding to F82 of SEQ ID NO: 2. In some embodiments, the amino acid sequence of SEQ ID NO: 402 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 404. In some embodiments, the variant ActRIIB polypeptide comprises an leucine at the position corresponding to F82 of SEQ ID NO: 2. In some embodiments, the amino acid sequence of SEQ ID NO: 404 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 405. In some embodiments, the variant ActRIIB polypeptide comprises a serine at the position corresponding to F82 of SEQ ID NO: 2. In some embodiments, the amino acid sequence of SEQ ID NO: 405 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 407. In some embodiments, the variant ActRIIB polypeptide comprises a serine at the position corresponding to F82 of SEQ ID NO: 2. In some embodiments, the amino acid sequence of SEQ ID NO: 407 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 408. In some embodiments, the variant ActRIIB polypeptide comprises a tyrosine at the position corresponding to F82 of SEQ ID NO: 2. In some embodiments, the amino acid sequence of SEQ ID NO: 408 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 410. In some embodiments, the variant ActRIIB polypeptide comprises a tyrosine at the position corresponding to F82 of SEQ ID NO: 2. In some embodiments, the amino acid sequence of SEQ ID NO: 410 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 411. In some embodiments, the variant ActRIIB polypeptide comprises a lysine at the position corresponding to E94 of SEQ ID NO: 2. In some embodiments, the amino acid sequence of SEQ ID NO: 411 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 413. In some embodiments, the variant ActRIIB polypeptide comprises a lysine at the position corresponding to E94 of SEQ ID NO: 2. In some embodiments, the amino acid sequence of SEQ ID NO: 413 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 522. In some embodiments, the variant ActRIIB polypeptide comprises a lysine at the position corresponding to F82 of SEQ ID NO: 2. In some embodiments, the amino acid sequence of SEQ ID NO: 522 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 524. In some embodiments, the variant ActRIIB polypeptide comprises a lysine at the position corresponding to F82 of SEQ ID NO: 2. In some embodiments, the amino acid sequence of SEQ ID NO: 524 may optionally be provided with the lysine removed from the C-terminus.

In some embodiments, any of the variant ActRIIB polypeptides disclosed herein comprises at least 2, 3, 4, 5, 6, 7, 8, 9, or 10 of any of the amino acid substitutions disclosed herein. In some embodiments, any of the variant ActRIIB polypeptides disclosed herein comprises 2 of any of the amino acid substitutions disclosed herein. In some embodiments, any of the variant ActRIIB polypeptides disclosed herein comprises 3 of any of the amino acid substitutions disclosed herein. In some embodiments, any of the variant ActRIIB polypeptides disclosed herein comprises 4 of any of the amino acid substitutions disclosed herein. In some embodiments, any of the variant ActRIIB polypeptides disclosed herein comprises 5 of any of the amino acid substitutions disclosed herein. In some embodiments, any of the variant ActRIIB polypeptides disclosed herein comprises 6 of any of the amino acid substitutions disclosed herein. In some embodiments, any of the variant ActRIIB polypeptides disclosed herein comprises 7 of any of the amino acid substitutions disclosed herein. In some embodiments, any of the variant ActRIIB polypeptides disclosed herein comprises 8 of any of the amino acid substitutions disclosed herein. In some embodiments, any of the variant ActRIIB polypeptides disclosed herein comprises 9 of any of the amino acid substitutions disclosed herein. In some embodiments, any of the variant ActRIIB polypeptides disclosed herein comprises 10 of any of the amino acid substitutions disclosed herein.

In certain aspects, the disclosure relates to a variant ActRIIB polypeptide comprising two or more amino acid substitutions as compared to the reference amino acid sequence of SEQ ID NO: 2. For example, in some embodiments, the variant ActRIIB polypeptide comprises an A24N substitution and a K74A substitution. In some embodiments, the variant ActRIIB polypeptide comprises a L79P substitution and a K74A substitution. In some embodiments, the variant ActRIIB polypeptide comprises a P129S substitution and a P130A substitution. In some embodiments, the variant ActRIIB polypeptide comprises a L38N substitution and a L79R substitution. In some embodiments, the variant ActRIIB polypeptide comprises a F82I substitution and a N83R substitution. In some embodiments, the variant ActRIIB polypeptide comprises a F82K substitution and a N83R substitution. In some embodiments, the variant ActRIIB polypeptide comprises a F82T substitution and a N83R substitution. In some embodiments, the variant ActRIIB polypeptide comprises a L79H substitution and a F82K substitution. In some embodiments, the variant ActRIIB polypeptide comprises a L79H substitution and a F82I substitution. In some embodiments, the variant ActRIIB polypeptide comprises a F82D substitution and a N83R substitution. In some embodiments, the variant ActRIIB polypeptide comprises a F82E substitution and a N83R substitution. In some embodiments, the variant ActRIIB polypeptide comprises a L79F substitution and a F82D substitution. In some embodiments, the variant ActRIIB polypeptide comprises a L79F substitution and a F82T substitution. In some embodiments, the variant ActRIIB polypeptide comprises a E52D substitution and a F82D substitution. In some embodiments, the variant ActRIIB polypeptide comprises an E52D substitution and a F82T substitution. In some embodiments, the variant ActRIIB polypeptide comprises a L57R substitution and a F82D substitution. In some embodiments, the variant ActRIIB polypeptide comprises a L57R substitution and a F82T substitution. In some embodiments, the variant ActRIIB polypeptide comprises a F82I substitution and an E94K substitution. In some embodiments, the variant ActRIIB polypeptide comprises a F82S substitution and a N83R substitution. In some embodiments, the variant ActRIIB polypeptide comprises a L57R substitution and a F82S substitution. In some embodiments, the variant ActRIIB polypeptide comprises a K74A substitution and a L79P substitution. In some embodiments, the variant ActRIIB polypeptide comprises a K55A substitution and a F82I substitution. In some embodiments, the variant ActRIIB polypeptide comprises a L79K substitution and a F82K substitution. In some embodiments, the variant ActRIIB polypeptide comprises a F82W substitution and a N83A substitution.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 276. In some embodiments, the variant ActRIIB polypeptide comprises an isoleucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to F82 of SEQ ID NO: 2 and an arginine at the position corresponding to N83 of SEQ ID NO: 2. In some embodiments, the amino acid sequence of SEQ ID NO: 276 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 278. In some embodiments, the variant ActRIIB polypeptide comprises an isoleucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to F82 of SEQ ID NO: 2 and an arginine at the position corresponding to N83 of SEQ ID NO: 2. In some embodiments, the amino acid sequence of SEQ ID NO: 278 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 279. In some embodiments, the variant ActRIIB polypeptide comprises an lysine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a lysine at the position corresponding to F82 of SEQ ID NO: 2 and an arginine at the position corresponding to N83 of SEQ ID NO: 2. In some embodiments, the amino acid sequence of SEQ ID NO: 279 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 332. In some embodiments, the variant ActRIIB polypeptide comprises an lysine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a lysine at the position corresponding to F82 of SEQ ID NO: 2 and an arginine at the position corresponding to N83 of SEQ ID NO: 2. In some embodiments, the amino acid sequence of SEQ ID NO: 332 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 333. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a threonine at the position corresponding to F82 of SEQ ID NO: 2 and an arginine at the position corresponding to N83 of SEQ ID NO: 2. In some embodiments, the amino acid sequence of SEQ ID NO: 333 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 335. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a threonine at the position corresponding to F82 of SEQ ID NO: 2 and an arginine at the position corresponding to N83 of SEQ ID NO: 2. In some embodiments, the amino acid sequence of SEQ ID NO: 335 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 339. In some embodiments, the variant ActRIIB polypeptide comprises a histidine at the position corresponding to L79 of SEQ ID NO: 2. In some embodiments, the variant ActRIIB polypeptide comprises an isoleucine at the position corresponding to F82 of SEQ ID NO: 2. In some embodiments, the variant ActRIIB polypeptide comprises a histidine at the position corresponding to L79 of SEQ ID NO: 2 and an isoleucine at the position corresponding to F82 of SEQ ID NO: 2. In some embodiments, the amino acid sequence of SEQ ID NO: 339 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 341. In some embodiments, the variant ActRIIB polypeptide comprises a histidine at the position corresponding to L79 of SEQ ID NO: 2. In some embodiments, the variant ActRIIB polypeptide comprises an isoleucine at the position corresponding to F82 of SEQ ID NO: 2. In some embodiments, the variant ActRIIB polypeptide comprises a histidine at the position corresponding to L79 of SEQ ID NO: 2 and an isoleucine at the position corresponding to F82 of SEQ ID NO: 2. In some embodiments, the amino acid sequence of SEQ ID NO: 341 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 345. In some embodiments, the variant ActRIIB polypeptide comprises a histidine at the position corresponding to L79 of SEQ ID NO: 2. In some embodiments, the variant ActRIIB polypeptide comprises a lysine at the position corresponding to F82 of SEQ ID NO: 2. In some embodiments, the variant ActRIIB polypeptide comprises a histidine at the position corresponding to L79 of SEQ ID NO: 2, and a lysine at the position corresponding to F82 of SEQ ID NO: 2. In some embodiments, the amino acid sequence of SEQ ID NO: 345 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 347. In some embodiments, the variant ActRIIB polypeptide comprises a histidine at the position corresponding to L79 of SEQ ID NO: 2. In some embodiments, the variant ActRIIB polypeptide comprises a lysine at the position corresponding to F82 of SEQ ID NO: 2. In some embodiments, the variant ActRIIB polypeptide comprises a histidine at the position corresponding to L79 of SEQ ID NO: 2, and a lysine at the position corresponding to F82 of SEQ ID NO: 2. In some embodiments, the amino acid sequence of SEQ ID NO: 347 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 351. In some embodiments, the variant ActRIIB polypeptide comprises an asparagine at the position corresponding to L38 of SEQ ID NO: 2. In some embodiments, the variant ActRIIB polypeptide comprises an arginine at the position corresponding to L79 of SEQ ID NO: 2. In some embodiments, the variant ActRIIB polypeptide comprises an asparagine at the position corresponding to L38 of SEQ ID NO: 2, and an arginine at the position corresponding to L79 of SEQ ID NO: 2. In some embodiments, the amino acid sequence of SEQ ID NO: 351 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 353. In some embodiments, the variant ActRIIB polypeptide comprises an asparagine at the position corresponding to L38 of SEQ ID NO: 2. In some embodiments, the variant ActRIIB polypeptide comprises an arginine at the position corresponding to L79 of SEQ ID NO: 2. In some embodiments, the variant ActRIIB polypeptide comprises an asparagine at the position corresponding to L38 of SEQ ID NO: 2, and an arginine at the position corresponding to L79 of SEQ ID NO: 2. In some embodiments, the amino acid sequence of SEQ ID NO: 353 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 414. In some embodiments, the variant ActRIIB polypeptide comprises an aspartic acid at the position corresponding to E52 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an aspartic acid at the position corresponding to E52 of SEQ ID NO: 2 and an aspartic acid at the position corresponding to F82 of SEQ ID NO: 2. In some embodiments, the amino acid sequence of SEQ ID NO: 414 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 416. In some embodiments, the variant ActRIIB polypeptide comprises an aspartic acid at the position corresponding to E52 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an aspartic acid at the position corresponding to E52 of SEQ ID NO: 2 and an aspartic acid at the position corresponding to F82 of SEQ ID NO: 2. In some embodiments, the amino acid sequence of SEQ ID NO: 416 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 417. In some embodiments, the variant ActRIIB polypeptide comprises an aspartic acid at the position corresponding to E52 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an aspartic acid at the position corresponding to E52 of SEQ ID NO: 2 and a threonine at the position corresponding to F82 of SEQ ID NO: 2. In some embodiments, the amino acid sequence of SEQ ID NO: 417 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 419. In some embodiments, the variant ActRIIB polypeptide comprises an aspartic acid at the position corresponding to E52 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an aspartic acid at the position corresponding to E52 of SEQ ID NO: 2 and a threonine at the position corresponding to F82 of SEQ ID NO: 2. In some embodiments, the amino acid sequence of SEQ ID NO: 419 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 420. In some embodiments, the variant ActRIIB polypeptide comprises an arginine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to L57 of SEQ ID NO: 2 and an aspartic acid at the position corresponding to F82 of SEQ ID NO: 2. In some embodiments, the amino acid sequence of SEQ ID NO: 420 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 422. In some embodiments, the variant ActRIIB polypeptide comprises an arginine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to L57 of SEQ ID NO: 2 and an aspartic acid at the position corresponding to F82 of SEQ ID NO: 2. In some embodiments, the amino acid sequence of SEQ ID NO: 422 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 423. In some embodiments, the variant ActRIIB polypeptide comprises an arginine at the position corresponding to L57 of SEQ ID NO: 2. In some embodiments, the variant ActRIIB polypeptide comprises a serine at the position corresponding to F82 of SEQ ID NO: 2. In some embodiments, the variant ActRIIB polypeptide comprises an arginine at the position corresponding to L57 of SEQ ID NO: 2 and a serine at the position corresponding to F82 of SEQ ID NO: 2. In some embodiments, the amino acid sequence of SEQ ID NO: 423 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 425. In some embodiments, the variant ActRIIB polypeptide comprises an arginine at the position corresponding to L57 of SEQ ID NO: 2. In some embodiments, the variant ActRIIB polypeptide comprises a serine at the position corresponding to F82 of SEQ ID NO: 2. In some embodiments, the variant ActRIIB polypeptide comprises an arginine at the position corresponding to L57 of SEQ ID NO: 2 and a serine at the position corresponding to F82 of SEQ ID NO: 2. In some embodiments, the amino acid sequence of SEQ ID NO: 425 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 426. In some embodiments, the variant ActRIIB polypeptide comprises an arginine at the position corresponding to L57 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to L57 of SEQ ID NO: 2 and a threonine at the position corresponding to F82 of SEQ ID NO: 2. In some embodiments, the amino acid sequence of SEQ ID NO: 426 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 428. In some embodiments, the variant ActRIIB polypeptide comprises an arginine at the position corresponding to L57 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to L57 of SEQ ID NO: 2 and a threonine at the position corresponding to F82 of SEQ ID NO: 2. In some embodiments, the amino acid sequence of SEQ ID NO: 428 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 429. In some embodiments, the variant ActRIIB polypeptide comprises a phenylalanine at the position corresponding to L79 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises a phenylalanine at the position corresponding to L79 of SEQ ID NO: 2 and an aspartic acid at the position corresponding to F82 of SEQ ID NO: 2. In some embodiments, the amino acid sequence of SEQ ID NO: 429 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 431. In some embodiments, the variant ActRIIB polypeptide comprises a phenylalanine at the position corresponding to L79 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises a phenylalanine at the position corresponding to L79 of SEQ ID NO: 2 and an aspartic acid at the position corresponding to F82 of SEQ ID NO: 2. In some embodiments, the amino acid sequence of SEQ ID NO: 431 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 432. In some embodiments, the variant ActRIIB polypeptide comprises a phenylalanine at the position corresponding to L79 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises a phenylalanine at the position corresponding to L79 of SEQ ID NO: 2 and a threonine at the position corresponding to F82 of SEQ ID NO: 2. In some embodiments, the amino acid sequence of SEQ ID NO: 432 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 434. In some embodiments, the variant ActRIIB polypeptide comprises a phenylalanine at the position corresponding to L79 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises a phenylalanine at the position corresponding to L79 of SEQ ID NO: 2 and a threonine at the position corresponding to F82 of SEQ ID NO: 2. In some embodiments, the amino acid sequence of SEQ ID NO: 434 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 435. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to F82 of SEQ ID NO: 2 and an arginine at the position corresponding to N83 of SEQ ID NO: 2. In some embodiments, the amino acid sequence of SEQ ID NO: 435 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 437. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to F82 of SEQ ID NO: 2 and an arginine at the position corresponding to N83 of SEQ ID NO: 2. In some embodiments, the amino acid sequence of SEQ ID NO: 437 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 438. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a glutamic acid at the position corresponding to F82 of SEQ ID NO: 2 and an arginine at the position corresponding to N83 of SEQ ID NO: 2. In some embodiments, the amino acid sequence of SEQ ID NO: 438 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 440. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a glutamic acid at the position corresponding to F82 of SEQ ID NO: 2 and an arginine at the position corresponding to N83 of SEQ ID NO: 2. In some embodiments, the amino acid sequence of SEQ ID NO: 440 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 441. In some embodiments, the variant ActRIIB polypeptide comprises a serine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a serine at the position corresponding to F82 of SEQ ID NO: 2 and an arginine at the position corresponding to N83 of SEQ ID NO: 2. In some embodiments, the amino acid sequence of SEQ ID NO: 441 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 443. In some embodiments, the variant ActRIIB polypeptide comprises a serine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a serine at the position corresponding to F82 of SEQ ID NO: 2 and an arginine at the position corresponding to N83 of SEQ ID NO: 2. In some embodiments, the amino acid sequence of SEQ ID NO: 443 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 444. In some embodiments, the variant ActRIIB polypeptide comprises a tryptophan at the position corresponding to F82 of SEQ ID NO: 2. In some embodiments, the variant ActRIIB polypeptide comprises an alanine at the position corresponding to N83 of SEQ ID NO: 2. In some embodiments, the variant ActRIIB polypeptide comprises a tryptophan at the position corresponding to F82 of SEQ ID NO: 2 and an alanine at the position corresponding to N83 of SEQ ID NO: 2. In some embodiments, the amino acid sequence of SEQ ID NO: 444 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 446. In some embodiments, the variant ActRIIB polypeptide comprises a tryptophan at the position corresponding to F82 of SEQ ID NO: 2. In some embodiments, the variant ActRIIB polypeptide comprises an alanine at the position corresponding to N83 of SEQ ID NO: 2. In some embodiments, the variant ActRIIB polypeptide comprises a tryptophan at the position corresponding to F82 of SEQ ID NO: 2 and an alanine at the position corresponding to N83 of SEQ ID NO: 2. In some embodiments, the amino acid sequence of SEQ ID NO: 446 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 447. In some embodiments, the variant ActRIIB polypeptide comprises an isoleucine at the position corresponding to F82 of SEQ ID NO: 2. In some embodiments, the variant ActRIIB polypeptide comprises a lysine at the position corresponding to E94 of SEQ ID NO: 2. In some embodiments, the variant ActRIIB polypeptide comprises an isoleucine at the position corresponding to F82 of SEQ ID NO: 2 and a lysine at the position corresponding to E94 of SEQ ID NO: 2. In some embodiments, the amino acid sequence of SEQ ID NO: 447 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 449. In some embodiments, the variant ActRIIB polypeptide comprises an isoleucine at the position corresponding to F82 of SEQ ID NO: 2. In some embodiments, the variant ActRIIB polypeptide comprises a lysine at the position corresponding to E94 of SEQ ID NO: 2. In some embodiments, the variant ActRIIB polypeptide comprises an isoleucine at the position corresponding to F82 of SEQ ID NO: 2 and a lysine at the position corresponding to E94 of SEQ ID NO: 2. In some embodiments, the amino acid sequence of SEQ ID NO: 449 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising three or more amino acid substitutions as compared to the reference amino acid sequence of SEQ ID NO: 2. In some embodiments, the variant ActRIIB polypeptide comprises a G68R substitution, a F82S substitution, and a N83R substitution. In some embodiments, the variant ActRIIB polypeptide comprises a G68R substitution, a W78Y substitution, and a F82Y substitution. In some embodiments, the variant ActRIIB polypeptide comprises a E52D substitution, a F82D substitution, and a N83R substitution. In some embodiments, the variant ActRIIB polypeptide comprises an E52Y substitution, a F82D substitution, and a N83R substitution. In some embodiments, the variant ActRIIB polypeptide comprises an E52D substitution, a F82E substitution, and a N83R substitution. In some embodiments, the variant ActRIIB polypeptide comprises an E52D substitution, a F82T substitution, and a N83R substitution. In some embodiments, the variant ActRIIB polypeptide comprises an E52N substitution, a F82I substitution, and a N83R substitution. In some embodiments, the variant ActRIIB polypeptide comprises an E52N substitution, a F82Y substitution, and a N83R substitution. In some embodiments, the variant ActRIIB polypeptide comprises an E50L substitution, a F82D substitution, and a N83R substitution. In some embodiments, the variant ActRIIB polypeptide comprises a L57I substitution, a F82D substitution, and a N83R substitution. In some embodiments, the variant ActRIIB polypeptide comprises a L57V substitution, a F82D substitution, and a N83R substitution. In some embodiments, the variant ActRIIB polypeptide comprises a L57R substitution, a F82D substitution, and a N83R substitution. In some embodiments, the variant ActRIIB polypeptide comprises a L57E substitution, a F82E substitution, and a N83R substitution. In some embodiments, the variant ActRIIB polypeptide comprises a L57R substitution, a F82E substitution, and a N83R substitution. In some embodiments, the variant ActRIIB polypeptide comprises a L57I substitution, a F82E substitution, and a N83R substitution. In some embodiments, the variant ActRIIB polypeptide comprises a L57R substitution, a F82L substitution, and a N83R substitution. In some embodiments, the variant ActRIIB polypeptide comprises a L57T substitution, a F82Y substitution, and a N83R substitution. In some embodiments, the variant ActRIIB polypeptide comprises a L57V substitution, a F82Y substitution, and a N83R substitution. In some embodiments, the variant ActRIIB polypeptide may comprise at least two of the amino acid substitutions described in any of the variant ActRIIB polypeptides above.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 450. In some embodiments, the variant ActRIIB polypeptide comprises a leucine at the position corresponding to E50 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a leucine at the position corresponding to E50 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 450 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 452. In some embodiments, the variant ActRIIB polypeptide comprises a leucine at the position corresponding to E50 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a leucine at the position corresponding to E50 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 452 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 453. In some embodiments, the variant ActRIIB polypeptide comprises an aspartic acid at the position corresponding to E52 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to E52 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 453 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 455. In some embodiments, the variant ActRIIB polypeptide comprises an aspartic acid at the position corresponding to E52 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to E52 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 455 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 456. In some embodiments, the variant ActRIIB polypeptide comprises an aspartic acid at the position corresponding to E52 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to E52 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 456 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 458. In some embodiments, the variant ActRIIB polypeptide comprises an aspartic acid at the position corresponding to E52 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to E52 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 458 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 459. In some embodiments, the variant ActRIIB polypeptide comprises an aspartic acid at the position corresponding to E52 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to E52 of SEQ ID NO: 2, a threonine at the position corresponding to F82 of SEQ ID NO: 2, and an arginine at the position corresponding to N83 of SEQ ID NO: 2. In some embodiments, the amino acid sequence of SEQ ID NO: 459 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 461. In some embodiments, the variant ActRIIB polypeptide comprises an aspartic acid at the position corresponding to E52 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to E52 of SEQ ID NO: 2, a threonine at the position corresponding to F82 of SEQ ID NO: 2, and an arginine at the position corresponding to N83 of SEQ ID NO: 2. In some embodiments, the amino acid sequence of SEQ ID NO: 461 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 462. In some embodiments, the variant ActRIIB polypeptide comprises an asparagine at the position corresponding to E52 of SEQ ID NO: 2. In some embodiments, the variant ActRIIB polypeptide comprises an isoleucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an asparagine at the position corresponding to E52 of SEQ ID NO: 2, an isoleucine at the position corresponding to F82 of SEQ ID NO: 2, and an arginine at the position corresponding to N83 of SEQ ID NO: 2. In some embodiments, the amino acid sequence of SEQ ID NO: 462 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 464. In some embodiments, the variant ActRIIB polypeptide comprises an asparagine at the position corresponding to E52 of SEQ ID NO: 2. In some embodiments, the variant ActRIIB polypeptide comprises an isoleucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an asparagine at the position corresponding to E52 of SEQ ID NO: 2, an isoleucine at the position corresponding to F82 of SEQ ID NO: 2, and an arginine at the position corresponding to N83 of SEQ ID NO: 2. In some embodiments, the amino acid sequence of SEQ ID NO: 464 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 465. In some embodiments, the variant ActRIIB polypeptide comprises an asparagine at the position corresponding to E52 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an asparagine at the position corresponding to E52 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 465 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 467. In some embodiments, the variant ActRIIB polypeptide comprises an asparagine at the position corresponding to E52 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an asparagine at the position corresponding to E52 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 467 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 468. In some embodiments, the variant ActRIIB polypeptide comprises a tyrosine at the position corresponding to E52 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a tyrosine at the position corresponding to E52 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 468 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 470. In some embodiments, the variant ActRIIB polypeptide comprises a tyrosine at the position corresponding to E52 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a tyrosine at the position corresponding to E52 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 470 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 471. In some embodiments, the variant ActRIIB polypeptide comprises a glutamic acid at the position corresponding to L57 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a glutamic acid at the position corresponding to L57 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 471 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 473. In some embodiments, the variant ActRIIB polypeptide comprises a glutamic acid at the position corresponding to L57 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a glutamic acid at the position corresponding to L57 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 473 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 474. In some embodiments, the variant ActRIIB polypeptide comprises an isoleucine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to L57 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 474 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 476. In some embodiments, the variant ActRIIB polypeptide comprises an isoleucine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to L57 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 476 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 477. In some embodiments, the variant ActRIIB polypeptide comprises an isoleucine at the position corresponding to L57 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to L57 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 477 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 479. In some embodiments, the variant ActRIIB polypeptide comprises an isoleucine at the position corresponding to L57 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to L57 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 479 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 480. In some embodiments, the variant ActRIIB polypeptide comprises an arginine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to L57 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 480 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 482. In some embodiments, the variant ActRIIB polypeptide comprises an arginine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to L57 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 482 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 483. In some embodiments, the variant ActRIIB polypeptide comprises an arginine at the position corresponding to L57 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to L57 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 483 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 485. In some embodiments, the variant ActRIIB polypeptide comprises an arginine at the position corresponding to L57 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to L57 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 485 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 486. In some embodiments, the variant ActRIIB polypeptide comprises an arginine at the position corresponding to L57 of SEQ ID NO: 2. In some embodiments, the variant ActRIIB polypeptide comprises a leucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to L57 of SEQ ID NO: 2, a leucine at the position corresponding to F82 of SEQ ID NO: 2, and an arginine at the position corresponding to N83 of SEQ ID NO: 2. In some embodiments, the amino acid sequence of SEQ ID NO: 486 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 488. In some embodiments, the variant ActRIIB polypeptide comprises an arginine at the position corresponding to L57 of SEQ ID NO: 2. In some embodiments, the variant ActRIIB polypeptide comprises a leucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to L57 of SEQ ID NO: 2, a leucine at the position corresponding to F82 of SEQ ID NO: 2, and an arginine at the position corresponding to N83 of SEQ ID NO: 2. In some embodiments, the amino acid sequence of SEQ ID NO: 488 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 489. In some embodiments, the variant ActRIIB polypeptide comprises a threonine at the position corresponding to L57 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a threonine at the position corresponding to L57 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 489 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 491. In some embodiments, the variant ActRIIB polypeptide comprises a threonine at the position corresponding to L57 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a threonine at the position corresponding to L57 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 491 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 492. In some embodiments, the variant ActRIIB polypeptide comprises a valine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a valine at the position corresponding to L57 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 492 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 494. In some embodiments, the variant ActRIIB polypeptide comprises a valine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a valine at the position corresponding to L57 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 494 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 495. In some embodiments, the variant ActRIIB polypeptide comprises a valine at the position corresponding to L57 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a valine at the position corresponding to L57 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 495 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 497. In some embodiments, the variant ActRIIB polypeptide comprises a valine at the position corresponding to L57 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a valine at the position corresponding to L57 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 497 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 498. In some embodiments, the variant ActRIIB polypeptide comprises an arginine at the position corresponding to G68 of SEQ ID NO: 2. In some embodiments, the variant ActRIIB polypeptide comprises a tyrosine at the position corresponding to W78 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to G68 of SEQ ID NO: 2, a tyrosine at the position corresponding to W78 of SEQ ID NO: 2, and a tyrosine at the position corresponding to F82 of SEQ ID NO: 2. In some embodiments, the amino acid sequence of SEQ ID NO: 498 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 500. In some embodiments, the variant ActRIIB polypeptide comprises an arginine at the position corresponding to G68 of SEQ ID NO: 2. In some embodiments, the variant ActRIIB polypeptide comprises a tyrosine at the position corresponding to W78 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to G68 of SEQ ID NO: 2, a tyrosine at the position corresponding to W78 of SEQ ID NO: 2, and a tyrosine at the position corresponding to F82 of SEQ ID NO: 2. In some embodiments, the amino acid sequence of SEQ ID NO: 500 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 501. In some embodiments, the variant ActRIIB polypeptide comprises an arginine at the position corresponding to G68 of SEQ ID NO: 2. In some embodiments, the variant ActRIIB polypeptide comprises a serine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to G68 of SEQ ID NO: 2, a serine at the position corresponding to F82 of SEQ ID NO: 2, and an arginine at the position corresponding to N83 of SEQ ID NO: 2. In some embodiments, the amino acid sequence of SEQ ID NO: 501 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 503. In some embodiments, the variant ActRIIB polypeptide comprises an arginine at the position corresponding to G68 of SEQ ID NO: 2. In some embodiments, the variant ActRIIB polypeptide comprises a serine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to G68 of SEQ ID NO: 2, a serine at the position corresponding to F82 of SEQ ID NO: 2, and an arginine at the position corresponding to N83 of SEQ ID NO: 2. In some embodiments, the amino acid sequence of SEQ ID NO: 503 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising four or more amino acid substitutions as compared to the reference amino acid sequence of SEQ ID NO: 2. For example, in some embodiments, the variant ActRIIB polypeptide comprises a G68R substitution, a L79E substitution, a F82Y substitution, and a N83R substitution. In some embodiments, the variant ActRIIB polypeptide comprises a G68R substitution, a L79E substitution, a F82T substitution, and a N83R substitution. In some embodiments, the variant ActRIIB polypeptide comprises a G68R substitution, a L79T substitution, a F82T substitution, and a N83R substitution. In some embodiments, the variant ActRIIB polypeptide comprises an E52N substitution, a G68R substitution, a F82Y substitution, and a N83R substitution. In some embodiments, the variant ActRIIB polypeptide may comprise at least two of the amino acid substitutions described in any of the variant ActRIIB polypeptides above. In some embodiments, the variant ActRIIB polypeptide may comprise at least three of the amino acid substitutions described in any of the variant ActRIIB polypeptides above.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 504. In some embodiments, the variant ActRIIB polypeptide comprises an asparagine at the position corresponding to E52 of SEQ ID NO: 2. In some embodiments, the variant ActRIIB polypeptide comprises an arginine at the position corresponding to G68 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an asparagine at the position corresponding to E52 of SEQ ID NO: 2, an arginine at the position corresponding to G68 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 504 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 506. In some embodiments, the variant ActRIIB polypeptide comprises an asparagine at the position corresponding to E52 of SEQ ID NO: 2. In some embodiments, the variant ActRIIB polypeptide comprises an arginine at the position corresponding to G68 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an asparagine at the position corresponding to E52 of SEQ ID NO: 2, an arginine at the position corresponding to G68 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 506 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 507. In some embodiments, the variant ActRIIB polypeptide comprises an arginine at the position corresponding to G68 of SEQ ID NO: 2. In some embodiments, the variant ActRIIB polypeptide comprises a glutamic acid at the position corresponding to L79 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to G68 of SEQ ID NO: 2, a glutamic acid at the position corresponding to L79 of SEQ ID NO: 2, a threonine at the position corresponding to F82 of SEQ ID NO: 2, and an arginine at the position corresponding to N83 of SEQ ID NO: 2. In some embodiments, the amino acid sequence of SEQ ID NO: 507 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 509. In some embodiments, the variant ActRIIB polypeptide comprises an arginine at the position corresponding to G68 of SEQ ID NO: 2. In some embodiments, the variant ActRIIB polypeptide comprises a glutamic acid at the position corresponding to L79 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to G68 of SEQ ID NO: 2, a glutamic acid at the position corresponding to L79 of SEQ ID NO: 2, a threonine at the position corresponding to F82 of SEQ ID NO: 2, and an arginine at the position corresponding to N83 of SEQ ID NO: 2. In some embodiments, the amino acid sequence of SEQ ID NO: 509 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 510. In some embodiments, the variant ActRIIB polypeptide comprises an arginine at the position corresponding to G68 of SEQ ID NO: 2. In some embodiments, the variant ActRIIB polypeptide comprises a glutamic acid at the position corresponding to L79 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to G68 of SEQ ID NO: 2, a glutamic acid at the position corresponding to L79 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 510 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 512. In some embodiments, the variant ActRIIB polypeptide comprises an arginine at the position corresponding to G68 of SEQ ID NO: 2. In some embodiments, the variant ActRIIB polypeptide comprises a glutamic acid at the position corresponding to L79 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to G68 of SEQ ID NO: 2, a glutamic acid at the position corresponding to L79 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 512 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 513. In some embodiments, the variant ActRIIB polypeptide comprises an arginine at the position corresponding to G68 of SEQ ID NO: 2. In some embodiments, the variant ActRIIB polypeptide comprises a threonine at the position corresponding to L79 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to G68 of SEQ ID NO: 2, a threonine at the position corresponding to L79 of SEQ ID NO: 2, a threonine at the position corresponding to F82 of SEQ ID NO: 2, and an arginine at the position corresponding to N83 of SEQ ID NO: 2. In some embodiments, the amino acid sequence of SEQ ID NO: 513 may optionally be provided with the lysine removed from the C-terminus. The G1Fc region may also comprise a mutation to an alanine at position 234 or a mutation to an alanine at position 235, or a combination thereof.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 515. In some embodiments, the variant ActRIIB polypeptide comprises an arginine at the position corresponding to G68 of SEQ ID NO: 2. In some embodiments, the variant ActRIIB polypeptide comprises a threonine at the position corresponding to L79 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to G68 of SEQ ID NO: 2, a threonine at the position corresponding to L79 of SEQ ID NO: 2, a threonine at the position corresponding to F82 of SEQ ID NO: 2, and an arginine at the position corresponding to N83 of SEQ ID NO: 2. In some embodiments, the amino acid sequence of SEQ ID NO: 515 may optionally be provided with the lysine removed from the C-terminus. The G1Fc region may also comprise a mutation to an alanine at position 234 or a mutation to an alanine at position 235, or a combination thereof.

In some embodiments, variant ActRIIB-Fc proteins display varying ligand binding profiles compared to an Fc fusion protein comprising unmodified ActRIIB extracellular domain. Accordingly, in some embodiments of the present disclosure, variant ActRIIB proteins be more useful than unmodified ActRIIB in applications where such selective antagonism profiles are advantageous. For example, in some embodiments, it may be desirable to use an ActRIIB variant of the present disclosure to retain antagonism of one or more of activin A, activin B, GDF8, GDF11, BMP6, and/or BMP10, while reducing antagonism of BMP9.

In some embodiments, binding profiles of an ActRIIB variant of the present disclosure are varied from ligand binding profiles of an unmodified ActRIIB-Fc protein (e.g., WT). In some embodiments, a “significant reduction” in ligand binding as used herein refers to a decrease of over 30 times that of the WT. In certain embodiments, a “significant reduction” in ligand binding as used herein refers to a decrease so significant that binding is not detected. In some embodiments, a “modest reduction” in ligand binding as used herein refers to a decrease of between 10 and 30 times that of the WT. In some embodiments, a “minor reduction” in ligand binding as used herein refers to a decrease of between 3 and 10 times that of the WT. In some embodiments, a “near-WT level” in ligand binding as used herein refers to a decrease of between 3 times or less than that of the WT.

In some embodiments, an ActRIIB variant of the present disclosure displays a significant reduction in activin A binding, a significant reduction in GDF 11 binding, no detected BMP10 binding, a minor reduction in BMP6 binding, and a significant reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions E50, F82, and/or N83, wherein the variant displays a significant reduction in activin A binding, a significant reduction in GDF11 binding, no detected BMP10 binding, a minor reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions E50, F82, and/or N83, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 437. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to F82 of SEQ ID NO: 2 and an arginine at the position corresponding to N83 of SEQ ID NO: 2. In some embodiments, the amino acid sequence of SEQ ID NO: 437 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 452. In some embodiments, the variant ActRIIB polypeptide comprises a leucine at the position corresponding to E50 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a leucine at the position corresponding to E50 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 452 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a significant reduction in activin A binding, near-WT levels of GDF11 binding, near-WT levels of BMP10 binding, a slight increase in BMP6 binding, and a modest reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions L79 and/or F82, wherein the variant displays a significant reduction in activin A binding, near-WT levels of GDF11 binding, near-WT levels of BMP10 binding, a slight increase in BMP6 binding, and a modest reduction in BMP9 binding, compared to binding profiles of an unmodified ActRIIB-Fc protein (e.g., WT). In some embodiments, an ActRIIB variant comprises a mutation at any one of positions L79 and/or F82, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 434. In some embodiments, the variant ActRIIB polypeptide comprises a phenylalanine at the position corresponding to L79 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises a phenylalanine at the position corresponding to L79 of SEQ ID NO: 2 and a threonine at the position corresponding to F82 of SEQ ID NO: 2. In some embodiments, the amino acid sequence of SEQ ID NO: 434 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a significant reduction in activin A binding, near-WT levels of GDF11 binding, a minor reduction in BMP10 binding, near-WT levels of BMP6 binding, and a minor reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at position L79, wherein the variant displays a significant reduction in activin A binding, near-WT levels of GDF11 binding, a minor reduction in BMP10 binding, near-WT levels of BMP6 binding, and a minor reduction in BMP9 binding, compared to binding profiles of an unmodified ActRIIB-Fc protein (e.g., WT). In some embodiments, an ActRIIB variant comprises a mutation at position L79, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 392. In some embodiments, the variant ActRIIB polypeptide comprises a serine at the position corresponding to L79 of SEQ ID NO: 2. In some embodiments, the amino acid sequence of SEQ ID NO: 392 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF11 binding, no detectable BMP10 binding, a minor reduction in BMP6 binding, and a significant reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions L57, F82, and/or N83, wherein the variant displays a modest reduction in activin A binding, a significant reduction in GDF11 binding, no detectable BMP10 binding, a minor reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions L57, F82, and/or N83 wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 488. In some embodiments, the variant ActRIIB polypeptide comprises an arginine at the position corresponding to L57 of SEQ ID NO: 2. In some embodiments, the variant ActRIIB polypeptide comprises a leucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to L57 of SEQ ID NO: 2, a leucine at the position corresponding to F82 of SEQ ID NO: 2, and an arginine at the position corresponding to N83 of SEQ ID NO: 2. In some embodiments, the amino acid sequence of SEQ ID NO: 488 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF 11 binding, no detectable BMP10 binding, a minor reduction in BMP6 binding, and a significant reduction in BMP9 binding, compared to binding profiles of an unmodified ActRIIB-Fc protein (e.g., WT). In some embodiments, an ActRIIB variant of the present disclosure displays a modest reduction in activin B binding, compared to binding profiles of an unmodified ActRIIB-Fc protein (e.g., WT). In some embodiments, an ActRIIB variant comprises a mutation at any one of positions L57, F82, and/or N83, wherein the variant displays a modest reduction in activin A binding, a significant reduction in GDF11 binding, no detectable BMP10 binding, a minor reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions L57, F82, and/or N83, wherein the variant exhibits a modest reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 494. In some embodiments, the variant ActRIIB polypeptide comprises a valine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a valine at the position corresponding to L57 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 494 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF11 binding, no detectable BMP10 binding, a significant reduction in BMP6 binding, and a significant reduction in BMP9 binding, compared to binding profiles of an unmodified ActRIIB-Fc protein (e.g., WT). In some embodiments, an ActRIIB variant of the present disclosure displays a modest reduction in activin B binding, compared to binding profiles of an unmodified ActRIIB-Fc protein (e.g., WT). In some embodiments, an ActRIIB variant comprises a mutation at any one of positions L57, F82, and/or N83, wherein the variant displays modest reduction in activin A binding, a significant reduction in GDF11 binding, no detectable BMP10 binding, a significant reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions L57, F82, and/or N83, wherein the variant exhibits a modest reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 482. In some embodiments, the variant ActRIIB polypeptide comprises an arginine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to L57 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 482 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 485. In some embodiments, the variant ActRIIB polypeptide comprises an arginine at the position corresponding to L57 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an arginine at the position corresponding to L57 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 485 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF 11 binding, no detectable BMP10 binding, a minor reduction in BMP6 binding, and a significant reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions E52, F82, and/or N83, wherein the variant displays a modest reduction in activin A binding, a significant reduction in GDF11 binding, no detectable BMP10 binding, a minor reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions E52, F82, and/or N83, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 443. In some embodiments, the variant ActRIIB polypeptide comprises a serine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a serine at the position corresponding to F82 of SEQ ID NO: 2 and an arginine at the position corresponding to N83 of SEQ ID NO: 2. In some embodiments, the amino acid sequence of SEQ ID NO: 443 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 467. In some embodiments, the variant ActRIIB polypeptide comprises an asparagine at the position corresponding to E52 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an asparagine at the position corresponding to E52 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 467 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF11 binding, minimal BMP10 binding, a minor reduction in BMP6 binding, and a significant reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions L57, F82, and/or N83, wherein the variant displays a modest reduction in activin A binding, a significant reduction in GDF11 binding, minimal BMP10 binding, a minor reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions L57, F82, and/or N83, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 491. In some embodiments, the variant ActRIIB polypeptide comprises a threonine at the position corresponding to L57 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a threonine at the position corresponding to L57 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 491 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF11 binding, minimal BMP10 binding, a minor reduction in BMP6 binding, and a significant reduction in BMP9 binding, compared to binding profiles of an unmodified ActRIIB-Fc protein (e.g., WT). In some embodiments, an ActRIIB variant of the present disclosure displays a modest reduction in activin B binding, compared to binding profiles of an unmodified ActRIIB-Fc protein (e.g., WT). In some embodiments, an ActRIIB variant comprises a mutation at any one of positions E52, F82, and/or N83, wherein the variant displays a modest reduction in activin A binding, a significant reduction in GDF11 binding, minimal BMP10 binding, a minor reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions E52, F82, and/or N83, wherein the variant exhibits a modest reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 455. In some embodiments, the variant ActRIIB polypeptide comprises an aspartic acid at the position corresponding to E52 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to E52 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 455 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF11 binding, minimal BMP10 binding, a modest reduction in BMP6 binding, and a significant reduction in BMP9 binding, compared to binding profiles of an unmodified ActRIIB-Fc protein (e.g., WT). In some embodiments, an ActRIIB variant of the present disclosure displays a modest reduction in activin B binding, compared to binding profiles of an unmodified ActRIIB-Fc protein (e.g., WT). In some embodiments, an ActRIIB variant comprises a mutation at any one of positions L57, F82, and/or N83, wherein the variant displays a modest reduction in activin A binding, a significant reduction in GDF11 binding, minimal BMP10 binding, a modest reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions L57, F82, and/or N83, wherein the variant exhibits a modest reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 476. In some embodiments, the variant ActRIIB polypeptide comprises an isoleucine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to L57 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 476 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF11 binding, a modest reduction in BMP10 binding, a minor reduction in BMP6 binding, and a significant reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions E52, F82, and/or N83, wherein the variant displays a modest reduction in activin A binding, a significant reduction in GDF11 binding, a modest reduction in BMP10 binding, a minor reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions E52, F82, and/or N83, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 461. In some embodiments, the variant ActRIIB polypeptide comprises an aspartic acid at the position corresponding to E52 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to E52 of SEQ ID NO: 2, a threonine at the position corresponding to F82 of SEQ ID NO: 2, and an arginine at the position corresponding to N83 of SEQ ID NO: 2. In some embodiments, the amino acid sequence of SEQ ID NO: 461 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF11 binding, a modest reduction in BMP10 binding, minimal BMP6 binding, and a significant reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions E52, F82, and/or N83, wherein the variant displays a modest reduction in activin A binding, a significant reduction in GDF11 binding, a modest reduction in BMP10 binding, minimal BMP6 binding, and a significant reduction in BMP9 binding, compared to binding profiles of an unmodified ActRIIB-Fc protein (e.g., WT). In some embodiments, an ActRIIB variant comprises a mutation at any one of positions E52, F82, and/or N83, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 464. In some embodiments, the variant ActRIIB polypeptide comprises an asparagine at the position corresponding to E52 of SEQ ID NO: 2. In some embodiments, the variant ActRIIB polypeptide comprises an isoleucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an asparagine at the position corresponding to E52 of SEQ ID NO: 2, an isoleucine at the position corresponding to F82 of SEQ ID NO: 2, and an arginine at the position corresponding to N83 of SEQ ID NO: 2. In some embodiments, the amino acid sequence of SEQ ID NO: 464 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a significant reduction in GDF11 binding, a modest reduction in BMP10 binding, minimal BMP6 binding, and a modest reduction in BMP9 binding, compared to binding profiles of an unmodified ActRIIB-Fc protein (e.g., WT). In some embodiments, an ActRIIB variant of the present disclosure displays a modest reduction in activin B binding, compared to binding profiles of an unmodified ActRIIB-Fc protein (e.g., WT). In some embodiments, an ActRIIB variant comprises a mutation at any one of positions E52, F82, and/or N83, wherein the variant displays a modest reduction in activin A binding, a significant reduction in GDF11 binding, a modest reduction in BMP10 binding, minimal BMP6 binding, and a modest reduction in BMP9 binding, compared to binding profiles of an unmodified ActRIIB-Fc protein (e.g., WT). In some embodiments, an ActRIIB variant comprises a mutation at any one of positions E52, F82, and/or N83, wherein the variant exhibits a modest reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 470. In some embodiments, the variant ActRIIB polypeptide comprises a tyrosine at the position corresponding to E52 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a tyrosine at the position corresponding to E52 of SEQ ID NO: 2, an aspartic acid at the position corresponding to F82 of SEQ ID NO: 2, and an arginine at the position corresponding to N83 of SEQ ID NO: 2. In some embodiments, the amino acid sequence of SEQ ID NO: 470 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, a modest reduction in GDF11 binding, no detectable BMP10 binding, a minor reduction in BMP6 binding, and a significant reduction in BMP9 binding, compared to binding profiles of an unmodified ActRIIB-Fc protein (e.g., WT). In some embodiments, an ActRIIB variant of the present disclosure displays a minor reduction in activin B binding, compared to binding profiles of an unmodified ActRIIB-Fc protein (e.g., WT). In some embodiments, an ActRIIB variant comprises a mutation at any one of positions E52, L57, F82, and/or N83, wherein the variant displays a modest reduction in activin A binding, a modest reduction in GDF11 binding, no detectable BMP10 binding, a minor reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions E52, L57, F82, and/or N83, wherein the variant exhibits a minor reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 440. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a glutamic acid at the position corresponding to F82 of SEQ ID NO: 2 and an arginine at the position corresponding to N83 of SEQ ID NO: 2. In some embodiments, the amino acid sequence of SEQ ID NO: 440 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 458. In some embodiments, the variant ActRIIB polypeptide comprises an aspartic acid at the position corresponding to E52 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an aspartic acid at the position corresponding to E52 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 458 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 479. In some embodiments, the variant ActRIIB polypeptide comprises an isoleucine at the position corresponding to L57 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to L57 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 479 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 473. In some embodiments, the variant ActRIIB polypeptide comprises a glutamic acid at the position corresponding to L57 of SEQ ID NO: 2. In some embodiments, the variant ActRIIB polypeptide comprises a glutamic acid at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a glutamic acid at the position corresponding to L57 of SEQ ID NO: 2, a glutamic acid at the position corresponding to F82 of SEQ ID NO: 2, and an arginine at the position corresponding to N83 of SEQ ID NO: 2. In some embodiments, the amino acid sequence of SEQ ID NO: 473 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, near-WT levels of GDF11 binding, near-WT levels of BMP10 binding, a slight increase in BMP6 binding, and near-WT levels of BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at position L79, wherein the variant displays a modest reduction in activin A binding, near-WT levels of GDF11 binding, near-WT levels of BMP10 binding, a slight increase in BMP6 binding, and near-WT levels of BMP9 binding, compared to binding profiles of an unmodified ActRIIB-Fc protein (e.g., WT). In some embodiments, an ActRIIB variant comprises a mutation at position L79, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 395. In some embodiments, the variant ActRIIB polypeptide comprises a tryptophan at the position corresponding to L79 of SEQ ID NO: 2. In some embodiments, the amino acid sequence of SEQ ID NO: 395 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a modest reduction in activin A binding, near-WT levels of GDF11 binding, near-WT levels of BMP10 binding, a slight increase in BMP6 binding, and near-WT levels of BMP9 binding, compared to binding profiles of an unmodified ActRIIB-Fc protein (e.g., WT). In some embodiments, an ActRIIB variant of the present disclosure displays a modest reduction in activin B binding, compared to binding profiles of an unmodified ActRIIB-Fc protein (e.g., WT). In some embodiments, an ActRIIB variant comprises a mutation at any one of positions L79, and/or F82, wherein the variant displays a modest reduction in activin A binding, near-WT levels of GDF11 binding, near-WT levels of BMP10 binding, a slight increase in BMP6 binding, and near-WT levels of BMP9 binding, compared to binding profiles of an unmodified ActRIIB-Fc protein (e.g., WT). In some embodiments, an ActRIIB variant comprises a mutation at any one of positions L79, and/or F82, wherein the variant exhibits a modest reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 431. In some embodiments, the variant ActRIIB polypeptide comprises a phenylalanine at the position corresponding to L79 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises a phenylalanine at the position corresponding to L79 of SEQ ID NO: 2 and an aspartic acid at the position corresponding to F82 of SEQ ID NO: 2. In some embodiments, the amino acid sequence of SEQ ID NO: 431 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays a minor reduction in activin A binding, a modest reduction in GDF11 binding, a modest reduction in BMP10 binding, a significant reduction in BMP6 binding, and a significant reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions L57, F82, and/or N83, wherein the variant displays a minor reduction in activin A binding, a modest reduction in GDF11 binding, a modest reduction in BMP10 binding, a significant reduction in BMP6 binding, and a significant reduction in BMP9 binding, compared to binding profiles of an unmodified ActRIIB-Fc protein (e.g., WT). In some embodiments, an ActRIIB variant comprises a mutation at any one of positions L57, F82, and/or N83, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 497. In some embodiments, the variant ActRIIB polypeptide comprises a valine at the position corresponding to L57 of SEQ ID NO: 2. In some embodiments, the variant ActRIIB polypeptide comprises a tyrosine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a valine at the position corresponding to L57 of SEQ ID NO: 2, a tyrosine at the position corresponding to F82 of SEQ ID NO: 2, and an arginine at the position corresponding to N83 of SEQ ID NO: 2. In some embodiments, the amino acid sequence of SEQ ID NO: 497 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays near-WT levels of activin A binding, near-WT levels of GDF11 binding, a modest reduction in BMP10 binding, a modest reduction in BMP6 binding, and a modest reduction in BMP9 binding, compared to binding profiles of an unmodified ActRIIB-Fc protein (e.g., WT). In some embodiments, an ActRIIB variant of the present disclosure displays a minor reduction in activin B binding, compared to binding profiles of an unmodified ActRIIB-Fc protein (e.g., WT). In some embodiments, an ActRIIB variant comprises a mutation at any one of positions L57, and/or F82, wherein the variant displays near-WT levels of activin A binding, near-WT levels of GDF11 binding, a modest reduction in BMP10 binding, a modest reduction in BMP6 binding, and a modest reduction in BMP9 binding, compared to binding profiles of an unmodified ActRIIB-Fc protein (e.g., WT). In some embodiments, an ActRIIB variant comprises a mutation at any one of positions L57, and/or F82, wherein the variant exhibits a minor reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 422. In some embodiments, the variant ActRIIB polypeptide comprises an arginine at the position corresponding to L57 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an arginine at the position corresponding to L57 of SEQ ID NO: 2 and an aspartic acid at the position corresponding to F82 of SEQ ID NO: 2. In some embodiments, the amino acid sequence of SEQ ID NO: 422 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays near-WT levels of activin A binding, near-WT levels of GDF11 binding, near-WT levels of BMP10 binding, a minimal binding to BMP6, and a minor reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at position E94, wherein the variant displays near-WT levels of activin A binding, near-WT levels of GDF11 binding, near-WT levels of BMP10 binding, a minimal binding to BMP6, and a minor reduction in BMP9 binding, compared to binding profiles of an unmodified ActRIIB-Fc protein (e.g., WT). In some embodiments, an ActRIIB variant comprises a mutation at position E94, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 413. In some embodiments, the variant ActRIIB polypeptide comprises a lysine at the position corresponding to E94 of SEQ ID NO: 2. In some embodiments, the amino acid sequence of SEQ ID NO: 413 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays near-WT levels of activin A binding, near-WT levels of GDF11 binding, near-WT levels of BMP10 binding, near-WT levels of BMP6 binding, and a minor reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions E52 and/or F82, wherein the variant displays near-WT levels of activin A binding, near-WT levels of GDF11 binding, near-WT levels of BMP10 binding, near-WT levels of BMP6 binding, and a minor reduction in BMP9 binding, compared to binding profiles of an unmodified ActRIIB-Fc protein (e.g., WT). In some embodiments, an ActRIIB variant comprises a mutation at any one of positions E52 and/or F82, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 398. In some embodiments, the variant ActRIIB polypeptide comprises an aspartic acid at the position corresponding to F82 of SEQ ID NO: 2. In some embodiments, the amino acid sequence of SEQ ID NO: 398 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 401. In some embodiments, the variant ActRIIB polypeptide comprises a glutamic acid at the position corresponding to F82 of SEQ ID NO: 2. In some embodiments, the amino acid sequence of SEQ ID NO: 401 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 407. In some embodiments, the variant ActRIIB polypeptide comprises a serine at the position corresponding to F82 of SEQ ID NO: 2. In some embodiments, the amino acid sequence of SEQ ID NO: 407 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 404. In some embodiments, the variant ActRIIB polypeptide comprises an leucine at the position corresponding to F82 of SEQ ID NO: 2. In some embodiments, the amino acid sequence of SEQ ID NO: 404 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 419. In some embodiments, the variant ActRIIB polypeptide comprises an aspartic acid at the position corresponding to E52 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an aspartic acid at the position corresponding to E52 of SEQ ID NO: 2 and a threonine at the position corresponding to F82 of SEQ ID NO: 2. In some embodiments, the amino acid sequence of SEQ ID NO: 419 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays near-WT levels of activin A binding, near-WT levels of GDF11 binding, near-WT levels of BMP10 binding, near-WT levels of BMP6 binding, and near-WT levels of BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions E52 and/or F82, wherein the variant displays near-WT levels of activin A binding, near-WT levels of GDF11 binding, near-WT levels of BMP10 binding, near-WT levels of BMP6 binding, and near-WT levels of BMP9 binding, compared to binding profiles of an unmodified ActRIIB-Fc protein (e.g., WT). In some embodiments, an ActRIIB variant comprises a mutation at any one of positions E52 and/or F82, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 371. In some embodiments, the variant ActRIIB polypeptide comprises an asparagine at the position corresponding to E52 of SEQ ID NO: 2. In some embodiments, the amino acid sequence of SEQ ID NO: 371 may optionally be provided with the lysine removed from the C-terminus. The G1Fc region may also comprise a mutation to an alanine at position 234 or a mutation to an alanine at position 235, or a combination thereof. In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 410. In some embodiments, the variant ActRIIB polypeptide comprises a tyrosine at the position corresponding to F82 of SEQ ID NO: 2. In some embodiments, the amino acid sequence of SEQ ID NO: 410 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays near-WT levels of activin A binding, near-WT levels of GDF11 binding, near-WT levels of BMP10 binding, near-WT levels of BMP6 binding, and a modest reduction in BMP9 binding, compared to binding profiles of an unmodified ActRIIB-Fc protein (e.g., WT). In some embodiments, an ActRIIB variant of the present disclosure displays a minor reduction in activin B binding, compared to binding profiles of an unmodified ActRIIB-Fc protein (e.g., WT). In some embodiments, an ActRIIB variant comprises a mutation at any one of positions E52 and/or F82, wherein the variant displays near-WT levels of activin A binding, near-WT levels of GDF11 binding, near-WT levels of BMP10 binding, near-WT levels of BMP6 binding, and a modest reduction in BMP9 binding, compared to binding profiles of an unmodified ActRIIB-Fc protein (e.g., WT). In some embodiments, an ActRIIB variant comprises a mutation at any one of positions E52 and/or F82, wherein the variant exhibits a minor reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 416. In some embodiments, the variant ActRIIB polypeptide comprises an aspartic acid at the position corresponding to E52 of SEQ ID NO: 2. In some embodiments, the variant ActRIIB polypeptide comprises an aspartic acid at the position corresponding to F82 of SEQ ID NO: 2. In some embodiments, the variant ActRIIB polypeptide comprises an aspartic acid at the position corresponding to E52 of SEQ ID NO: 2 and an aspartic acid at the position corresponding to F82 of SEQ ID NO: 2. In some embodiments, the amino acid sequence of SEQ ID NO: 416 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays near-WT levels of activin A binding, near-WT levels of GDF11 binding, a minor reduction in BMP10 binding, near-WT levels of BMP6 binding, and a modest reduction in BMP9 binding, compared to binding profiles of an unmodified ActRIIB-Fc protein (e.g., WT). In some embodiments, an ActRIIB variant of the present disclosure displays a minor reduction in activin B binding, compared to binding profiles of an unmodified ActRIIB-Fc protein (e.g., WT). In some embodiments, an ActRIIB variant comprises a mutation at any one of positions L57 and/or F82, wherein the variant displays near-WT levels of activin A binding, near-WT levels of GDF11 binding, a minor reduction in BMP10 binding, near-WT levels of BMP6 binding, and a modest reduction in BMP9 binding, compared to binding profiles of an unmodified ActRIIB-Fc protein (e.g., WT). In some embodiments, an ActRIIB variant comprises a mutation at any one of positions L57 and/or F82, wherein the variant exhibits a minor reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 428. In some embodiments, the variant ActRIIB polypeptide comprises an arginine at the position corresponding to L57 of SEQ ID NO: 2. In some embodiments, the variant ActRIIB polypeptide comprises a threonine at the position corresponding to F82 of SEQ ID NO: 2. In some embodiments, the variant ActRIIB polypeptide comprises an arginine at the position corresponding to L57 of SEQ ID NO: 2 and a threonine at the position corresponding to F82 of SEQ ID NO: 2. In some embodiments, the amino acid sequence of SEQ ID NO: 428 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays near-WT levels of activin A binding, near-WT levels of GDF11 binding, a minor reduction in BMP10 binding, near-WT levels of BMP6 binding, and a modest reduction in BMP9 binding, compared to binding profiles of an unmodified ActRIIB-Fc protein (e.g., WT). In some embodiments, an ActRIIB variant of the present disclosure displays near-WT levels of activin B binding, compared to binding profiles of an unmodified ActRIIB-Fc protein (e.g., WT). In some embodiments, an ActRIIB variant comprises a mutation at any one of positions L57 and/or F82, wherein the variant displays near-WT levels of activin A binding, near-WT levels of GDF11 binding, a minor reduction in BMP10 binding, near-WT levels of BMP6 binding, and a modest reduction in BMP9 binding, compared to binding profiles of an unmodified ActRIIB-Fc protein (e.g., WT). In some embodiments, an ActRIIB variant comprises a mutation at any one of positions L57 and/or F82, wherein the variant exhibits near-WT levels of activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 425. In some embodiments, the variant ActRIIB polypeptide comprises an arginine at the position corresponding to L57 of SEQ ID NO: 2. In some embodiments, the variant ActRIIB polypeptide comprises a serine at the position corresponding to F82 of SEQ ID NO: 2. In some embodiments, the variant ActRIIB polypeptide comprises an arginine at the position corresponding to L57 of SEQ ID NO: 2 and a serine at the position corresponding to F82 of SEQ ID NO: 2. In some embodiments, the amino acid sequence of SEQ ID NO: 425 may optionally be provided with the lysine removed from the C-terminus. The G1Fc region may also comprise a mutation to an alanine at position 234 or a mutation to an alanine at position 235, or a combination thereof.

In some embodiments, an ActRIIB variant of the present disclosure displays near-WT levels of activin A binding, near-WT levels of GDF11 binding, near-WT levels of BMP10 binding, a minor reduction in BMP6 binding, and a modest reduction in BMP9 binding, compared to binding profiles of an unmodified ActRIIB-Fc protein (e.g., WT). In some embodiments, an ActRIIB variant of the present disclosure displays a minor reduction in activin B binding, compared to binding profiles of an unmodified ActRIIB-Fc protein (e.g., WT). In some embodiments, an ActRIIB variant comprises a mutation at any one of positions F82 and/or E94, wherein the variant displays near-WT levels of activin A binding, near-WT levels of GDF11 binding, near-WT levels of BMP10 binding, a minor reduction in BMP6 binding, and a modest reduction in BMP9 binding, compared to binding profiles of an unmodified ActRIIB-Fc protein (e.g., WT). In some embodiments, an ActRIIB variant comprises a mutation at any one of positions F82 and/or E94, wherein the variant exhibits a minor reduction in activin B binding, compared to binding profiles of an unmodified ActRIIB-Fc protein (e.g., WT). In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 449. In some embodiments, the variant ActRIIB polypeptide comprises an isoleucine at the position corresponding to F82 of SEQ ID NO: 2. In some embodiments, the variant ActRIIB polypeptide comprises a lysine at the position corresponding to E94 of SEQ ID NO: 2. In some embodiments, the variant ActRIIB polypeptide comprises an isoleucine at the position corresponding to F82 of SEQ ID NO: 2 and a lysine at the position corresponding to E94 of SEQ ID NO: 2. In some embodiments, the amino acid sequence of SEQ ID NO: 449 may optionally be provided with the lysine removed from the C-terminus. The G1Fc region may also comprise a mutation to an alanine at position 234 or a mutation to an alanine at position 235, or a combination thereof.

In certain embodiments, the present disclosure contemplates further mutations of the variant ActRIIB polypeptides so as to alter the glycosylation of the polypeptide. Exemplary glycosylation sites in variant ActRIIB polypeptides are illustrated in FIG. 4. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (where “X” is any amino acid) which is specifically recognized by appropriate cellular glycosylation enzymes. The alteration may also be made by the addition of, or substitution by, one or more serine or threonine residues to the sequence of the wild-type ActRIIB polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a variant ActRIIB polypeptide is by chemical or enzymatic coupling of glycosides to the variant ActRIIB polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on a variant ActRIIB polypeptide may be accomplished chemically and/or enzymatically. Chemical deglycosylation may involve, for example, exposure of the variant ActRIIB polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on variant ActRIIB polypeptides can be achieved by the use of a variety of endo- and exo-glycosidases as described by Thotakura et al. (1987) Meth. Enzymol. 138:350. The sequence of a variant ActRIIB polypeptide may be adjusted, as appropriate, depending on the type of expression system used, as mammalian, yeast, insect, and plant cells may all introduce differing glycosylation patterns that can be affected by the amino acid sequence of the peptide. In general, variant ActRIIB proteins for use in humans will be expressed in a mammalian cell line that provides proper glycosylation, such as HEK293 or CHO cell lines, although other mammalian expression cell lines are expected to be useful as well.

This disclosure further contemplates a method of generating variants, particularly sets of combinatorial variants of an ActRIIB polypeptide, including, optionally, truncation variants; pools of combinatorial mutants are especially useful for identifying functional variant sequences. The purpose of screening such combinatorial libraries may be to generate, for example, variant ActRIIB polypeptides which have altered properties, such as altered pharmacokinetics, or altered ligand binding. A variety of screening assays are provided below, and such assays may be used to evaluate variants. For example, a variant ActRIIB polypeptide may be screened for ability to bind to an ActRIIB polypeptide, to prevent binding of an ActRIIB ligand to an ActRIIB polypeptide.

The activity of an ActRIIB polypeptide or its variants may also be tested in a cell-based or in vivo assay. For example, the effect of a variant ActRIIB polypeptide on the expression of genes involved in bone production in an osteoblast or precursor may be assessed. This may, as needed, be performed in the presence of one or more recombinant ActRIIB ligand protein (e.g., BMP7), and cells may be transfected so as to produce an ActRIIB polypeptide and/or variants thereof, and optionally, an ActRIIB ligand. Likewise, an ActRIIB polypeptide may be administered to a mouse or other animal, and one or more bone properties, such as density or volume may be assessed. The healing rate for bone fractures may also be evaluated. Similarly, the activity of an ActRIIB polypeptide or its variants may be tested in muscle cells, adipocytes, and neuronal cells for any effect on growth of these cells, for example, by the assays as described below. Such assays are well known and routine in the art. A SMAD-responsive reporter gene may be used in such cell lines to monitor effects on downstream signaling.

Combinatorially-derived variants can be generated which have a selective potency relative to a naturally occurring ActRIIB polypeptide. Such variant proteins, when expressed from recombinant DNA constructs, can be used in gene therapy protocols. Likewise, mutagenesis can give rise to variants which have intracellular half-lives dramatically different than the corresponding a wild-type ActRIIB polypeptide. For example, the altered protein can be rendered either more stable or less stable to proteolytic degradation or other processes which result in destruction of, or otherwise inactivation of a native ActRIIB polypeptide. Such variants, and the genes which encode them, can be utilized to alter ActRIIB polypeptide levels by modulating the half-life of the ActRIIB polypeptides. For instance, a short half-life can give rise to more transient biological effects and, when part of an inducible expression system, can allow tighter control of recombinant ActRIIB polypeptide levels within the cell.

In certain embodiments, the variant ActRIIB polypeptides of the disclosure may further comprise post-translational modifications in addition to any that are naturally present in the variant ActRIIB polypeptides. Such modifications include, but are not limited to, acetylation, carboxylation, glycosylation, phosphorylation, lipidation, and acylation. As a result, the modified variant ActRIIB polypeptides may contain non-amino acid elements, such as polyethylene glycols, lipids, poly- or mono-saccharide, and phosphates. Effects of such non-amino acid elements on the functionality of a variant ActRIIB polypeptide may be tested as described herein for other variant ActRIIB polypeptides. When a variant ActRIIB polypeptide is produced in cells by cleaving a nascent form of the variant ActRIIB polypeptide, post-translational processing may also be important for correct folding and/or function of the protein. Different cells (such as CHO, HeLa, MDCK, 293, WI38, NIH-3T3 or HEK293) have specific cellular machinery and characteristic mechanisms for such post-translational activities and may be chosen to ensure the correct modification and processing of the variant ActRIIB polypeptides.

In certain aspects, variant ActRIIB polypeptides include fusion proteins having at least a portion of the variant ActRIIB polypeptides and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (e.g., an Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt- conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the variant ActRIIB polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for factor Xa or thrombin, which allows the relevant protease to partially digest the fusion proteins and thereby liberate the recombinant proteins therefrom. The liberated proteins can then be isolated from the fusion domain by subsequent chromatographic separation. In certain preferred embodiments, a variant ActRIIB polypeptide is fused with a domain that stabilizes the variant ActRIIB polypeptide in vivo (a “stabilizer” domain). By “stabilizing” is meant anything that increases serum half life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains (that confer an additional biological function, such as further stimulation of muscle growth).

In certain aspects the polypeptides disclosed herein may form homomeric variant ActRIIB polypeptides. In some embodiments, each fusion polypeptide chain in the protein complex comprises the same variant ActRIIB polypeptide as any other such chain in the complex. In certain aspects, the polypeptides disclosed herein may form heteromultimers comprising at least one variant ActRIIB polypeptide associated, covalently or non-covalently, with at least one unmodified ActRIIB polypeptide or at least one variant ActRIIB polypeptide different from the first variant ActRIIB polypeptide. For example, in some embodiments the disclosure provides for an ActRIIB heteromultimer (e.g., dimer), wherein the heteromultimer comprises: a) a first variant ActRIIB polypeptide comprising one or more of any of the amino acid substitutions disclosed herein multimerizes (e.g., dimerizes), and b) a second variant ActRIIB polypeptide having a different amino acid substitution or a different combination of amino acid substitutions as the first ActRIIB polypeptide.

In certain aspects, the polypeptides disclosed herein may form heteromultimers comprising at least one variant ActRIIB polypeptide associated, covalently or non-covalently, with at least one ALK4 polypeptide, including fragments and variants thereof. In some embodiments, the polypeptides disclosed herein may form heteromultimers comprising at least one variant ActRIIB polypeptide associated, covalently or non-covalently, with at least one ALK7 polypeptide, including fragments and variants thereof. In some embodiments, heteromeric polypeptides disclosed herein form heterodimers, although higher order heteromultimers are also included such as, but not limited to, heterotrimers, heterotetramers, and further oligomeric structures.

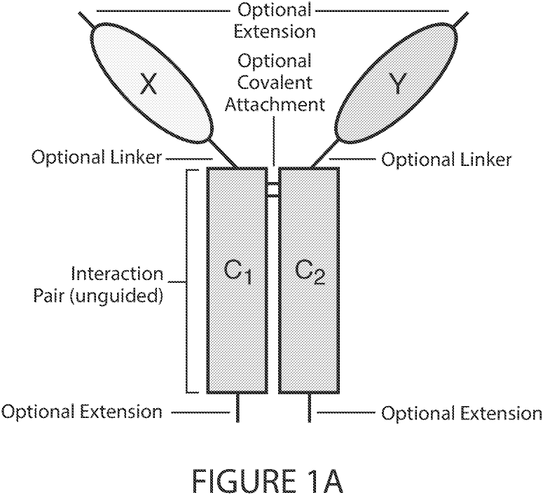

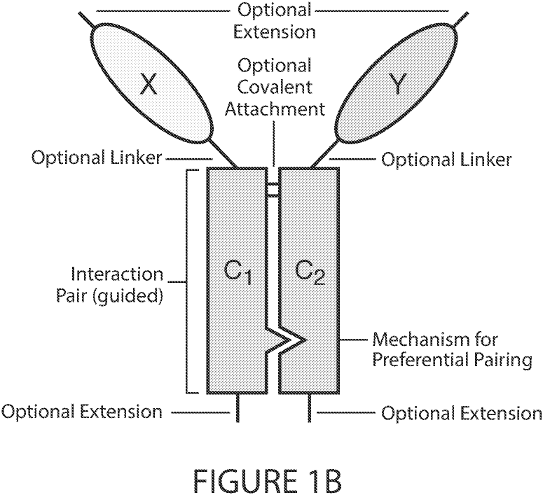

In some embodiments, variant ActRIIB polypeptides of the present disclosure comprise at least one multimerization domain. As disclosed herein, the term “multimerization domain” refers to an amino acid or sequence of amino acids that promote covalent or non-covalent interaction between at least a first polypeptide and at least a second polypeptide. Variant ActRIIB polypeptides disclosed herein may be joined covalently or non-covalently to a multimerization domain. Preferably, a multimerization domain promotes interaction between a first polypeptide (e.g., variant ActRIIB polypeptide) and a second polypeptide (e.g., an ALK4 polypeptide or an ALK7 polypeptide) to promote heteromultimer formation (e.g., heterodimer formation), and optionally hinders or otherwise disfavors homomultimer formation (e.g., homodimer formation), thereby increasing the yield of desired heteromultimer (see, e.g., FIG. 1B). In some embodiments, variant ActRIIB polypeptide of the disclosure form homodimers. In some embodiments, variant ActRIIB polypeptides may from heterodimers through covalent interactions. In some embodiments, variant ActRIIB polypeptides may from heterodimers through non-covalent interactions. In some embodiments, variant ActRIIB polypeptides may from heterodimers through both covalent and non-covalent interactions.

In certain aspects, a variant ActRIIB polypeptide, including homomultimers thereof (e.g., homodimers), binds to one or more TGF-beta superfamily ligands. In some embodiments, variant ActRIIB polypeptide, including homomultimers thereof, binds to one or more TGF-beta superfamily ligands with a K_{D} of at least 1 × 10⁻⁷ M. In some embodiments, the one or more TGF-beta superfamily ligands is selected from the group consisting of: activin A, activin B, GDF8, GDF11, and BMP10.

In certain aspects, a variant ActRIIB polypeptide, including homomultimers thereof (e.g., homodimers), inhibits one or more TGF-beta super family ligands. In some embodiments, variant ActRIIB polypeptide, including homomultimers thereof, inhibits signaling of one or more TGF-beta super family ligands. In some embodiments, variant ActRIIB polypeptide, including homomultimers thereof, inhibits Smad signaling of one or more TGF-beta super family ligands. In some embodiments, variant ActRIIB polypeptide, including homomultimers thereof, inhibits signaling of one or more TGF-beta super family ligands in a cell-based assay. In some embodiments, variant ActRIIB polypeptide, including homomultimers thereof, inhibits one or more TGF-beta super family ligands selected from the group consisting of: activin A, activin B, GDF8, GDF11, and BMP10.

In certain embodiments, the disclosure relates to a heteromultimer comprising a first variant ActRIIB-Fc fusion protein and a second variant ActRIIB-Fc fusion protein, wherein the first variant ActRIIB polypeptide does not comprise the amino acid sequence of the second variant ActRIIB polypeptide. In some embodiments, an ActRIIB-Fc:ActRIIB-Fc heteromultimer binds to one or more TGF-beta superfamily ligands such as those described herein. In some embodiments, an ActRIIB-Fc:ActRIIB-Fc heteromultimer inhibits signaling of one or more TGF-beta superfamily ligands such as those described herein. In some embodiments, an ActRIIB-Fc:ActRIIB-Fc heteromultimer is a heterodimer.

In some embodiments, the first ActRIIB polypeptide comprises one or more amino acid substitutions at the amino acid positions corresponding to any one of N35, E50, E52, K55, L57, Y60, G68, K74, W78, L79, F82, N83, and E94 of SEQ ID NO: 2. In some embodiments, the first ActRIIB polypeptide comprises one or more amino acid substitutions at the amino acid positions corresponding to any one of L79, A24, K74, R64, P129, P130, E37, R40, D54, R56, W78, D80, and F82 of SEQ ID NO: 2. In some embodiments, the first ActRIIB polypeptide comprises one or more amino acid substitutions at the amino acid positions corresponding to any one of L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the one or more amino acid substitutions is selected from the group consisting of: L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G. In some embodiments, the first ActRIIB polypeptide comprises one or more amino acid substitutions at the amino acid positions correspond to any one of A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132. In some embodiments, the one or more amino acid substitutions of selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, the second ActRIIB polypeptide comprises one or more amino acid substitutions at the amino acid positions corresponding to any one of L79, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G of SEQ ID NO: 2. In some embodiments, the one or more amino acid substitutions is selected from the group consisting of: L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G. In some embodiments, the second ActRIIB polypeptide comprises one or more amino acid substitutions at the amino acid positions correspond to any one of A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132. In some embodiments, the one or more amino acid substitutions of selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, the first ActRIIB polypeptide and/or the second ActRIIB polypeptide comprise one or more amino acid modification that promote heteromultimer formation. In some embodiments, the first ActRIIB polypeptide and/or the second ActRIIB polypeptide comprise one or more amino acid modification that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 36, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB polypeptide does not comprise the amino acid sequence of the second ActRIIB polypeptide. In some embodiments, the first ActRIIB polypeptide comprises a glutamic acid at the amino acid position corresponding to 55 of SEQ ID NO: 2. In some embodiments, the second ActRIIB polypeptide does not comprise a glutamic acid at the amino acid position corresponding to 55 of SEQ ID NO: 2. In some embodiments, the second ActRIIB polypeptide comprises a lysine at the amino acid position corresponding to 55 of SEQ ID NO: 2. In some embodiments, the first ActRIIB polypeptide comprises one or more amino acid substitutions at the amino acid positions corresponding to any one of F82, L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of L79, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the first ActRIIB polypeptide comprises one or more amino acid substitutions at the amino acid positions corresponding to any one of A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, the second ActRIIB polypeptide comprises one or more amino acid substitutions at the amino acid positions corresponding to any one of L79, A24, K74, R64, P129, P130, E37, R40, D54, R56, W78, D80, and F82 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, the first ActRIIB polypeptide and/or the second ActRIIB polypeptide comprise one or more amino acid modification that promote heteromultimer formation. In some embodiments, the first ActRIIB polypeptide and/or the second ActRIIB polypeptide comprise one or more amino acid modification that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 39, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB polypeptide does not comprise the amino acid sequence of the second ActRIIB polypeptide. In some embodiments, the first ActRIIB polypeptide comprises an isoleucine at the amino acid position corresponding to 82 of SEQ ID NO: 2. In some embodiments, the second ActRIIB polypeptide does not comprise an isoleucine acid at the amino acid position corresponding to 82 of SEQ ID NO: 2. In some embodiments, the second ActRIIB polypeptide comprises a phenylalanine at the amino acid position corresponding to 82 of SEQ ID NO: 2. In some embodiments, the first ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the second ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the first ActRIIB polypeptide comprises one or more amino acid substitutions at the amino acid positions corresponding to any one of A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, 129N, P129S, P130A, P130R, and A132N.. In some embodiments, the second ActRIIB polypeptide comprises one or more amino acid substitutions at the amino acid positions corresponding to any one of L79, A24, K74, R64, 129, P130, E37, R40, D54, R56, W78, D80, and F82 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, 130A, P130R, and A132N. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modification that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 42, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB polypeptide does not comprise the amino acid sequence of the second ActRIIB polypeptide. In some embodiments, first ActRIIB polypeptide comprises a lysine at the amino acid position corresponding to 82 of SEQ ID NO: 2. In some embodiments, the second ActRIIB polypeptide does not comprise a lysine at the amino acid position corresponding to 82 of SEQ ID NO: 2. In some embodiments, the second ActRIIB polypeptide comprises a phenylalanine at the amino acid position corresponding to 82 of SEQ ID NO: 2. In some embodiments, the first ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the second ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, 130R, E37A, R40A, D54A, R56A, K74F, K74I, K74Y, W78A, D80A, D80F, D80G, D80I, D80K, D80M, D80M, D80N, and D80R. In some embodiments, the first ActRIIB polypeptide comprises one or more amino acid substitutions at the amino acid positions corresponding to any one of A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.. In some embodiments, the second ActRIIB polypeptide comprises one or more amino acid substitutions at the amino acid positions corresponding to any one of L79, A24, K74, R64, P129, P130, E37, R40, D54, R56, W78, D80, and F82 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modifications that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 524, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB polypeptide does not comprise the amino acid sequence of the second ActRIIB polypeptide. In some embodiments, first ActRIIB polypeptide comprises a lysine at the amino acid position corresponding to 82 of SEQ ID NO: 2. In some embodiments, the second ActRIIB polypeptide does not comprise a lysine at the amino acid position corresponding to 82 of SEQ ID NO: 2. In some embodiments, the second ActRIIB polypeptide comprises a phenylalanine at the amino acid position corresponding to 82 of SEQ ID NO: 2. In some embodiments, the first ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the second ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the first ActRIIB polypeptide comprises one or more amino acid substitutions at the amino acid positions corresponding to any one of A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.. In some embodiments, the second ActRIIB polypeptide comprises one or more amino acid substitutions at the amino acid positions corresponding to any one of L79, A24, K74, R64, P129, P130, E37, R40, D54, R56, W78, D80, and F82 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modifications that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 45, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 48, wherein the first ActRIIB polypeptide does not comprise the amino acid sequence of the second ActRIIB polypeptide. In some embodiments, the first ActRIIB polypeptide comprises an acidic amino acid position corresponding to 79 of SEQ ID NO: 2. In some embodiments, the acidic amino acid is an aspartic acid. In some embodiments, the acidic amino acid is a glutamic acid. In some embodiments, the second ActRIIB polypeptide does not comprise an acidic acid (e.g., aspartic acid or glutamic acid) at the amino acid position corresponding to 79 of SEQ ID NO: 2. In some embodiments, the second ActRIIB polypeptide comprises a leucine at the amino acid position corresponding to 79 of SEQ ID NO: 2. In some embodiments, the first ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the first ActRIIB polypeptide comprises one or more amino acid substitutions at the amino acid positions corresponding to any one of A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.. In some embodiments, the second ActRIIB polypeptide comprises one or more amino acid substitutions at the amino acid positions corresponding to any one of L79, A24, K74, R64, P129, P130, E37, R40, D54, R56, W78, D80, and F82 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modifications that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 50, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 52, wherein the first ActRIIB polypeptide does not comprise the amino acid sequence of the second ActRIIB polypeptide. In some embodiments, the first ActRIIB polypeptide comprises an acidic amino acid position corresponding to 79 of SEQ ID NO: 2. In some embodiments, the acidic amino acid is an aspartic acid. In some embodiments, the acidic amino acid is a glutamic acid. In some embodiments, the second ActRIIB polypeptide does not comprise an acidic acid (e.g., aspartic acid or glutamic acid) at the amino acid position corresponding to 79 of SEQ ID NO: 2. In some embodiments, the second ActRIIB polypeptide comprises a leucine at the amino acid position corresponding to 79 of SEQ ID NO: 2. In some embodiments, the first ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the first ActRIIB polypeptide comprises one or more amino acid substitutions at the amino acid positions corresponding to any one of A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.. In some embodiments, the second ActRIIB polypeptide comprises one or more amino acid substitutions at the amino acid positions corresponding to any one of L79, A24, K74, R64, P129, P130, E37, R40, D54, R56, W78, D80, and F82 of SEQ ID NO: 2. In some embodiments, the one or more amino acid substitutions is selected from the group consisting of: A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modifications that inhibit heteromultimer formation. In some embodiments, the heteromultimer is a heterodimer. In certain embodiments, the disclosure relates to a heteromultimer comprising a first variant ActRIIB-Fc fusion protein and a second variant ActRIIB-Fc fusion protein, wherein the second variant ActRIIB-Fc fusion protein differs from that present in the first polypeptide. In some embodiments, an ActRIIB-Fc:ActRIIB-Fc heteromultimers binds to one or more TGF-beta superfamily ligands such as those described herein. In some embodiments, an ActRIIB-Fc:ActRIIB-Fc heteromultimers inhibit signaling of one or more TGF-beta superfamily ligands such as those described herein. In some embodiments, an ActRIIB-Fc:ActRIIB-Fc heteromultimers is a heterodimer.

In certain embodiments, the disclosure relates to a heteromultimer comprising at least one ALK7-Fc fusion protein and at least one ActRIIB-Fc fusion protein. In some embodiments, an ALK7-Fc:ActRIIB-Fc heteromultimers binds to one or more TGF-beta superfamily ligands such as those described herein. In some embodiments, an ALK7-Fc:ActRIIB-Fc heteromultimers inhibit signaling of one or more TGF-beta superfamily ligands such as those described herein. In some embodiments, an ALK7-Fc:ActRIIB-Fc heteromultimers is a heterodimer.

In certain embodiments, the disclosure relates to a heteromultimer comprising at least one ALK4-Fc fusion protein and at least one ActRIIB-Fc fusion protein. In some embodiments, an ALK4-Fc:ActRIIB-Fc heteromultimers binds to one or more TGF-beta superfamily ligands such as those described herein. In some embodiments, an ALK4-Fc:ActRIIB-Fc heteromultimers inhibit signaling of one or more TGF-beta superfamily ligands such as those described herein. In some embodiments, an ALK4-Fc:ActRIIB-Fc heteromultimers is a heterodimer.

In certain aspects, the present disclosure relates to protein complexes that comprise an ALK4 polypeptide. As used herein, the term “ALK4” refers to a family of activin receptor-like kinase-4 proteins from any species and variants derived from such ALK4 proteins by mutagenesis or other modification. Reference to ALK4 herein is understood to be a reference to any one of the currently identified forms. Members of the ALK4 family are generally transmembrane proteins, composed of a ligand-binding extracellular domain with a cysteine-rich region, a transmembrane domain, and a cytoplasmic domain with predicted serine/threonine kinase activity.

The term “ALK4 polypeptide” includes polypeptides comprising any naturally occurring polypeptide of an ALK4 family member as well as any variants thereof (including mutants, fragments, fusions, and peptidomimetic forms) that retain a useful activity.

A human ALK4 precursor protein sequence (NCBI Ref Seq NP_004293) is as follows:

1 MAESAGASSF FPLVVLLLAG SGGSGPRGVQ ALLCACTSCL QANYTCETDG ACMVSIFNLD
61 GMEHHVRTCI PKVELVPAGK PFYCLSSEDL RNTHCCYTDY CNRIDLRVPS GHLKEPEHPS
121 MWGPVELVGI IAGPVFLLFL IIIIVFLVIN YHQRVYHNRQ RLDMEDPSCE MCLSKDKTLQ
181 DLVYDLSTSG SGSGLPLFVQ RTVARTIVLQ EIIGKGRFGE VWRGRWRGGD VAVKIFSSRE
241 ERSWFREAEI YQTVMLRHEN ILGFIAADNK DNGTWTQLWL VSDYHEHGSL FDYLNRYTVT
301 IEGMIKLALS AASGLAHLHM EIVGTQGKPG IAHRDLKSKN ILVKKNGMCA IADLGLAVRH
361 DAVTDTIDIA PNQRVGTKRY MAPEVLDETI NMKHFDSFKC ADIYALGLVY WEIARRCNSG
421 GVHEEYQLPY YDLVPSDPSI EEMRKVVCDQ KLRPNIPNWW QSYEALRVMG KMMRECWYAN
481 GAARLTALRI KKTLSQLSVQ EDVKI (SEQ ID NO: 84)

The signal peptide is indicated by a single underline and the extracellular domain is indicated in bold font.

A processed extracellular human ALK4 polypeptide sequence is as follows:

SGPRGVQALLCACTSCLQANYTCETDGACMVSIFNLDGMEHHVRTCIPKV
ELVPAGKPFYCLSSEDLRNTHCCYTDYCNRIDLRVPSGHLKEPEHPSMWG
PVE (SEQ ID NO: 86)

A nucleic acid sequence encoding an ALK4 precursor protein is shown in SEQ ID NO: 221), corresponding to nucleotides 78-1592 of Genbank Reference Sequence NM_004302.4. A nucleic acid sequence encoding the extracellular ALK4 polypeptide is shown in SEQ ID NO: 222.

An alternative isoform of human ALK4 precursor protein sequence, isoform C (NCBI Ref Seq NP_064733.3), is as follows:

1 MAESAGASSF FPLVVLLLAG SGGSGPRGVQ ALLCACTSCL QANYTCETDG ACMVSIFNLD
61 GMEHHVRTCI PKVELVPAGK PFYCLSSEDL RNTHCCYTDY CNRIDLRVPS GHLKEPEHPS
121 MWGPVELVGI IAGPVFLLFL IIIIVFLVIN YHQRVYHNRQ RLDMEDPSCE MCLSKDKTLQ
181 DLVYDLSTSG SGSGLPLFVQ RTVARTIVLQ EIIGKGRFGE VWRGRWRGGD VAVKIFSSRE
241 ERSWFREAEI YQTVMLRHEN ILGFIAADNK ADCSFLTLPW EVVMVSAAPK LRSLRLQYKG
301 GRGRARFLFP LNNGTWTQLW LVSDYHEHGS LFDYLNRYTV TIEGMIKLAL SAASGLAHLH
361 MEIVGTQGKP GIAHRDLKSK NILVKKNGMC AIADLGLAVR HDAVTDTIDI APNQRVGTKR
421 YMAPEVLDET INMKHFDSFK CADIYALGLV YWEIARRCNS GGVHEEYQLP YYDLVPSDPS
481 IEEMRKVVCD QKLRPNIPNW WQSYEALRVM GKMMRECWYA NGAARLTALR IKKTLSQLSV
541 QEDVKI (SEQ ID NO: 85)

The signal peptide is indicated by a single underline and the extracellular domain is indicated in bold font.

A processed extracellular ALK4 polypeptide sequence (isoform C) is as follows:

SGPRGVQALLCACTSCLQANYTCETDGACMVSIFNLDGMEHHVRTCIPKV
ELVPAGKPFYCLSSEDLRNTHCCYTDYCNRIDLRVPSGHLKEPEHPSMWG
PVE (SEQ ID NO: 87)

A nucleic acid sequence encoding an ALK4 precursor protein (isoform C) is shown in SEQ ID NO: 223, corresponding to nucleotides 78-1715 of Genbank Reference Sequence NM_020328.3. A nucleic acid sequence encoding the extracellular ALK4 polypeptide (isoform C) is shown in SEQ ID NO: 224.

In certain embodiments, the disclosure relates to heteromultimers that comprise at least one ALK4 polypeptide, which includes fragments, functional variants, and modified forms thereof. Preferably, ALK4 polypeptides for use as disclosed herein (e.g., heteromultimers comprising an ALK4 polypeptide and uses thereof) are soluble (e.g., an extracellular domain of ALK4). In other preferred embodiments, ALK4 polypeptides for use as disclosed herein bind to and/or inhibit (antagonize) activity (e.g., induction of Smad signaling) of one or more TGF-beta superfamily ligands. In some embodiments, heteromultimers of the disclosure comprise at least one ALK4 polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 84, 85, 86, 87, 88, 89, 92, or 93. In some embodiments, heteromultimers of the disclosure consist or consist essentially of at least one ALK4 polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 84, 85, 86, 87, 88, 89, 92, or 93.

In certain aspects, the disclosure relates to a heteromultimer that comprises an ALK4-Fc fusion protein. In some embodiments, the ALK4-Fc fusion protein comprises an ALK4 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23-34 (e.g., amino acid residues 23, 24, 25, 26, 27, 28, 29, 30, 31, 32, 33, 34) SEQ ID NO: 84 or 85, and ends at any one of amino acids 101-126 (e.g., amino acid residues 101, 102, 103, 104, 105, 106, 107, 108, 109, 110, 111, 112, 113, 114, 115, 116, 117, 118, 119, 120, 121, 122, 123, 124, 125, and 126) of SEQ ID NO: 84 or 85. In some embodiments, the ALK4-Fc fusion protein comprises an ALK4 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 34-101 of SEQ ID NOs: 84 or 85. In some embodiments, the ALK4-Fc fusion protein comprises an ALK4 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 23-126 of SEQ ID Nos: 84 or 85. In some embodiments, the ALK4-Fc fusion protein comprises an ALK4 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of any one of SEQ ID Nos: 84, 86, 85, 87, 88, 89, 90, 91, 92, 93, 94, and 95.

In certain embodiments, the polypeptide comprises an ALK4-Fc fusion polypeptide (SEQ ID NO: 88) as follows:

1 MDAMKRGLCC VLLLCGAVFV SPGASGPRGV QALLCACTSC LQANYTCETD
51 GACMVSIFNL DGMEHHVRTC IPKVELVPAG KPFYCLSSED LRNTHCCYTD
101 YCNRIDLRVP SGHLKEPEHP SMWGPVETGG GTHTCPPCPA PELLGGPSVF
151 LFPPKPKDTL MISRTPEVTC VVVDVSHEDP EVKFNWYVDG VEVHNAKTKP
201 REEQYNSTYR VVSVLTVLHQ DWLNGKEYKC KVSNKALPAP IEKTISKAKG
251 QPREPQVYTL PPSREEMTKN QVSLTCLVKG FYPSDIAVEW ESNGQPENNY
301 DTTPPVLDSD GSFFLYSDLT VDKSRWQQGN VFSCSVMHEA LHNHYTQKSL
351 SLSPG (SEQ ID NO: 88)

The leader sequence and linker sequence are underlined. To guide heterodimer formation with certain Fc fusion polypeptides of the disclosure, two amino acid substitutions (replacing lysines with aspartic acids) can be introduced into the Fc domain of the ALK4-Fc fusion polypeptide as indicated by double underline above. The amino acid sequence of SEQ ID NO: 88 may optionally be provided with lysine added at the C-terminus.

This ALK4-Fc fusion protein is encoded by the following nucleic acid (SEQ ID NO: 243):

1 ATGGATGCAA TGAAGAGAGG GCTCTGCTGT GTGCTGCTGC TGTGTGGAGC
51 AGTCTTCGTT TCGCCCGGCG CCTCCGGGCC CCGGGGGGTC CAGGCTCTGC
101 TGTGTGCGTG CACCAGCTGC CTCCAGGCCA ACTACACGTG TGAGACAGAT
151 GGGGCCTGCA TGGTTTCCAT TTTCAATCTG GATGGGATGG AGCACCATGT
201 GCGCACCTGC ATCCCCAAAG TGGAGCTGGT CCCTGCCGGG AAGCCCTTCT
251 ACTGCCTGAG CTCGGAGGAC CTGCGCAACA CCCACTGCTG CTACACTGAC
301 TACTGCAACA GGATCGACTT GAGGGTGCCC AGTGGTCACC TCAAGGAGCC
351 TGAGCACCCG TCCATGTGGG GCCCGGTGGA GACCGGTGGT GGAACTCACA
401 CATGCCCACC GTGCCCAGCA CCTGAACTCC TGGGGGGACC GTCAGTCTTC
451 CTCTTCCCCC CAAAACCCAA GGACACCCTC ATGATCTCCC GGACCCCTGA
501 GGTCACATGC GTGGTGGTGG ACGTGAGCCA CGAAGACCCT GAGGTCAAGT
551 TCAACTGGTA CGTGGACGGC GTGGAGGTGC ATAATGCCAA GACAAAGCCG
601 CGGGAGGAGC AGTACAACAG CACGTACCGT GTGGTCAGCG TCCTCACCGT
651 CCTGCACCAG GACTGGCTGA ATGGCAAGGA GTACAAGTGC AAGGTCTCCA
701 ACAAAGCCCT CCCAGCCCCC ATCGAGAAAA CCATCTCCAA AGCCAAAGGG
751 CAGCCCCGAG AACCACAGGT GTACACCCTG CCCCCATCCC GGGAGGAGAT
801 GACCAAGAAC CAGGTCAGCC TGACCTGCCT GGTCAAAGGC TTCTATCCCA
851 GCGACATCGC CGTGGAGTGG GAGAGCAATG GGCAGCCGGA GAACAACTAC
901 GACACCACGC CTCCCGTGCT GGACTCCGAC GGCTCCTTCT TCCTCTATAG
951 CGACCTCACC GTGGACAAGA GCAGGTGGCA GCAGGGGAAC GTCTTCTCAT
1001 GCTCCGTGAT GCATGAGGCT CTGCACAACC ACTACACGCA GAAGAGCCTC
1051 TCCCTGTCTC CGGGT (SEQ ID NO: 243)

The mature ALK4-Fc fusion protein sequence (SEQ ID NO: 89) is as follows and may optionally be provided with lysine added at the C-terminus.

1 SGPRGVQALL CACTSCLQAN YTCETDGACM VSIFNLDGME HHVRTCIPKV
51 ELVPAGKPFY CLSSEDLRNT HCCYTDYCNR IDLRVPSGHL KEPEHPSMWG
101 PVETGGGTHT CPPCPAPELL GGPSVFLFPP KPKDTLMISR TPEVTCVVVD
151 VSHEDPEVKF NWYVDGVEVH NAKTKPREEQ YNSTYRVVSV LTVLHQDWLN
201 GKEYKCKVSN KALPAPIEKT ISKAKGQPRE PQVYTLPPSR EEMTKNQVSL
251 TCLVKGFYPS DIAVEWESNG QPENNYDTTP PVLDSDGSFF LYSDLTVDKS
301 RWQQGNVFSC SVMHEALHNH YTQKSLSLSP G (SEQ ID NO: 89)

In some embodiments, the ALK4-Fc fusion polypeptide (or any Fc fusion polypeptide disclosed herein) employs the tissue plasminogen activator (TPA)leader:

MDAMKRGLCCVLLLCGAVFVSP (SEQ ID NO: 246).

In some embodiments, the ALK4-Fc fusion polypeptide (SEQ ID NO: 92) is as follows and may optionally be provided with lysine removed from the C-terminus.

1 MDAMKRGLCC VLLLCGAVFV SPGASGPRGV QALLCACTSC LQANYTCETD
51 GACMVSIFNL DGMEHHVRTC IPKVELVPAG KPFYCLSSED LRNTHCCYTD
101 YCNRIDLRVP SGHLKEPEHP SMWGPVETGG GTHTCPPCPA PELLGGPSVF
151 LFPPKPKDTL MISRTPEVTC VVVDVSHEDP EVKFNWYVDG VEVHNAKTKP
201 REEQYNSTYR VVSVLTVLHQ DWLNGKEYKC KVSNKALPAP IEKTISKAKG
251 QPREPQVCTL PPSREEMTKN QVSLSCAVKG FYPSDIAVEW ESNGQPENNY
301 KTTPPVLDSD GSFFLVSKLT VDKSRWQQGN VFSCSVMHEA LHNHYTQKSL
351 SLSPGK (SEQ ID NO: 92)

The leader sequence and the linker are underlined. To guide heterodimer formation with certain Fc fusion polypeptides disclosed herein, four amino acid substitutions can be introduced into the Fc domain of the ALK4 fusion polypeptide as indicated by double underline above. The amino acid sequence of SEQ ID NO: 92 may optionally be provided with lysine removed from the C-terminus.

The mature ALK4-Fc fusion protein sequence is as follows and may optionally be provided with lysine removed from the C-terminus.

1 SGPRGVQALL CACTSCLQAN YTCETDGACM VSIFNLDGME HHVRTCIPKV
51 ELVPAGKPFY CLSSEDLRNT HCCYTDYCNR IDLRVPSGHL KEPEHPSMWG
101 PVETGGGTHT CPPCPAPELL GGPSVFLFPP KPKDTLMISR TPEVTCVVVD
151 VSHEDPEVKF NWYVDGVEVH NAKTKPREEQ YNSTYRVVSV LTVLHQDWLN
201 GKEYKCKVSN KALPAPIEKT ISKAKGQPRE PQVCTLPPSR EEMTKNQVSL
251 SCAVKGFYPS DIAVEWESNG QPENNYKTTP PVLDSDGSFF LVSKLTVDKS
301 RWQQGNVFSC SVMHEALHNH YTQKSLSLSP GK (SEQ ID NO: 93)

Purification of various ActRIIB-Fc:ALK4-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

In some embodiments, the ALK4-Fc fusion polypeptide (SEQ ID NO: 247) is as follows and may optionally be provided with lysine removed from the C-terminus.

1 MDAMKRGLCC VLLLCGAVFV SPGASGPRGV QALLCACTSC LQANYTCETD
51 GACMVSIFNL DGMEHHVRTC IPKVELVPAG KPFYCLSSED LRNTHCCYTD
101 YCNRIDLRVP SGHLKEPEHP SMWGPVETGG GTHTCPPCPA PELLGGPSVF
151 LFPPKPKDTL MISRTPEVTC VVVDVSHEDP EVKFNWYVDG VEVHNAKTKP
201 REEQYNSTYR VVSVLTVLHQ DWLNGKEYKC KVSNKALPAP IEKTISKAKG
251 QPREPQVCTL PPSREEMTKN QVSLSCAVKG FYPSDIAVEW ESRGQPENNY
301 KTTPPVLDSR GSFFLVSKLT VDKSRWQQGN VFSCSVMHEA LHNHYTQKSL
351 SLSPGK (SEQ ID NO: 247)

The leader sequence and the linker are underlined. To guide heterodimer formation with certain Fc fusion polypeptides disclosed herein, four amino acid substitutions (replacing a tyrosine with a cysteine, a threonine with a serine, a leucine with an alanine, and a tyrosine with a valine) can be introduced into the Fc domain of the ALK4 fusion polypeptide as indicated by double underline above. To facilitate purification of the ALK4-Fc:ActRIIB-Fc heterodimer, two amino acid substitutions (replacing an asparagine with an arginine and an aspartate with an arginine) can also be introduced into the Fc domain of the ALK4-Fc fusion polypeptide as indicated by double underline above. The amino acid sequence of SEQ ID NO: 247 may optionally be provided with lysine removed from the C-terminus.

This ALK4-Fc fusion polypeptide is encoded by the following nucleic acid (SEQ ID NO: 248):

1 ATGGATGCAA TGAAGAGAGG GCTCTGCTGT GTGCTGCTGC TGTGTGGAGC
51 AGTCTTCGTT TCGCCCGGCG CCTCCGGGCC CCGGGGGGTC CAGGCTCTGC
101 TGTGTGCGTG CACCAGCTGC CTCCAGGCCA ACTACACGTG TGAGACAGAT
151 GGGGCCTGCA TGGTTTCCAT TTTCAATCTG GATGGGATGG AGCACCATGT
201 GCGCACCTGC ATCCCCAAAG TGGAGCTGGT CCCTGCCGGG AAGCCCTTCT
251 ACTGCCTGAG CTCGGAGGAC CTGCGCAACA CCCACTGCTG CTACACTGAC
301 TACTGCAACA GGATCGACTT GAGGGTGCCC AGTGGTCACC TCAAGGAGCC
351 TGAGCACCCG TCCATGTGGG GCCCGGTGGA GACCGGTGGT GGAACTCACA
401 CATGCCCACC GTGCCCAGCA CCTGAACTCC TGGGGGGACC GTCAGTCTTC
451 CTCTTCCCCC CAAAACCCAA GGACACCCTC ATGATCTCCC GGACCCCTGA
501 GGTCACATGC GTGGTGGTGG ACGTGAGCCA CGAAGACCCT GAGGTCAAGT
551 TCAACTGGTA CGTGGACGGC GTGGAGGTGC ATAATGCCAA GACAAAGCCG
601 CGGGAGGAGC AGTACAACAG CACGTACCGT GTGGTCAGCG TCCTCACCGT
651 CCTGCACCAG GACTGGCTGA ATGGCAAGGA GTACAAGTGC AAGGTCTCCA
701 ACAAAGCCCT CCCAGCCCCC ATCGAGAAAA CCATCTCCAA AGCCAAAGGG
751 CAGCCCCGAG AACCACAGGT GTGCACCCTG CCCCCATCCC GGGAGGAGAT
801 GACCAAGAAC CAGGTCAGCC TGTCCTGCGC CGTCAAAGGC TTCTATCCCA
851 GCGACATCGC CGTGGAGTGG GAGAGCCGCG GGCAGCCGGA GAACAACTAC
901 AAGACCACGC CTCCCGTGCT GGACTCCCGC GGCTCCTTCT TCCTCGTGAG
951 CAAGCTCACC GTGGACAAGA GCAGGTGGCA GCAGGGGAAC GTCTTCTCAT
1001 GCTCCGTGAT GCATGAGGCT CTGCACAACC ACTACACGCA GAAGAGCCTC
1051 TCCCTGTCTC CGGGTAAA (SEQ ID NO: 248)

The mature ALK4-Fc fusion polypeptide sequence is as follows (SEQ ID NO: 249) and may optionally be provided with lysine removed from the C-terminus.

1 SGPRGVQALL CACTSCLQAN YTCETDGACM VSIFNLDGME HHVRTCIPKV
51 ELVPAGKPFY CLSSEDLRNT HCCYTDYCNR IDLRVPSGHL KEPEHPSMWG
101 PVETGGGTHT CPPCPAPELL GGPSVFLFPP KPKDTLMISR TPEVTCVVVD
151 VSHEDPEVKF NWYVDGVEVH NAKTKPREEQ YNSTYRVVSV LTVLHQDWLN
201 GKEYKCKVSN KALPAPIEKT ISKAKGQPRE PQVCTLPPSR EEMTKNQVSL
251 SCAVKGFYPS DIAVEWESRG QPENNYKTTP PVLDSRGSFF LVSKLTVDKS
301 RWQQGNVFSC SVMHEALHNH YTQKSLSLSP GK (SEQ ID NO: 249)

This ALK4-Fc fusion polypeptide is encoded by the following nucleic acid (SEQ ID NO: 250):

1 TCCGGGCCCC GGGGGGTCCA GGCTCTGCTG TGTGCGTGCA CCAGCTGCCT
51 CCAGGCCAAC TACACGTGTG AGACAGATGG GGCCTGCATG GTTTCCATTT
101 TCAATCTGGA TGGGATGGAG CACCATGTGC GCACCTGCAT CCCCAAAGTG
151 GAGCTGGTCC CTGCCGGGAA GCCCTTCTAC TGCCTGAGCT CGGAGGACCT
201 GCGCAACACC CACTGCTGCT ACACTGACTA CTGCAACAGG ATCGACTTGA
251 GGGTGCCCAG TGGTCACCTC AAGGAGCCTG AGCACCCGTC CATGTGGGGC
301 CCGGTGGAGA CCGGTGGTGG AACTCACACA TGCCCACCGT GCCCAGCACC
351 TGAACTCCTG GGGGGACCGT CAGTCTTCCT CTTCCCCCCA AAACCCAAGG
401 ACACCCTCAT GATCTCCCGG ACCCCTGAGG TCACATGCGT GGTGGTGGAC
451 GTGAGCCACG AAGACCCTGA GGTCAAGTTC AACTGGTACG TGGACGGCGT
501 GGAGGTGCAT AATGCCAAGA CAAAGCCGCG GGAGGAGCAG TACAACAGCA
551 CGTACCGTGT GGTCAGCGTC CTCACCGTCC TGCACCAGGA CTGGCTGAAT
601 GGCAAGGAGT ACAAGTGCAA GGTCTCCAAC AAAGCCCTCC CAGCCCCCAT
651 CGAGAAAACC ATCTCCAAAG CCAAAGGGCA GCCCCGAGAA CCACAGGTGT
701 GCACCCTGCC CCCATCCCGG GAGGAGATGA CCAAGAACCA GGTCAGCCTG
751 TCCTGCGCCG TCAAAGGCTT CTATCCCAGC GACATCGCCG TGGAGTGGGA
801 GAGCCGCGGG CAGCCGGAGA ACAACTACAA GACCACGCCT CCCGTGCTGG
851 ACTCCCGCGG CTCCTTCTTC CTCGTGAGCA AGCTCACCGT GGACAAGAGC
901 AGGTGGCAGC AGGGGAACGT CTTCTCATGC TCCGTGATGC ATGAGGCTCT
951 GCACAACCAC TACACGCAGA AGAGCCTCTC CCTGTCTCCG GGTAAA
(SEQ ID NO: 250)

In certain embodiments, the ALK4-Fc fusion polypeptide is SEQ ID NO: 92 (shown above), which contains four amino acid substitutions to guide heterodimer formation certain Fc fusion polypeptides disclosed herein, and may optionally be provided with lysine removed from the C-terminus.

This ALK4-Fc fusion polypeptide is encoded by the following nucleic acid (SEQ ID NO: 251):

1 ATGGATGCAA TGAAGAGAGG GCTCTGCTGT GTGCTGCTGC TGTGTGGAGC
51 AGTCTTCGTT TCGCCCGGCG CCTCCGGGCC CCGGGGGGTC CAGGCTCTGC
101 TGTGTGCGTG CACCAGCTGC CTCCAGGCCA ACTACACGTG TGAGACAGAT
151 GGGGCCTGCA TGGTTTCCAT TTTCAATCTG GATGGGATGG AGCACCATGT
201 GCGCACCTGC ATCCCCAAAG TGGAGCTGGT CCCTGCCGGG AAGCCCTTCT
251 ACTGCCTGAG CTCGGAGGAC CTGCGCAACA CCCACTGCTG CTACACTGAC
301 TACTGCAACA GGATCGACTT GAGGGTGCCC AGTGGTCACC TCAAGGAGCC
351 TGAGCACCCG TCCATGTGGG GCCCGGTGGA GACCGGTGGT GGAACTCACA
401 CATGCCCACC GTGCCCAGCA CCTGAACTCC TGGGGGGACC GTCAGTCTTC
451 CTCTTCCCCC CAAAACCCAA GGACACCCTC ATGATCTCCC GGACCCCTGA
501 GGTCACATGC GTGGTGGTGG ACGTGAGCCA CGAAGACCCT GAGGTCAAGT
551 TCAACTGGTA CGTGGACGGC GTGGAGGTGC ATAATGCCAA GACAAAGCCG
601 CGGGAGGAGC AGTACAACAG CACGTACCGT GTGGTCAGCG TCCTCACCGT
651 CCTGCACCAG GACTGGCTGA ATGGCAAGGA GTACAAGTGC AAGGTCTCCA
701 ACAAAGCCCT CCCAGCCCCC ATCGAGAAAA CCATCTCCAA AGCCAAAGGG
751 CAGCCCCGAG AACCACAGGT GTGCACCCTG CCCCCATCCC GGGAGGAGAT
801 GACCAAGAAC CAGGTCAGCC TGTCCTGCGC CGTCAAAGGC TTCTATCCCA
851 GCGACATCGC CGTGGAGTGG GAGAGCAATG GGCAGCCGGA GAACAACTAC
901 AAGACCACGC CTCCCGTGCT GGACTCCGAC GGCTCCTTCT TCCTCGTGAG
951 CAAGCTCACC GTGGACAAGA GCAGGTGGCA GCAGGGGAAC GTCTTCTCAT
1001 GCTCCGTGAT GCATGAGGCT CTGCACAACC ACTACACGCA GAAGAGCCTC
1051 TCCCTGTCTC CGGGTAAA (SEQ ID NO: 251)

The mature ALK4-Fc fusion polypeptide sequence is SEQ ID NO: 93 (shown above) and may optionally be provided with lysine removed from the C-terminus.

This ALK4-Fc fusion polypeptide is encoded by the following nucleic acid (SEQ ID NO: 252):

1 TCCGGGCCCC GGGGGGTCCA GGCTCTGCTG TGTGCGTGCA CCAGCTGCCT
51 CCAGGCCAAC TACACGTGTG AGACAGATGG GGCCTGCATG GTTTCCATTT
101 TCAATCTGGA TGGGATGGAG CACCATGTGC GCACCTGCAT CCCCAAAGTG
151 GAGCTGGTCC CTGCCGGGAA GCCCTTCTAC TGCCTGAGCT CGGAGGACCT
201 GCGCAACACC CACTGCTGCT ACACTGACTA CTGCAACAGG ATCGACTTGA
251 GGGTGCCCAG TGGTCACCTC AAGGAGCCTG AGCACCCGTC CATGTGGGGC
301 CCGGTGGAGA CCGGTGGTGG AACTCACACA TGCCCACCGT GCCCAGCACC
351 TGAACTCCTG GGGGGACCGT CAGTCTTCCT CTTCCCCCCA AAACCCAAGG
401 ACACCCTCAT GATCTCCCGG ACCCCTGAGG TCACATGCGT GGTGGTGGAC
451 GTGAGCCACG AAGACCCTGA GGTCAAGTTC AACTGGTACG TGGACGGCGT
501 GGAGGTGCAT AATGCCAAGA CAAAGCCGCG GGAGGAGCAG TACAACAGCA
551 CGTACCGTGT GGTCAGCGTC CTCACCGTCC TGCACCAGGA CTGGCTGAAT
601 GGCAAGGAGT ACAAGTGCAA GGTCTCCAAC AAAGCCCTCC CAGCCCCCAT
651 CGAGAAAACC ATCTCCAAAG CCAAAGGGCA GCCCCGAGAA CCACAGGTGT
701 GCACCCTGCC CCCATCCCGG GAGGAGATGA CCAAGAACCA GGTCAGCCTG
751 TCCTGCGCCG TCAAAGGCTT CTATCCCAGC GACATCGCCG TGGAGTGGGA
801 GAGCAATGGG CAGCCGGAGA ACAACTACAA GACCACGCCT CCCGTGCTGG
851 ACTCCGACGG CTCCTTCTTC CTCGTGAGCA AGCTCACCGT GGACAAGAGC
901 AGGTGGCAGC AGGGGAACGT CTTCTCATGC TCCGTGATGC ATGAGGCTCT
951 GCACAACCAC TACACGCAGA AGAGCCTCTC CCTGTCTCCG GGTAAA
(SEQ ID NO: 252)

Purification of various ALK4-Fc:ActRIIB-Fc complexes could be achieved by a series of column chromatography steps, including, for example, three or more of the following, in any order: protein A chromatography, Q sepharose chromatography, phenylsepharose chromatography, size exclusion chromatography and epitope-based affinity chromatography (e.g., with an antibody or functionally equivalent ligand directed against an epitope on ALK4 or ActRIIB), and multimodal chromatography (e.g., with resin containing both electrostatic and hydrophobic ligands). The purification could be completed with viral filtration and buffer exchange.

In certain aspects, the present disclosure relates to protein complexes that comprise an ALK7 polypeptide. As used herein, the term “ALK7” refers to a family of activin receptor-like kinase-7 proteins from any species and variants derived from such ALK7 proteins by mutagenesis or other modification. Reference to ALK7 herein is understood to be a reference to any one of the currently identified forms. Members of the ALK7 family are generally transmembrane proteins, composed of a ligand-binding extracellular domain with a cysteine-rich region, a transmembrane domain, and a cytoplasmic domain with predicted serine/threonine kinase activity.

The term “ALK7 polypeptide” includes polypeptides comprising any naturally occurring polypeptide of an ALK7 family member as well as any variants thereof (including mutants, fragments, fusions, and peptidomimetic forms) that retain a useful activity.

Four naturally occurring isoforms of human ALK7 have been described. The sequence of human ALK7 isoform 1 precursor protein (NCBI Ref Seq NP_660302.2) is as follows:

1 MTRALCSALR QALLLLAAAA ELSPGLKCVC LLCDSSNFTC QTEGACWASV MLTNGKEQVI
61 KSCVSLPELN AQVFCHSSNN VTKTECCFTD FCNNITLHLP TASPNAPKLG PMELAIIITV
121 PVCLLSIAAM LTVWACQGRQ CSYRKKKRPN VEEPLSECNL VNAGKTLKDL IYDVTASGSG
181 SGLPLLVQRT IARTIVLQEI VGKGRFGEVW HGRWCGEDVA VKIFSSRDER SWFREAEIYQ
241 TVMLRHENIL GFIAADNKDN GTWTQLWLVS EYHEQGSLYD YLNRNIVTVA GMIKLALSIA
301 SGLAHLHMEI VGTQGKPAIA HRDIKSKNIL VKKCETCAIA DLGLAVKHDS ILNTIDIPQN
361 PKVGTKRYMA PEMLDDTMNV NIFESFKRAD IYSVGLVYWE IARRCSVGGI VEEYQLPYYD
421 MVPSDPSIEE MRKVVCDQKF RPSIPNQWQS CEALRVMGRI MRECWYANGA ARLTALRIKK
481 TISQLCVKED CKA (SEQ ID NO: 120)

The signal peptide is indicated by a single underline and the extracellular domain is indicated in bold font.

A processed extracellular ALK7 isoform 1 polypeptide sequence is as follows:

ELSPGLKCVCLLCDSSNFTCQTEGACWASVMLTNGKEQVIKSCVSLPELN
AQVFCHSSNNVTKTECCFTDFCNNITLHLPTASPNAPKLGPME
(SEQ ID NO: 123)

A nucleic acid sequence encoding human ALK7 isoform 1 precursor protein is shown below in SEQ ID NO: 233, corresponding to nucleotides 244-1722 of Genbank Reference Sequence NM_145259.2. A nucleic acid sequence encoding the processed extracellular ALK7 polypeptide (isoform 1) is show in in SEQ ID NO: 234.

An amino acid sequence of an alternative isoform of human ALK7, isoform 2 (NCBI Ref Seq NP_001104501.1), is shown in its processed form as follows (SEQ ID NO: 124), where the extracellular domain is indicated in bold font.

1 MLTNGKEQVI KSCVSLPELN AQVFCHSSNN VTKTECCFTD FCNNITLHLP TASPNAPKLG
61 PMELAIIITV PVCLLSIAAM LTVWACQGRQ CSYRKKKRPN VEEPLSECNL VNAGKTLKDL
121 IYDVTASGSG SGLPLLVQRT IARTIVLQEI VGKGRFGEVW HGRWCGEDVA VKIFSSRDER
181 SWFREAEIYQ TVMLRHENIL GFIAADNKDN GTWTQLWLVS EYHEQGSLYD YLNRNIVTVA
241 GMIKLALSIA SGLAHLHMEI VGTQGKPAIA HRDIKSKNIL VKKCETCAIA DLGLAVKHDS
301 ILNTIDIPQN PKVGTKRYMA PEMLDDTMNV NIFESFKRAD IYSVGLVYWE IARRCSVGGI
361 VEEYQLPYYD MVPSDPSIEE MRKVVCDQKF RPSIPNQWQS CEALRVMGRI MRECWYANGA
421 ARLTALRIKK TISQLCVKED CKA (SEQ ID NO: 124)

An amino acid sequence of the extracellular ALK7 polypeptide (isoform 2) is as follows:

MLTNGKEQVIKSCVSLPELNAQVFCHSSNNVTKTECCFTDFCNNITLHLP
TASPNAPKLGPME (SEQ ID NO: 125).

A nucleic acid sequence encoding the processed ALK7 polypeptide (isoform 2) is shown below in SEQ ID NO: 235, corresponding to nucleotides 279-1607 of NCBI Reference Sequence NM_001111031.1.

A nucleic acid sequence encoding an extracellular ALK7 polypeptide (isoform 2) is shown in SEQ ID NO: 236.

An amino acid sequence of an alternative human ALK7 precursor protein, isoform 3 (NCBI Ref Seq NP_001104502.1), is shown as follows (SEQ ID NO: 121), where the signal peptide is indicated by a single underline.

1 MTRALCSALR QALLLLAAAA ELSPGLKCVC LLCDSSNFTC QTEGACWASV MLTNGKEQVI
61 KSCVSLPELN AQVFCHSSNN VTKTECCFTD FCNNITLHLP TGLPLLVQRT IARTIVLQEI
121 VGKGRFGEVW HGRWCGEDVA VKIFSSRDER SWFREAEIYQ TVMLRHENIL GFIAADNKDN
181 GTWTQLWLVS EYHEQGSLYD YLNRNIVTVA GMIKLALSIA SGLAHLHMEI VGTQGKPAIA
241 HRDIKSKNIL VKKCETCAIA DLGLAVKHDS ILNTIDIPQN PKVGTKRYMA PEMLDDTMNV
301 NIFESFKRAD IYSVGLVYWE IARRCSVGGI VEEYQLPYYD MVPSDPSIEE MRKVVCDQKF
361 RPSIPNQWQS CEALRVMGRI MRECWYANGA ARLTALRIKK TISQLCVKED CKA
(SEQ ID NO: 121)

The amino acid sequence of a processed ALK7 polypeptide (isoform 3) is as follows (SEQ ID NO: 126). This isoform lacks a transmembrane domain and is therefore proposed to be soluble in its entirety (Roberts et al., 2003, Biol Reprod 68:1719-1726). N-terminal variants of SEQ ID NO: 126 are predicted as described below.

1 ELSPGLKCVC LLCDSSNFTC QTEGACWASV MLTNGKEQVI KSCVSLPELN AQVFCHSSNN
61 VTKTECCFTD FCNNITLHLP TGLPLLVQRT IARTIVLQEI VGKGRFGEVW HGRWCGEDVA
121 VKIFSSRDER SWFREAEIYQ TVMLRHENIL GFIAADNKDN GTWTQLWLVS EYHEQGSLYD
181 YLNRNIVTVA GMIKLALSIA SGLAHLHMEI VGTQGKPAIA HRDIKSKNIL VKKCETCAIA
241 DLGLAVKHDS ILNTIDIPQN PKVGTKRYMA PEMLDDTMNV NIFESFKRAD IYSVGLVYWE
301 IARRCSVGGI VEEYQLPYYD MVPSDPSIEE MRKVVCDQKF RPSIPNQWQS CEALRVMGRI
361 MRECWYANGA ARLTALRIKK TISQLCVKED CKA (SEQ ID NO: 126)

A nucleic acid sequence encoding an unprocessed ALK7 polypeptide precursor protein (isoform 3) is shown in SEQ ID NO: 237, corresponding to nucleotides 244-1482 of NCBI Reference Sequence NM_001111032.1. A nucleic acid sequence encoding a processed ALK7 polypeptide (isoform 3) is shown in SEQ ID NO: 238.

An amino acid sequence of an alternative human ALK7 precursor protein, isoform 4 (NCBI Ref Seq NP_001104503.1), is shown as follows (SEQ ID NO: 122), where the signal peptide is indicated by a single underline.

1 MTRALCSALR QALLLLAAAA ELSPGLKCVC LLCDSSNFTC QTEGACWASV MLTNGKEQVI
61 KSCVSLPELN AQVFCHSSNN VTKTECCFTD FCNNITLHLP TDNGTWTQLW LVSEYHEQGS
121 LYDYLNRNIV TVAGMIKLAL SIASGLAHLH MEIVGTQGKP AIAHRDIKSK NILVKKCETC
181 AIADLGLAVK HDSILNTIDI PQNPKVGTKR YMAPEMLDDT MNVNIFESFK RADIYSVGLV
241 YWEIARRCSV GGIVEEYQLP YYDMVPSDPS IEEMRKWCD QKFRPSIPNQ WQSCEALRVM
301 GRIMRECWYA NGAARLTALR IKKTISQLCV KEDCKA (SEQ ID NO: 122)

An amino acid sequence of a processed ALK7 polypeptide (isoform 4) is as follows (SEQ ID NO: 127). Like ALK7 isoform 3, isoform 4 lacks a transmembrane domain and is therefore proposed to be soluble in its entirety (Roberts et al., 2003, Biol Reprod 68:1719-1726). N-terminal variants of SEQ ID NO: 127 are predicted as described below.

1 ELSPGLKCVC LLCDSSNFTC QTEGACWASV MLTNGKEQVI KSCVSLPELN AQVFCHSSNN
61 VTKTECCFTD FCNNITLHLP TDNGTWTQLW LVSEYHEQGS LYDYLNRNIV TVAGMIKLAL
121 SIASGLAHLH MEIVGTQGKP AIAHRDIKSK NILVKKCETC AIADLGLAVK HDSILNTIDI
181 PQNPKVGTKR YMAPEMLDDT MNVNIFESFK RADIYSVGLV YWEIARRCSV GGIVEEYQLP
240 YYDMVPSDPS IEEMRKWCD QKFRPSIPNQ WQSCEALRVM GRIMRECWYA NGAARLTALR
301 IKKTISQLCV KEDCKA (SEQ ID NO: 127)

A nucleic acid sequence encoding the unprocessed ALK7 polypeptide precursor protein (isoform 4) is shown in SEQ ID NO: 239, corresponding to nucleotides 244-1244 of NCBI Reference Sequence NM_001111033.1. A nucleic acid sequence encoding the processed ALK7 polypeptide (isoform 4) is shown in SEQ ID NO: 240.

Based on the signal sequence of full-length ALK7 (isoform 1) in the rat (see NCBI Reference Sequence NP_620790.1) and on the high degree of sequence identity between human and rat ALK7, it is predicted that a processed form of human ALK7 isoform 1 is as follows (SEQ ID NO: 128).

1 LKCVCLLCDS SNFTCQTEGA CWASVMLTNG KEQVIKSCVS LPELNAQVFC HSSNNVTKTE
61 CCFTDFCNNI TLHLPTASPN APKLGPME (SEQ ID NO: 128)

Active variants of processed ALK7 isoform 1 are predicted in which SEQ ID NO: 123 is truncated by 1, 2, 3, 4, 5, 6, or 7 amino acids at the N-terminus and SEQ ID NO: 128 is truncated by 1 or 2 amino acids at the N-terminus. Consistent with SEQ ID NO: 128, it is further expected that leucine is the N-terminal amino acid in the processed forms of human ALK7 isoform 3 (SEQ ID NO: 126) and human ALK7 isoform 4 (SEQ ID NO: 127).

In certain embodiments, the disclosure relates to heteromultimers that comprise at least one ALK7 polypeptide, which includes fragments, functional variants, and modified forms thereof. Preferably, ALK7 polypeptides for use as disclosed herein (e.g., heteromultimers comprising an ALK7 polypeptide and uses thereof) are soluble (e.g., an extracellular domain of ALK7). In other preferred embodiments, ALK7 polypeptides for use as disclosed herein bind to and/or inhibit (antagonize) activity (e.g., induction of Smad signaling) of one or more TGF-beta superfamily ligands. In some embodiments, heteromultimers of the disclosure comprise at least one ALK7 polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 120, 121, 122, 123, 124, 125, 126, 127, 128, 129, 130, 133, or 134. In some embodiments, heteromultimers of the disclosure consist or consist essentially of at least one ALK7 polypeptide that is at least 70%, 75%, 80%, 85%, 90%, 91%, 92%, 93%, 94%, 95%, 96%, 97%, 98%, 99%, or 100% identical to the amino acid sequence of SEQ ID NO: 120, 121, 122, 123, 124, 125, 126, 127, 128, 129, 130, 133, or 134.

In certain aspects, the disclosure relates to a heteromultimer that comprises an ALK7-Fc fusion protein. In some embodiments, the ALK7-Fc fusion protein comprises an ALK7 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21-28 (e.g., amino acid residues 21, 22, 23, 24, 25, 26, 27, and 28) SEQ ID NO: 120, 121, or 122, and ends at any one of amino acids 92-113 (e.g., amino acid residues 92, 93, 94, 95, 96, 97, 98, 99, 100, 101, 102, 103, 104, 105, 106, 107, 108, 109, 110, 111, 112, and 113) of SEQ ID NO: 120, 121, or 122. In some embodiments, the ALK7-Fc fusion protein comprises an ALK7 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 28-92 of SEQ ID NOs: 120, 121, or 122. In some embodiments, the ALK7-Fc fusion protein comprises an ALK7 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to amino acids 21-113 of SEQ ID NOs: 120, 121, or 122. In some embodiments, the ALK7-Fc fusion protein comprises an ALK7 domain comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of any one of SEQ ID Nos: 120, 123, 124, 125, 121, 126, 122, 127, 128, 129, 130, 131, 132, 133, 134, 135, and 136.

In some embodiments, the ALK7-Fc fusion protein employs the TPA leader and is as follows (SEQ ID NO: 129):

1 MDAMKRGLCC VLLLCGAVFV SPGAGLKCVC LLCDSSNFTC QTEGACWASV
51 MLTNGKEQVI KSCVSLPELN AQVFCHSSNN VTKTECCFTD FCNNITLHLP
101 TASPNAPKLG PMETGGGTHT CPPCPAPELL GGPSVFLFPP KPKDTLMISR
151 TPEVTCVVVD VSHEDPEVKF NWYVDGVEVH NAKTKPREEQ YNSTYRVVSV
201 LTVLHQDWLN GKEYKCKVSN KALPAPIEKT ISKAKGQPRE PQVYTLPPSR
251 EEMTKNQVSL TCLVKGFYPS DIAVEWESNG QPENNYDTTP PVLDSDGSFF
301 LYSDLTVDKS RWQQGNVFSC SVMHEALHNH YTQKSLSLSP G
(SEQ ID NO: 129)

The signal sequence and linker sequence are underlined. To promote formation of the ActRIIB-Fc:ALK7-Fc heterodimer rather than either of the possible homodimeric complexes, two amino acid substitutions (replacing lysines with aspartic acids) can be introduced into the Fc domain of the fusion protein as indicated by double underline above. The amino acid sequence of SEQ ID NO: 129 may optionally be provided with a lysine added at the C-terminus.

This ALK7-Fc fusion protein is encoded by the following nucleic acid (SEQ ID NO: 255):

1 ATGGATGCAA TGAAGAGAGG GCTCTGCTGT GTGCTGCTGC TGTGTGGAGC
51 AGTCTTCGTT TCGCCCGGCG CCGGACTGAA GTGTGTATGT CTTTTGTGTG
101 ATTCTTCAAA CTTTACCTGC CAAACAGAAG GAGCATGTTG GGCATCAGTC
151 ATGCTAACCA ATGGAAAAGA GCAGGTGATC AAATCCTGTG TCTCCCTTCC
201 AGAACTGAAT GCTCAAGTCT TCTGTCATAG TTCCAACAAT GTTACCAAAA
251 CCGAATGCTG CTTCACAGAT TTTTGCAACA ACATAACACT GCACCTTCCA
301 ACAGCATCAC CAAATGCCCC AAAACTTGGA CCCATGGAGA CCGGTGGTGG
351 AACTCACACA TGCCCACCGT GCCCAGCACC TGAACTCCTG GGGGGACCGT
401 CAGTCTTCCT CTTCCCCCCA AAACCCAAGG ACACCCTCAT GATCTCCCGG
451 ACCCCTGAGG TCACATGCGT GGTGGTGGAC GTGAGCCACG AAGACCCTGA
501 GGTCAAGTTC AACTGGTACG TGGACGGCGT GGAGGTGCAT AATGCCAAGA
551 CAAAGCCGCG GGAGGAGCAG TACAACAGCA CGTACCGTGT GGTCAGCGTC
601 CTCACCGTCC TGCACCAGGA CTGGCTGAAT GGCAAGGAGT ACAAGTGCAA
651 GGTCTCCAAC AAAGCCCTCC CAGCCCCCAT CGAGAAAACC ATCTCCAAAG
701 CCAAAGGGCA GCCCCGAGAA CCACAGGTGT ACACCCTGCC CCCATCCCGG
751 GAGGAGATGA CCAAGAACCA GGTCAGCCTG ACCTGCCTGG TCAAAGGCTT
801 CTATCCCAGC GACATCGCCG TGGAGTGGGA GAGCAATGGG CAGCCGGAGA
851 ACAACTACGA CACCACGCCT CCCGTGCTGG ACTCCGACGG CTCCTTCTTC
901 CTCTATAGCG ACCTCACCGT GGACAAGAGC AGGTGGCAGC AGGGGAACGT
951 CTTCTCATGC TCCGTGATGC ATGAGGCTCT GCACAACCAC TACACGCAGA
1001 AGAGCCTCTC CCTGTCTCCG GGT (SEQ ID NO: 255)

The mature ALK7-Fc fusion protein sequence (SEQ ID NO: 130) is expected to be as follows and may optionally be provided with a lysine added at the C-terminus.

1 GLKCVCLLCD SSNFTCQTEG ACWASVMLTN GKEQVIKSCV SLPELNAQVF
51 CHSSNNVTKT ECCFTDFCNN ITLHLPTASP NAPKLGPMET GGGTHTCPPC
101 PAPELLGGPS VFLFPPKPKD TLMISRTPEV TCVVVDVSHE DPEVKFNWYV
151 DGVEVHNAKT KPREEQYNST YRVVSVLTVL HQDWLNGKEY KCKVSNKALP
201 APIEKTISKA KGQPREPQVY TLPPSREEMT KNQVSLTCLV KGFYPSDIAV
251 EWESNGQPEN NYDTTPPVLD SDGSFFLYSD LTVDKSRWQQ GNVFSCSVMH
301 EALHNHYTQK SLSLSPG (SEQ ID NO: 130)

The complementary form of ALK7-Fc fusion polypeptide (SEQ ID NO: 133) is as follows:

1 MDAMKRGLCC VLLLCGAVFV SPGAGLKCVC LLCDSSNFTC QTEGACWASV
51 MLTNGKEQVI KSCVSLPELN AQVFCHSSNN VTKTECCFTD FCNNITLHLP
101 TASPNAPKLG PMETGGGTHT CPPCPAPELL GGPSVFLFPP KPKDTLMISR
151 TPEVTCVVVD VSHEDPEVKF NWYVDGVEVH NAKTKPREEQ YNSTYRVVSV
201 LTVLHQDWLN GKEYKCKVSN KALPAPIEKT ISKAKGQPRE PQVCTLPPSR
251 EEMTKNQVSL SCAVKGFYPS DIAVEWESNG QPENNYKTTP PVLDSDGSFF
301 LVSKLTVDKS RWQQGNVFSC SVMHEALHNH YTQKSLSLSP GK
(SEQ ID NO: 133)

The leader sequence and linker sequence are underlined. To guide heterodimer formation with certain Fc fusion polypeptides disclosed herein, four amino acid substitutions can be introduced into the Fc domain of the ALK7 fusion polypeptide as indicated by double underline above. Furthermore, the C-terminal lysine residue of the Fc domain can be deleted. The amino acid sequence of SEQ ID NO: 133 may optionally be provided with the lysine removed from the C-terminus.

The mature ALK7-Fc fusion protein sequence (SEQ ID NO: 134) is expected to be as follows and may optionally be provided with the lysine removed from the C-terminus.

1 GLKCVCLLCD SSNFTCQTEG ACWASVMLTN GKEQVIKSCV SLPELNAQVF
51 CHSSNNVTKT ECCFTDFCNN ITLHLPTASP NAPKLGPMET GGGTHTCPPC
101 PAPELLGGPS VFLFPPKPKD TLMISRTPEV TCVVVDVSHE DPEVKFNWYV
151 DGVEVHNAKT KPREEQYNST YRVVSVLTVL HQDWLNGKEY KCKVSNKALP
201 APIEKTISKA KGQPREPQVC TLPPSREEMT KNQVSLSCAV KGFYPSDIAV
251 EWESNGQPEN NYKTTPPVLD SDGSFFLVSK LTVDKSRWQQ GNVFSCSVMH
301 EALHNHYTQK SLSLSPGK (SEQ ID NO: 134)

In certain embodiments, the disclosure provides for a heteromultimer protein comprising any of the variant ActRIIB polypeptides disclosed herein and a second polypeptide selected from the group consisting of: an ALK4 or ALK7 polypeptide, or functional fragments thereof. In some embodiments, the heteromultimer protein complex comprises a variant ActRIIB polypeptide and an ALK4 polypeptide, or a functional fragment thereof. In some embodiments, the heteromultimer protein complex comprises a variant ActRIIB polypeptide and an ALK7 polypeptide, or a functional fragment thereof. In some embodiments, the second polypeptide is an ALK4 polypeptide or a functional fragment thereof. In some embodiments, the ALK4 polypeptide or functional fragment thereof comprises an amino acid sequence that is at least 70%, 75%, 80%, 85%, 90%, 95%, 96%, 97%, 98%, 99%, or 100% identical to the amino acid sequence of SEQ ID NO: 84 or 85, or functional fragments thereof. In some embodiments, the ALK4 polypeptide or functional fragment thereof comprises an amino acid sequence that is at least 70%, 75%, 80%, 85%, 90%, 95%, 96%, 97%, 98%, 99%, or 100% identical to the amino acid sequence of SEQ ID NO: 84, 85, 86, 87, 88, 89, 92, and 93, or functional fragments thereof. In some embodiments, the second polypeptide is an ALK7 polypeptide or a functional fragment thereof. In some embodiments, the ALK7 polypeptide or functional fragment thereof comprises an amino acid sequence that is at least 70%, 75%, 80%, 85%, 90%, 95%, 96%, 97%, 98%, 99%, or 100% identical to the amino acid sequence of SEQ ID NO: 120, 121, or 122, or functional fragments thereof. In some embodiments, the ALK7 polypeptide or functional fragment thereof comprises an amino acid sequence that is at least 70%, 75%, 80%, 85%, 90%, 95%, 96%, 97%, 98%, 99%, or 100% identical to the amino acid sequence of SEQ ID NO: 120, 121, 122, 123, 124, 125, 126, 127, 128, 129, 130, 133, and 134, or functional fragments thereof.

In some embodiments, the present disclosure contemplates making functional variants by modifying the structure of an ALK4 polypeptide, ALK7 polypeptide, and/or a variant ActRIIB polypeptide for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more TGF-beta superfamily ligands including, for example, activin A, activin B, GDF8, GDF11 and BMP10.

In some embodiments, the present disclosure contemplates making functional variants by modifying the structure of an ALK4 polypeptide, ALK7 polypeptide, and/or a variant ActRIIB polypeptide for such purposes as enhancing therapeutic efficacy or stability (e.g., increased shelf-life and/or increased resistance to proteolytic degradation).

In certain embodiments, the present disclosure contemplates specific mutations of an ALK4 polypeptide, ALK7 polypeptide, and/or a variant ActRIIB polypeptide of the disclosure so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine, or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, ALK4 polypeptide, ALK7 polypeptide, and/or a variant ActRIIB polypeptide complexes of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of an ALK4 polypeptide, ALK7 polypeptide, and/or a variant ActRIIB polypeptide of the disclosure, as well as truncation mutants. Pools of combinatorial mutants are especially useful for identifying functionally active (e.g., ligand binding) ALK4, ALK7, and/or a variant ActRIIB polypeptide sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, variant ALK4, ALK7, and/or a variant ActRIIB polypeptide sequences may be screened for ability to bind to a TGF-beta superfamily ligand (e.g., activin A, activin B, GDF8, GDF11, and BMP10), to prevent binding of a TGF-beta superfamily ligand to a TGF-beta superfamily receptor, and/or to interfere with signaling caused by an TGF-beta superfamily ligand.

The activity of ALK4, ALK7, and/or a variant ActRIIB polypeptide heteromultimer of the disclosure also may be tested, for example in a cell-based or in vivo assay. For example, the effect of a heteromultimer complex on the expression of genes or the activity of proteins involved in muscle production in a muscle cell may be assessed. This may, as needed, be performed in the presence of one or more recombinant TGF-beta superfamily ligand proteins (e.g., activin A, activin B, GDF8, GDF11, and BMP10), and cells may be transfected so as to produce an ALK4, ALK7, and/or variant ActRIIB polypeptide complex, and optionally, a TGF-beta superfamily ligand. Likewise, a heteromultimer complex of the disclosure may be administered to a mouse or other animal, and one or more measurements, such as muscle formation and strength may be assessed using art-recognized methods. Similarly, the activity of a heteromultimer, or variants thereof, may be tested in osteoblasts, adipocytes, and/or neuronal cells for any effect on growth of these cells, for example, by the assays as described herein and those of common knowledge in the art. A SMAD-responsive reporter gene may be used in such cell lines to monitor effects on downstream signaling.

In certain aspects, heteromultimers of the disclosure bind to one or more TGF-beta superfamily ligands. In some embodiments, heteromultimers of the disclosure bind to one or more TGF-beta superfamily ligands with a K_{D} of at least 1 × 10⁻⁷ M. In some embodiments, the one or more TGF-beta superfamily ligands is selected from the group consisting of: activin A, activin B, GDF8, GDF11, and BMP10.

In certain aspects, heteromultimers of the disclosure inhibits one or more TGF-beta super family ligands. In some embodiments, heteromultimers of the disclosure inhibits signaling of one or more TGF-beta super family ligands. In some embodiments, heteromultimers of the disclosure inhibits Smad signaling of one or more TGF-beta super family ligands. In some embodiments, heteromultimers of the disclosure inhibits signaling of one or more TGF-beta super family ligands in a cell-based assay. In some embodiments, heteromultimers of the disclosure inhibits one or more TGF-beta super family ligands selected from the group consisting of: activin A, activin B, GDF8, GDF11, and BMP10.

Combinatorial-derived variants can be generated which have increased selectivity or generally increased potency relative to a reference ALK4, ALK7, and/or variant ActRIIB polypeptide heteromultimer. Such variants, when expressed from recombinant DNA constructs, can be used in gene therapy protocols. Likewise, mutagenesis can give rise to variants which have intracellular half-lives dramatically different than the corresponding unmodified ALK4, ALK7, and/or variant ActRIIB polypeptide heteromultimer. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter one or more activities of the ALK4, ALK7, and/or variant ActRIIB polypeptide heteromultimer complex including, for example, immunogenicity, half-life, and solubility.

Many methods known in the art can be used to generate heteromultimers of the disclosure. For example, non-naturally occurring disulfide bonds may be constructed by replacing on a first polypeptide (e.g., a variant ActRIIB polypeptide) a naturally occurring amino acid with a free thiol-containing residue, such as cysteine, such that the free thiol interacts with another free thiol-containing residue on a second polypeptide (e.g., an ALK4 polypeptide or an ALK7 polypeptide) such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S.8,592,562; coiled-coil interactions such as described in Christensen et al., U.S.20120302737; leucine zippers such as described in Pack & Plueckthun,(1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

In certain aspects, a multimerization domain may comprise one component of an interaction pair. In some embodiments, the polypeptides disclosed herein may form protein complexes comprising a first polypeptide covalently or non-covalently associated with a second polypeptide, wherein the first polypeptide comprises the amino acid sequence of a variant ActRIIB polypeptide and the amino acid sequence of a first member of an interaction pair; and the second polypeptide comprises the amino acid sequence of an ALK4 polypeptide or an ALK7 polypeptide, and the amino acid sequence of a second member of an interaction pair. The interaction pair may be any two polypeptide sequences that interact to form a complex, particularly a heterodimeric complex although operative embodiments may also employ an interaction pair that can form a homodimeric complex. An interaction pair may be selected to confer an improved property/activity such as increased serum half-life, or to act as an adaptor on to which another moiety is attached to provide an improved property/activity. For example, a polyethylene glycol moiety may be attached to one or both components of an interaction pair to provide an improved property/activity such as improved serum half-life.

The first and second members of the interaction pair may be an asymmetric pair, meaning that the members of the pair preferentially associate with each other rather than self-associate. Accordingly, first and second members of an asymmetric interaction pair may associate to form a heterodimeric complex (see, e.g., FIG. 1B). Alternatively, the interaction pair may be unguided, meaning that the members of the pair may associate with each other or self-associate without substantial preference and thus may have the same or different amino acid sequences (see, e.g., FIG. 1A). Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

As specific examples, the present disclosure provides fusion proteins comprising a variant ActRIIB polypeptide or an unmodified ActRIIB polypeptide fused to a polypeptide comprising a constant domain of an immunoglobulin, such as a CH1, CH2, or CH3 domain of an immunoglobulin or an Fc domain. Fc domains derived from human IgG1, IgG2, IgG3, and IgG4 are provided herein. Other mutations are known that decrease either CDC or ADCC activity, and collectively, any of these variants are included in the disclosure and may be used as advantageous components of a heteromultimers of the disclosure. Optionally, the IgG1 Fc domain of SEQ ID NO: 13 has one or more mutations at residues such as Asp-265, Lys-322, and Asn-434 (numbered in accordance with the corresponding full-length IgG1). In certain cases, the variant Fc domain having one or more of these mutations (e.g., Asp-265 mutation) has reduced ability of binding to the Fcy receptor relative to a wildtype Fc domain. In other cases, the variant Fc domain having one or more of these mutations (e.g., Asn-434 mutation) has increased ability of binding to the MHC class I-related Fc-receptor (FcRN) relative to a wild-type Fc domain.

An example of a native amino acid sequence that may be used for the Fc portion of human IgG1 (G1Fc) is shown below (SEQ ID NO: 13). Dotted underline indicates the hinge region, and solid underline indicates positions with naturally occurring variants. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 13. In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13. Naturally occurring variants in G1Fc would include E134D and M136L according to the numbering system used in SEQ ID NO: 13 (see Uniprot P01857).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLYSKLTV DKSRWQQGNV
201 FSCSVMHEAL HNHYTQKSLS LSPGK (SEQ ID NO: 13)

An example of a native amino acid sequence that may be used for the Fc portion of human IgG2 (G2Fc) is shown below (SEQ ID NO: 14). Dotted underline indicates the hinge region and double underline indicates positions where there are data base conflicts in the sequence (according to UniProt P01859). In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 14. In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14.

1 VECPPCPAPP VAGPSVFLFP PKPKDTLMIS RTPEVTCVVV DVSHEDPEVQ
51 FNWYVDGVEV HNAKTKPREE QFNSTFRVVS VLTVVHQDWL NGKEYKCKVS
101 NKGLPAPIEK TISKTKGQPR EPQVYTLPPS REEMTKNQVS LTCLVKGFYP
151 SDIAVEWESN GQPENNYKTT PPMLDSDGSF FLYSKLTVDK SRWQQGNVFS
201 CSVMHEALHN HYTQKSLSLS PGK (SEQ ID NO: 14)

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 15) contains a short hinge region consisting of a single 15-residue segment, whereas the second G3Fc sequence (SEQ ID NO: 16) contains a full-length hinge region. In each case, dotted underline indicates the hinge region, and solid underline indicates positions with naturally occurring variants according to UniProt P01859. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NOs: 15 and 16. In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15. In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16.

1 EPKSCDTPPP CPRCPAPELL GGPSVFLFPP KPKDTLMISR TPEVTCVVVD
51 VSHEDPEVQF KWYVDGVEVH NAKTKPREEQ YNSTFRVVSV LTVLHQDWLN
101 GKEYKCKVSN KALPAPIEKT ISKTKGQPRE PQVYTLPPSR EEMTKNQVSL
151 TCLVKGFYPS DIAVEWESSG QPENNYNTTP PMLDSDGSFF LYSKLTVDKS
201 RWQQGNIFSC SVMHEALHNR FTQKSLSLSP GK (SEQ ID NO: 15)

1 ELKTPLGDTT HTCPRCPEPK SCDTPPPCPR CPEPKSCDTP PPCPRCPEPK
51 SCDTPPPCPR CPAPELLGGP SVFLFPPKPK DTLMISRTPE VTCVVVDVSH
101 EDPEVQFKWY VDGVEVHNAK TKPREEQYNS TFRVVSVLTV LHQDWLNGKE
151 YKCKVSNKAL PAPIEKTISK TKGQPREPQV YTLPPSREEM TKNQVSLTCL
201 VKGFYPSDIA VEWESSGQPE NNYNTTPPML DSDGSFFLYS KLTVDKSRWQ
251 QGNIFSCSVM HEALHNRFTQ KSLSLSPGK (SEQ ID NO: 16)

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 15, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 17). Dotted underline indicates the hinge region. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 17. In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17.

1 ESKYGPPCPS CPAPEFLGGP SVFLFPPKPK DTLMISRTPE VTCVVVDVSQ
51 EDPEVQFNWY VDGVEVHNAK TKPREEQFNS TYRVVSVLTV LHQDWLNGKE
101 YKCKVSNKGL PSSIEKTISK AKGQPREPQV YTLPPSQEEM TKNQVSLTCL
151 VKGFYPSDIA VEWESNGQPE NNYKTTPPVL DSDGSFFLYS RLTVDKSRWQ
201 EGNVFSCSVM HEALHNHYTQ KSLSLSLGK (SEQ ID NO: 17)

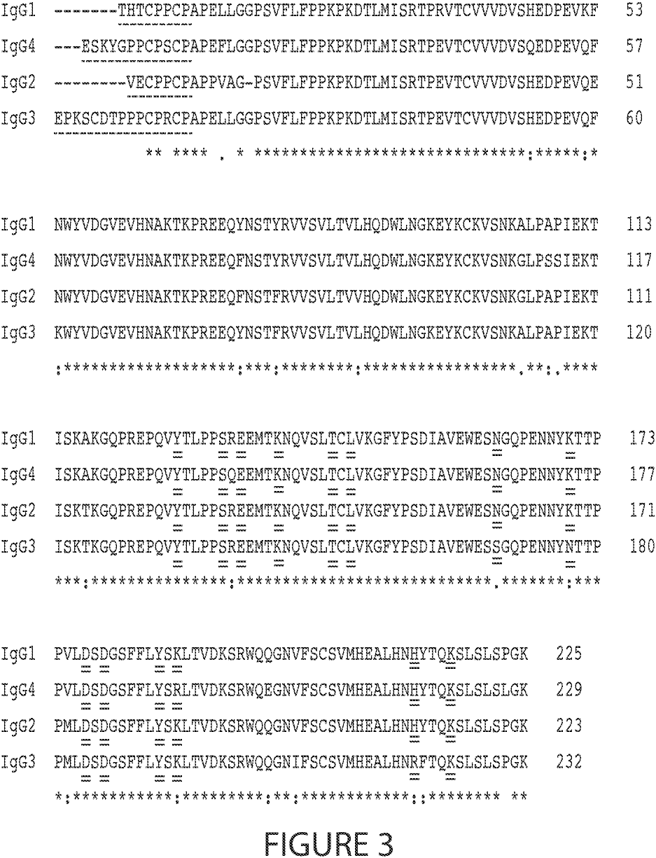

A variety of engineered mutations in the Fc domain are presented herein with respect to the G1Fc sequence (SEQ ID NO: 13), and analogous mutations in G2Fc, G3Fc, and G4Fc can be derived from their alignment with G1Fc in FIG. 3. Due to unequal hinge lengths, analogous Fc positions based on isotype alignment (FIG. 3) possess different amino acid numbers in SEQ ID NOs: 13, 14, 15, and 17. It can also be appreciated that a given amino acid position in an immunoglobulin sequence consisting of hinge, C_{H}2, and C_{H}3 regions (e.g., SEQ ID NOs: 13, 14, 15, 16, or 17) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 13), the human IgG1 heavy chain constant domain (Uniprot P01857), and the human IgG1 heavy chain is as follows.

Correspondence of C_{H}3 Positions in Different Numbering Systems

| | | |
|---|---|---|
| G1Fc (Numbering begins at first threonine in hinge region) | IgG1 heavy chain constant domain (Numbering begins at C_{H}1) | IgG1 heavy chain (EU numbering scheme of Kabat et al., 1991*) |
| Y127 | Y232 | Y349 |
| S132 | 5237 | 5354 |
| E134 | E239 | E356 |
| K138 | K243 | K360 |
| T144 | T249 | T366 |
| L146 | L251 | L368 |
| N162 | N267 | N384 |
| K170 | K275 | K392 |
| D177 | D282 | D399 |
| D179 | D284 | D401 |
| Y185 | Y290 | Y407 |
| K187 | K292 | K409 |
| H213 | H318 | H435 |
| K217 | K322 | K439 |

* Kabat et al. (eds) 1991; pp. 688-696 in Sequences of Proteins of Immunological lnterest, 5^{th} ed., Vol. 1, NlH, Bethesda, MD.

A problem that arises in large-scale production of asymmetric immunoglobulin-based proteins from a single cell line is known as the “chain association issue”. As confronted prominently in the production of bispecific antibodies, the chain-association issue concerns the challenge of efficiently producing a desired multichain protein from among the multiple combinations that inherently result when different heavy chains and/or light chains are produced in a single cell line [see, for example, Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

Various methods are known in the art that increase desired pairing of Fc-containing fusion polypeptide chains in a single cell line to produce a preferred asymmetric fusion protein at acceptable yields [see, for example, Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing. See, for example, Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; US5932448; WO 1993/011162; WO 2009/089004, and WO 2011/034605. As described herein, these methods may be used to generate heterodimers comprising a variant ActRIIB polypeptide and another, optionally different, variant ActRIIB polypeptide or an unmodified ActRIIB polypeptide.

For example, one means by which interaction between specific polypeptides may be promoted is by engineering protuberance-into-cavity (knob-into-holes) complementary regions such as described in Arathoon et al., U.S.7,183,076 and Carter et al., U.S.5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged, and lysine, arginine, and histidine are positively charged. These charged residues can be used to promote heterodimer formation and at the same time hinder homodimer formation. Attractive interactions take place between opposite charges and repulsive interactions occur between like charges. In part, protein complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (′)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will be represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399′ and D399-K409E′). A similar mutation switching the charge polarity (D399K′; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance heteromultimer formation of the heteromultimers disclosed herein.

Examples of Pair-Wise Charged Residue Mutations to Enhance Heterodimer Formation

| | | | |
|---|---|---|---|
| Position in first chain | Mutation in first chain | Interacting position in second chain | Corresponding mutation in second chain |
| Lys409 | Asp or Glu | Asp399′ | Lys, Arg, or His |
| Lys392 | Asp or Glu | Asp399′ | Lys, Arg, or His |
| Lys439 | Asp or Glu | Asp356′ | Lys, Arg, or His |
| Lys370 | Asp or Glu | Glu357′ | Lys, Arg, or His |
| Asp399 | Lys, Arg, or His | Lys409′ | Asp or Glu |
| Asp399 | Lys, Arg, or His | Lys392′ | Asp or Glu |
| Asp356 | Lys, Arg, or His | Lys439′ | Asp or Glu |
| Glu357 | Lys, Arg, or His | Lys370′ | Asp or Glu |

In some embodiments, one or more residues that make up the CH3-CH3 interface in a fusion protein of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus, in certain embodiments, Fc fusion proteins of the disclosure with mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In another embodiment, the antibody or Fc fusion protein comprises an IgG1 framework, wherein the Ala-Ala mutation would describe a mutation(s) from leucine to alanine at position 234 and/or a mutation from leucine to alanine at position 235. While alanine substitutions at these sites are effective in reducing ADCC in both human and murine antibodies, these substitutions are less effective at reducing CDC activity. Another single variant P329A, identified by a random mutagenesis approach to map the Clq binding site of the Fc, is highly effective at reducing CDC activity while retaining ADCC activity. A combination of L234A, L235A, and P329A (LALA-PG, Kabat positions) substitutions have been shown to effectively silence the effector function of human IgG1 antibodies. For a detailed discussion of LALA, LALA-PG, and other mutations, see Lo et al. (2017) 1 Biol. Chem. 292:3900-3908, the contents of which are hereby incorporated herein by reference in their entirety. In some embodiments, Fc fusion proteins of the disclosure comprise L234A, L235A, and P329G mutations (LALA-PG; Kabat positions) in the Fc region of the heavy chain. The antibody or Fc fusion protein may alternatively or additionally carry other mutations, including the point mutation K322A in the CH2 domain (Hezareh et al. 2001 J Virol. 75: 12161-8).

In particular embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively, or additionally, cysteine residue(s) may be introduced in the Fc region, thereby allowing interchain disulfide bond formation in this region. The homodimeric antibody or Fc fusion protein thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. No. 5,648,260; U.S. Pat. No. 5,624,821; and WO94/29351.

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to a variant ActRIIB polypeptide, ALK4 polypeptide, or an ALK7 polypeptide of the construct, with or without an optional linker, to generate a variant ActRIIB-Fc, ALK4-Fc, or ALK7-Fc fusion polypeptide. This single chain can be coexpressed in a cell of choice along with the Fc sequence complementary to the first Fc sequence to favor generation of the desired multichain construct (e.g., a variant ActRIIB-Fc:ALK4 heteromultimer). In this example based on electrostatic steering, SEQ ID NO: 18 [human G1Fc(E134K/D177K)] and SEQ ID NO: 19 [human G1Fc(K170D/K187D)] are examples of complementary Fc sequences in which the engineered amino acid substitutions are double underlined, and a variant ActRIIB polypeptide, ALK4 polypeptide, or an ALK7 polypeptide of the construct can be fused to either SEQ ID NO: 18 or SEQ ID NO: 19, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair below (SEQ ID NOs: 18 and 19).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSRKEMTKNQ VSLTCLVKGF
151 YPSDIAVEWE SNGQPENNYK TTPPVLKSDG SFFLYSKLTV DKSRWQQGNV
201 FSCSVMHEAL HNHYTQKSLS LSPGK (SEQ ID NO: 18)

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYD TTPPVLDSDG SFFLYSDLTV DKSRWQQGNV
201 FSCSVMHEAL HNHYTQKSLS LSPGK (SEQ ID NO: 19)

In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18. In some embodiments, the disclosure relates to ActRIIB heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19.

In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19.

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to a variant ActRIIB polypeptide, an ALK4 polypeptide, or an ALK7 polypeptide of the construct, with or without an optional linker, to generate a variant ActRIIB-Fc, ALK4-Fc, or ALK7-Fc fusion polypeptide. This single chain can be coexpressed in a cell of choice along with the Fc sequence complementary to the first Fc sequence to favor generation of the desired multichain construct. In this example based on knobs-into-holes pairing, SEQ ID NO: 20 [human G1Fc(T144Y)] and SEQ ID NO: 21 [human G1Fc(Y185T)] are examples of complementary Fc sequences in which the engineered amino acid substitutions are double underlined, and a variant ActRIIB polypeptide, ALK4 polypeptide, or ALK7 polypeptide of the construct can be fused to either SEQ ID NO: 20 or SEQ ID NO: 21, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair below (SEQ ID NOs: 20 and 21).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLYCLVKGF
151 YPSDIAVEWE SNGQPENNYK TTPPVLDSDG SFFLYSKLTV DKSRWQQGNV
201 FSCSVMHEAL HNHYTQKSLS LSPGK (SEQ ID NO: 20)

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLTSKLTV DKSRWQQGNV
201 FSCSVMHEAL HNHYTQKSLS LSPGK (SEQ ID NO: 21)

In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20. In some embodiments, the disclosure relates to variant ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21.

In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21.

An example of Fc complementarity based on knobs-into-holes pairing combined with an engineered disulfide bond is disclosed in SEQ ID NO: 22 [hG1Fc(S132C/T144W)] and SEQ ID NO: 23 [hG1Fc(Y127C/T144S/L146A/Y185V)]. The engineered amino acid substitutions in these sequences are double underlined, and variant ActRIIB polypeptide, ALK4 polypeptide, or ALK7 polypeptide of the construct can be fused to either SEQ ID NO: 22 or SEQ ID NO: 23, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair below (SEQ ID NOs: 22 and 23).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PCREEMTKNQ VSLWCLVKGF
151 YPSDIAVEWE SNGQPENNYK TTPPVLDSDG SFFLYSKLTV DKSRWQQGNV
201 FSCSVMHEAL HNHYTQKSLS LSPGK (SEQ ID NO: 22)

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVCTLP PSREEMTKNQ VSLSCAVKGF
151 YPSDIAVEWE SNGQPENNYK TTPPVLDSDG SFFLVSKLTV DKSRWQQGNV
201 FSCSVMHEAL HNHYTQKSLS LSPGK (SEQ ID NO: 23)

In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22. In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23.

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion proteins [see, for example, Davis et al (2010) Protein Eng Design Sel 23:195-202]. One of a pair of Fc sequences with SEEDbody complementarity can be arbitrarily fused to a first variant ActRIIB polypeptide, second variant ActRIIB polypeptide, or unmodified ActRIIB polypeptide of the construct, with or without an optional linker, to generate a variant ActRIIB-Fc or unmodified ActRIIB-Fc fusion polypeptide. This single chain can be coexpressed in a cell of choice along with the Fc sequence complementary to the first Fc sequence to favor generation of the desired multichain construct. In this example based on SEEDbody (Sb) pairing, SEQ ID NO: 24 [hG1Fc(Sb_{AG})] and SEQ ID NO: 25 [hG1Fc(Sb_{GA})] are examples of complementary IgG Fc sequences in which the engineered amino acid substitutions from IgA Fc are double underlined, and a first variant ActRIIB polypeptide, second variant ActRIIB polypeptide, or unmodified ActRIIB polypeptide of the construct can be fused to either SEQ ID NO: 24 or SEQ ID NO: 25, but not both. Given the high degree of amino acid sequence identity between native hG1Fc, native hG2Fc, native hG3Fc, and native hG4Fc, it can be appreciated that amino acid substitutions at corresponding positions in hG1Fc, hG2Fc, hG3Fc, or hG4Fc (see FIG. 3) will generate an Fc monomer which may be used in the complementary IgG-IgA pair below (SEQ ID NOs: 24 and 25).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PFRPEVHLLP PSREEMTKNQ VSLTCLARGF
151 YPKDIAVEWE SNGQPENNYK TTPSRQEPSQ GTTTFAVTSK LTVDKSRWQQ
201 GNVFSCSVMH EALHNHYTQK TISLSPGK (SEQ ID NO: 24)

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PPSEELALNE LVTLTCLVKG
151 FYPSDIAVEW ESNGQELPRE KYLTWAPVLD SDGSFFLYSI LRVAAEDWKK
201 GDTFSCSVMH EALHNHYTQK SLDRSPGK (SEQ ID NO: 25)

In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24. In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 24, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25.

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains. See, e.g., Wranik et al (2012) J Biol Chem 287:43331-43339. As disclosed herein, one of a pair of Fc sequences attached to a leucine zipper-forming strand can be arbitrarily fused to a first variant ActRIIB polypeptide, second variant ActRIIB polypeptide, or unmodified ActRIIB polypeptide of the construct, with or without an optional linker, to generate a variant ActRIIB-Fc or unmodified ActRIIB-Fc fusion polypeptide. This single chain can be coexpressed in a cell of choice along with the Fc sequence attached to a complementary leucine zipper-forming strand to favor generation of the desired multichain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 26 [hG1Fc-Ap1 (acidic)] and SEQ ID NO: 27 [hG1Fc-Bp1 (basic)] are examples of complementary IgG Fc sequences in which the engineered complimentary leucine zipper sequences are underlined, and a first variant ActRIIB polypeptide, second variant ActRIIB polypeptide, or wild-type ActRIIB polypeptide of the construct can be fused to either SEQ ID NO: 26 or SEQ ID NO: 27, but not both. Given the high degree of amino acid sequence identity between native hG1Fc, native hG2Fc, native hG3Fc, and native hG4Fc, it can be appreciated that leucine zipper-forming sequences attached, with or without an optional linker, to hG1Fc, hG2Fc, hG3Fc, or hG4Fc (see FIG. 3) will generate an Fc monomer which may be used in the complementary leucine zipper-forming pair below (SEQ ID NOs: 26 and 27).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLYSKLTV DKSRWQQGNV
201 FSCSVMHEAL HNHYTQKSLS LSPGKGGSAQ LEKELQALEK ENAQLEWELQ
251 ALEKELAQGA T (SEQ ID NO: 26)

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLYSKLTV DKSRWQQGNV
201 FSCSVMHEAL HNHYTQKSLS LSPGKGGSAQ LKKKLQALKK KNAQLKWKLQ
251 ALKKKLAQGA T (SEQ ID NO: 27)

In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26. In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 26, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27.

In part, the disclosure provides desired pairing of asymmetric Fc-containing polypeptide chains by methods described above in combination with additional mutations in the Fc domain which facilitate purification of the desired heteromeric species. An example uses complementarity of Fc domains based on knobs-into-holes pairing combined with an engineered disulfide bond, as disclosed in SEQ ID NOs: 22 and 23, plus additional substitution of two negatively charged amino acids (aspartic acid or glutamic acid) in one Fc-containing polypeptide chain and two positively charged amino acids (e.g., arginine) in the complementary Fc-containing polypeptide chain (SEQ ID NOs: 28-29). These four amino acid substitutions facilitate selective purification of the desired heteromeric fusion protein from a heterogeneous polypeptide mixture based on differences in isoelectric point or net molecular charge. The engineered amino acid substitutions in these sequences are double underlined below, and a variant ActRIIB polypeptide, an ALK4 polypeptide, or an ALK7 polypeptide of the construct can be fused to either SEQ ID NO: 28 or SEQ ID NO: 29, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair below (SEQ ID NOs: 28-29).

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PCREEMTENQ VSLWCLVKGF
151 YPSDIAVEWE SNGQPENNYK TTPPVLDSDG SFFLYSKLTV DKSRWQQGNV
201 FSCSVMHEAL HNHYTQDSLS LSPGK (SEQ ID NO: 28)

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVCTLP PSREEMTKNQ VSLSCAVKGF
151 YPSDIAVEWE SRGQPENNYK TTPPVLDSRG SFFLVSKLTV DKSRWQQGNV
201 FSCSVMHEAL HNHYTQKSLS LSPGK (SEQ ID NO: 29)

In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29. In some embodiments, the variant ActRIIB-Fc fusion protein Fc domain comprises a cysteine at amino acid position 132, glutamic acid at amino acid position 138, a tryptophan at amino acid position 144, and an aspartic acid at amino acid position 217. In some embodiments, the ALK4-Fc fusion protein Fc domain comprises a cysteine at amino acid position 127, a serine at amino acid position 144, an alanine at amino acid position 146, an arginine at amino acid position 162, an arginine at amino acid position 179, and a valine at amino acid position 185. In some embodiments, the ALK7-Fc fusion protein Fc domain comprises a cysteine at amino acid position 127, a serine at amino acid position 144, an alanine at amino acid position 146, an arginine at amino acid position 162, an arginine at amino acid position 179, and a valine at amino acid position 185.

In some embodiments, the disclosure relates to ActRIIB:ALK4 heteromultimer proteins comprising a variant ActRIIB-Fc fusion protein and an ALK4-Fc fusion protein wherein the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29. In some embodiments, the disclosure relates to ActRIIB:ALK7 heteromultimer proteins comprising a variant ActRIIB-Fc fusion protein and an ALK7-Fc fusion protein wherein the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 28, and the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29. In some embodiments, the ALK4-Fc fusion protein Fc domain comprises a cysteine at amino acid position 132, glutamic acid at amino acid position 138, a tryptophan at amino acid position 144, and an aspartic acid at amino acid position 217. In some embodiments, the variant ActRIIB-Fc fusion protein Fc domain comprises a cysteine at amino acid position 127, a serine at amino acid position 144, an alanine at amino acid position 146, an arginine at amino acid position 162, an arginine at amino acid position 179, and a valine at amino acid position 185. In some embodiments, the ALK7-Fc fusion protein Fc domain comprises a cysteine at amino acid position 132, glutamic acid at amino acid position 138, a tryptophan at amino acid position 144, and an aspartic acid at amino acid position 217. In some embodiments, the variant ActRIIB-Fc fusion protein Fc domain comprises a cysteine at amino acid position 127, a serine at amino acid position 144, an alanine at amino acid position 146, an arginine at amino acid position 162, an arginine at amino acid position 179, and a valine at amino acid position 185.

Another example involves complementarity of Fc domains based on knobs-into-holes pairing combined with an engineered disulfide bond, as disclosed in SEQ ID NOs: 22-23, plus a histidine-to-arginine substitution at position 213 in one Fc-containing polypeptide chain (SEQ ID NO: 30). This substitution (denoted H435R in the numbering system of Kabat et al.) facilitates separation of desired heteromer from undesirable homodimer based on differences in affinity for protein A. The engineered amino acid substitution is indicated by double underline, and a variant ActRIIB polypeptide, ALK4 polypeptide, or ALK7 polypeptide of the construct can be fused to either SEQ ID NO: 30 or SEQ ID NO: 23, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair of SEQ ID NO: 30 (below) and SEQ ID NO: 23.

1 THTCPPCPAP ELLGGPSVFL FPPKPKDTLM ISRTPEVTCV VVDVSHEDPE
51 VKFNWYVDGV EVHNAKTKPR EEQYNSTYRV VSVLTVLHQD WLNGKEYKCK
101 VSNKALPAPI EKTISKAKGQ PREPQVYTLP PCREEMTKNQ VSLWCLVKGF
151 YPSDIAVEWE SNGQPENNYK TTPPVLDSDG SFFLYSKLTV DKSRWQQGNV
201 FSCSVMHEAL HNRYTQKSLS LSPGK (SEQ ID NO: 30)

In some embodiments, the disclosure relates to ActRIIB:ALK4 heteromultimer proteins comprising a variant ActRIIB-Fc fusion protein and an ALK4-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23. In some embodiments, the disclosure relates to ActRIIB:ALK7 heteromultimer proteins comprising a variant ActRIIB-Fc fusion protein and an ALK7-Fc fusion protein wherein the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23. In some embodiments, the variant ActRIIB-Fc fusion protein Fc domain comprises a cysteine at amino acid position 132, a tryptophan at amino acid position 144, and an arginine at amino acid position 435. In some embodiments, the ALK4-Fc fusion protein Fc domain comprises cysteine at amino acid position 127, a serine at amino acid position 144, an alanine at amino acid position 146, and a valine at amino acid position 185. In some embodiments, the ALK7-Fc fusion protein Fc domain comprises cysteine at amino acid position 127, a serine at amino acid position 144, an alanine at amino acid position 146, and a valine at amino acid position 185.

In some embodiments, the disclosure relates to ActRIIB:ALK4 heteromultimer proteins comprising a variant ActRIIB-Fc fusion protein and an ALK4-Fc fusion protein wherein the ALK4-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23. In some embodiments, the disclosure relates to ActRIIB:ALK7 heteromultimer proteins comprising a variant ActRIIB-Fc fusion protein and an ALK7-Fc fusion protein wherein the ALK7-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and the variant ActRIIB-Fc fusion protein comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23 In some embodiments, the ALK4-Fc fusion protein Fc domain comprises a cysteine at amino acid position 132, a tryptophan at amino acid position 144, and an arginine at amino acid position 435. In some embodiments, the ALK7-Fc fusion protein Fc domain comprises a cysteine at amino acid position 132, a tryptophan at amino acid position 144, and an arginine at amino acid position 435. In some embodiments, the variant ActRIIB-Fc fusion protein Fc domain comprises cysteine at amino acid position 127, a serine at amino acid position 144, an alanine at amino acid position 146, and a valine at amino acid position 185.

A variety of engineered mutations in the Fc domain are presented above with respect to the G1Fc sequence (SEQ ID NO: 13). Analogous mutations in G2Fc, G3Fc, and G4Fc can be derived from their alignment with G1Fc in FIG. 3. Due to unequal hinge lengths, analogous Fc positions based on isotype alignment (FIG. 3) possess different amino acid numbers in SEQ ID NOs: 13, 14, 15, 16, and 17 as summarized in the following table.

Correspondence between C_{H}3 Positions for Human Fc lsotypes∗

| IgG1 | IgG4 | IgG2 | IgG3 |
|---|---|---|---|
| SEQ ID NO: 13 | SEQ ID NO: 17 | SEQ ID NO: 14 | SEQ ID NO: 15 |
| Numbering begins at THT... | Numbering begins at ESK... | Numbering begins at VEC... | Numbering begins at EPK... |
| Y127 | Y131 | Y125 | Y134 |
| S132 | S136 | S130 | 5139 |
| E134 | E138 | E132 | E141 |
| K138 | K142 | K136 | K145 |
| T144 | T148 | T142 | T151 |
| L146 | L150 | L144 | L153 |
| N162 | N166 | N160 | 5169 |
| K170 | K174 | K168 | N177 |
| D177 | D181 | D175 | D184 |
| D179 | D183 | D177 | D186 |
| Y185 | Y189 | Y183 | Y192 |
| K187 | R191 | K185 | K194 |
| H213 | H217 | H211 | R220 |
| K217 | K221 | K215 | K224 |

^{∗} Numbering based on multiple sequence alignment shown in FIG. 3

It is understood that different elements of the fusion proteins (e.g., immunoglobulin Fc fusion proteins) may be arranged in any manner that is consistent with desired functionality. For example, a variant ActRIIB polypeptide domain may be placed C-terminal to a heterologous domain, or alternatively, a heterologous domain may be placed C-terminal to a variant ActRIIB polypeptide domain. The variant ActRIIB polypeptide domain and the heterologous domain need not be adjacent in a fusion protein, and additional domains or amino acid sequences may be included C- or N-terminal to either domain or between the domains.

For example, a variant ActRIIB polypeptide may comprise an amino acid sequence as set forth in the formula A-B-C. The B portion corresponds to a variant ActRIIB polypeptide domain. The A and C portions may be independently zero, one, or more than one amino acid, and both the A and C portions when present are heterologous to B. The A and/or C portions may be attached to the B portion via a linker sequence. In certain embodiments, a variant ActRIIB fusion protein comprises an amino acid sequence as set forth in the formula A-B-C, wherein A is a leader (signal) sequence, B consists of a variant ActRIIB polypeptide domain, and C is a polypeptide portion that enhances one or more of in vivo stability, in vivo half-life, uptake/administration, tissue localization or distribution, formation of protein complexes, and/or purification. In certain embodiments, a variant ActRIIB fusion protein comprises an amino acid sequence as set forth in the formula A-B-C, wherein A is a TPA leader sequence, B consists of a variant ActRIIB polypeptide domain, and C is an immunoglobulin Fc domain.

In certain embodiments, the variant ActRIIB polypeptides of the present disclosure contain one or more modifications that are capable of stabilizing the variant ActRIIB polypeptides. For example, such modifications enhance the in vitro half-life of the variant ActRIIB polypeptides, enhance circulatory half-life of the variant ActRIIB polypeptides or reducing proteolytic degradation of the variant ActRIIB polypeptides. Such stabilizing modifications include, but are not limited to, fusion proteins (including, for example, fusion proteins comprising a variant ActRIIB polypeptide and a stabilizer domain), modifications of a glycosylation site (including, for example, addition of a glycosylation site to a variant ActRIIB polypeptide), and modifications of carbohydrate moiety (including, for example, removal of carbohydrate moieties from a variant ActRIIB polypeptide). In the case of fusion proteins, a variant ActRIIB polypeptide is fused to a stabilizer domain such as an IgG molecule (e.g., an Fc domain). As used herein, the term “stabilizer domain” not only refers to a fusion domain (e.g., Fc) as in the case of fusion proteins, but also includes nonproteinaceous modifications such as a carbohydrate moiety, or nonproteinaceous polymer, such as polyethylene glycol.

In certain embodiments, the present disclosure makes available isolated and/or purified forms of the variant ActRIIB polypeptides, which are isolated from, or otherwise substantially free of, other proteins.

In certain embodiments, variant ActRIIB polypeptides of the disclosure can be produced by a variety of art-known techniques. For example, such variant ActRIIB polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User’s Guide, W. H. Freeman and Company, New York (1992). In addition, automated peptide synthesizers are commercially available (e.g., Advanced ChemTech Model 396; Milligen/Biosearch 9600). Alternatively, the variant ActRIIB polypeptides, fragments or variants thereof may be recombinantly produced using various expression systems (e.g., E. coli, Chinese Hamster Ovary cells, COS cells, baculovirus) as is well known in the art (also see below). In a further embodiment, the variant ActRIIB polypeptides may be produced by digestion of naturally occurring or recombinantly produced full-length variant ActRIIB polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites. Alternatively, such variant ActRIIB polypeptides may be produced from naturally occurring or recombinantly produced full-length variant ActRIIB polypeptides such as standard techniques known in the art, such as by chemical cleavage (e.g., cyanogen bromide, hydroxylamine).

In certain embodiments, variant ActRIIB polypeptides of the disclosure can include a purification subsequence, such as an epitope tag, a FLAG tag, a polyhistidine sequence, and a GST fusion. Optionally, a variant ActRIIB polypeptide includes one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent.

In some embodiments, the disclosure relates to variant ActRIIB polypeptides, including variant ActRIIB polypeptides as well as homomultimer and heteromultimers comprising the same, that comprise one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety. In some embodiments, variant ActRIIB polypeptides of the disclosure are glycosylated and have a glycosylation pattern obtainable from of the polypeptide in a CHO cell.

### 3. Linkers

The disclosure provides for variant ActRIIB polypeptides that may be fused to any of the other polypeptides disclosed herein, or that may be fused to a heterologous portion (e.g., an Fc portion). In these embodiments, the polypeptide portion (e.g. a variant ActRIIB polypeptide) is connected to the other polypeptide (e.g., a TGFβRII polypeptide) and/or to the heterologous portion (e.g., Fc portion) by means of a linker. In some embodiments, the linkers are glycine and serine rich linkers. In some embodiments, the linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 261), GGGG (SEQ ID NO: 262), TGGGG(SEQ ID NO: 263), SGGGG(SEQ ID NO: 264), TGGG(SEQ ID NO: 265), or SGGG(SEQ ID NO: 266) singlets, or repeats. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In preferred embodiments, the linker comprises the amino acid sequence GlyGlyGlyGlySer (GGGGS) (SEQ ID NO: 267), or repetitions thereof (GGGGS)n, where n ≥ 2. In particular embodiments n ≥ 3, or n = 3-10. In some embodiments, n ≥ 4, or n = 4-10. In some embodiments, n is not greater than 4 in a (GGGGS)n linker. In some embodiments, n = 4-10, 4-9, 4-8, 4-7, 4-6, 4-5, 5-8, 5-7, or 5-6. In some embodiments, n = 3, 4, 5, 6, or 7. In particular embodiments, n = 4. In some embodiments, a linker comprising a (GGGGS)n sequence also comprises an N-terminal threonine. In some embodiments, the linker is any one of the following:

GGGGSGGGGS (SEQ ID NO: 268)

TGGGGSGGGGS (SEQ ID NO: 269)

TGGGGSGGGGSGGGGS (SEQ ID NO: 270)

TGGGGSGGGGSGGGGSGGGGS (SEQ ID NO: 271)

TGGGGSGGGGSGGGGSGGGGSGGGGS (SEQ ID NO: 272)

TGGGGSGGGGSGGGGSGGGGSGGGGSGGGGS
(SEQ ID NO: 273) or

TGGGGSGGGGSGGGGSGGGGSGGGGSGGGGSGGGGS
(SEQ ID NO: 274).

In some embodiments, the linker comprises the amino acid sequence of TGGGPKSCDK (SEQ ID NO: 275). In some embodiments, the linker is any one of SEQ ID NOs: 268-275 lacking the N-terminal threonine. In some embodiments, the linker does not comprise the amino acid sequence of SEQ ID NO: 273 or 274.

In some embodiments, a polypeptide described (e.g., variant ActRIIB proteins in either homomeric or heteromeric forms) herein may include a polypeptide fused to a moiety by way of a linker. In some embodiments, the moiety increases stability of the polypeptide. In some embodiments, the moiety is selected from the group consisting of an Fc domain monomer, a wild-type Fc domain, an Fc domain with amino acid substitutions (e.g., one or more substitutions that reduce dimerization), an albumin-binding peptide, a fibronectin domain, or a human serum albumin. Suitable peptide linkers are known in the art, and include, for example, peptide linkers containing flexible amino acid residues such as glycine, alanine, and serine. In some embodiments, a linker can contain motifs, e.g., multiple or repeating motifs, of GA, GS, GG, GGA, GGS, GGG, GGGA (SEQ ID NO: 280), GGGS (SEQ ID NO: 281), GGGG (SEQ ID NO: 262), GGGGA (SEQ ID NO: 282), GGGGS (SEQ ID NO: 267), GGGGG (SEQ ID NO: 283), GGAG (SEQ ID NO: 284), GGSG (SEQ ID NO: 285), AGGG (SEQ ID NO: 286), or SGGG (SEQ ID NO: 266). In some embodiments, a linker can contain 2 to 12 amino acids including motifs of GA or GS, e.g., GA, GS, GAGA (SEQ ID NO: 287), GSGS (SEQ ID NO: 288), GAGAGA (SEQ ID NO: 289), GSGSGS (SEQ ID NO: 290), GAGAGAGA (SEQ ID NO: 291), GSGSGSGS (SEQ ID NO: 292), GAGAGAGAGA (SEQ ID NO: 293), GSGSGSGSGS (SEQ ID NO: 294), GAGAGAGAGAGA (SEQ ID NO: 295), and GSGSGSGSGSGS (SEQ ID NO: 296). In some embodiments, a linker can contain 3 to 12 amino acids including motifs of GGA or GGS, e.g., GGA, GGS, GGAGGA (SEQ ID NO: 297), GGSGGS (SEQ ID NO: 298), GGAGGAGGA (SEQ ID NO: 299), GGSGGSGGS (SEQ ID NO: 300), GGAGGAGGAGGA (SEQ ID NO: 301), and GGSGGSGGSGGS (SEQ ID NO: 302). In some embodiments, a linker can contain 4 to 12 amino acids including motifs of GGAG (SEQ ID NO: 303), GGSG (SEQ ID NO: 304), GGAGGGAG (SEQ ID NO: 305), GGSGGGSG (SEQ ID NO: 306), GGAGGGAGGGAG (SEQ ID NO: 307), and GGSGGGSGGGSG (SEQ ID NO: 308). In some embodiments, a linker can contain motifs of GGGGA (SEQ ID NO: 309) or GGGGS (SEQ ID NO: 267), e.g., GGGGAGGGGAGGGGA (SEQ ID NO: 310) and GGGGSGGGGSGGGGS (SEQ ID NO: 311). In some embodiments, an amino acid linker between a moiety (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a polypeptide (e.g., variant ActRIIB proteins in either homomeric or heteromeric forms) may be GGG, GGGA (SEQ ID NO: 280), GGGG (SEQ ID NO: 262), GGGAG (SEQ ID NO: 312), GGGAGG (SEQ ID NO: 313), or GGGAGGG (SEQ ID NO: 314).

In some embodiments, a linker can also contain amino acids other than glycine, alanine, and serine, e.g., AAAL (SEQ ID NO: 315), AAAK (SEQ ID NO: 316), AAAR (SEQ ID NO: 317), EGKSSGSGSESKST (SEQ ID NO: 318), GSAGSAAGSGEF (SEQ ID NO: 319), AEAAAKEAAAKA (SEQ ID NO: 320), KESGSVSSEQLAQFRSLD (SEQ ID NO: 321), GENLYFQSGG (SEQ ID NO: 322), SACYCELS (SEQ ID NO: 323), RSIAT (SEQ ID NO: 324), RPACKIPNDLKQKVMNH (SEQ ID NO: 325), GGSAGGSGSGSSGGSSGASGTGTAGGTGSGSGTGSG (SEQ ID NO: 326), AAANSSIDLISVPVDSR (SEQ ID NO: 327), or GGSGGGSEGGGSEGGGSEGGGSEGGGSEGGGSGGGS (SEQ ID NO: 328). In some embodiments, a linker can contain motifs, e.g., multiple or repeating motifs, of EAAAK (SEQ ID NO: 329). In some embodiments, a linker can contain motifs, e.g., multiple or repeating motifs, of praline-rich sequences such as (XP)n, in which X may be any amino acid (e.g., A, K, or E) and n is from 1-5, and PAPAP(SEQ ID NO: 330).

The length of the peptide linker and the amino acids used can be adjusted depending on the two proteins involved and the degree of flexibility desired in the final protein fusion polypeptide. The length of the linker can be adjusted to ensure proper protein folding and avoid aggregate formation.

### 4. Nucleic Acids Encoding ActRIIB Polypeptides

In certain aspects, the disclosure provides isolated and/or recombinant nucleic acids encoding any of the variant ActRIIB polypeptides (e.g., soluble ActRIIB polypeptides), including any of the variants disclosed herein. For example, SEQ ID NO: 4 encodes a naturally occurring ActRIIB precursor polypeptide, while SEQ ID NO: 3 encodes a soluble ActRIIB polypeptide. The subject nucleic acids may be single-stranded or double stranded. Such nucleic acids may be DNA or RNA molecules. These nucleic acids are may be used, for example, in methods for making ActRIIB polypeptides or as direct therapeutic agents (e.g., in a gene therapy approach).

In certain aspects, the disclosure relates to isolated and/or recombinant nucleic acids comprising a coding sequence for one or more of the variant ActRIIB polypeptide(s) as described herein. For example, in some embodiments, the disclosure relates to an isolated and/or recombinant nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, 47, 277, 331, 334, 337, 340, 343, 346, 349, 352, 355, 367, 370, 373, 376, 379, 382, 385, 388, 391, 394, 397, 400, 403, 406, 409, 412, 415, 418, 421, 424, 427, 430, 433, 436, 439, 442, 445, 448, 451, 454, 457, 460, 463, 466,469,472,475,478,481,484,487,490,493,496,499, 502, 505, 508, 511, 514, 517, 521, and 523. In some embodiments, an isolated and/or recombinant polynucleotide sequence of the disclosure comprises a promoter sequence operably linked to a coding sequence described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, 47, 277, 331, 334, 337, 340, 343, 346, 349, 352, 355, 367, 370, 373, 376, 379, 382, 385, 388, 391, 394, 397, 400, 403, 406, 409, 412, 415, 418, 421, 424, 427, 430, 433, 436, 439, 442, 445, 448, 451, 454, 457,460,463,466,469,472,475,478,481,484,487,490,493,496,499,502,505,508, 511, 514, 517, 521, and 523). In some embodiments, the disclosure relates to vectors comprising an isolated and/or recombinant nucleic acid described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10,32,35,38,41,44,47,277,331,334,337,340,343,346,349,352,355,367,370,373, 376,379,382,385,388,391,394,397,400,403,406,409,412,415,418,421,424,427, 430,433,436,439,442,445,448,451,454,457,460,463,466,469,472,475,478,481, 484, 487, 490, 493, 496, 499, 502, 505, 508, 511, 514, 517, 521, and 523). In some embodiments, the disclosure relates to a cell comprising an isolated and/or recombinant polynucleotide sequence described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, 47, 277, 331, 334, 337, 340, 343, 346, 349, 352, 355, 367, 370, 373, 376, 379, 382, 385, 388, 391, 394, 397, 400, 403, 406, 409, 412, 415, 418, 421, 424, 427, 430, 433, 436, 439, 442, 445,448,451,454,457,460,463,466,469,472,475,478,481,484,487,490,493,496, 499, 502, 505, 508, 511, 514, 517, 521, and 523). In some embodiments, the cell is a CHO cell. In some embodiments, the cell is a COS cell.

In certain embodiments, nucleic acids encoding variant ActRIIB (or homomultimers or heteromultimers thereof), ALK4 or ALK7 polypeptides of the disclosure are understood to include nucleic acids that are variants of any one of SEQ ID NOs: 3, 4, 10, 32, 35, 38, 41, 44, 47, 221, 222, 223, 224, 233, 234, 235, 236, 237, 238, 239, 240, 255, 277, 331, 334, 337, 340, 343,346,349,352,355,367,370,373,376,379,382,385,388,391,394,397,400,403, 406,409,412,415,418,421,424,427,430,433,436,439,442,445,448,451,454,457, 460,463,466,469,472,475,478,481,484,487,490,493,496,499,502,505,508,511, 514, 517, 521, and 523. Variant nucleotide sequences include sequences that differ by one or more nucleotide substitutions, additions, or deletions including allelic variants, and therefore, will include coding sequence that differ from the nucleotide sequence designated in any one of SEQ ID NOs: 3, 4, 10, 32, 35, 38, 41, 44, 47, 221, 222, 223, 224, 233, 234, 235, 236, 237, 238, 239, 240, 255, 277, 331, 334, 337, 340, 343, 346, 349, 352, 355, 367, 370, 373, 376, 379,382,385,388,391,394,397,400,403,406,409,412,415,418,421,424,427,430, 433,436,439,442,445,448,451,454,457,460,463,466,469,472,475,478,481,484, 487, 490, 493, 496, 499, 502, 505, 508, 511, 514, 517, 521, and 523.

In certain embodiments, variant ActRIIB (or homomultimers or heteromultimers thereof), ALK4, or ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to any one of SEQ ID NOs: 3, 4, 10,32,35,38,41,44,47,221,222,223,224,233,234,235,236,237,238,239,240,255, 277,331,334,337,340,343,346,349,352,355,367,370,373,376,379,382,385,388, 391,394,397,400,403,406,409,412,415,418,421,424,427,430,433,436,439,442, 445,448,451,454,457,460,463,466,469,472,475,478,481,484,487,490,493,496, 499, 502, 505, 508, 511, 514, 517, 521, and 523. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 3. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 4. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 10. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 32. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 35. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 38. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 41. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 44. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 47. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 277. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 331. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 334. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 337. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 340. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 343. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 346. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 349. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 352. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 355. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 367. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 370. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 373. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 376. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 379. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 382. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 385. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 388. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 391. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 394. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 397. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 400. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 403. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 406. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 409. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 412. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 415. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 418. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 421. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 424. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 427. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 430. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 433. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 436. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 439. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 442. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 445. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 448. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 451. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 454. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 457. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 460. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 463. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 466. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 469. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 472. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 475. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 478. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 481. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 484. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 487. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 490. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 493. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 496. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 499. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 502. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 505. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 508. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 511. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 514. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 517. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 521. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 523.

In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 221. In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 222. In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 223. In certain embodiments, ALK4 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 224.

In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 233. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 234. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 235. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 236. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 237. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 238. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 239. In certain embodiments, ALK7 polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 240.

In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 243. In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 248. In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 250. In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 251. In certain embodiments, ALK4-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 252. In certain embodiments, ALK7-Fc fusion polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 255.

In certain aspects, the subject nucleic acids encoding variant ActRIIB polypeptides are further understood to include nucleic acids that are variants of SEQ ID NO: 3. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NO: 4.

In certain embodiments, the disclosure provides isolated or recombinant nucleic acid sequences that are at least 75%, 80%, 85%, 90%, 91%, 92%, 93%, 94%, 95%, 96%, 97%, 98%, 99%, or 100% identical to SEQ ID NO: 3. One of ordinary skill in the art will appreciate that nucleic acid sequences complementary to SEQ ID NO: 3, and variants of SEQ ID NO: 3 are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to nucleic acids encoding variant ActRIIB proteins in either homomeric or heteromeric forms, ALK4, or ALK7 polypeptides of the disclosure (e.g., SEQ ID NO: 3), the complement sequence, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0 x sodium chloride/sodium citrate (SSC) at about 45° C., followed by a wash of 2.0 x SSC at 50° C. For example, the salt concentration in the wash step can be selected from a low stringency of about 2.0 x SSC at 50° C. to a high stringency of about 0.2 x SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6 x SSC at room temperature followed by a wash at 2 x SSC at room temperature.

Isolated nucleic acids which differ from the nucleic acids as set forth in SEQ ID NO: 3 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects, the subject nucleic acid is provided in an expression vector comprising a nucleotide sequence encoding a variant ActRIIB polypeptide and operably linked to at least one regulatory sequence. Regulatory sequences are art-recognized and are selected to direct expression of the variant ActRIIB polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding a variant ActRIIB polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda , the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector’s copy number, the ability to control that copy number and the expression of any other protein encoded by the vector, such as antibiotic markers, should also be considered.

A recombinant nucleic acid of the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant variant ActRIIB polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In a preferred embodiment, a vector will be designed for production of the subject variant ActRIIB polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject variant ActRIIB polypeptides in cells propagated in culture, e.g., to produce proteins, including fusion proteins or variant proteins, for purification.

In certain embodiments, the disclosure relates to methods of making ActRIIB polypeptides, including variant ActRIIB polypeptides as well as homomultimer and heteromultimers comprising the same, as described herein. Such a method may include expressing any of the nucleic acids disclosed herein in a suitable cell (e.g., a CHO cell or COS cell). Such a method may comprise: a) culturing a cell under conditions suitable for expression of the soluble ActRIIB polypeptide, wherein said cell comprise with an ActRIIB polypeptide expression construct. In some embodiments, the method further comprises recovering the expressed ActRIIB polypeptide. ActRIIB polypeptides may be recovered as crude, partially purified or highly purified fractions using any of the well-known techniques for obtaining protein from cell cultures.

This disclosure also pertains to a host cell transfected with a recombinant gene including a coding sequence (e.g., SEQ ID NO: 4) for one or more of the subject variant ActRIIB polypeptides. The host cell may be any prokaryotic or eukaryotic cell. For example, a variant ActRIIB polypeptide of the disclosure may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject variant ActRIIB polypeptides. For example, a host cell transfected with an expression vector encoding a variant ActRIIB polypeptide can be cultured under appropriate conditions to allow expression of the ActRIIB polypeptide to occur. The variant ActRIIB polypeptide may be secreted and isolated from a mixture of cells and medium containing the variant ActRIIB polypeptide. Alternatively, the variant ActRIIB polypeptide may be retained cytoplasmically or in a membrane fraction and the cells harvested, lysed and the protein isolated. A cell culture includes host cells, media, and other byproducts. Suitable media for cell culture are well known in the art. The subject variant ActRIIB polypeptides can be isolated from cell culture medium, host cells, or both, using techniques known in the art for purifying proteins, including ion-exchange chromatography, gel filtration chromatography, ultrafiltration, electrophoresis, and immunoaffinity purification with antibodies specific for particular epitopes of the variant ActRIIB polypeptides. In a preferred embodiment, the variant ActRIIB polypeptide is a fusion protein containing a domain which facilitates its purification.

In another embodiment, a fusion gene coding for a purification leader sequence, such as a poly-(His)/enterokinase cleavage site sequence at the N-terminus of the desired portion of the recombinant variant ActRIIB polypeptide, can allow purification of the expressed fusion protein by affinity chromatography using a Ni²⁺ metal resin. The purification leader sequence can then be subsequently removed by treatment with enterokinase to provide the purified variant ActRIIB polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### 5. Screening Assays

In certain aspects, the present disclosure relates to the use of the subject variant ActRIIB polypeptides (e.g., variant ActRIIB polypeptides) to identify compounds (agents) which are agonist or antagonists of the variant ActRIIB polypeptides. Compounds identified through this screening can be tested in tissues such as renal, pulmonary, cardiac, bone, cartilage, muscle, fat, and/or neurons, to assess their ability to modulate tissue growth in vitro. Additionally, compounds identified through this screening can be tested for efficacy in treating human or animal disorders or conditions such as pulmonary disorders (e.g., pulmonary hypertension, interstitial lung disease, idiopathic pulmonary fibrosis), renal diseases or conditions (e.g., Alport syndrome, focal segmental glomerulosclerosis (FSGS), polycystic kidney disease, chronic kidney disease), peripheral neuropathy, Charcot-Marie-Tooth disease, and anemia (e.g., anemia associated with myelodysplastic syndrome, thalassemia or myelofibrosis). Optionally, these compounds can further be tested in animal models to assess their ability to modulate tissue growth in vivo.

There are numerous approaches to screening for therapeutic agents for modulating tissue growth by targeting the variant ActRIIB polypeptides. In certain embodiments, high-throughput screening of compounds can be carried out to identify agents that perturb ActRIIB-mediated effects on growth of renal tissue, pulmonary tissue, cardiac tissue, bone, cartilage, muscle, fat, and/or neurons. In certain embodiments, the assay is carried out to screen and identify compounds that specifically inhibit or reduce binding of a variant ActRIIB polypeptide to its binding partner, such as a ligand of wildtype ActRIIB (e.g., activin A, activin B, GDF8, GDF11, and BMP10). Alternatively, the assay can be used to identify compounds that enhance binding of a variant ActRIIB polypeptide to its binding partner such as an ActRIIB ligand. In a further embodiment, the compounds can be identified by their ability to interact with a variant ActRIIB polypeptide.

A variety of assay formats will suffice, and, in light of the present disclosure, those not expressly described herein will nevertheless be comprehended by one of ordinary skill in the art. As described herein, the test compounds (agents) of the disclosure may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants, or other organisms (e.g., natural products), produced chemically (e.g., small molecules, including peptidomimetics), or produced recombinantly. Test compounds contemplated by the present disclosure include non-peptidyl organic molecules, peptides, polypeptides, peptidomimetics, sugars, hormones, and nucleic acid molecules. In a specific embodiment, the test agent is a small organic molecule having a molecular weight of less than about 2,000 daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers, and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatable crosslinkers or any combinations thereof.

In many drug screening programs that test libraries of compounds and natural extracts; high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a variant ActRIIB polypeptide and its binding protein (e.g., an ActRIIB ligand).

Merely to illustrate, in an exemplary screening assay of the present disclosure, the compound of interest is contacted with an isolated and purified ActRIIB polypeptide which is ordinarily capable of binding to an ActRIIB ligand, as appropriate for the intention of the assay. To the mixture of the compound and ActRIIB polypeptide is then added a composition containing an ActRIIB ligand. Detection and quantification of ActRIIB/ActRIIB ligand complexes provides a means for determining the compound’s efficacy at inhibiting (or potentiating) complex formation between the ActRIIB polypeptide and its binding protein. The efficacy of the compound can be assessed by generating dose response curves from data obtained using various concentrations of the test compound. Moreover, a control assay can also be performed to provide a baseline for comparison. For example, in a control assay, isolated and purified ActRIIB ligand is added to a composition containing the ActRIIB polypeptide, and the formation of ActRIIB/ActRIIB ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between the ActRIIB polypeptide and its binding protein may be detected by a variety of techniques. For instance, modulation of the formation of complexes can be quantitated using, for example, detectably labeled proteins such as radiolabeled (e.g., ³²P, ³⁵S, ¹⁴C or ³H), fluorescently labeled (e.g., FITC), or enzymatically labeled ActRIIB polypeptide or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between an ActRIIB polypeptide and its binding protein. Further, other modes of detection, such as those based on optical waveguides (PCT Publication WO 96/26432 and U.S. Pat. No. 5,677,196), surface plasmon resonance (SPR), surface charge sensors, and surface force sensors, are compatible with many embodiments described herein.

Moreover, the present disclosure contemplates the use of an interaction trap assay, also known as the “two hybrid assay,” for identifying agents that disrupt or potentiate interaction between an ActRIIB polypeptide and its binding protein. See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two hybrid systems to identify compounds (e.g., small molecules or peptides) that dissociate interactions between an ActRIIB polypeptide and its binding protein. See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368.

In certain embodiments, the subject compounds are identified by their ability to interact with a variant ActRIIB polypeptide of the disclosure. The interaction between the compound and the variant ActRIIB polypeptide may be covalent or non-covalent. For example, such interaction can be identified at the protein level using in vitro biochemical methods, including photo-crosslinking, radiolabeled ligand binding, and affinity chromatography (Jakoby WB et al., 1974, Methods in Enzymology 46: 1). In certain cases, the compounds may be screened in a mechanism based assay, such as an assay to detect compounds which bind to a variant ActRIIB polypeptide. This may include a solid phase or fluid phase binding event. Alternatively, the gene encoding a variant ActRIIB polypeptide can be transfected with a reporter system (e.g., β-galactosidase, luciferase, or green fluorescent protein) into a cell and screened against the library preferably by a high throughput screening or with individual members of the library. Other mechanism based binding assays may be used, for example, binding assays which detect changes in free energy. Binding assays can be performed with the target fixed to a well, bead or chip or captured by an immobilized antibody or resolved by capillary electrophoresis. The bound compounds may be detected usually using colorimetric or fluorescence or surface plasmon resonance.

In certain aspects, the present disclosure provides methods and agents for decreasing pulmonary hypertension and decreasing the pathogenic mechanism contributing to pulmonary hypertension, for example, by antagonizing functions of an ActRIIB polypeptide and/or an ActRIIB ligand. Therefore, any compound (e.g. variant ActRIIB polypeptides) identified can be tested in whole cells or tissues, in vitro or in vivo, to confirm their ability to decrease pulmonary hypertension. Various methods known in the art can be utilized for this purpose. For example, methods of the disclosure are performed such that the signal transduction through an ActRIIB protein activated by binding to an ActRIIB ligand (e.g., activin A, activin B, GDF8, GDF11, or BMP10) has been reduced or inhibited.

The present disclosure also contemplates in vivo assays to measure the effects of any compound (e.g. variant ActRIIB polypeptides) described herein on pulmonary hypertension. Various animal models of pulmonary hypertension (e.g. pulmonary arterial hypertension) known in the art can be utilized for this purpose. These include animal models such as chronic hypoxia induced pulmonary hypertension (e.g. Sugen Hypoxia model) and monocrotaline induced pulmonary hypertension. The effects of any compound on these animal models can assessed by measuring various parameters such as vessel muscularity, pulmonary artery cross sectional area, right ventricular stroke volume, right ventricular hypertrophy, right ventricular systolic pressure, and survival. These parameters can be assessed, in part, by removing and measuring (e.g. weight and length) the left ventricle (LV), septum (S), and right ventricle (RV) of each animal. Hypertrophy can be assessed, in part, by calculating RV/LV + S. Histopathologic scoring can also be used to measure the vessel muscularity. In some embodiments, methods and agents of the present disclosure can be screened in combination with additional active agents and/or supportive therapies that are currently used in treating pulmonary hypertension (e.g. sildenafil). In some embodiments, methods and agents of the present disclosure can be compared to active agents and/or supportive therapies that are currently used in treating pulmonary hypertension (e.g. sildenafil).

In certain aspects, the present disclosure provides methods and agents for decreasing renal diseases or conditions (e.g., Alport syndrome, focal segmental glomerulosclerosis (FSGS), polycystic kidney diseases, chronic kidney disease) and decreasing the pathogenic mechanism contributing to such diseases or conditions, for example, by antagonizing functions of an ActRIIB polypeptide and/or an ActRIIB ligand. Therefore, any compound (e.g. variant ActRIIB polypeptides) identified can be tested in whole cells or tissues, in vitro or in vivo, to confirm their ability to decrease renal diseases or conditions (e.g., Alport syndrome, focal segmental glomerulosclerosis (FSGS), polycystic kidney diseases, chronic kidney disease). Various methods known in the art can be utilized for this purpose. For example, methods of the disclosure are performed such that the signal transduction through an ActRIIB protein activated by binding to an ActRIIB ligand (e.g., activin A, activin B, GDF8, GDF11, or BMP10) has been reduced or inhibited.

The present disclosure also contemplates in vivo assays to measure the effects of any compound (e.g. variant ActRIIB polypeptides) described herein on renal diseases or conditions (e.g., Alport syndrome, focal segmental glomerulosclerosis (FSGS), polycystic kidney diseases, chronic kidney disease). Various animal models of renal diseases or conditions (e.g., Alport syndrome, focal segmental glomerulosclerosis (FSGS), polycystic kidney diseases, chronic kidney disease) known in the art can be utilized for this purpose. These include animal models such as Unilateral Ureter Obstruction (UUO) model for renal fibrosis and Col4a3-/- Alport model. The effects of any compound on these animal models can assessed by measuring various parameters such as. the expression of various fibrotic genes (Fibronectin, PAI-1, CTGF, Col-I, Col-III, and a-SMA), inflammatory genes (MCP-1 and TNFα), Thrombospondin 1 (Thbs1), kidney injury gene (NGAL), and TGFβ superfamily ligands (TGFβ1, TGFβ2, TGFβ3, and activin A). Upregulation of these TGFβ superfamily ligands is highly associated with kidney fibrosis/kidney dysfunction, and they serve as a good indicator of kidney damage. In general, several fibrotic genes are upregulated by TGFβ during fibrosis. Thbs1 is a direct downstream target of TGFβ, and also plays a role in regulating TGFβ activation, including during fibrosis. Measuring expression levels of Thbs1 gives an indication of the level of fibrosis, as an increase in Thbs1 expression likely means an increase in TGFβ expression. In some embodiments, methods and agents of the present disclosure can be screened in combination with additional active agents and/or supportive therapies that are currently used in treating renal diseases or conditions (e.g., Alport syndrome, focal segmental glomerulosclerosis (FSGS), polycystic kidney diseases, chronic kidney disease). In some embodiments, methods and agents of the present disclosure can be compared to active agents and/or supportive therapies that are currently used in treating renal diseases or conditions (e.g., Alport syndrome, focal segmental glomerulosclerosis (FSGS), polycystic kidney diseases, chronic kidney disease).

In certain aspects, the present disclosure provides methods and agents for treating, preventing, or reducing the progression rate and/or severity of a renal diseases or conditions (e.g., Alport syndrome, focal segmental glomerulosclerosis (FSGS), polycystic kidney diseases, chronic kidney disease), for example, by antagonizing functions of an ActRIIB polypeptide and/or an ActRIIB ligand. Therefore, any compound (e.g. variant ActRIIB polypeptides) identified can be tested in whole cells or tissues, in vitro or in vivo, to confirm their ability to treat a renal diseases or conditions (e.g., Alport syndrome, focal segmental glomerulosclerosis (FSGS), polycystic kidney diseases, chronic kidney disease). Various methods known in the art can be utilized for this purpose. For example, methods of the disclosure are performed such that the signal transduction through an ActRIIB protein activated by binding to an ActRIIB ligand (e.g., activin A, activin B, GDF8, GDF11, or BMP10) has been reduced or inhibited.

In certain aspects, the present disclosure provides methods and agents for stimulating muscle growth and increasing muscle mass, for example, by antagonizing functions of an ActRIIB polypeptide and/or an ActRIIB ligand. Therefore, any compound identified can be tested in whole cells or tissues, in vitro or in vivo, to confirm their ability to modulate muscle growth. Various methods known in the art can be utilized for this purpose. For example, methods of the disclosure are performed such that the signal transduction through an ActRIIB protein activated by binding to an ActRIIB ligand (e.g., activin A, activin B, GDF8, GDF11, or BMP10) has been reduced or inhibited. It will be recognized that the growth of muscle tissue in the organism would result in an increased muscle mass in the organism as compared to the muscle mass of a corresponding organism (or population of organisms) in which the signal transduction through an ActRIIB protein had not been so effected.

For example, the effect of the variant ActRIIB polypeptides or test compounds on muscle cell growth/proliferation can be determined by measuring gene expression of Pax-3 and Myf-5 which are associated with proliferation of myogenic cells, and gene expression of MyoD which is associated with muscle differentiation (e.g., Amthor et al., Dev Biol. 2002, 251:241-57). It is known that GDF8 down-regulates gene expression of Pax-3 and Myf-5, and prevents gene expression of MyoD. The variant ActRIIB polypeptides or test compounds are expected to antagonize this activity of GDF8. Another example of cell-based assays includes measuring the proliferation of myoblasts such as C(2)C(12) myoblasts in the presence of the variant ActRIIB polypeptides or test compounds (e.g., Thomas et al., J Biol Chem. 2000, 275:40235-43).

The present disclosure also contemplates in vivo assays to measure muscle mass and strength. For example, Whittemore et al. (Biochem Biophys Res Commun. 2003, 300:965-71) discloses a method of measuring increased skeletal muscle mass and increased grip strength in mice. Optionally, this method can be used to determine therapeutic effects of test compounds (e.g., variant ActRIIB polypeptides) on muscle diseases or conditions, for example those diseases for which muscle mass is limiting.

In certain aspects, the present disclosure provides methods and agents for modulating (stimulating or inhibiting) bone formation and increasing bone mass. Therefore, any compound identified can be tested in whole cells or tissues, in vitro or in vivo, to confirm their ability to modulate bone or cartilage growth. Various methods known in the art can be utilized for this purpose. For example, the effect of the variant ActRIIB polypeptides or test compounds on bone or cartilage growth can be determined by measuring induction of Msx2 or differentiation of osteoprogenitor cells into osteoblasts in cell based assays (see, e.g., Daluiski et al., Nat Genet. 2001, 27(1):84-8; Hino et al., Front Biosci. 2004, 9: 1520-9). Another example of cell-based assays includes analyzing the osteogenic activity of the subject variant ActRIIB polypeptides and test compounds in mesenchymal progenitor and osteoblastic cells. To illustrate, recombinant adenoviruses expressing an ActRIIB polypeptide were constructed to infect pluripotent mesenchymal progenitor C3H10T1/2 cells, preosteoblastic C2C12 cells, and osteoblastic TE-85 cells. Osteogenic activity is then determined by measuring the induction of alkaline phosphatase, osteocalcin, and matrix mineralization (see, e.g., Cheng et al., J bone Joint Surg Am. 2003, 85-A(8):1544-52).

The present disclosure also contemplates in vivo assays to measure bone or cartilage growth. For example, Namkung-Matthai et al., Bone, 28:80-86 (2001) discloses a rat osteoporotic model in which bone repair during the early period after fracture is studied. Kubo et al., Steroid Biochemistry & Molecular Biology, 68:197-202 (1999) also discloses a rat osteoporotic model in which bone repair during the late period after fracture is studied. These references are incorporated by reference herein in their entirety for their disclosure of rat model for study on osteoporotic bone fracture. In certain aspects, the present disclosure makes use of fracture healing assays that are known in the art. These assays include fracture technique, histological analysis, and biomechanical analysis, which are described in, for example, U.S. Pat. No. 6,521,750, which is incorporated by reference in its entirety for its disclosure of experimental protocols for causing as well as measuring the extent of fractures, and the repair process.

In certain aspects, the present disclosure provides methods and agents for controlling weight gain and obesity. At the cellular level, adipocyte proliferation and differentiation is critical in the development of obesity, which leads to the generation of additional fat cells (adipocytes). Therefore, any compound identified can be tested in whole cells or tissues, in vitro or in vivo, to confirm their ability to modulate adipogenesis by measuring adipocyte proliferation or differentiation. Various methods known in the art can be utilized for this purpose. For example, the effect of a variant ActRIIB polypeptide (e.g., a soluble variant ActRIIB polypeptide) or test compounds on adipogenesis can be determined by measuring differentiation of 3T3-L1 preadipocytes to mature adipocytes in cell based assays, such as, by observing the accumulation of triacylglycerol in Oil Red O staining vesicles and by the appearance of certain adipocyte markers such as FABP (aP2/422) and PPARy2. See, for example, Reusch et al., 2000, Mol Cell Biol. 20:1008-20; Deng et al., 2000, Endocrinology. 141:2370-6; Bell et al., 2000, Obes Res. 8:249-54. Another example of cell-based assays includes analyzing the role of variant ActRIIB polypeptides and test compounds in proliferation of adipocytes or adipocyte precursor cells (e.g., 3T3-L1 cells), such as, by monitoring bromodeoxyuridine (BrdU)-positive cells. See, for example, Pico et al., 1998, Mol Cell Biochem. 189:1-7; Masuno et al., 2003, Toxicol Sci. 75:314-20.

It is understood that the screening assays of the present disclosure apply to not only the subject variant ActRIIB polypeptides, but also any test compounds including agonists and antagonist of the ActRIIB polypeptides. Further, these screening assays are useful for drug target verification and quality control purposes.

### 6. Exemplary Therapeutic Uses

In certain embodiments, compositions of the present disclosure (e.g., variant ActRIIB proteins in either homomeric or heteromeric forms) can be used for treating or preventing a disease or condition that is associated with abnormal activity of a TGFβ superfamily ligand, such as an ActRIIB and/or an ActRIIB ligand (e.g., activin A, activin B, GDF8, GDF11, or BMP10). These diseases, disorders or conditions are generally referred to herein as “ActRIIB-associated conditions.” In certain embodiments, the present disclosure provides methods of treating or preventing an individual in need thereof through administering to the individual a therapeutically effective amount of a variant ActRIIB protein as described herein. These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The terms “treatment”, “treating”, “alleviation” and the like are used herein to generally mean obtaining a desired pharmacologic and/or physiologic effect, and may also be used to refer to improving, alleviating, and/or decreasing the severity of one or more clinical complication of a condition being treated. The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure is achieved by administering one or more variant ActRIIB proteins of the present disclosure (e.g., variant ActRIIB proteins in either homomeric or heteromeric forms) in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of a variant ActRIIB protein in either homomeric or heteromeric form, in combination with one or more additional active agents or other supportive therapy for treating or preventing a disease or condition (e.g., anemia, renal disease, pulmonary hypertension, pulmonary arterial hypertension, and ILD). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more a variant ActRIIB proteins of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the variant ActRIIB protein in either homomeric or heteromeric form of the present disclosure with the additional active agent or therapy and/or the desired effect.

Endogenous complexes between ActRIIB and ActRIIB ligands play essential roles in tissue growth as well as early developmental processes such as the correct formation of various structures or in one or more post-developmental capacities including sexual development, pituitary hormone production, and creation of bone and cartilage. Thus, ActRIIB-associated conditions include abnormal tissue growth and developmental defects. In addition, ActRIIB-associated conditions include, but are not limited to, disorders of cell growth and differentiation such as pulmonary disorders (e.g., pulmonary hypertension, interstitial lung disease, idiopathic pulmonary fibrosis), renal disease (e.g., Alport syndrome and focal segmental glomerulosclerosis), peripheral neuropathy, Charcot-Marie-Tooth disease, anemia (e.g., anemia associated with myelodysplastic syndrome, thalassemia or myelofibrosis), inflammation, allergy, autoimmune diseases, infectious diseases, and tumors. In certain embodiments, the present disclosure provides methods of promoting growth of a tissue or diminishing or preventing a loss of a tissue in a human. Exemplary tissues include renal tissue, pulmonary tissue, cardiac tissue, bone, cartilage, muscle, fat, and nervous tissue.

Exemplary ActRIIB-associated conditions include muscle disorders (e.g. neuromuscular disorders, musculodegenerative disorders, muscular dystrophy, muscle atrophy, muscle wasting associated with COPD, muscle wasting syndrome, sarcopenia, and cachexia), adipose tissue disorders (e.g., obesity), type 2 diabetes, bone degenerative disease (e.g., osteoporosis), pulmonary disorders (e.g., pulmonary hypertension, pulmonary arterial hypertension, interstitial lung disease, idiopathic pulmonary fibrosis, congestive obstructive pulmonary disease (COPD)), renal disease (e.g., Alport syndrome and focal segmental glomerulosclerosis), peripheral neuropathy, and Charcot-Marie-Tooth disease Other exemplary ActRIIB-associated conditions include anemia (e.g., anemia associated with myelodysplastic syndrome, anemia associated with thalassemia, or anemia associated with myelofibrosis), tissue repair (e.g., wound healing), neurodegenerative diseases (e.g., amyotrophic lateral sclerosis, spinal muscular atrophy), immunologic disorders (e.g., disorders related to abnormal proliferation or function of lymphocytes), and obesity or disorders related to abnormal proliferation of adipocytes.

In some embodiments, the disclosure relate to methods for treating a muscle-related disorder in a patient, comprising administering a patient in need thereof an ActRIIB polypeptide, including variant ActRIIB polypeptides as well as homomultimer and heteromultimers comprising the same, as described herein. In some embodiments, the disorder is associated with undesirably low muscle growth and/or muscle weakness. Such disorders include muscle atrophy, muscular dystrophy, amyotrophic lateral sclerosis (ALS), spinal muscular atrophy, muscle wasting disorders, cachexia, anorexia, DMD syndrome, BMD syndrome, AIDS wasting syndrome, muscular dystrophies, neuromuscular diseases, motor neuron diseases, diseases of the neuromuscular junction, facioscapulohumeral muscular dystrophy, Charcot-Marie-Tooth disease, peripheral neuropathy, and inflammatory myopathies.

In certain embodiments, ActRIIB polypeptides described herein (e.g., variant ActRIIB proteins in either homomeric or heteromeric forms) are used as part of a treatment for a muscular dystrophy. The term “muscular dystrophy” refers to a group of degenerative muscle diseases characterized by gradual weakening and deterioration of skeletal muscles and sometimes the heart and respiratory muscles. Muscular dystrophies are genetic disorders characterized by progressive muscle wasting and weakness that begin with microscopic changes in the muscle. As muscles degenerate over time, the person’s muscle strength declines. Exemplary muscular dystrophies that can be treated with a regimen including the subject variant ActRIIB proteins include: Duchenne muscular dystrophy (DMD), Becker muscular dystrophy (BMD), Emery-Dreifuss muscular dystrophy (EDMD), limb-girdle muscular dystrophy (LGMD), facioscapulohumeral muscular dystrophy (FSH or FSHD) (also known as Landouzy-Dejerine), myotonic dystrophy (MMD) (also known as Steinert’s disease), oculopharyngeal muscular dystrophy (OPMD), distal muscular dystrophy (DD), congenital muscular dystrophy (CMD).

Duchenne muscular dystrophy (DMD) was first described by the French neurologist Guillaume Benjamin Amand Duchenne in the 1860 s. Becker muscular dystrophy (BMD) is named after the German doctor Peter Emil Becker, who first described this variant of DMD in the 1950 s. DMD is one of the most frequent inherited diseases in males, affecting one in 3,500 boys. DMD occurs when the dystrophin gene, located on the short arm of the X chromosome, is broken. Since males only carry one copy of the X chromosome, they only have one copy of the dystrophin gene. Without the dystrophin protein, muscle is easily damaged during cycles of contraction and relaxation. While early in the disease muscle compensates by regeneration, later on muscle progenitor cells cannot keep up with the ongoing damage and healthy muscle is replaced by non-functional fibro-fatty tissue.

BMD results from different mutations in the dystrophin gene. BMD patients have some dystrophin, but it is either insufficient in quantity or poor in quality. Having some dystrophin protects the muscles of those with BMD from degenerating as badly or as quickly as those of people with DMD.

For example, studies demonstrate that blocking or eliminating function of GDF8 in vivo can effectively treat at least certain symptoms in DMD and BMD patients. Thus, the subject variant ActRIIB proteins (e.g., variant ActRIIB proteins in either homomeric or heteromeric forms) may act as GDF8 inhibitors (antagonists), and constitute an alternative means of blocking the functions of GDF8 and/or ActRIIB in vivo in DMD and BMD patients.

In other embodiments, the subject ActRIIB polypeptides (e.g., variant ActRIIB proteins in either homomeric or heteromeric forms) can be used to form pharmaceutical compositions that can be beneficially used to prevent, treat, or alleviate symptoms of a host of diseases involving neurodegeneration. While not wishing to be bound by any particular theory, the subject ActRIIB polypeptides (e.g., variant ActRIIB proteins in either homomeric or heteromeric forms) may antagonize the inhibitory feedback mechanism mediated through the type I receptor ALK7, thus allowing new neuronal growth and differentiation. The subject ActRIIB polypeptides (e.g., variant ActRIIB proteins in either homomeric or heteromeric forms) as pharmaceutical compositions can be beneficially used to prevent, treat, or alleviate symptoms of diseases with neurodegeneration, including Alzheimer’s Disease (AD), Parkinson’s Disease (PD), Amyotrophic Lateral Sclerosis (ALS), Spinal Muscular Atrophy (SMA), and Huntington’s disease (HD).

AD is a chronic, incurable, and unstoppable central nervous system (CNS) disorder that occurs gradually, resulting in memory loss, unusual behavior, personality changes, and a decline in thinking abilities. These losses are related to the death of specific types of brain cells and the breakdown of connections between them. AD has been described as childhood development in reverse. In most people with AD, symptoms appear after the age 60. The earliest symptoms include loss of recent memory, faulty judgment, and changes in personality. Later in the disease, those with AD may forget how to do simple tasks like washing their hands. Eventually people with AD lose all reasoning abilities and become dependent on other people for their everyday care. Finally, the disease becomes so debilitating that patients are bedridden and typically develop coexisting illnesses. AD patients most commonly die from pneumonia, 8 to 20 years from disease onset.

PD is a chronic, incurable, and unstoppable CNS disorder that occurs gradually and results in uncontrolled body movements, rigidity, tremor, and gait difficulties. These motor system problems are related to the death of brain cells in an area of the brain that produces dopamine, a chemical that helps control muscle activity. In most people with PD, symptoms appear after age 50. The initial symptoms of PD are a pronounced tremor affecting the extremities, notably in the hands or lips. Subsequent characteristic symptoms of PD are stiffness or slowness of movement, a shuffling walk, stooped posture, and impaired balance. There are wide ranging secondary symptoms such as memory loss, dementia, depression, emotional changes, swallowing difficulties, abnormal speech, sexual dysfunction, and bladder and bowel problems. These symptoms will begin to interfere with routine activities, such as holding a fork or reading a newspaper. Finally, people with PD become so profoundly disabled that they are bedridden. People with PD usually die from pneumonia.

The causes of these neurological diseases have remained largely unknown. They are conventionally defined as distinct diseases, yet clearly show extraordinary similarities in basic processes and commonly demonstrate overlapping symptoms far greater than would be expected by chance alone. Current disease definitions fail to properly deal with the issue of overlap and a new classification of the neurodegenerative disorders has been called for.

HD is another neurodegenerative disease resulting from genetically programmed degeneration of neurons in certain areas of the brain. This degeneration causes uncontrolled movements, loss of intellectual faculties, and emotional disturbance. HD is a familial disease, passed from parent to child through a dominant mutation in the wild-type gene. Some early symptoms of HD are mood swings, depression, irritability, or trouble driving, learning new things, remembering a fact, or making a decision. As the disease progresses, concentration on intellectual tasks becomes increasingly difficult and the patient may have difficulty feeding himself or herself and swallowing. The rate of disease progression and the age of onset vary from person to person.

Tay-Sachs disease and Sandhoff disease are glycolipid storage diseases caused by the lack of lysosomal β-hexosaminidase (Gravel et al., in The Metabolic Basis of Inherited Disease, eds. Scriver et al., McGraw-Hill, New York, pp. 2839-2879, 1995). In both disorders, GM2 ganglioside and related glycolipid substrates for β-hexosaminidase accumulate in the nervous system and trigger acute neurodegeneration. In the most severe forms, the onset of symptoms begins in early infancy. A precipitous neurodegenerative course then ensues, with affected infants exhibiting motor dysfunction, seizure, visual loss, and deafness. Death usually occurs by 2-5 years of age. Neuronal loss through an apoptotic mechanism has been demonstrated (Huang et al., Hum. Mol. Genet. 6: 1879-1885, 1997).

It is well known that apoptosis plays a role in AIDS pathogenesis in the immune system. However, HIV-1 also induces neurological disease. Shi et al. (J. Clin. Invest. 98: 1979-1990, 1996) examined apoptosis induced by HIV-1 infection of the central nervous system (CNS) in an in vitro model and in brain tissue from AIDS patients, and found that HIV-1 infection of primary brain cultures induced apoptosis in neurons and astrocytes in vitro. Apoptosis of neurons and astrocytes was also detected in brain tissue from 10/11 AIDS patients, including 5/5 patients with HIV-1 dementia and ⅘ nondemented patients.

Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats.

The subject ActRIIB polypeptides (e.g., variant ActRIIB proteins in either homomeric or heteromeric forms) may also be useful to prevent, treat, and alleviate symptoms of various peripheral nervous system (PNS) disorders, such as the ones described below. The PNS is composed of the nerves that lead to or branch off from the CNS. The peripheral nerves handle a diverse array of functions in the body, including sensory, motor, and autonomic functions. When an individual has a peripheral neuropathy, nerves of the PNS have been damaged. Nerve damage can arise from a number of causes, such as disease, physical injury, poisoning, or malnutrition. These agents may affect either afferent or efferent nerves. Depending on the cause of damage, the nerve cell axon, its protective myelin sheath, or both may be injured or destroyed.

The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used.

Peripheral neuropathy is a widespread disorder, and there are many underlying causes. Some of these causes are common, such as diabetes, and others are extremely rare, such as acrylamide poisoning and certain inherited disorders. The most common worldwide cause of peripheral neuropathy is leprosy. Leprosy is caused by the bacterium Mycobacterium leprae, which attacks the peripheral nerves of affected people. While leprosy is rare in the United States, according to statistics gathered by the World Health Organization, an estimated 1.15 million people have leprosy worldwide.

In the United States, diabetes is the most commonly known cause of peripheral neuropathy. It has been estimated that more than 17 million people in the United States and Europe have diabetes-related polyneuropathy. Many neuropathies are idiopathic - no known cause can be found. The most common of the inherited peripheral neuropathies in the United States is Charcot-Marie-Tooth disease (CMT), which affects approximately 125,000 persons.

Another of the better known peripheral neuropathies is Guillain-Barre syndrome, which arises from complications associated with viral illnesses, such as cytomegalovirus, Epstein-Barr virus, and human immunodeficiency virus (HIV), or bacterial infection, including Campylobacter jejuni and Lyme disease. The worldwide incidence rate is approximately 1.7 cases per 100,000 people annually. Other well-known causes of peripheral neuropathies include chronic alcoholism, infection of the varicella-zoster virus, botulism, and poliomyelitis. Peripheral neuropathy may develop as a primary symptom, or it may be due to another disease. For example, peripheral neuropathy is only one symptom of diseases such as amyloid neuropathy, certain cancers, or inherited neurologic disorders. Such diseases may affect the peripheral nervous system (PNS) and the central nervous system (CNS), as well as other body tissues.

Other PNS diseases treatable with the subject ActRIIB polypeptides (e.g., variant ActRIIB proteins in either homomeric or heteromeric forms) include: Brachial Plexus Neuropathies (diseases of the cervical and first thoracic roots, nerve trunks, cords, and peripheral nerve components of the brachial plexus. Clinical manifestations include regional pain, paresthesia, muscle weakness, and decreased sensation in the upper extremity. These disorders may be associated with trauma, including birth injuries; thoracic outlet syndrome; neoplasms, neuritis, radiotherapy; and other conditions. See Adams et al., Principles of Neurology, 6th ed, pp1351-2); Diabetic Neuropathies (peripheral, autonomic, and cranial nerve disorders that are associated with diabetes mellitus). These conditions usually result from diabetic microvascular injury involving small blood vessels that supply nerves (vasa nervorum). Relatively common conditions which may be associated with diabetic neuropathy include third nerve palsy; mononeuropathy; mononeuropathy multiplex; diabetic amyotrophy; a painful polyneuropathy; autonomic neuropathy; and thoracoabdominal neuropathy (see Adams et al., Principles of Neurology, 6th ed, p1325); mononeuropathies (disease or trauma involving a single peripheral nerve in isolation, or out of proportion to evidence of diffuse peripheral nerve dysfunction). Mononeuropathy multiplex refers to a condition characterized by multiple isolated nerve injuries. Mononeuropathies may result from a wide variety of causes, including ischemia; traumatic injury; compression; connective tissue diseases; cumulative trauma disorders; and other conditions); Neuralgia (intense or aching pain that occurs along the course or distribution of a peripheral or cranial nerve); Peripheral Nervous System Neoplasms (neoplasms which arise from peripheral nerve tissue. This includes neurofibromas; Schwannomas; granular cell tumors; and malignant peripheral nerve sheath tumors. See DeVita Jr et al., Cancer: Principles and Practice of Oncology, 5th ed, pp1750-1); Nerve Compression Syndromes (mechanical compression of nerves or nerve roots from internal or external causes. These may result in a conduction block to nerve impulses, due to, for example, myelin sheath dysfunction, or axonal loss. The nerve and nerve sheath injuries may be caused by ischemia; inflammation; a direct mechanical effect; or Neuritis (a general term indicating inflammation of a peripheral or cranial nerve). Clinical manifestation may include pain; paresthesia; paresis; or hyperthesia; Polyneuropathies (diseases of multiple peripheral nerves). The various forms are categorized by the type of nerve affected (e.g., sensory, motor, or autonomic), by the distribution of nerve injury (e.g., distal vs. proximal), by nerve component primarily affected (e.g., demyelinating vs. axonal), by etiology, or by pattern of inheritance.

Similarly, the subject ActRIIB proteins (e.g., variant ActRIIB proteins in either homomeric or heteromeric forms) provide an effective means to increase muscle mass in other disease conditions that are in need of muscle growth. For example, ALS, also called Lou Gehrig’s disease (motor neuron disease) is a chronic, incurable, and unstoppable CNS disorder that attacks the motor neurons, components of the CNS that connect the brain to the skeletal muscles. In ALS, the motor neurons deteriorate and eventually die, and though a person’s brain normally remains fully functioning and alert, the command to move never reaches the muscles. Most people who get ALS are between 40 and 70 years old. The first motor neurons that weaken are those leading to the arms or legs. Those with ALS may have trouble walking, they may drop things, fall, slur their speech, and laugh or cry uncontrollably. Eventually the muscles in the limbs begin to atrophy from disuse. This muscle weakness will become debilitating and a person will need a wheel chair or become unable to function out of bed. Most ALS patients die from respiratory failure or from complications of ventilator assistance like pneumonia, 3-5 years from disease onset.

ActRIIB protein-induced increased muscle mass using the subject ActRIIB proteins (e.g., variant ActRIIB proteins in either homomeric or heteromeric forms) might also benefit those suffering from muscle wasting diseases. Gonzalez-Cadavid et al. (supra) reported that that GDF8 expression correlates inversely with fat-free mass in humans and that increased expression of the GDF8 gene is associated with weight loss in men with AIDS wasting syndrome. By inhibiting the function of GDF8 in AIDS patients, at least certain symptoms of AIDS may be alleviated, if not completely eliminated, thus significantly improving quality of life in AIDS patients.

Since loss of GDF8 function is also associated with fat loss without diminution of nutrient intake (Zimmers et al., supra; McPherron and Lee, supra), the subject ActRIIB proteins (e.g., variant ActRIIB proteins in either homomeric or heteromeric forms) may further be used as a therapeutic agent for slowing or preventing the development of obesity and type 2 diabetes.

Cancer anorexia-cachexia syndrome is among the most debilitating and life-threatening aspects of cancer. Progressive weight loss in cancer anorexia-cachexia syndrome is a common feature of many types of cancer and is responsible not only for a poor quality of life and poor response to chemotherapy, but also a shorter survival time than is found in patients with comparable tumors without weight loss. Associated with anorexia, fat and muscle tissue wasting, psychological distress, and a lower quality of life, cachexia arises from a complex interaction between the cancer and the host. It is one of the most common causes of death among cancer patients and is present in 80% at death. It is a complex example of metabolic chaos effecting protein, carbohydrate, and fat metabolism. Tumors produce both direct and indirect abnormalities, resulting in anorexia and weight loss. Currently, there is no treatment to control or reverse the process. Cancer anorexia-cachexia syndrome affects cytokine production, release of lipid-mobilizing and proteolysis-inducing factors, and alterations in intermediary metabolism. Although anorexia is common, a decreased food intake alone is unable to account for the changes in body composition seen in cancer patients, and increasing nutrient intake is unable to reverse the wasting syndrome. Cachexia should be suspected in patients with cancer if an involuntary weight loss of greater than five percent of premorbid weight occurs within a six-month period. ActRIIB polypeptides (e.g., variant ActRIIB proteins in either homomeric or heteromeric forms) disclosed herein may be useful for treating an activin-dependent cancer, such as ovarian cancer.

Since systemic overexpression of GDF8 in adult mice was found to induce profound muscle and fat loss analogous to that seen in human cachexia syndromes (Zimmers et al., supra), the subject ActRIIB proteins (e.g., variant ActRIIB proteins in either homomeric or heteromeric forms) as pharmaceutical compositions can be beneficially used to prevent, treat, or alleviate the symptoms of the cachexia syndrome, where muscle growth is desired.

In other embodiments, the present disclosure provides methods of inducing bone and/or cartilage formation, preventing bone loss, increasing bone mineralization, or preventing the demineralization of bone. For example, the subject ActRIIB proteins (e.g., variant ActRIIB proteins in either homomeric or heteromeric forms) have application in treating osteoporosis and the healing of bone fractures and cartilage defects in humans and other animals. ActRIIB proteins (e.g., variant ActRIIB proteins in either homomeric or heteromeric forms) may be useful in patients that are diagnosed with subclinical low bone density, as a protective measure against the development of osteoporosis.

In one specific embodiment, methods and compositions of the present disclosure may find medical utility in the healing of bone fractures and cartilage defects in humans and other animals. The subject methods and compositions may also have prophylactic use in closed as well as open fracture reduction and also in the improved fixation of artificial joints. De novo bone formation induced by an osteogenic agent contributes to the repair of congenital, trauma-induced, or oncologic resection induced craniofacial defects, and also is useful in cosmetic plastic surgery. Further, methods and compositions of the disclosure may be used in the treatment of periodontal disease, and in other tooth repair processes. In certain cases, the subject ActRIIB proteins (e.g., variant ActRIIB proteins in either homomeric or heteromeric forms) may provide an environment to attract bone-forming cells, stimulate growth of bone-forming cells or induce differentiation of progenitors of bone-forming cells. ActRIIB proteins described herein (e.g., variant ActRIIB proteins in either homomeric or heteromeric forms) may also be useful in the treatment of osteoporosis. Further, ActRIIB proteins (e.g., variant ActRIIB proteins in either homomeric or heteromeric forms) may be used in cartilage defect repair and prevention/reversal of osteoarthritis.

In another specific embodiment, the disclosure provides a therapeutic method and composition for repairing fractures and other conditions related to cartilage and/or bone defects or periodontal diseases. The disclosure further provides therapeutic methods and compositions for wound healing and tissue repair. The types of wounds include, but are not limited to, burns, incisions and ulcers. See e.g., PCT Publication No. WO84/01106. Such compositions comprise a therapeutically effective amount of at least one of the ActRIIB proteins disclosed herein (e.g., variant ActRIIB proteins in either homomeric or heteromeric forms) in admixture with a pharmaceutically acceptable vehicle, carrier, or matrix.

In another specific embodiment, ActRIIB proteins described herein (e.g., variant ActRIIB proteins in either homomeric or heteromeric forms) can be applied to conditions causing bone loss such as osteoporosis, hyperparathyroidism, Cushing’s disease, thyrotoxicosis, chronic diarrheal state or malabsorption, renal tubular acidosis, or anorexia nervosa. Many people know that being female, having a low body weight, and leading a sedentary lifestyle are risk factors for osteoporosis (loss of bone mineral density, leading to fracture risk). However, osteoporosis can also result from the long-term use of certain medications. Osteoporosis resulting from drugs or another medical condition is known as secondary osteoporosis. In a condition known as Cushing’s disease, the excess amount of cortisol produced by the body results in osteoporosis and fractures. The most common medications associated with secondary osteoporosis are the corticosteroids, a class of drugs that act like cortisol, a hormone produced naturally by the adrenal glands. Although adequate levels of thyroid hormones (which are produced by the thyroid gland) are needed for the development of the skeleton, excess thyroid hormone can decrease bone mass over time. Antacids that contain aluminum can lead to bone loss when taken in high doses by people with kidney problems, particularly those undergoing dialysis. Other medications that can cause secondary osteoporosis include phenytoin (Dilantin) and barbiturates that are used to prevent seizures; methotrexate (Rheumatrex, Immunex, Folex PFS), a drug for some forms of arthritis, cancer, and immune disorders; cyclosporine (Sandimmune, Neoral), a drug used to treat some autoimmune diseases and to suppress the immune system in organ transplant patients; luteinizing hormone-releasing hormone agonists (Lupron, Zoladex), used to treat prostate cancer and endometriosis; heparin (Calciparine, Liquaemin), an anticlotting medication; and cholestyramine (Questran) and colestipol (Colestid), used to treat high cholesterol. Gum disease causes bone loss because these harmful bacteria in our mouths force our bodies to defend against them. The bacteria produce toxins and enzymes under the gum-line, causing a chronic infection.

In a further embodiment, ActRIIB polypeptides of the present disclosure (e.g., variant ActRIIB proteins in either homomeric or heteromeric forms) provide methods and therapeutic agents for treating diseases or disorders associated with abnormal or unwanted bone growth. For example, patients having the disease known as fibrodysplasia ossificans progressiva (FOP) grow an abnormal “second skeleton” that prevents any movement. Additionally, abnormal bone growth can occur after hip replacement surgery and thus ruin the surgical outcome. This is a more common example of pathological bone growth and a situation in which the subject methods and compositions may be therapeutically useful. The same methods and compositions may also be useful for treating other forms of abnormal bone growth (e.g., pathological growth of bone following trauma, burns or spinal cord injury), and for treating or preventing the undesirable conditions associated with the abnormal bone growth seen in connection with metastatic prostate cancer or osteosarcoma.

In other embodiments, ActRIIB polypeptides of the present disclosure (e.g., variant ActRIIB proteins in either homomeric or heteromeric forms) provide compositions and methods for regulating body fat content in an animal and for treating or preventing conditions related thereto, and particularly, health-compromising conditions related thereto. According to the present disclosure, to regulate (control) body weight can refer to reducing or increasing body weight, reducing or increasing the rate of weight gain, or increasing or reducing the rate of weight loss, and also includes actively maintaining, or not significantly changing body weight (e.g., against external or internal influences which may otherwise increase or decrease body weight). One embodiment of the present disclosure relates to regulating body weight by administering to an animal (e.g., a human) in need thereof an ActRIIB polypeptide (e.g., variant ActRIIB proteins in either homomeric or heteromeric forms).

In some embodiments, the disclosure relate to methods for decreasing the body fat content or reducing the rate of increase in body fat content, and for treating a disorder associated with undesirable body weight gain, such as obesity, non-insulin dependent diabetes mellitus (NIDDM), cardiovascular disease, cancer, hypertension, osteoarthritis, stroke, respiratory problems, and gall bladder disease, comprising administering a patient in need thereof a ActRIIB polypeptide (e.g., variant ActRIIB proteins in either homomeric or heteromeric forms), as described herein.

In one specific embodiment, the present disclosure relates to methods and compounds for reducing body weight and/or reducing weight gain in an animal, and more particularly, for treating or ameliorating obesity in patients at risk for or suffering from obesity. In another specific embodiment, the present disclosure is directed to methods and compounds for treating an animal that is unable to gain or retain weight (e.g., an animal with a wasting syndrome). Such methods are effective to increase body weight and/or mass, or to reduce weight and/or mass loss, or to improve conditions associated with or caused by undesirably low (e.g., unhealthy) body weight and/or mass.

In some embodiments, ActRIIB polypeptides of the present disclosure (e.g., variant ActRIIB proteins in either homomeric or heteromeric forms) can be used to increase red blood cell levels, increase hemoglobin levels, treat or prevent an anemia, and/or treat or prevent ineffective erythropoiesis in a subject in need thereof. In some embodiments, variant ActRIIB polypeptides as well as homomultimer and heteromultimers thereof of the present disclosure may be used in combination with conventional therapeutic approaches for increasing red blood cell levels, particularly those used to treat anemias of multifactorial origin. Conventional therapeutic approaches for increasing red blood cell levels include, for example, red blood cell transfusion, administration of one or more EPO receptor activators, hematopoietic stem cell transplantation, immunosuppressive biologics and drugs (e.g., corticosteroids). In certain embodiments, ActRIIB polypeptides of the present disclosure (e.g., variant ActRIIB proteins in either homomeric or heteromeric forms) can be used to treat or prevent an anemia in a subject in need thereof. In certain embodiments, ActRIIB polypeptides of the present disclosure (e.g., variant ActRIIB proteins in either homomeric or heteromeric forms) can be used to treat or prevent ineffective erythropoiesis and/or the disorders associated with ineffective erythropoiesis in a subject in need thereof. In certain aspects, ActRIIB polypeptides of the present disclosure (e.g., variant ActRIIB proteins in either homomeric or heteromeric forms) can be used in combination with conventional therapeutic approaches for treating or preventing an anemia or ineffective erythropoiesis disorder, particularly those used to treat anemias of multifactorial origin. In some embodiments, the disclosure relates to methods for increasing red blood cell levels, increasing hemoglobin levels, treating or preventing an anemia, and/or treating or preventing ineffective erythropoiesis in a patient comprising administering a patient in need thereof an ActRIIB polypeptide (e.g., variant ActRIIB proteins in either homomeric or heteromeric forms), as described herein.

In certain embodiments, one or more ActRIIB proteins of the disclosure (e.g., variant ActRIIB proteins in either homomeric or heteromeric forms), optionally combined with an EPO receptor activator, may be used to increase red blood cell, hemoglobin, or reticulocyte levels in healthy individuals and selected patient populations. Examples of appropriate patient populations include those with undesirably low red blood cell or hemoglobin levels, such as patients having an anemia, and those that are at risk for developing undesirably low red blood cell or hemoglobin levels, such as those patients who are about to undergo major surgery or other procedures that may result in substantial blood loss. In one embodiment, a patient with adequate red blood cell levels is treated with one or more ActRIIB proteins (e.g., variant ActRIIB proteins in either homomeric or heteromeric forms) to increase red blood cell levels, and then blood is drawn and stored for later use in transfusions.

One or more ActRIIB proteins of the disclosure (e.g., variant ActRIIB proteins in either homomeric or heteromeric forms), optionally combined with an EPO receptor activator, may be used to increase red blood cell levels, hemoglobin levels, and/or hematocrit levels in a patient having an anemia. When observing hemoglobin and/or hematocrit levels in humans, a level of less than normal for the appropriate age and gender category may be indicative of anemia, although individual variations are taken into account. For example, a hemoglobin level from 10-12.5 g/dl, and typically about 11.0 g/dl is considered to be within the normal range in health adults, although, in terms of therapy, a lower target level may cause fewer cardiovascular side effects [see, e.g., Jacobs et al. (2000) Nephrol Dial Transplant 15, 15-19]. Alternatively, hematocrit levels (percentage of the volume of a blood sample occupied by the cells) can be used as a measure for anemia. Hematocrit levels for healthy individuals range from about 41-51% for adult males and from 35-45% for adult females. In certain embodiments, a patient may be treated with a dosing regimen intended to restore the patient to a target level of red blood cells, hemoglobin, and/or hematocrit. As hemoglobin and hematocrit levels vary from person to person, optimally, the target hemoglobin and/or hematocrit level can be individualized for each patient.

Anemia is frequently observed in patients having a tissue injury, an infection, and/or a chronic disease, particularly cancer. In some subjects, anemia is distinguished by low erythropoietin levels and/or an inadequate response to erythropoietin in the bone marrow [see, e.g., Adamson (2008) Harrison’s Principles of Internal Medicine, 17th ed.; McGraw Hill, New York, pp 628-634]. Potential causes of anemia include, for example, blood loss, nutritional deficits (e.g. reduced dietary intake of protein), medication reaction, various problems associated with the bone marrow, and many diseases. More particularly, anemia has been associated with a variety of disorders and conditions that include, for example, bone marrow transplantation; solid tumors (e.g., breast cancer, lung cancer, and colon cancer); tumors of the lymphatic system (e.g., chronic lymphocyte leukemia, non-Hodgkins lymphoma, and Hodgkins lymphoma); tumors of the hematopoietic system (e.g., leukemia, a myelodysplastic syndrome and multiple myeloma); radiation therapy; chemotherapy (e.g., platinum containing regimens); inflammatory and autoimmune diseases, including, but not limited to, rheumatoid arthritis, other inflammatory arthritides, systemic lupus erythematosis (SLE), acute or chronic skin diseases (e.g., psoriasis), inflammatory bowel disease (e.g., Crohn’s disease and ulcerative colitis); acute or chronic renal disease or failure, including idiopathic or congenital conditions; acute or chronic liver disease; acute or chronic bleeding; situations where transfusion of red blood cells is not possible due to patient allo- or auto-antibodies and/or for religious reasons (e.g., some Jehovah’s Witnesses); infections (e.g., malaria and osteomyelitis); hemoglobinopathies including, for example, sickle cell disease (anemia), thalassemias; drug use or abuse (e.g., alcohol misuse); pediatric patients with anemia from any cause to avoid transfusion; and elderly patients or patients with underlying cardiopulmonary disease with anemia who cannot receive transfusions due to concerns about circulatory overload [see, e.g., Adamson (2008) Harrison’s Principles of Internal Medicine, 17th ed.; McGraw Hill, New York, pp 628-634]. In some embodiments, one or more ActRIIB proteins of the disclosure (e.g., variant ActRIIB proteins in either homomeric or heteromeric forms) could be used to treat or prevent anemia associated with one or more of the disorders or conditions disclosed herein.

Many factors can contribute to cancer-related anemia. Some are associated with the disease process itself and the generation of inflammatory cytokines such as interleukin-1, interferon-gamma, and tumor necrosis factor [Bron et al. (2001) Semin Oncol 28(Suppl 8): 1-6]. Among its effects, inflammation induces the key iron-regulatory peptide hepcidin, thereby inhibiting iron export from macrophages and generally limiting iron availability for erythropoiesis [see, e.g., Ganz (2007) J Am Soc Nephrol 18:394-400]. Blood loss through various routes can also contribute to cancer-related anemia. The prevalence of anemia due to cancer progression varies with cancer type, ranging from 5% in prostate cancer up to 90% in multiple myeloma. Cancer-related anemia has profound consequences for patients, including fatigue and reduced quality of life, reduced treatment efficacy, and increased mortality. In some embodiments, one or more ActRIIB proteins of the disclosure (e.g., variant ActRIIB proteins in either homomeric or heteromeric forms), optionally combined with an EPO receptor activator, could be used to treat a cancer-related anemia.

A hypoproliferative anemia can result from primary dysfunction or failure of the bone marrow. Hypoproliferative anemias include: anemia of chronic disease, anemia of kidney disease, anemia associated with hypometabolic states, and anemia associated with cancer. In each of these types, endogenous erythropoietin levels are inappropriately low for the degree of anemia observed. Other hypoproliferative anemias include: early-stage iron-deficient anemia, and anemia caused by damage to the bone marrow. In these types, endogenous erythropoietin levels are appropriately elevated for the degree of anemia observed. Prominent examples would be myelosuppression caused by cancer and/or chemotherapeutic drugs or cancer radiation therapy. A broad review of clinical trials found that mild anemia can occur in 100% of patients after chemotherapy, while more severe anemia can occur in up to 80% of such patients [see, e.g., Groopman et al. (1999) J Natl Cancer Inst 91: 1616-1634]. Myelosuppressive drugs include, for example: 1) alkylating agents such as nitrogen mustards (e.g., melphalan) and nitrosoureas (e.g., streptozocin); 2) antimetabolites such as folic acid antagonists (e.g., methotrexate), purine analogs (e.g., thioguanine), and pyrimidine analogs (e.g., gemcitabine); 3) cytotoxic antibiotics such as anthracyclines (e.g., doxorubicin); 4) kinase inhibitors (e.g., gefitinib); 5) mitotic inhibitors such as taxanes (e.g., paclitaxel) and vinca alkaloids (e.g., vinorelbine); 6) monoclonal antibodies (e.g., rituximab); and 7) topoisomerase inhibitors (e.g., topotecan and etoposide). In addition, conditions resulting in a hypometabolic rate can produce a mild-to-moderate hypoproliferative anemia. Among such conditions are endocrine deficiency states. For example, anemia can occur in Addison’s disease, hypothyroidism, hyperparathyroidism, or males who are castrated or treated with estrogen. In some embodiments, one or more ActRIIB proteins of the disclosure (e.g., variant ActRIIB proteins in either homomeric or heteromeric forms), optionally combined with an EPO receptor activator, could be used to treat a hyperproliferative anemia.

Chronic kidney disease is sometimes associated with hypoproliferative anemia, and the degree of the anemia varies in severity with the level of renal impairment. Such anemia is primarily due to inadequate production of erythropoietin and reduced survival of red blood cells. Chronic kidney disease usually proceeds gradually over a period of years or decades to end-stage (Stage-5) disease, at which point dialysis or kidney transplantation is required for patient survival. Anemia often develops early in this process and worsens as disease progresses. The clinical consequences of anemia of kidney disease are well-documented and include development of left ventricular hypertrophy, impaired cognitive function, reduced quality of life, and altered immune function [see, e.g., Levin et al. (1999) Am J Kidney Dis 27:347-354; Nissenson (1992) Am J Kidney Dis 20(Suppl 1):21-24; Revicki et al. (1995) Am J Kidney Dis 25:548-554; Gafter et al., (1994) Kidney Int 45:224-231]. In some embodiments, one or more ActRIIB proteins (e.g., variant ActRIIB proteins in either homomeric or heteromeric forms), optionally combined with an EPO receptor activator, could be used to treat anemia associated with acute or chronic renal disease or failure.

Anemia resulting from acute blood loss of sufficient volume, such as from trauma or postpartum hemorrhage, is known as acute post-hemorrhagic anemia. Acute blood loss initially causes hypovolemia without anemia since there is proportional depletion of RBCs along with other blood constituents. However, hypovolemia will rapidly trigger physiologic mechanisms that shift fluid from the extravascular to the vascular compartment, which results in hemodilution and anemia. If chronic, blood loss gradually depletes body iron stores and eventually leads to iron deficiency. In some embodiments, one or more ActRIIB proteins (e.g., variant ActRIIB proteins in either homomeric or heteromeric forms), optionally combined with an EPO receptor activator, could be used to treat anemia resulting from acute blood loss.

Iron-deficiency anemia is the final stage in a graded progression of increasing iron deficiency which includes negative iron balance and iron-deficient erythropoiesis as intermediate stages. Iron deficiency can result from increased iron demand, decreased iron intake, or increased iron loss, as exemplified in conditions such as pregnancy, inadequate diet, intestinal malabsorption, acute or chronic inflammation, and acute or chronic blood loss. With mild-to-moderate anemia of this type, the bone marrow remains hypoproliferative, and RBC morphology is largely normal; however, even mild anemia can result in some microcytic hypochromic RBCs, and the transition to severe iron-deficient anemia is accompanied by hyperproliferation of the bone marrow and increasingly prevalent microcytic and hypochromic RBCs [see, e.g., Adamson (2008) Harrison’s Principles of Internal Medicine, 17th ed.; McGraw Hill, New York, pp 628-634]. Appropriate therapy for iron-deficiency anemia depends on its cause and severity, with oral iron preparations, parenteral iron formulations, and RBC transfusion as major conventional options. In some embodiments, one or more ActRIIB proteins of the disclosure (e.g., variant ActRIIB proteins in either homomeric or heteromeric forms), optionally combined with an EPO receptor activator, could be used to treat a chronic iron-deficiency.

Myelodysplastic syndrome (MDS) is a diverse collection of hematological conditions characterized by ineffective production of myeloid blood cells and risk of transformation to acute myelogenous leukemia. In MDS patients, blood stem cells do not mature into healthy red blood cells, white blood cells, or platelets. MDS disorders include, for example, refractory anemia, refractory anemia with ringed sideroblasts, refractory anemia with excess blasts, refractory anemia with excess blasts in transformation, refractory cytopenia with multilineage dysplasia, and myelodysplastic syndrome associated with an isolated 5q chromosome abnormality. As these disorders manifest as irreversible defects in both quantity and quality of hematopoietic cells, most MDS patients are afflicted with chronic anemia. Therefore, MDS patients eventually require blood transfusions and/or treatment with growth factors (e.g., erythropoietin or G-CSF) to increase red blood cell levels. However, many MDS patients develop side-effects due to frequency of such therapies. For example, patients who receive frequent red blood cell transfusion can exhibit tissue and organ damage from the buildup of extra iron. Accordingly, one or more ActRIIB proteins of the disclosure (e.g., variant ActRIIB proteins in either homomeric or heteromeric forms), may be used to treat patients having MDS. In certain embodiments, patients suffering from MDS may be treated using one or more ActRIIB proteins of the disclosure (e.g., variant ActRIIB proteins in either homomeric or heteromeric forms), optionally in combination with an EPO receptor activator. In other embodiments, a patient suffering from MDS may be treated using a combination of one or more ActRIIB proteins of the disclosure (e.g., variant ActRIIB proteins in either homomeric or heteromeric forms) and one or more additional therapeutic agents for treating MDS including, for example, thalidomide, lenalidomide, azacitadine, decitabine, erythropoietins, deferoxamine, antithymocyte globulin, and filgrastim (G-CSF).

Originally distinguished from aplastic anemia, hemorrhage, or peripheral hemolysis on the basis of ferrokinetic studies [see, e.g., Ricketts et al. (1978) Clin Nucl Med 3:159-164], ineffective erythropoiesis describes a diverse group of anemias in which production of mature RBCs is less than would be expected given the number of erythroid precursors (erythroblasts) present in the bone marrow [Tanno et al. (2010) Adv Hematol 2010:358283]. In such anemias, tissue hypoxia persists despite elevated erythropoietin levels due to ineffective production of mature RBCs. A vicious cycle eventually develops in which elevated erythropoietin levels drive massive expansion of erythroblasts, potentially leading to splenomegaly (spleen enlargement) due to extramedullary erythropoiesis [see, e.g., Aizawa et al. (2003) Am J Hematol 74:68-72], erythroblast-induced bone pathology [see, e.g., Di Matteo et al. (2008) J Biol Regul Homeost Agents 22:211-216], and tissue iron overload, even in the absence of therapeutic RBC transfusions [see, e.g., Pippard et al. (1979) Lancet 2:819-821]. Thus, by boosting erythropoietic effectiveness, an ActRIIB protein of the present disclosure (e.g., variant ActRIIB proteins in either homomeric or heteromeric forms) may break the aforementioned cycle and thus alleviate not only the underlying anemia but also the associated complications of elevated erythropoietin levels, splenomegaly, bone pathology, and tissue iron overload. In some embodiments, one or more ActRIIB proteins (e.g., variant ActRIIB proteins in either homomeric or heteromeric forms) can be used to treat or prevent ineffective erythropoiesis, including anemia and elevated EPO levels as well as complications such as splenomegaly, erythroblast-induced bone pathology, iron overload, and their attendant pathologies. With splenomegaly, such pathologies include thoracic or abdominal pain and reticuloendothelial hyperplasia. Extramedullary hematopoiesis can occur not only in the spleen but potentially in other tissues in the form of extramedullary hematopoietic pseudotumors [see, e.g., Musallam et al. (2012) Cold Spring Harb Perspect Med 2:a013482]. With erythroblast-induced bone pathology, attendant pathologies include low bone mineral density, osteoporosis, and bone pain [see, e.g., Haidar et al. (2011) Bone 48:425-432]. With iron overload, attendant pathologies include hepcidin suppression and hyperabsorption of dietary iron [see, e.g., Musallam et al. (2012) Blood Rev 26(Suppl 1):S16-S19], multiple endocrinopathies and liver fibrosis/cirrhosis [see, e.g., Galanello et al. (2010) Orphanet J Rare Dis 5:11], and iron-overload cardiomyopathy [Lekawanvijit et al., 2009, Can J Cardiol 25:213-218].

The most common causes of ineffective erythropoiesis are the thalassemia syndromes, hereditary hemoglobinopathies in which imbalances in the production of intact alpha- and beta-hemoglobin chains lead to increased apoptosis during erythroblast maturation [see, e.g., Schrier (2002) Curr Opin Hematol 9:123-126]. Thalassemias are collectively among the most frequent genetic disorders worldwide, with changing epidemiologic patterns predicted to contribute to a growing public health problem in both the U.S. and globally [Vichinsky (2005) Ann NY Acad Sci 1054:18-24]. Thalassemia syndromes are named according to their severity. Thus, α-thalassemias include α-thalassemia minor (also known as α-thalassemia trait; two affected α-globin genes), hemoglobin H disease (three affected α-globin genes), and α-thalassemia major (also known as hydrops fetalis; four affected α-globin genes). β-Thalassemias include (β-thalassemia minor (also known as (β-thalassemia trait; one affected β-globin gene), (β-thalassemia intermedia (two affected β-globin genes), hemoglobin E thalassemia (two affected β-globin genes), and (β-thalassemia major (also known as Cooley’s anemia; two affected β-globin genes resulting in a complete absence of β-globin protein). β-Thalassemia impacts multiple organs, is associated with considerable morbidity and mortality, and currently requires life-long care. Although life expectancy in patients with β-thalassemia has increased in recent years due to use of regular blood transfusions in combination with iron chelation, iron overload resulting both from transfusions and from excessive gastrointestinal absorption of iron can cause serious complications such as heart disease, thrombosis, hypogonadism, hypothyroidism, diabetes, osteoporosis, and osteopenia [see, e.g., Rund et al. (2005) N Engl J Med 353:1135-1146]. In certain embodiments, one or more ActRIIB proteins of the disclosure (e.g., variant ActRIIB proteins in either homomeric or heteromeric forms), optionally combined with an EPO receptor activator, can be used to treat or prevent a thalassemia syndrome.

In some embodiments, one or more ActRIIB proteins of the disclosure (e.g., variant ActRIIB proteins in either homomeric or heteromeric forms), optionally combined with an EPO receptor activator, can be used for treating disorders of ineffective erythropoiesis besides thalassemia syndromes. Such disorders include sideroblastic anemia (inherited or acquired); dyserythropoietic anemia (types I and II); sickle cell anemia; hereditary spherocytosis; pyruvate kinase deficiency; megaloblastic anemias, potentially caused by conditions such as folate deficiency (due to congenital diseases, decreased intake, or increased requirements), cobalamin deficiency (due to congenital diseases, pernicious anemia, impaired absorption, pancreatic insufficiency, or decreased intake), certain drugs, or unexplained causes (congenital dyserythropoietic anemia, refractory megaloblastic anemia, or erythroleukemia); myelophthisic anemias including, for example, myelofibrosis (myeloid metaplasia) and myelophthisis; congenital erythropoietic porphyria; and lead poisoning. In particular embodiments, one or more ActRIIB proteins of the disclosure (e.g., variant ActRIIB proteins in either homomeric or heteromeric forms) may be used to treat myelofibrosis.

In certain embodiments, one or more ActRIIB proteins of the disclosure (e.g., variant ActRIIB proteins in either homomeric or heteromeric forms) may be used in combination with supportive therapies for ineffective erythropoiesis. Such therapies include transfusion with either red blood cells or whole blood to treat anemia. In chronic or hereditary anemias, normal mechanisms for iron homeostasis are overwhelmed by repeated transfusions, eventually leading to toxic and potentially fatal accumulation of iron in vital tissues such as heart, liver, and endocrine glands. Thus, supportive therapies for patients chronically afflicted with ineffective erythropoiesis also include treatment with one or more iron-chelating molecules to promote iron excretion in the urine and/or stool and thereby prevent, or reverse, tissue iron overload [see, e.g., Hershko (2006) Haematologica 91:1307-1312; Cao et al. (2011), Pediatr Rep 3(2):e17]. Effective iron-chelating agents should be able to selectively bind and neutralize ferric iron, the oxidized form of non-transferrin bound iron which likely accounts for most iron toxicity through catalytic production of hydroxyl radicals and oxidation products [see, e.g., Esposito et al. (2003) Blood 102:2670-2677]. These agents are structurally diverse, but all possess oxygen or nitrogen donor atoms able to form neutralizing octahedral coordination complexes with individual iron atoms in stoichiometries of 1:1 (hexadentate agents), 2:1 (tridentate), or 3:1 (bidentate) [Kalinowski et al. (2005) Pharmacol Rev 57:547-583]. In general, effective iron-chelating agents also are relatively low molecular weight (e.g., less than 700 daltons), with solubility in both water and lipids to enable access to affected tissues. Specific examples of iron-chelating molecules include deferoxamine, a hexadentate agent of bacterial origin requiring daily parenteral administration, and the orally active synthetic agents deferiprone (bidentate) and deferasirox (tridentate). Combination therapy consisting of same-day administration of two iron-chelating agents shows promise in patients unresponsive to chelation monotherapy and also in overcoming issues of poor patient compliance with dereroxamine alone [Cao et al. (2011) Pediatr Rep 3(2):e17; Galanello et al. (2010) Ann NY Acad Sci 1202:79-86].

In certain embodiments, one or more ActRIIB proteins of the disclosure (e.g., variant ActRIIB proteins in either homomeric or heteromeric forms) may be used in combination with hepcidin or a hepcidin agonist for ineffective erythropoiesis. A circulating polypeptide produced mainly in the liver, hepcidin is considered a master regulator of iron metabolism by virtue of its ability to induce the degradation of ferroportin, an iron-export protein localized on absorptive enterocytes, hepatocytes, and macrophages. Broadly speaking, hepcidin reduces availability of extracellular iron, so hepcidin agonists may be beneficial in the treatment of ineffective erythropoiesis [see, e.g., Nemeth (2010) Adv Hematol 2010:750643]. This view is supported by beneficial effects of increased hepcidin expression in a mouse model of β-thalassemia [Gardenghi et al. (2010) J Clin Invest 120:4466-4477].

One or more ActRIIB proteins of the disclosure (e.g., variant ActRIIB proteins in either homomeric or heteromeric forms), optionally combined with an EPO receptor activator, would also be appropriate for treating anemias of disordered RBC maturation, which are characterized in part by undersized (microcytic), oversized (macrocytic), misshapen, or abnormally colored (hypochromic) RBCs.

In certain embodiments, the present disclosure provides methods of treating or preventing anemia in an individual in need thereof by administering to the individual a therapeutically effective amount of one or more ActRIIB proteins of the disclosure (e.g., variant ActRIIB proteins in either homomeric or heteromeric forms) and an EPO receptor activator. In certain embodiments, one or more ActRIIB proteins of the disclosure (e.g., variant ActRIIB proteins in either homomeric or heteromeric forms) may be used in combination with EPO receptor activators to reduce the required dose of these activators in patients that are susceptible to adverse effects of EPO. These methods may be used for therapeutic and prophylactic treatments of a patient.

One or more ActRIIB proteins of the disclosure (e.g., variant ActRIIB proteins in either homomeric or heteromeric forms) may be used in combination with EPO receptor activators to achieve an increase in red blood cells, particularly at lower dose ranges of EPO receptor activators. This may be beneficial in reducing the known off-target effects and risks associated with high doses of EPO receptor activators. The primary adverse effects of EPO include, for example, an excessive increase in the hematocrit or hemoglobin levels and polycythemia. Elevated hematocrit levels can lead to hypertension (more particularly aggravation of hypertension) and vascular thrombosis. Other adverse effects of EPO which have been reported, some of which relate to hypertension, are headaches, influenza-like syndrome, obstruction of shunts, myocardial infarctions and cerebral convulsions due to thrombosis, hypertensive encephalopathy, and red cell blood cell aplasia. See, e.g., Singibarti (1994) J. Clin Investig 72(suppl 6), S36-S43; Horl et al. (2000) Nephrol Dial Transplant 15(suppl 4), 51-56; Delanty et al. (1997) Neurology 49, 686-689; and Bunn (2002) N Engl J Med 346(7), 522-523).

Provided that variant ActRIIB proteins of the present disclosure act by a different mechanism than EPO, these antagonists may be useful for increasing red blood cell and hemoglobin levels in patients that do not respond well to EPO. For example, an antagonist of the present disclosure may be beneficial for a patient in which administration of a normal-to-increased dose of EPO (>300 IU/kg/week) does not result in the increase of hemoglobin level up to the target level. Patients with an inadequate EPO response are found in all types of anemia, but higher numbers of non-responders have been observed particularly frequently in patients with cancers and patients with end-stage renal disease. An inadequate response to EPO can be either constitutive (observed upon the first treatment with EPO) or acquired (observed upon repeated treatment with EPO).

In certain embodiments, the present disclosure provides methods for managing a patient that has been treated with, or is a candidate to be treated with, one or more ActRIIB proteins of the disclosure (e.g., variant ActRIIB proteins in either homomeric or heteromeric forms) by measuring one or more hematologic parameters in the patient. The hematologic parameters may be used to evaluate appropriate dosing for a patient who is a candidate to be treated with the antagonist of the present disclosure, to monitor the hematologic parameters during treatment, to evaluate whether to adjust the dosage during treatment with one or more antagonist of the disclosure, and/or to evaluate an appropriate maintenance dose of one or more antagonists of the disclosure. If one or more of the hematologic parameters are outside the normal level, dosing with one or more ActRIIB proteins of the disclosure (e.g., variant ActRIIB proteins in either homomeric or heteromeric forms) may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art-recognized methods. Such parameters may be determined using a blood sample from a patient. Increases in red blood cell levels, hemoglobin levels, and/or hematocrit levels may cause increases in blood pressure.

In one embodiment, if one or more hematologic parameters are outside the normal range or on the high side of normal in a patient who is a candidate to be treated with one or more ActRIIB proteins of the disclosure (e.g., variant ActRIIB proteins in either homomeric or heteromeric forms), then onset of administration of the one or more variant ActRIIB proteins of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient’s blood pressure. Any blood pressure lowering agent appropriate for the individual patient’s condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient’s iron stores have returned to a normal or acceptable level. For patients having higher than normal red blood cell levels and/or hemoglobin levels, then administration of the one or more ActRIIB proteins of the disclosure (e.g., variant ActRIIB proteins in either homomeric or heteromeric forms) may be delayed until the levels have returned to a normal or acceptable level.

In certain embodiments, if one or more hematologic parameters are outside the normal range or on the high side of normal in a patient who is a candidate to be treated with one or more ActRIIB proteins of the disclosure (e.g., variant ActRIIB proteins in either homomeric or heteromeric forms), then the onset of administration may not be delayed. However, the dosage amount or frequency of dosing of the one or more variant ActRIIB proteins of the disclosure may be set at an amount that would reduce the risk of an unacceptable increase in the hematologic parameters arising upon administration of the one or more variant ActRIIB proteins of the disclosure. Alternatively, a therapeutic regimen may be developed for the patient that combines one or more variant ActRIIB proteins of the disclosure with a therapeutic agent that addresses the undesirable level of the hematologic parameter. For example, if the patient has elevated blood pressure, then a therapeutic regimen involving administration of one or more variant ActRIIB proteins of the disclosure and a blood pressure-lowering agent may be designed. For a patient having lower than desired iron stores, a therapeutic regimen of one or more variant ActRIIB proteins and iron supplementation may be developed.

In one embodiment, baseline parameter(s) for one or more hematologic parameters may be established for a patient who is a candidate to be treated with one or more ActRIIB proteins of the disclosure (e.g., variant ActRIIB proteins in either homomeric or heteromeric forms) and an appropriate dosing regimen established for that patient based on the baseline value(s). Alternatively, established baseline parameters based on a patient’s medical history could be used to inform an appropriate antagonist-dosing regimen for a patient. For example, if a healthy patient has an established baseline blood pressure reading that is above the defined normal range it may not be necessary to bring the patient’s blood pressure into the range that is considered normal for the general population prior to treatment with the one or more antagonist of the disclosure. A patient’s baseline values for one or more hematologic parameters prior to treatment with one or more variant ActRIIB proteins of the disclosure (e.g., variant ActRIIB proteins in either homomeric or heteromeric forms) may also be used as the relevant comparative values for monitoring any changes to the hematologic parameters during treatment with the one or more antagonists of the disclosure.

In certain embodiments, one or more hematologic parameters are measured in patients who are being treated with a one or more ActRIIB proteins of the disclosure (e.g., variant ActRIIB proteins in either homomeric or heteromeric forms). The hematologic parameters may be used to monitor the patient during treatment and permit adjustment or termination of the dosing with the one or more antagonists of the disclosure or additional dosing with another therapeutic agent. For example, if administration of one or more variant ActRIIB proteins of the disclosure results in an increase in blood pressure, red blood cell level, or hemoglobin level, or a reduction in iron stores, then the dose of the one or more variant ActRIIB proteins of the disclosure may be reduced in amount or frequency in order to decrease the effects of the one or more variant ActRIIB proteins of the disclosure on the one or more hematologic parameters. If administration of one or more variant ActRIIB proteins of the disclosure results in a change in one or more hematologic parameters that is adverse to the patient, then the dosing of the one or more variant ActRIIB proteins of the disclosure may be terminated either temporarily, until the hematologic parameter(s) return to an acceptable level, or permanently. Similarly, if one or more hematologic parameters are not brought within an acceptable range after reducing the dose or frequency of administration of the one or more variant ActRIIB proteins of the disclosure, then the dosing may be terminated. As an alternative, or in addition to, reducing or terminating the dosing with the one or more variant ActRIIB proteins of the disclosure, the patient may be dosed with an additional therapeutic agent that addresses the undesirable level in the hematologic parameter(s), such as, for example, a blood pressure-lowering agent or an iron supplement. For example, if a patient being treated with one or more variant ActRIIB proteins of the disclosure has elevated blood pressure, then dosing with the one or more variant ActRIIB proteins of the disclosure may continue at the same level and a blood pressure-lowering agent is added to the treatment regimen, dosing with the one or more variant ActRIIB proteins of the disclosure may be reduced (e.g., in amount and/or frequency) and a blood pressure-lowering agent is added to the treatment regimen, or dosing with the one or more variant ActRIIB proteins of the disclosure may be terminated and the patient may be treated with a blood pressure-lowering agent.

In some embodiments, the present disclosure relates to methods of treating pulmonary hypertension (e.g., pulmonary arterial hypertension), a kidney-associated disease (e.g., Alport syndrome or focal segmental glomerulosclerosis (FSGS)), and/or an interstitial lung disease (ILD) (e.g., idiopathic pulmonary fibrosis) comprising administering to a patient in need thereof an effective amount of any of, or any combination of, one or more variant ActRIIB proteins of the present disclosure (e.g., variant ActRIIB proteins in either homomeric or heteromeric forms). In some embodiments, the patient is administered any of the variant ActRIIB polypeptides and/or fragments thereof in either homomeric or heteromeric forms disclosed herein. In some embodiments, the disclosure contemplates methods of treating one or more complications of pulmonary hypertension (e.g., smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis) comprising administering to a patient in need thereof an effective amount of a variant ActRIIB protein in either homomeric or heteromeric form. In some embodiments, the disclosure contemplates methods of preventing one or more complications of pulmonary hypertension comprising administering to a patient in need thereof an effective amount of a variant ActRIIB protein in either homomeric or heteromeric form. In some embodiments, the disclosure contemplates methods of reducing the progression rate of pulmonary hypertension comprising administering to a patient in need thereof an effective amount of a variant ActRIIB protein in either homomeric or heteromeric form. In some embodiments, the disclosure contemplates methods of reducing the progression rate of one or more complications of pulmonary hypertension comprising administering to a patient in need thereof an effective amount of a variant ActRIIB protein in either homomeric or heteromeric form. In some embodiments, the disclosure contemplates methods of reducing the severity of pulmonary hypertension comprising administering to a patient in need thereof an effective amount of a variant ActRIIB protein in either homomeric or heteromeric form. In some embodiments, the disclosure contemplates methods of reducing the severity of one or more complications of pulmonary hypertension comprising administering to a patient in need thereof an effective amount of a variant ActRIIB protein in either homomeric or heteromeric form. Optionally, methods disclosed herein for treating, preventing, or reducing the progression rate and/or severity of pulmonary hypertension, particularly treating, preventing, or reducing the progression rate and/or severity of one or more complications of pulmonary hypertension, may further comprise administering to the patient one or more supportive therapies or additional active agents for treating pulmonary hypertension. For example, the patient also may be administered one or more supportive therapies or active agents selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-_{K}B antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-0-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1—>-3)-β-D-glucopyranosiduronic acid (CS1); oleanolic acid 3-O-β-D-glucopyranosyl (1—>-3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate) lung and/or heart transplantation

In some embodiments, the present disclosure relates to methods of treating an interstitial lung disease (e.g., idiopathic pulmonary fibrosis) comprising administering to a patient in need thereof an effective amount of any of the variant ActRIIB proteins in either homomeric or heteromeric form disclosed herein. In some embodiments, the patient is administered any of the ActRIIB polypeptides or variants and/or fragments thereof in either homomeric or heteromeric form disclosed herein. In some embodiments, the interstitial lung disease is pulmonary fibrosis. In some embodiments, the interstitial lung disease is caused by any one of the following: silicosis, asbestosis, berylliosis, hypersensitivity pneumonitis, drug use (e.g., antibiotics, chemotherapeutic drugs, antiarrhythmic agents, statins), systemic sclerosis, polymyositis, dermatomyositis, systemic lupus erythematosus, rheumatoid arthritis, an infection (e.g., atypical pneumonia, pneumocystis pneumonia, tuberculosis, chlamydia trachomatis, and/or respiratory syncytial virus), lymphangitic carcinomatosis, cigarette smoking, or developmental disorders. In some embodiments, the interstitial lung disease is idiopathic (e.g., sarcoidosis, idiopathic pulmonary fibrosis, Hamman-Rich syndrome, and/or antisynthetase syndrome). In particular embodiments, the interstitial lung disease is idiopathic pulmonary fibrosis (IPF). In some embodiments, the treatment for idiopathic pulmonary fibrosis is administered in combination with an additional therapeutic agent. In some embodiments, the additional therapeutic agent is selected from the group consisting of: pirfenidone, N-acetylcysteine, prednisone, azathioprine, nintedanib, derivatives thereof and combinations thereof.

Pulmonary hypertension (PH) has been previously classified as primary (idiopathic) or secondary. Recently, the World Health Organization (WHO) has classified pulmonary hypertension into five groups: Group 1: pulmonary arterial hypertension (PAH); Group 2: pulmonary hypertension with left heart disease; Group 3: pulmonary hypertension with lung disease and/or hypoxemia; Group 4: pulmonary hypertension due to chronic thrombotic and/or embolic disease; and Group 5: miscellaneous conditions (e.g., sarcoidosis, histiocytosis X, lymphangiomatosis and compression of pulmonary vessels). See, for example, Rubin (2004) Chest 126:7-10.

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of a variant ActRIIB protein in either homomeric or heteromeric form. In some embodiments, the patient is administered any of the ActRIIB polypeptides or variants and/or fragments thereof in either homomeric or heteromeric form disclosed herein. In some embodiments, the method relates to pulmonary hypertension patients that have pulmonary arterial hypertension. In some embodiments, the method relates pulmonary hypertension patients that have pulmonary hypertension with left heart disease. In some embodiments, the method relates to pulmonary hypertension patients that have lung disease and/or hypoxemia. In some embodiments, the method relates to pulmonary hypertension patients that have chronic thrombotic and/or embolic disease. In some embodiments, the method relates to pulmonary hypertension patients that have sarcoidosis, histiocytosis X, or lymphangiomatosis and compression of pulmonary vessels.

Pulmonary arterial hypertension is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in pulmonary vascular resistance (PVR) and sustained elevations in pulmonary artery pressure (PAP), which ultimately lead to right ventricular failure and death. Patients diagnosed with PAH have a poor prognosis and equally compromised quality of life, with a mean life expectancy of 2 to 5 years from the time of diagnosis if untreated.

A variety of factors contribute to the pathogenesis of pulmonary hypertension including proliferation of pulmonary cells which can contribute to vascular remodeling (i.e., hyperplasia). For example, pulmonary vascular remodeling occurs primarily by proliferation of arterial endothelial cells and smooth muscle cells of patients with pulmonary hypertension. Overexpression of various cytokines is believed to promote pulmonary hypertension. Further, it has been found that pulmonary hypertension may rise from the hyperproliferation of pulmonary arterial smooth cells and pulmonary endothelial cells. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of a variant ActRIIB protein in either homomeric or heteromeric form, wherein the patient has resting pulmonary arterial pressure (PAP) of at least 25 mm Hg (e.g., 25, 30, 35, 40, 45, or 50 mm Hg). In some embodiments, the patient is administered any of the ActRIIB polypeptides or variants and/or fragments thereof in either homomeric or heteromeric form disclosed herein. In some embodiments, the method relates to patients having a resting PAP of at least 25 mm Hg. In some embodiments, the method relates to patients having a resting PAP of at least 30 mm Hg. In some embodiments, the method relates to patients having a resting PAP of at least 35 mm Hg. In some embodiments, the method relates to patients having a resting PAP of at least 40 mm Hg. In some embodiments, the method relates to patients having a resting PAP of at least 45 mm Hg. In some embodiments, the method relates to patients having a resting PAP of at least 50 mm Hg.

In some embodiments, the disclosure relates to methods of adjusting one or more hemodynamic parameters in the PH patient toward a more normal level (e.g., normal as compared to healthy people of similar age and sex), comprising administering to a patient in need thereof an effective amount of a variant ActRIIB protein in either homomeric or heteromeric form. In some embodiments, the patient is administered any of the ActRIIB polypeptides or variants and/or fragments thereof in either homomeric or heteromeric form disclosed herein. In some embodiments, the method relates to reducing PAP. In some embodiments, the method relates to reducing the patient’s PAP by at least 3 mmHg. In certain embodiments, the method relates to reducing the patient’s PAP by at least 5 mmHg. In certain embodiments, the method relates to reducing the patient’s PAP by at least 7 mmHg. In certain embodiments, the method relates to reducing the patient’s PAP by at least 10 mmHg. In certain embodiments, the method relates to reducing the patient’s PAP by at least 12 mmHg. In certain embodiments, the method relates to reducing the patient’s PAP by at least 15 mmHg. In certain embodiments, the method relates to reducing the patient’s PAP by at least 20 mmHg. In certain embodiments, the method relates to reducing the patient’s PAP by at least 25 mmHg. In some embodiments, the method relates to reducing pulmonary vascular resistance (PVR). In some embodiments, the method relate to increasing pulmonary capillary wedge pressure (PCWP). In some embodiments, the method relate to increasing left ventricular end-diastolic pressure (LVEDP).

In certain aspects, the disclosure relates to methods of treating, preventing, or reducing the progression rate and/or severity of one or more complications of pulmonary hypertension comprising administering to a patient in need thereof an effective amount of a variant ActRIIB protein in either homomeric or heteromeric form. In some embodiments, the method relates to treating, preventing, or reducing the progression rate and/or severity of cell proliferation in the pulmonary artery of a pulmonary hypertension patient. In some embodiments, the patient is administered any of the ActRIIB polypeptides or variants and/or fragments thereof in either homomeric or heteromeric form disclosed herein. In some embodiments, the method relates to treating, preventing, or reducing the progression rate and/or severity of smooth muscle and/or endothelial cells proliferation in the pulmonary artery of a pulmonary hypertension patient. In some embodiments, the method relates to treating, preventing, or reducing the progression rate and/or severity of angiogenesis in the pulmonary artery of a pulmonary hypertension patient. In some embodiments, the method relates to increasing physical activity of a patient having pulmonary hypertension. In some embodiments, the method relates to treating, preventing, or reducing the progression rate and/or severity of dyspnea in a pulmonary hypertension patient. In some embodiments, the method relates to treating, preventing, or reducing the progression rate and/or severity of chest pain in a pulmonary hypertension patient. In some embodiments, the method relates to treating, preventing, or reducing the progression rate and/or severity of fatigue in a pulmonary hypertension patient. In some embodiments, the method relates to treating, preventing, or reducing the progression rate and/or severity of pulmonary fibrosis in a pulmonary hypertension patient. In some embodiments, the method relates to treating, preventing, or reducing the progression rate and/or severity of fibrosis in a pulmonary hypertension patient. In some embodiments, the method relates to treating, preventing, or reducing the progression rate and/or severity of pulmonary vascular remodeling in a pulmonary hypertension patient. In some embodiments, the method relates to treating, preventing, or reducing the progression rate and/or severity of right ventricular hypertrophy in a pulmonary hypertension patient.

In certain aspects, the disclosure relates to methods of increasing exercise capacity in a patient having pulmonary hypertension comprising administering to a patient in need thereof an effective amount of a variant ActRIIB protein in either homomeric or heteromeric form. In some embodiments, the patient is administered any of the ActRIIB polypeptides or variants and/or fragments thereof in either homomeric or heteromeric form disclosed herein. Any suitable measure of exercise capacity can be used. For example, exercise capacity in a 6-minute walk test (6MWT), which measures how far the subject can walk in 6 minutes, i.e., the 6-minute walk distance (6MWD), is frequently used to assess pulmonary hypertension severity and disease progression. The Borg dyspnea index (BDI) is a numerical scale for assessing perceived dyspnea (breathing discomfort). It measures the degree of breathlessness, for example, after completion of the 6MWT, where a BDI of 0 indicates no breathlessness and 10 indicates maximum breathlessness. In some embodiments, the method relates to increasing 6MWD by at least 10 meters in the patient having pulmonary hypertension. In some embodiments, the method relates to increasing 6MWD by at least 20 meters in the patient having pulmonary hypertension. In some embodiments, the method relates to increasing 6MWD by at least 30 meters in the patient having pulmonary hypertension. In some embodiments, the method relates to increasing 6MWD by at least 40 meters in the patient having pulmonary hypertension. In some embodiments, the method relates to increasing 6MWD by at least 50 meters in the patient having pulmonary hypertension. In some embodiments, the method relates to increasing 6MWD by at least 60 meters in the patient having pulmonary hypertension. In some embodiments, the method relates to increasing 6MWD by at least 70 meters in the patient having pulmonary hypertension. In some embodiments, the method relates to increasing 6MWD by at least 80 meters in the patient having pulmonary hypertension. In some embodiments, the method relates to increasing 6MWD by at least 90 meters in the patient having pulmonary hypertension. In some embodiments, the method relates to increasing 6MWD by at least 100 meters in the patient having pulmonary hypertension. In some embodiments, the method relate to lowering BDI by at least 0.5 index points in the patient having pulmonary hypertension. In some embodiments, the method relate to lowering BDI by at least 1 index points in the patient having pulmonary hypertension. In some embodiments, the method relate to lowering BDI by at least 1.5 index points in the patient having pulmonary hypertension. In some embodiments, the method relate to lowering BDI by at least 2 index points in the patient having pulmonary hypertension. In some embodiments, the method relate to lowering BDI by at least 2.5 index points in the patient having pulmonary hypertension. In some embodiments, the method relate to lowering BDI by at least 3 index points in the patient having pulmonary hypertension. In some embodiments, the method relate to lowering BDI by at least 3.5 index points in the patient having pulmonary hypertension. In some embodiments, the method relate to lowering BDI by at least 4 index points in the patient having pulmonary hypertension. In some embodiments, the method relate to lowering BDI by at least 4.5 index points in the patient having pulmonary hypertension. In some embodiments, the method relate to lowering BDI by at least 5 index points in the patient having pulmonary hypertension. In some embodiments, the method relate to lowering BDI by at least 5.5 index points in the patient having pulmonary hypertension. In some embodiments, the method relate to lowering BDI by at least 6 index points in the patient having pulmonary hypertension. In some embodiments, the method relate to lowering BDI by at least 6.5 index points in the patient having pulmonary hypertension. In some embodiments, the method relate to lowering BDI by at least 7 index points in the patient having pulmonary hypertension. In some embodiments, the method relate to lowering BDI by at least 7.5 index points in the patient having pulmonary hypertension. In some embodiments, the method relate to lowering BDI by at least 8 index points in the patient having pulmonary hypertension. In some embodiments, the method relate to lowering BDI by at least 8.5 index points in the patient having pulmonary hypertension. In some embodiments, the method relate to lowering BDI by at least 9 index points in the patient having pulmonary hypertension. In some embodiments, the method relate to lowering BDI by at least 9.5 index points in the patient having pulmonary hypertension. In some embodiments, the method relate to lowering BDI by at least 3 index points in the patient having pulmonary hypertension. In some embodiments, the method relate to lowering BDI by 10 index points in the patient having pulmonary hypertension.

Pulmonary hypertension at baseline can be mild, moderate or severe, as measured for example by World Health Organization (WHO) functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Class I: pulmonary hypertension without resulting limitation of physical activity; ordinary physical activity does not cause undue dyspnea or fatigue, chest pain or near syncope; Class II: pulmonary hypertension resulting in slight limitation of physical activity; patient comfortable at rest; ordinary physical activity causes undue dyspnea or fatigue, chest pain or near syncope; Class III: pulmonary hypertension resulting in marked limitation of physical activity; patient comfortable at rest; less than ordinary activity causes undue dyspnea or fatigue, chest pain or near syncope; Class IV: pulmonary hypertension resulting in inability to carry out any physical activity without symptoms; patient manifests signs of right-heart failure; dyspnea and/or fatigue may be present even at rest; discomfort is increased by any physical activity.

In certain aspects, the disclosure relates to methods of treating, preventing, or reducing the progression rate and/or severity of pulmonary hypertension (e.g., treating, preventing, or reducing the progression rate and/or severity of one or more complications of pulmonary hypertension) comprising administering to a patient in need thereof a variant ActRIIB protein in either homomeric or heteromeric form, wherein the patient has Class I, Class II, Class III, or Class IV pulmonary hypertension as recognized by the WHO. In some embodiments, the patient is administered any of the ActRIIB polypeptides or variants and/or fragments thereof in either homomeric or heteromeric form disclosed herein. In some embodiments, the method relates to a patient that has Class I pulmonary hypertension as recognized by the WHO. In some embodiments, the method relates to a patient that has Class II pulmonary hypertension as recognized by the WHO. In some embodiments, the method relates to preventing or delaying patient progression from Class I pulmonary hypertension to Class II pulmonary hypertension as recognized by the WHO. In some embodiments, the method relates to promoting or increasing patient regression from Class II pulmonary hypertension to Class I pulmonary hypertension as recognized by the WHO. In some embodiments, the method relates to a patient that has Class III pulmonary hypertension as recognized by the WHO. In some embodiments, the method relates to preventing or delaying patient progression from Class II pulmonary hypertension to Class III pulmonary hypertension as recognized by the WHO. In some embodiments, the method relates to promoting or increasing patient regression from Class III pulmonary hypertension to Class II pulmonary hypertension as recognized by the WHO. In some embodiments, the method relates to promoting or increasing patient regression from Class III pulmonary hypertension to Class I pulmonary hypertension as recognized by the WHO. In some embodiments, the method relates to a patient that has Class IV pulmonary hypertension as recognized by the WHO. In some embodiments, the method relates to preventing or delaying patient progression from Class III pulmonary hypertension to Class IV pulmonary hypertension as recognized by the WHO. In some embodiments, the method relates to promoting or increasing patient regression from Class IV pulmonary hypertension to Class III pulmonary hypertension as recognized by the WHO. In some embodiments, the method relates to promoting or increasing patient regression from Class IV pulmonary hypertension to Class II pulmonary hypertension as recognized by the WHO. In some embodiments, the method relates to promoting or increasing patient regression from Class IV pulmonary hypertension to Class I pulmonary hypertension as recognized by the WHO.

There is no known cure for pulmonary hypertension; current methods of treatment focus on prolonging patient lifespan and enhancing patient quality of life. Current methods of treatment of pulmonary hypertension include administration of: vasodilators such as prostacyclin, epoprostenol, and sildenafil; endothelin receptor antagonists such as bosentan; calcium channel blockers such as amlodipine, diltiazem, and nifedipine; anticoagulants such as warfarin; and diuretics. Treatment of pulmonary hypertension has also been carried out using oxygen therapy, atrial septostomy, pulmonary thromboendarterectomy, and lung and/or heart transplantation. Each of these methods, however, suffers from one or multiple drawbacks which may include lack of effectiveness, serious side effects, low patient compliance, and high cost. In certain aspects, the method relate to treating, preventing, or reducing the progression rate and/or severity of pulmonary hypertension (e.g., treating, preventing, or reducing the progression rate and/or severity of one or more complications of pulmonary hypertension) comprising administering to a patient in need thereof an effective amount of a variant ActRIIB protein in either homomeric or heteromeric form disclosed herein in combination (e.g., administered at the same time or different times, but generally in such a manner as to achieve overlapping pharmacological/physiological effects) with one or more additional active agents and/or supportive therapies for treating pulmonary hypertension (e.g., vasodilators such as prostacyclin, epoprostenol, and sildenafil; endothelin receptor antagonists such as bosentan; calcium channel blockers such as amlodipine, diltiazem, and nifedipine; anticoagulants such as warfarin; diuretics; oxygen therapy; atrial septostomy; pulmonary thromboendarterectomy: and lung and/or heart transplantation); bardoxolone methyl or a derivative thereof; oleanolic acid or derivative thereof.

In part, the present disclosure relates to methods of treating renal diseases or conditions (e.g., Alport syndrome, focal segmental glomerulosclerosis (FSGS), polycystic kidney diseases, chronic kidney disease), comprising administering to a patient in need thereof an effective amount of a variant ActRIIB polypeptide (e.g., a variant ActRIIB homomultimer protein or a variant ActRIIB heteromultimer protein) of the present disclosure, that can be used to treat or prevent a disease or condition that is associated with abnormal activity of a ActRIIA or ActRIIB polypeptide, and/or an ActRIIA or ActRIIB ligand (e.g., Activin A, activin B, GDF11, GDF8, GDF3, BMP5, BMP6, and BMP10).

The terms “renal” and “kidney” are used interchangeably herein.

In some embodiments, any of the variant ActRIIB polypeptides in either homomeric or heteromeric form disclosed herein may be used, alone or in combination with one or more supportive therapies or active agents, to treat, prevent, or reduce the progression rate and/or severity of a kidney-associated disease or condition. As used herein, “kidney-associated disease or condition” can refer to any disease, disorder, or condition that affects the kidneys or the renal system. Examples of kidney-associated diseases or conditions include, but are not limited to, chronic kidney diseases (or failure), acute kidney diseases (or failure), primary kidney diseases, non-diabetic kidney diseases, glomerulonephritis, interstitial nephritis, diabetic kidney diseases, diabetic nephropathy, glomerulosclerosis, rapid progressive glomerulonephritis, renal fibrosis, Alport syndrome, IDDM nephritis, mesangial proliferative glomerulonephritis, membranoproliferative glomerulonephritis, crescentic glomerulonephritis, renal interstitial fibrosis, focal segmental glomerulosclerosis (FSGS), membranous nephropathy, minimal change disease, pauci-immune rapid progressive glomerulonephritis, IgA nephropathy, polycystic kidney disease (PKD), Dent’s disease, nephrocytinosis, Heymann nephritis, autosomal dominant (adult) polycystic kidney disease (ADPKD), autosomal recessive (childhood) polycystic kidney disease (ARPKD), acquired cystic kidney disease (ACKD), polycystic kidney syndrome (PKS), acute kidney injury, nephrotic syndrome, renal ischemia, podocyte diseases or disorders, proteinuria, glomerular diseases, membranous glomerulonephritis, focal segmental glomerulonephritis, pre-eclampsia, eclampsia, kidney lesions, collagen vascular diseases, benign orthostatic (postural) proteinuria, IgM nephropathy, membranous nephropathy, sarcoidosis, diabetes mellitus, kidney damage due to drugs, Fabry’s disease, aminoaciduria, Fanconi syndrome, hypertensive nephrosclerosis, interstitial nephritis, Sickle cell disease, hemoglobinuria, myoglobinuria, Wegener’s Granulomatosis, Glycogen Storage Disease Type 1, chronic kidney disease, chronic renal failure, low Glomerular Filtration Rate (GFR), nephroangiosclerosis, lupus nephritis, ANCA-positive pauci-immune crescentic glomerulonephritis, chronic allograft nephropathy, nephrotoxicity, renal toxicity, kidney necrosis, kidney damage, glomerular and tubular injury, kidney dysfunction, nephritic syndrome, acute renal failure, chronic renal failure, proximal tubal dysfunction, acute kidney transplant rejection, chronic kidney transplant rejection, non-IgA mesangioproliferative glomerulonephritis, postinfectious glomerulonephritis, vasculitides with renal involvement of any kind, any hereditary renal disease, any interstitial nephritis, renal transplant failure, kidney cancer, kidney disease associated with other conditions (e.g., hypertension, diabetes, and autoimmune disease), Dent’s disease, nephrocytinosis, Heymann nephritis, a primary kidney disease, a collapsing glomerulopathy, a dense deposit disease, a cryoglobulinemia-associated glomerulonephritis, an Henoch-Schonlein disease, a postinfectious glomerulonephritis, a bacterial endocarditis, a microscopic polyangitis, a Churg-Strauss syndrome, an anti-GBM-antibody mediated glomerulonephritis, amyloidosis, a monoclonal immunoglobulin deposition disease, a fibrillary glomerulonephritis, an immunotactoid glomerulopathy, ischemic tubular injury, a medication-induced tubulo-interstitial nephritis, a toxic tubulo-interstitial nephritis, an infectious tubulo-interstitial nephritis, a bacterial pyelonephritis, a viral infectious tubulo-interstitial nephritis which results from a polyomavirus infection or an HIV infection, a metabolic-induced tubulo-interstitial disease, a mixed connective disease, a cast nephropathy, a crystal nephropathy which may results from urate or oxalate or drug-induced crystal deposition, an acute cellular tubulo-interstitial allograft rejection, a tumoral infiltrative disease which results from a lymphoma or a post-transplant lymphoproliferative disease, an obstructive disease of the kidney, vascular disease, a thrombotic microangiopathy, a nephroangiosclerosis, an atheroembolic disease, a mixed connective tissue disease, a polyarteritis nodosa, a calcineurin-inhibitor induced-vascular disease, an acute cellular vascular allograft rejection, an acute humoral allograft rejection, early renal function decline (ERFD), end stage renal disease (ESRD), renal vein thrombosis, acute tubular necrosis, acute interstitial nephritis, established chronic kidney disease, renal artery stenosis, ischemic nephropathy, uremia, drug and toxin-induced chronic tubulointerstitial nephritis, reflux nephropathy, kidney stones, Goodpasture’s syndrome, normocytic normochromic anemia, renal anemia, diabetic chronic kidney disease, IgG4-related disease, von Hippel-Lindau syndrome, tuberous sclerosis, nephronophthisis, medullary cystic kidney disease, renal cell carcinoma, adenocarcinoma, nephroblastoma, lymphoma, leukemia, hyposialylation disorder, chronic cyclosporine nephropathy, renal reperfusion injury, renal dysplasia, azotemia, bilateral arterial occlusion, acute uric acid nephropathy, hypovolemia, acute bilateral obstructive uropathy, hypercalcemic nephropathy, hemolytic uremic syndrome, acute urinary retention, malignant nephrosclerosis, postpartum glomerulosclerosis, scleroderma, non-Goodpasture’s anti-GBM disease, microscopic polyarteritis nodosa, allergic granulomatosis, acute radiation nephritis, post-streptococcal glomerulonephritis, Waldenstrom’s macroglobulinemia, analgesic nephropathy, arteriovenous fistula, arteriovenous graft, dialysis, ectopic kidney, medullary sponge kidney, renal osteodystrophy, solitary kidney, hydronephrosis, microalbuminuria, uremia, haematuria, hyperlipidemia, hypoalbuminaemia, lipiduria, acidosis, hyperkalemia, and edema.

In some embodiments, the disclosure contemplates methods of treating one or more complications of a renal disease or condition comprising administering to a subject in need thereof an effective amount of a variant ActRIIB polypeptide (e.g., a variant ActRIIB homomultimer protein or a variant ActRIIB heteromultimer protein). In some embodiments, the disclosure contemplates methods of preventing one or more complications of a renal disease or condition comprising administering to a subject in need thereof an effective amount of a variant ActRIIB polypeptide (e.g., a variant ActRIIB homomultimer protein or a variant ActRIIB heteromultimer protein). In some embodiments, the disclosure contemplates methods of reducing the progression rate of a renal disease or condition comprising administering to a subject in need thereof an effective amount of a variant ActRIIB polypeptide (e.g., a variant ActRIIB homomultimer protein or a variant ActRIIB heteromultimer protein). In some embodiments, the disclosure contemplates methods of reducing the progression rate of one or more complications of a renal disease or condition comprising administering to a subject in need thereof an effective amount of a variant ActRIIB polypeptide (e.g., a variant ActRIIB homomultimer protein or a variant ActRIIB heteromultimer protein). In some embodiments, the disclosure contemplates methods of reducing the severity of a renal disease or condition comprising administering to a subject in need thereof an effective amount of a variant ActRIIB polypeptide (e.g., a variant ActRIIB homomultimer protein or a variant ActRIIB heteromultimer protein). In some embodiments, the disclosure contemplates methods of reducing the severity of one or more complications of a renal disease or condition comprising administering to a subject in need thereof an effective amount of a variant ActRIIB polypeptide (e.g., a variant ActRIIB homomultimer protein or a variant ActRIIB heteromultimer protein). In some embodiments, a renal disease or condition is selected from the group consisting of Alport syndrome, focal segmental glomerulosclerosis (FSGS), polycystic kidney disease, and chronic kidney disease. In some embodiments, a renal disease or condition is Alport syndrome. In some embodiments, a renal disease or condition is focal segmental glomerulosclerosis (FSGS). In some embodiments, a renal disease or condition is polycystic kidney disease. In some embodiments, a renal disease or condition is chronic kidney disease. In some embodiments, a subject has a decline in kidney function. In some embodiments, methods of the present disclosure slow kidney function decline.

In certain aspects, the disclosure relates to methods of treating, preventing, or reducing the progression rate and/or severity of a renal disease or condition comprising administering to a subject in need thereof an effective amount of a variant ActRIIB polypeptide (e.g., a variant ActRIIB homomultimer protein or a variant ActRIIB heteromultimer protein). In some embodiments, the method relates to renal disease or condition in subjects that have Alport syndrome.

Alport syndrome, also known as hereditary nephritis, is a genetically heterogeneous disease that results from mutations in genes encoding alpha-3, alpha-4, and alpha-5 chains of type IV collagen. Type IV collagen alpha chains are normally located in various basement membranes throughout the body, including the kidneys. Abnormalities in these chains can result in defective basement membranes at these sites, which in turn lead to clinical features of Alport syndrome (e.g., progressive glomerular disease).

Transmission of Alport syndrome can be X-linked, autosomal recessive, or autosomal dominant. In some embodiments, a subject has X-linked Alport syndrome. In some embodiments, the disclosure relates to methods of treating a subject that has X-linked Alport syndrome. X-linked transmission accounts for the majority of affected patients and arises from mutations in the COL4A5 gene on the X chromosome. In some embodiments, a subject has genetic defects in the COL4A5 gene. In some embodiments, the disclosure relates to methods of treating a subject that has one or more genetic defects in the COL4A5 gene. Autosomal recessive variant accounts for approximately 15 percent of patients with Alport syndrome and arises from genetic defects in either the COL4A3 or COL4A4 genes. In some embodiments, a subject has autosomal recessive Alport syndrome. Autosomal dominant disease appears to account for between about 20 to about 30 percent of patients with Alport syndrome and arises from heterozygous mutations in the COL4A3 or COL4A4 genes. In some embodiments, a subject has autosomal dominant Alport syndrome. In some embodiments, a subject has heterozygous mutations in the COL4A3 gene. In some embodiments, a subject has heterozygous mutations in the COL4A4 gene. In some embodiments, a subject has genetic defects in the COL4A3 gene. In some embodiments, the disclosure relates to methods of treating a subject that has one or more genetic defects in the COL4A3 gene. In some embodiments, a subject has genetic defects in the COL4A4 gene. In some embodiments, the disclosure relates to methods of treating a subject that has one or more genetic defects in the COL4A4 gene. In some embodiments, a subject has genetic defects in the COL4A3 and COLA4A genes. In some embodiments, the disclosure relates to methods of treating a subject that has one or more genetic defects in the COL4A3 and COL4A4 genes. Some families exhibit digenic inheritance due to transmission of mutations in two of the three genes (COL4A3, COL4A4, COL4A5). In some embodiments, a subject has mutations in two of the three genes (COL4A3, COL4A4, COL4A5). In some embodiments, the disclosure relates to methods of treating a subject that has one or more genetic defects in the COL4A3, COL4A4, and/or COL4A5 genes.

The classical presentation of Alport syndrome is based upon clinical manifestations of affected males with X-linked disease. In some embodiments, a subject with X-linked disease has one or more of glomerular disease that progresses to end-stage renal disease (ESRD). Clinical presentation and course in patients with autosomal recessive disease is similar to those with X-linked disease. Patients with autosomal dominant disease generally exhibit more gradual loss of renal function.

Initially, renal manifestation of Alport syndrome is typically asymptomatic persistent microscopic hematuria (e.g., presence of blood in the urine), which is usually present in early childhood in affected patients. Since screening urinalysis is seldom performed in routine pediatric primary care, microscopic hematuria may not be detected unless the patient is screened because of an affected family member or found as an incidental finding for another issue. Gross hematuria may be the initial presenting finding and often occurs after an upper respiratory infection. However, recurrent episodes of gross hematuria are not uncommon especially during childhood. In some embodiments, the disclosure relates to methods of treating a subject that has asymptomatic persistent microscopic hematuria. In some embodiments, the disclosure relates to methods of treating a subject that has gross hematuria. In some embodiments, the disclosure relates to methods of treating a subject that has recurring episodes of gross hematuria. In some embodiments, the disclosure relates to methods of reducing the severity, occurrence, and/or duration of asymptomatic persistent microscopic hematuria, gross hematuria, or persistent microscopic hematuria in a subject in need thereof (e.g., a subject with Alport syndrome).

Patients with Alport syndrome typically have normal C3 levels, which is a component of the complement pathway that plays an integral role in the body’s immune defenses. Decreased C3 may be associated with acute glomerulonephritis, membranoproliferative glomerulonephritis, immune complex disease, active systemic lupus erythematosus, septic shock, and end-stage liver disease, among other conditions. In early childhood, serum creatinine and blood pressure measurements are usually at normal levels as well. In some embodiments, the disclosure relates to methods of treating a subject with Alport syndrome that has normal levels of C3. In some embodiments, the disclosure relates to methods of treating a subject with Alport syndrome that has decreased levels of C3 compared to a baseline measurement. In some embodiments, the disclosure relates to methods of increasing C3 levels in a subject in need thereof (e.g., a subject with Alport syndrome).

Proteinuria, hypertension, and progressive renal insufficiency may develop in a subject with Alport syndrome. Proteinuria comprises a presence of excess proteins in urine. Albumin is a protein produced by the liver which makes up roughly 50%-60% of the proteins in the blood. Due to this, the concentration of albumin in the urine is one of the most sensitive indicators of any kidney disease, particularly for subjects with diabetes or hypertension, compared to a routine proteinuria examination. This measurement is often referred to as albuminuria. In some embodiments, the disclosure relates to methods of treating a subject that has proteinuria. In some embodiments, the disclosure relates to methods of treating a subject that has hypertension. In some embodiments, the disclosure relates to methods of treating a subject that has progressive renal insufficiency. In some embodiments, the disclosure relates to methods of reducing the severity, occurrence, and/or duration of one or more of proteinuria, hypertension, and progressive renal insufficiency in a subject in need thereof (e.g., a subject with Alport syndrome).

Subjects with Alport syndrome may develop end-stage renal disease (ESRD). ESRD usually occurs between the ages of 16 and 35 years in patients with X-linked or autosomal recessive Alport syndrome, among many other renal diseases and conditions. In some families, the course is more indolent with kidney failure being delayed until age 45 to 60, especially in those with autosomal dominant Alport syndrome. Females with X-linked Alport syndrome may have recurrent episodes of gross hematuria, proteinuria, and diffuse glomerular basement membrane (GBM) thickening are associated with more severe kidney dysfunction and ESRD at an earlier age. In some embodiments, the disclosure relates to methods of treating subjects with Alport syndrome that have ESRD. In some embodiments, the disclosure relates to methods of treating females with X-linked Alport syndrome. In some embodiments, the disclosure relates to methods of reducing severity, occurrence and/or duration of one or more of gross hematuria, proteinuria, and diffuse glomerular basement membrane (GBM) thickening are associated with more severe kidney dysfunction and ESRD in a subject in need thereof (e.g., a subject with Alport syndrome).

A diagnosis of Alport syndrome may be made by molecular genetic testing, or by skin or renal biopsy. Molecular genetic next generation analysis is a preferred method to make a diagnosis for patients with a positive family history for persistent hematuria and/or end-stage renal disease (ESRD) and for patients with chronic kidney disease (CKD), regardless of family history. Alport syndrome can be distinguished from other glomerular diseases by presence of a characteristic finding of lamination of the glomerular basement membrane (GBM) in samples from a renal biopsy, or abnormalities of type IV collagen by immunostaining, or by identification of one or more mutations in COL4A3, COL4A4, or COL4A5. Thin glomerular basement membranes in a subject with a COL4A3, COL4A4, or COL4A5 mutation, with or without the manifestation of FSGS, is properly diagnosed as Alport syndrome. In some embodiments, the disclosure relates to methods of treating subjects with Alport syndrome that have a positive family history for persistent hematuria and/or end-stage renal disease (ESRD) and/or for patients with chronic kidney disease (CKD).

In certain aspects, the disclosure relates to methods of treating, preventing, or reducing the progression rate and/or severity of a renal disease or condition comprising administering to a subject in need thereof an effective amount of a variant ActRIIB polypeptide (e.g., a variant ActRIIB homomultimer protein or a variant ActRIIB heteromultimer protein) to subjects that have focal segmental glomerulosclerosis (FSGS).

FSGS is a glomerular scarring disease characterized by an effacement of the podocyte foot on a kidney biopsy. When urine samples from subjects suffering FSGS are analyzed, a massive urine protein loss is typically observed, which can progress to a renal failure. FSGS is a common histopathologic lesion among adults with idiopathic nephrotic syndrome in the United States, accounting for about 35 percent of all cases. FSGS is also the most common primary glomerular disease identified in patients with end-stage renal disease (ESRD) in the United States. Prevalence of FSGS as a lesion associated with ESRD has risen. FSGS is characterized by the presence of sclerosis in parts (segmental) of at least one glomerulus (focal) of a kidney biopsy specimen, when examined by light microscopy (LM), immunofluorescence (IF), or electron microscopy (EM). In some embodiments, the disclosure relates to methods of reducing severity, occurrence and/or duration of urine protein loss in a subject in need thereof (e.g., a subject with FSGS). In some embodiments, the disclosure relates to methods of reducing severity, occurrence and/or duration of renal failure in a subject in need thereof (e.g., a subject with FSGS). In some embodiments, the disclosure relates to methods of reducing severity, occurrence and/or duration of end stage renal disease (ESRD) in a subject in need thereof (e.g., a subject with FSGS). In some embodiments, the disclosure relates to methods of reducing severity, occurrence and/or duration of sclerosis in a glomerulus of a kidney in a subject in need thereof (e.g., a subject with FSGS).

FSGS arises as a consequence of multiple pathways either individually or collectively resulting in injury to a podocyte, which is a cell in the Bowman’s capsule in the kidneys that wraps around capillaries of the glomerulus. There are five known etiologies, and a suggested sixth etiology, associated with FSGS. Etiologies of FSGS comprise primary (e.g., idiopathic), secondary (e.g., adaptive), genetic, virus-associated, medication-associated, and APOL1 risk allele-associated. Primary or idiopathic FSGS is associated with a plasma factor with responsiveness to immunosuppressive therapy and a risk of recurrence after kidney transplant. In primary FSGS, a putative circulating factor that is toxic to a podocyte causes generalized podocyte dysfunction. Primary FSGS most often presents with the nephrotic system. Secondary (e.g., adaptive) FSGS is associated with excessive nephron workload due to increased body size, reduced nephron capacity, or single glomerular hyperfiltration associated with certain diseases. Secondary FSGS generally occurs as an adaptive phenomenon that results from a reduction in nephron mass, or can be considered as medicated-induced by direct toxicity from drugs (e.g., heroin, interferon, and pamidronate) or virus-induced by viral infections (e.g., HIV). Secondary FSGS often presents with non-nephrotic proteinuria, and/or with some degree of renal insufficiency. Secondary FSGS most commonly refers to FSGS that develops as an adaptive response to glomerular hypertrophy or hyperfiltration. Additional etiologies are recognized as drivers of FSGS, including high-penetrance genetic FSGS due to mutations in one of nearly 40 genes (genetic FSGS), virus-associated FSGS, and medication-associated FSGS. Emerging data support the identification of a sixth etiology: APOL1 risk allele-associated FSGS in individuals with sub-Saharan ancestry. Sometimes, secondary FSGS encompasses virus-associated FSGS and/or medication-associated FSGS. In some embodiments, the disclosure relates to methods of treating a subject with primary or idiopathic FSGS. In some embodiments, the disclosure relates to methods of treating a subject with secondary or adaptive FSGS. In some embodiments, the disclosure relates to methods of treating a subject with genetic FSGS. In some embodiments, the disclosure relates to methods of treating a subject with virus-associated FSGS. In some embodiments, the disclosure relates to methods of treating a subject with medication-associated FSGS. In some embodiments, the disclosure relates to methods of treating a subject with APOL1 risk allele-associated FSGS.

Primary FSGS comprises several prototypical characteristics. Primary FSGS is the most common form of FSGS in adolescents and young adults, and is commonly associated with nephrotic-range proteinuria (sometimes massive proteinuria, e.g., >10 g protein/day in the urine), reduced plasma albumin levels, and/or hyperlipidemia. In some embodiments, nephrotic-range proteinuria comprises proteinuria >3.5 g protein/day, and/or hypoalbuminemia <3.5 g albumin/dL urine (<35 g/L), and/or other manifestations of the nephrotic syndrome (e.g., edema, hyperlipidemia). In some embodiments, the disclosure relates to methods of reducing severity, occurrence and/or duration of one or more of nephrotic range proteinuria, reduced plasma albumin levels, or hyperlipidemia in a subject in need thereof (e.g., a subject with primary FSGS). In some embodiments, the disclosure relates to methods of reducing severity, occurrence and/or duration of proteinuria in a subject in need thereof (e.g., a subject with primary FSGS).

A subject with secondary or adaptive FSGS typically presents with slowly increasing proteinuria and renal insufficiency over time. Proteinuria in subjects with secondary FSGS often presents in the non-nephrotic range (e.g., nephrotic range is typically a loss of 3 grams or more of protein in the urine per day, and/or presence of 2 grams of protein per gram of creatinine in the urine). Sometimes, proteinuria in subjects with secondary FSGS comprises serum albumin levels that are normal. A subject with secondary FSGS may have s a glomerular filtration rate (GFR) that is elevated, which is a measurement of the flow rate of filtered fluid through the kidney. In some embodiments, a subject with secondary FSGS and an increase in GFR may have one or more additional and/or associated conditions selected from the group consisting of congenital cyanotic heart disease, sickle cell anemia, obesity, androgen abuse, sleep apnea, and high-protein diet. In some embodiments, the disclosure relates to methods of treating a subject with secondary FSGS with a normal GFR. In some embodiments, the disclosure relates to methods of treating a subject with secondary FSGS that has a decreased GFR. In some embodiments, the disclosure relates to methods of reducing severity, occurrence and/or duration of proteinuria and/or renal insufficiency in a subject in need thereof (e.g., a subject with primary FSGS).

Viruses have been implicated in causing FSGS. HIV-1 may be associated with FSGS, particularly the collapsing glomerulopathy variant. Other viruses that have been implicated in causing FSGS include, but are not limited to, cytomegalovirus, parvovirus B19, and Epstein-Barr virus. Parasites have also been associated with FSGS, which include, but are not limited to, Plasmodium (malaria), Schistosoma mansoni, and filiariasis. In some embodiments, the disclosure relates to methods of treating a subject with FSGS associated with HIV-1. In some embodiments, the disclosure relates to methods of treating a subject with FSGS associated with one or more of HIV-1, cytomegalovirus, parvovirus B19, and Epstein-Barr virus. In some embodiments, the disclosure relates to methods of treating a subject with FSGS associated with parasites including, but not limited to, Plasmodium (malaria), Schistosoma mansoni, and filiariasis. In some embodiments, the disclosure relates to methods of treating subjects with FSGS associated with an infection including, but not limited to, HIV, cytomegalovirus, parvovirus B19, Epstein-Barr virus, pulmonary tuberculosis, leishmaniasis, and malaria.

In some embodiments, the disclosure relates to methods of treating subjects with FSGS associated with autoimmune disorders implicated in causing FSGS including, but not limited to Adult Still’s disease, systemic lupus erythematosus, and mixed connective tissue disorder.

In some embodiments, the disclosure relates to methods of treating subjects with FSGS associated with malignancies implicated in causing FSGS including, but not limited to hemophagocytic lymphohistiocytosis, multiple myeloma, and acute monoblastic leukemia.

In some embodiments, the disclosure relates to methods of treating subjects with FSGS associated with acute glomerular ischemias implicated in causing FSGS including, but not limited to thrombotic microangiopathy, renal infarction, atheroembolism, and hydrophilic polymer embolism.

In some embodiments, the disclosure relates to methods of treating subjects with FSGS associated with genetic disorders implicated in causing FSGS including, but not limited to APOL1 high-risk alleles, sickle cell disease, mitochondrial disorders (coenzyme Q deficiency), acute myoclonus-renal failure syndrome, and Galloway-Mowat syndrome.

In some embodiments, the disclosure relates to methods of treating subjects with FSGS associated with post transplantation events implicated in causing FSGS including, but not limited to Arteriopathy/thrombotic microangiopathy, acute rejection, and viral infection (cytomegalovirus, Epstein-Barr virus, BK polyomavirus).

In some embodiments, the disclosure relates to methods of treating subjects with FSGS associated with certain medications. In some embodiments, IFN-α, -β, or -γ therapy has been associated with development of collapsing glomerulopathy. In some embodiments, the disclosure relates to methods of treating subjects with FSGS associated with one or more of podocyte injury, including MCD, FSGS, and particularly, collapsing FSGS (collapsing glomerulopathy) who have taken and/or are still taking bisphosphonates. In some embodiments, the disclosure relates to methods of treating subjects with FSGS who have been on and/or are currently on lithium therapy. In some embodiments, the disclosure relates to methods of treating subjects with FSGS who have taken and/or are still taking sirolimus. In some embodiments, the disclosure relates to methods of treating subjects with FSGS who have taken and/or are still taking anthracycline medications, including doxorubicin (Adriamcyin) and daunomycin. In some embodiments, the disclosure relates to methods of treating subjects with FSGS who have taken and/or are still taking medications implicated in causing FSGS including, but not limited to bisphosphonates, interferons (alpha, beta, or gamma), anabolic steroids, calcineurin inhibitors, and mammalian (mechanistic) target of rapamycin (mTOR) inhibitors.

Genetic FSGS takes two forms. In some embodiments, the disclosure relates to methods of treating subjects with genetic FSGS associated with one or more variants in susceptibility genes (i.e., some individuals with a particular variant will develop FSGS, and other individuals will not). In some embodiments, the disclosure relates to methods of treating subjects with FSGS associated with one or more susceptibility genes including, but not limited to APOL1 and PDSS1. In some embodiments, the disclosure relates to methods of treating subjects with genetic FSGS associated with one or more high-penetrance mutations that manifest either Mendelian inheritance (for nuclear genes) or maternal inheritance (for genes encoded by mitochondrial DNA). The number of genes associated with FSGS rises every year, in large part because of the dissemination of whole-exome sequencing. At least 38 genes have been identified in relation to genetic FSGS. In some embodiments, the disclosure relates to methods of treating subjects with FSGS associated with one or more genes involved in genetic FSGS comprising COL4A3, COL4A4, COL4A5, ITGB4, LAMB2, NPHS, NPHS2, CD2AP, PTPRO, MYO1E, ACTN4, INF2, AHRGP24, AHRGDIA, MYH9, INF1, MT-TL1, MT-TL2, MT-TY, COQ2, COQ6, PDSS2, ADCK, WT1, NUP95, NUP203, XP05, NXFS, PAX2, LMX1B, SMARCAL1, NXF5, EYA1, WDR73, LMNA, PLCE1, TRPC6, KANK4, SCARB2, and TTC21B.

In some embodiments, a subject suspected of FSGS is administered a kidney biopsy. A kidney biopsy may be analyzed by light microscopy to determine one or more of glomerular size, histologic variant of FSGS, microcystic tubular changes, and tubular hypertrophy. Further, a kidney biopsy may be analyzed by immunofluorescence to rule out other primary glomerulopathies and/or by electron microscopy to determine one or more of an extent of podocyte foot process effacement, podocyte microvillous transformation, and tubuloreticular inclusions. A complete assessment of renal histology is important for establishing the parenchymal setting of segmental glomerulosclerosis, distinguishing FSGS associated with one of many other glomerular diseases from the clinical-pathologic syndrome of FSGS. In some embodiments, genetic testing is used to further analyze a subject for a genetic FSGS etiology.

Traditionally, FSGS was classified based upon the Columbia classification, which defined five morphologic variants of FSGS lesions based upon LM examination. This classification system was designed to rely solely on pathologic criteria and does not integrate these findings with clinical and/or genetic information. In general, morphologic characteristics seen on kidney biopsy cannot distinguish between genetic and nongenetic forms of FSGS. Exceptions include distinctive features associated with NPHS1 and actinin alpha 4 gene mutations and the disease-specific lesions of Fabry disease, Alport syndrome, and lecithin-cholesterol acyl transferase deficiency. Histologic variants of FSGS comprise FSGS not otherwise specified (NOS) (formerly called classic FSGS, which is the most common form); collapsing variant, tip variant; perihilar variant; and cellular variant. Although the appearance of a glomerulus on LM, by definition, differs among these forms, they all share ultrastructural findings of podocyte alterations. Tip lesions affect the portion of the glomerular tuft juxtaposed to the tubular pole, and a tip lesion abnormality includes one or more of adhesion to Bowman’s capsule at the tip, hypercellularity, presence of foam cells, and/or sclerosis. A collapsing variant shows segmental or global mesangial consolidation and loss of endocapillary patency in association with extracapillary epithelial hypertrophy and/or proliferation. Perihilar and NOS variants are determined by whether the segmental sclerosis/segmental obliteration of capillary loops with matrix increase (with or without hyalinosis) involves the segment near the hilum or the specific segment cannot be determined, respectively. A cellular lesion is the most difficult lesion to identify reproducibly. A cellular lesion shows segmental endocapillary hypercellularity occluding lumens with or without foam cells and karyorrhexis.

In certain aspects, the disclosure relates to methods of treating, preventing, or reducing the progression rate and/or severity of a renal disease or condition comprising administering to a subject in need thereof an effective amount of a variant ActRIIB polypeptide (e.g., a variant ActRIIB homomultimer protein or a variant ActRIIB heteromultimer protein), wherein the renal disease or condition is polycystic kidney disease (PKD).

Polycystic Kidney Disease occurs in two forms: autosomal recessive (ARPKD) and autosomal dominant (ADPKD). The two forms of the disease have distinct genetic basis and two genes involved in ADPKD has been identified one gene involved in ARPKD has been identified. The manifestations of the two different types of disease are very similar, and both result from a hyperproliferation of tubule epithelial cells that ultimately results in destruction of tubular structure with cyst formation leading to chronic renal failure. In some embodiments, the disclosure relates to methods of treating subjects with autosomal recessive polycystic kidney disease (ARPKD). In some embodiments, the disclosure relates to methods of treating subjects with autosomal dominant polycystic kidney disease (ADPKD).

Autosomal dominant polycystic kidney disease (ADPKD) is a hereditary disorder of the kidneys characterized by markedly enlarged kidneys with extensive cyst formation throughout. These cysts progressively enlarge with age, as kidney function gradually declines. A diagnosis of ADPKD is based on family history and ultrasonographic evaluation. In as many as 25% of patients with ADPKD, no family history is identified, which may be related to subclinical disease or a new genetic mutation in about 5% of such cases. A defining feature of ADPKD is marked bilateral, renal enlargement. Patients with ADPKD typically progress to end-stage renal disease (ESRD) by the fifth or sixth decade of life. The rate of progression of ADPKD is related directly to kidney volume, and therapies aim to slow the decline in renal volume to delay progression. In some embodiments, the disclosure relates to methods of reducing severity, occurrence and/or duration of cysts on the kidney in a subject in need thereof. In some embodiments, the disclosure relates to methods of reducing severity, occurrence and/or duration of renal enlargement in a subject in need thereof. In some embodiments, the disclosure relates to methods of reducing severity, occurrence and/or duration of an increase in kidney volume (e.g., total kidney volume) in a subject in need thereof.

ADPKD can be attributed to an abnormality on chromosome 16 (PKD1 locus) or chromosome 4 (PKD2 locus). PKD1 mutations comprise about 78% of ADPKD cases, while PKD2 mutations comprise about 14% of cases. PKD1 patients tend to progress to ESRD at an earlier age than PKD2 patients. In some embodiments, the disclosure relates to methods of treating a subject with ADPKD that has a mutation in the PKD1 locus. In some embodiments, the disclosure relates to methods of treating a subject with ADPKD that has a mutation in the PKD2 locus.

The PKD1 and PKD2 genes encode the proteins polycystin-1 and polycystin-2, respectively. These polycystins are integral membrane proteins and are found in renal tubular epithelia. It is postulated that abnormalities in polycystin-1 impair cell-cell and cell-matrix interactions in the renal tubular epithelia, while abnormalities in polycystin-2 impair calcium signaling in the cells.

Resultant changes in renal pathophysiology due to PKD include, but are not limited to, hematuria (often gross), a concentrating defect (resulting in polyuria and increased thirst), mild proteinuria, nephrolithiasis (in about 25% of ADPKD patients), flank pain, and abdominal pain. Furthermore, cyst rupture, hemorrhage, and infection are common complications. Progressive renal decline often results in end-stage renal disease. Hypertension is the most prevalent initial clinical presentation, occurring in about 50% to about 70% of cases, and is the most common feature directly associated with the rate of decline to ESRD and cardiovascular complications. In some embodiments, the disclosure relates to methods of reducing severity, occurrence and/or duration of one or more of hematuria, a concentrating defect, proteinuria, nephrolithiasis, flank pain, and abdominal pain in a subject in need thereof. In some embodiments, the disclosure relates to methods of reducing severity, occurrence and/or duration of one or more of cyst rupture, hemorrhage, and infection in a subject in need thereof. In some embodiments, the disclosure relates to methods of reducing severity, occurrence and/or duration of end-stage renal disease (ESRD) in a subject in need thereof. In some embodiments, the disclosure relates to methods of reducing severity, occurrence and/or duration of hypertension in a subject in need thereof.

Multiple extra-renal manifestations are often present in a subject with polycystic kidney disease. Cerebral aneurysms occur in about 5% of young adults, and as many as 20% of patients over the age of 60. Risk of a cerebral aneurysm or subarachnoid hemorrhage is highest in subjects with a family history of the same. Extrarenal cysts are common in ADPKD. Hepatic cysts are often noted in these patients, and prevalence increases with age. As many as 94% of patients over the age of 35 have been reported to have hepatic cysts. Total cyst prevalence and volume is higher in women versus men. Hepatic cysts in ADPKD patients rarely cause liver dysfunction. Rarely, patients develop pain from an acute cyst infection or hemorrhage. In addition, between about 7% and about 36% of ADPKD patients develop pancreatic cysts, with a higher prevalence in ADPKD patients with PKD2 mutations. Cardiac valvular disease has been noted in 25% to 30% of ADPKD patients. Cardiovascular complications, particularly cardiac hypertrophy and coronary artery disease, are leading causes of death in patients with ADPKD. In some embodiments, the disclosure relates to methods of reducing severity, occurrence and/or duration of a cerebral aneurysm in a subject in need thereof. In some embodiments, the disclosure relates to methods of reducing severity, occurrence and/or duration of extrarenal cysts in a subject in need thereof. In some embodiments, the disclosure relates to methods of reducing severity, occurrence and/or duration of hepatic cysts in a subject in need thereof. In some embodiments, the disclosure relates to methods of reducing severity, occurrence and/or duration of pancreatic cysts in a subject in need thereof. In some embodiments, the disclosure relates to methods of reducing severity, occurrence and/or duration of cardiovascular complications (e.g., cardiac hypertrophy, coronary artery disease) in a subject. in need thereof

Autosomal recessive polycystic kidney disease (ARPKD) is a cause of significant renal and liver-related morbidity and mortality in children. A majority of subjects with ARPKD present in the neonatal period with enlarged echogenic kidneys. Renal disease is characterized by nephromegaly, hypertension, and varying degrees of renal dysfunction. More than 50% of affected individuals with ARPKD progress to end-stage renal disease (ESRD) within the first decade of life, and subjects with ARPKD whom progressed to ESRD may require kidney transplantation. In some embodiments, the disclosure relates to methods of reducing severity, occurrence and/or duration of one or more nephromegaly, hypertension, and renal dysfunction in a subject in need thereof. In some embodiments, the disclosure relates to methods of reducing severity, occurrence and/or duration of end stage renal disease in a subject in need thereof, preventing a need for kidney transplantation.

ARPKD can be attributed to mutations in the PKHD1 gene located on chromosome 6p21, which contains at least 66 exons and encodes fibrocystin (also referred to as polyductin), a large integral membrane protein. Although the function of fibrocystin is presently unknown, it is found in the cortical and medullary collecting ducts and the thick ascending limb of the kidney, and in the epithelial cells of the hepatic bile duct. In some embodiments, the disclosure relates to methods of treating a subject with ARPKD that is associated with one or more mutations in PKHD1.

Because of the diversity of PKIID1 mutations, it can be challenging to correlate genotype with phenotype in cases of ARPKD. Subjects with two truncation mutations may have more severe renal involvement and are possibly at risk for early neonatal death. Subjects who are homozygotes for a missense mutation, or who have a missense mutation paired with a truncating mutation, may also have a severe phenotype. Subjects who are heterozygotes with two missense mutations typically have milder disease. Subjects who survive the neonatal period most often have at least one missense mutation. In some embodiments, the present disclosure relates to methods of treating a subject with ARPKD comprising two truncation mutations. In some embodiments, the present disclosure relates to methods of treating a subject with ARPKD comprising one or more missense mutations.

Two primary organ systems affected in ARPKD are the kidney and hepatobiliary tract. Kidneys may increase in size and/or have microcysts (usually less than 2 mm in size), which radiate from the medulla to the cortex, and are visible as pinpoint dots on the capsular surface. Severity of renal disease is proportional to the percentage of nephrons affected by cysts. Larger renal cysts (up to 1 cm) and interstitial fibrosis develop, which contribute to the progressive deterioration of renal function seen in subjects who survive beyond the neonatal period. ARPKD is associated with biliary dysgenesis due to a developmental defect comprising varying degrees of dilatation of the intrahepatic bile ducts and hepatic fibrosis. In some embodiments, the disclosure relates to methods of reducing severity, occurrence and/or duration of an increase in kidney size and/or presence of cysts.

Clinical presentation of ARPKD varies based on the age of onset of symptoms and the predominance of hepatic or renal involvement. ARPKD is often detected by routine antenatal ultrasonography in fetuses after 24 weeks of gestation. A presumptive diagnosis is based on the presence of characteristic findings of markedly enlarged echogenic kidneys with poor corticomedullary differentiation. Discrete cysts ranging in size from 5 to 7 mm in diameter may be detected; however, larger cysts are unusual, especially those >10 mm in diameter. Subjects with ARPKD are typically monitored for blood pressure changes, renal function, serum electrolyte concentrations, hydration status, nutritional status, and growth. In some embodiments, the disclosure relates to methods of treating a subject with ARPKD further comprising monitoring one or more of blood pressure, renal function, serum electrolyte concentration, hydration status, nutritional status, and growth.

During the neonatal period, infants can present with renal manifestations, which may or may not be accompanied by respiratory distress. An infant with ARPKD may present with bilateral markedly enlarged kidneys, which may impact pulmonary function or lead to difficulty in feeding due to renal compression of the stomach. An infant with ARPKD may present with renal function impairment reflected by increased serum/plasma concentrations of creatinine and blood urea nitrogen (BUN). Neonates with end-stage renal disease (ESRD) may require renal replacement therapy (RRT) for survival. An infant with ARPKD may present with one or more of hypertension and hyponatremia (due to the inability to dilute urine maximally). In some embodiments, the disclosure relates to methods of reducing severity, occurrence and/or duration of renal function impairment reflected by increased serum/plasma concentrations of creatinine and blood urea nitrogen (BUN) in a subject in need thereof. In some embodiments, the disclosure relates to methods of reducing severity, occurrence and/or duration of hypertension and/or hyponatremia in a subject in need thereof.

For patients who survive beyond the neonatal period, there can be improvement of renal function due to continued renal maturation. However, over time, progressive deterioration of renal function can develop, which may be rapid or slow and may result in ESRD. An adolescent subject with ARPKD may have one or more of progressive deterioration of renal function (usually beginning with signs of tubular dysfunction or injury, polyuria and/or polydipsia due to a reduced concentrating ability, a maximal urine osmolality below 500 mosmol/kg, metabolic acidosis due to decreased urinary acidification capacity, hypertension, recurrent episodes of urinary tract infections, urinary abnormalities (including, but not limited to, mild proteinuria, glucosuria, hyperphosphaturia, and/or increased urinary excretion of magnesium), progressive renal impairment, and decreased kidney growth rate and/or kidney size. In some embodiments, the present disclosure relates to methods of reducing severity, occurrence and/or duration of one or more of progressive deterioration of renal function, progressive renal impairment, and decreased kidney growth rate and/or kidney size in a subject in need thereof.

Ultrasound findings of ARPKD are characterized by bilateral large echogenic kidneys with poor corticomedullary differentiation. In patients with only medullary involvement, standard-resolution ultrasonography may be normal; however, high-resolution ultrasonography is able to detect ductal dilations confined to the medulla. Macrocysts, typically seen in subjects with autosomal dominant disease, are not usually present during infancy in patients with ARPKD, but may appear in older children. As a result, in older subjects, it may be more challenging to differentiate ARPKD from autosomal dominant polycystic kidney disease (ADPKD) by ultrasound. In some embodiments, the present disclosure relates to methods of treating a subject with ARPKD or ADPKD, further comprising differentiation of disease by ultrasound.

In certain aspects, the disclosure relates to methods of treating, preventing, or reducing the progression rate and/or severity of a renal disease or condition comprising administering to a subject in need thereof an effective amount of a variant ActRIIB polypeptide (e.g., a variant ActRIIB homomultimer protein or a variant ActRIIB heteromultimer protein) to a subject that has chronic kidney disease (CKD).

Chronic kidney disease (CKD) is a condition in which the kidneys are damaged and cannot filter blood as well as healthy kidneys. A subject with CKD typically has excess fluid and waste from blood remaining in the body. In some embodiments, the disclosure provides methods of treating a subject with CKD. In some embodiments, the disclosure relates to treating a subject with CKD, wherein the subject also has one or more other health conditions selected from the group consisting of anemia or low number of red blood cells, increased occurrence of infections, low calcium levels, high potassium levels, and high phosphorus levels in the blood, loss of appetite or eating less, depression or lower quality of life.

CKD has varying levels of seriousness and typically gets worse over time, though treatment has been shown to slow progression. If left untreated, CKD can progress to kidney failure, end stage renal disease (ESRD), and/or early cardiovascular disease, potentially leading to dialysis or kidney transplant for survival. In some embodiments, the present disclosure relates to methods of reducing severity, occurrence and/or duration of kidney failure, end stage renal disease (ESRD), and/or early cardiovascular disease in a subject in need thereof.

Diagnosis of CKD is typically accomplished by blood tests to measure the estimated glomerular filtration rate (eGFR), and/or a urine test to measure albumin and/or overall protein in the urine. Typically, an increase in protein in the urine indicates CKD. Ultrasound or kidney biopsy may be performed to determine an underlying cause.

In some embodiments, CKD manifests initially without symptoms, and is usually detected on routine screening blood work by either an increase in serum creatinine, and/or protein in the urine. As kidney function of a subject with CKD decreases, blood pressure increases due to fluid overload and production of vasoactive hormones created by the kidney via the renin-angiotensin system, thereby increasing the risk of developing hypertension and heart failure. As urea accumulates in a subject with CKD, azotemia and ultimately uremia (symptoms ranging from lethargy to pericarditis and encephalopathy) may arise. Due to its high systemic concentration, urea is excreted in eccrine sweat at high concentrations and crystallizes on skin as the sweat evaporates (e.g., “uremic frost”). In a subject with CKD, potassium may accumulate in the blood (e.g., hyperkalemia with a range of symptoms including malaise and potentially fatal cardiac arrhythmias). Hyperkalemia usually does not develop in a subject with CKD until the glomerular filtration rate (GFR) falls to less than about 20 to about 25 ml/min/1.73 m², at which point the kidneys have decreased ability to excrete potassium. Hyperkalemia in CKD can be exacerbated by acidemia (which leads to extracellular shift of potassium) and from lack of insulin. A subject with CKD may have hyperphosphatemia, which can result from poor phosphate elimination in the kidney. Hyperphosphatemia contributes to increased cardiovascular risk by causing vascular calcification. A subject with CKD may have hypocalcemia. A subject with CKD may have one or more changes in mineral and bone metabolism that may cause abnormalities of calcium, phosphorus (phosphate), parathyroid hormone, or vitamin D metabolism; abnormalities in bone turnover, mineralization, volume, linear growth, or strength (kidney osteodystrophy); and/or vascular or other soft-tissue calcification. A subject with CKD may have metabolic acidosis that may result from decreased capacity to generate enough ammonia from the cells of the proximal tubule. A subject with CKD may have anemia. In later stages of CKD, a subject may develop cachexia, leading to unintentional weight loss, muscle wasting, weakness and anorexia. Subjects with CKD are more likely than the general population to develop atherosclerosis with consequent cardiovascular disease. In some embodiments, the present disclosure relates to methods of reducing severity, occurrence and/or duration of one or more conditions or complications of CKD selected from the group consisting of blood pressure increase, hypertension and/or heart failure, azotemia, uremia, “uremic frost”, hyperkalemia, decreased ability of the kidney to excrete potassium, acidemia, hyperphosphatemia, vascular calcification, hypocalcemia. changes in mineral and bone metabolism (particularly changes that may cause abnormalities of calcium, phosphorus (phosphate), parathyroid hormone, or vitamin D metabolism), abnormalities in bone turnover, mineralization, volume, linear growth, or strength (kidney osteodystrophy), vascular or other soft-tissue calcification, metabolic acidosis, anemia, cachexia (particularly cachexia that may lead to unintentional weight loss, muscle wasting, weakness and anorexia), and atherosclerosis (which may lead to cardiovascular disease).

Common causes of CKD are diabetes mellitus, hypertension, and glomerulonephritis. About one of five adults with hypertension and one of three adults with diabetes have CKD. CKD may also be caused by one or more of vascular diseases (including but not limited to, large vessel disease such as bilateral kidney artery stenosis and small vessel disease such as ischemic nephropathy, hemolytic-uremic syndrome, vasculitis), primary glomerular disease (focal segmental glomerulosclerosis (FSGS) and/or IgA nephropathy (or nephritis)), secondary glomerular disease (such as diabetic nephropathy and lupus nephritis), tubulointerstitial disease (which includes drug- and toxin-induced chronic tubulointerstitial nephritis, and reflux nephropathy), obstructive nephropathy (as exemplified by bilateral kidney stones and benign prostatic hyperplasia of the prostate gland), and congenital disease (such as polycystic kidney disease). Rarely, pinworms infecting the kidney can cause obstructive nephropathy. In some embodiments, the present disclosure relates to methods of treating a subject with CKD caused by one or more of diabetes mellitus, hypertension, and glomerulonephritis, vascular diseases (including but not limited to, large vessel disease such as bilateral kidney artery stenosis and small vessel disease such as ischemic nephropathy, hemolytic-uremic syndrome, vasculitis), primary glomerular disease (focal segmental glomerulosclerosis (FSGS) and/or IgA nephropathy (or nephritis)), secondary glomerular disease (such as diabetic nephropathy and lupus nephritis), tubulointerstitial disease (which includes drug- and toxin-induced chronic tubulointerstitial nephritis, and reflux nephropathy), obstructive nephropathy (as exemplified by bilateral kidney stones and benign prostatic hyperplasia of the prostate gland), and congenital disease (such as polycystic kidney disease). Rarely, pinworms infecting the kidney can cause obstructive nephropathy.

In some embodiments, any of the variant ActRIIB proteins in either homomeric or heteromeric form disclosed herein may be used, alone or in combination with one or more supportive therapies or active agents, to treat, prevent, or reduce the progression rate and/or severity of chronic kidney disease (e.g., tissue damage, inflammation, and/or fibrosis). Chronic kidney disease (CKD), also known as chronic renal disease, is a progressive loss in renal function over a period of months or years. The symptoms of worsening kidney function may include feeling generally unwell and experiencing a reduced appetite. Often, chronic kidney disease is diagnosed as a result of screening of people known to be at risk of kidney problems, such as those with high blood pressure or diabetes and those with a blood relative with CKD. This disease may also be identified when it leads to one of its recognized complications, such as cardiovascular disease, anemia, or pericarditis. Recent professional guidelines classify the severity of CKD in five stages, with stage 1 being the mildest and usually causing few symptoms and stage 5 being a severe illness with poor life expectancy if untreated. Stage 5 CKD is often called end-stage kidney disease, end-stage renal disease, or end-stage kidney failure, and is largely synonymous with the now outdated terms chronic renal failure or chronic kidney failure; and usually means the patient requires renal replacement therapy, which may involve a form of dialysis, but ideally constitutes a kidney transplant. CKD is initially without specific symptoms and is generally only detected as an increase in serum creatinine or protein in the urine. As the kidney function decreases, various symptoms may manifest as described below. Blood pressure may be increased due to fluid overload and production of vasoactive hormones created by the kidney via the renin-angiotensin system, increasing one’s risk of developing hypertension and/or suffering from congestive heart failure. Urea may accumulate, leading to azotemia and ultimately uremia (symptoms ranging from lethargy to pericarditis and encephalopathy). Due to its high systemic circulation, urea is excreted in eccrine sweat at high concentrations and crystallizes on skin as the sweat evaporates (“uremic frost”). Potassium may accumulate in the blood (hyperkalemia with a range of symptoms including malaise and potentially fatal cardiac arrhythmias). Hyperkalemia usually does not develop until the glomerular filtration rate falls to less than 20-25 ml/min/1.73 m2, at which point the kidneys have decreased ability to excrete potassium. Hyperkalemia in CKD can be exacerbated by acidemia (which leads to extracellular shift of potassium) and from lack of insulin. Erythropoietin synthesis may be decreased causing anemia. Fluid volume overload symptoms may occur, ranging from mild edema to life-threatening pulmonary edema. Hyperphosphatemia, due to reduced phosphate excretion, may occur generally following the decrease in glomerular filtration. Hyperphosphatemia is associated with increased cardiovascular risk, being a direct stimulus to vascular calcification. Hypocalcemia may manifest, which is generally caused by stimulation of fibroblast growth factor-23. Osteocytes are responsible for the increased production of FGF23, which is a potent inhibitor of the enzyme 1-alpha-hydroxylase (responsible for the conversion of 25-hydroxycholecalciferol into 1,25 dihydroxyvitamin D3). Later, this progresses to secondary hyperparathyroidism, renal osteodystrophy, and vascular calcification that further impairs cardiac function. Metabolic acidosis (due to accumulation of sulfates, phosphates, uric acid etc.) may occur and cause altered enzyme activity by excess acid acting on enzymes; and also increased excitability of cardiac and neuronal membranes by the promotion of hyperkalemia due to excess acid (acidemia). Acidosis is also due to decreased capacity to generate enough ammonia from the cells of the proximal tubule. Iron deficiency anemia, which increases in prevalence as kidney function decreases, is especially prevalent in those requiring haemodialysis. It is multifactoral in cause, but includes increased inflammation, reduction in erythropoietin, and hyperuricemia leading to bone marrow suppression. People with CKD suffer from accelerated atherosclerosis and are more likely to develop cardiovascular disease than the general population. Patients afflicted with CKD and cardiovascular disease tend to have significantly worse prognoses than those suffering only from the latter. In some embodiments, the chronic kidney disease is a chronic kidney disease mineral bone disorder, a broad syndrome of interrelated skeletal, cardiovascular, and mineral-metabolic disorders arising from kidney disease. CKD-MBD encompasses various skeletal pathologies often referred to as renal osteodystrophy (ROD), which is a preferred embodiment for treatment with any of the polypeptides disclosed herein, or combinations with one or more supportive therapies or active agents. Depending on the relative contribution of different pathogenic factors, ROD is manifested as diverse pathologic patterns of bone remodeling (Hruska et al., 2008, Chronic kidney disease mineral bone disorder (CKD-MBD); in Rosen et al. (ed) Primer on the Metabolic Bone Diseases and Disorders of Mineral Metabolism, 7th ed. American Society for Bone and Mineral Research, Washington D.C., pp 343-349). At one end of the spectrum is ROD with uremic osteodystrophy and low bone turnover, characterized by a low number of active remodeling sites, profoundly suppressed bone formation, and low bone resorption. At the other extreme is ROD with hyperparathyroidism, high bone turnover, and osteitis fibrosa.

In some embodiments, the disclosure contemplates methods of treating one or more complications of a kidney-associated disease (e.g., Alport syndrome or focal segmental glomerulosclerosis) comprising administering to a patient in need thereof an effective amount of a variant ActRIIB protein in either homomeric or heteromeric form. In some embodiments, the disclosure contemplates methods of preventing one or more complications of a kidney-associated disease (e.g., Alport syndrome or focal segmental glomerulosclerosis) comprising administering to a patient in need thereof a variant ActRIIB protein in either homomeric or heteromeric form. In some embodiments, the disclosure contemplates methods of reducing the progression rate of a kidney-associated disease (e.g., Alport syndrome or focal segmental glomerulosclerosis) comprising administering to a patient in need thereof an effective amount of a variant ActRIIB protein in either homomeric or heteromeric form. In some embodiments, the disclosure contemplates methods of reducing the progression rate of one or more complications of a kidney-associated disease (e.g., Alport syndrome or focal segmental glomerulosclerosis) comprising administering to a patient in need thereof a variant ActRIIB protein in either homomeric or heteromeric form. In some embodiments, the disclosure contemplates methods of reducing the severity of a kidney-associated disease (e.g., Alport syndrome or focal segmental glomerulosclerosis) comprising administering to a patient in need thereof a variant ActRIIB protein in either homomeric or heteromeric form. In some embodiments, the disclosure contemplates methods of reducing the severity of one or more complications of a kidney-associated disease (e.g., Alport syndrome or focal segmental glomerulosclerosis) comprising administering to a patient in need thereof an effective amount of a variant ActRIIB protein in either homomeric or heteromeric form. Optionally, methods disclosed herein for treating, preventing, or reducing the progression rate and/or severity of a kidney-associated disease (e.g., Alport syndrome or focal segmental glomerulosclerosis), particularly treating, preventing, or reducing the progression rate and/or severity of one or more complications of a kidney-associated disease, may further comprise administering to the patient one or more supportive therapies or additional active agents for treating the kidney-associated disease.

In some embodiments, the disclosure relates to methods of monitoring a subject with a renal disease or condition (e.g., Alport syndrome, focal segmental glomerulosclerosis (FSGS), polycystic kidney disease, chronic kidney disease) is for albuminuria and/or proteinuria. Elevated protein levels in urine is a hallmark of many renal diseases or conditions. Annual monitoring for albuminuria and proteinuria are initiated beginning at one year of age for at-risk children. Proteinuria comprises a presence of abnormal quantities of protein in the urine. The most sensitive marker of proteinuria is elevated urine albumin (e.g., albuminuria). Albumin typically circulates in the blood, and only a trace of albumin is found in urine of subjects without a renal disease or condition. Moderate albuminuria is typically called microalbuminuria, while severe albuminuria is typically called macroalbuminuria. An albumin level above the upper limit value is called severe albuminuria or macroalbuminuria. In some embodiments, the present disclosure provides methods of treating a subject with one or more of albuminuria, proteinuria, microalbuminuria, and macroalbuminuria. In some embodiments, the present disclosure relates to methods of reducing severity, occurrence and/or duration of one or more of albuminuria, proteinuria, microalbuminuria, and macroalbuminuria in a subject in need thereof.

Measurements of albumin can have different units depending on how such measurements were taken. In some embodiments, albumin in urine is measured as a mass of albumin per time period of urine collected (e.g., mg/24 hr). In some embodiments, albumin in urine is measured as a mass of albumin per volume of urine collected (e.g., mg/L). In some embodiments, albumin in urine is measured as a mass of albumin per mass of creatinine in the urine (e.g., µg/mg of creatinine, termed albumin-creatine ratio, or ACR).

In some embodiments, a subject is administered a urine test to determine presence of a kidney disease or condition (e.g., Alport syndrome, focal segmental glomerulosclerosis (FSGS), polycystic kidney disease, chronic kidney disease). In some embodiments, a urine test comprises collection of urine over a specific time period (e.g., 24 hours). Moderate albuminuria or microalbuminuria comprises a level of albumin detected in the urine from a 24-hour urine collection that is between about 30 and about 300 mg albumin/24 hours and/or a level of albumin detected in the urine from a one minute urine collection that is between about 20 and about 200 µg albumin/1 minute. Severe albuminuria or macroalbuminuria comprises a level of albumin detected in the urine from a 24-hour urine collection that is above about 300 mg albumin/24 hours and/or a level of albumin detected in the urine from a 1 minute urine collection that is above about 200 µg albumin/1 minute. In some embodiments, the disclosure relates to methods of treating a subject with moderate albuminuria or microalbuminuria comprising a level of albumin detected in the urine from a 24-hour urine collection that is between about 30 and about 300 mg albumin/24 hours. In some embodiments, the disclosure relates to methods of treating a subject with moderate albuminuria or microalbuminuria comprising a level of albumin detected in the urine from a one minute urine collection that is between about 20 and about 200 µg albumin/1 minute. In some embodiments, the disclosure relates to methods of treating a subject with severe albuminuria or macroalbuminuria comprising a level of albumin detected in the urine from a 24-hour urine collection that is above about 300 mg albumin/24 hours. In some embodiments, the disclosure relates to methods of treating a subject with severe albuminuria or macroalbuminuria comprising a level of albumin detected in the urine from a 1 minute urine collection that is above about 200 µg albumin/1 minute.

In some embodiments, a urine test comprises a spot test using a single sample of urine. In some embodiments, a urine test comprises a dipstick test. In some embodiments, a urine dipstick test may provide an estimate of the level of albuminuria. In some embodiments, moderate albuminuria or microalbuminuria comprises a level of albumin detected in the urine from a spot sample that is between about 20 and about 200 mg albumin/L urine. In some embodiments, severe albuminuria or macroalbuminuria comprises a level of albumin detected in the urine from a spot sample that is above about 200 mg albumin/L urine. In some embodiments, the disclosure relates to methods of treating a subject with moderate albuminuria or microalbuminuria comprising a level of albumin detected in a urine from a spot sample that is between about 20 and about 200 mg albumin/L urine. In some embodiments, the disclosure relates to methods of treating a subject with severe albuminuria or macroalbuminuria comprising a level of albumin detected in the urine from a spot sample that is above about 200 mg albumin/L urine.

To compensate for variations in urine concentration in spot-check samples (versus a larger sample collection and/or a sample collection over time), comparing the amount of albumin in the sample against the urine concentration of creatinine is useful. This is called the albumin/creatinine ratio (ACR). In some embodiments, presence and/or severity of albuminuria is determined by a ratio of albumin to creatinine in the urine (e.g., albumin-creatinine ratio, ACR). ACR lower and upper limits can vary between men and women. ACR is measured as a unit of mass of albumin per a unit of mass of creatinine in the urine. In some embodiments, the disclosure provides methods of treating a subject with moderate albuminuria or microalbuminuria comprising an ACR of between about 30 and about 300 mg albumin/g of creatinine. In some embodiments, the disclosure provides methods of treating a subject with severe albuminuria or macroalbuminuria comprising an ACR of above about 300 mg albumin/g of creatinine. In some embodiments, a normal ACR is typically below 30 mg albumin/g creatinine. It is important to note that the units of measure for any albuminuria measurement can differ. For example, ACR may be measured as µg of albumin per mg of creatinine. Units of mg albumin/g creatinine are interchangeable with units of µg albumin/mg creatinine. ACR is sometimes provided without units, if both albumin and creatinine are provided as measurements of mass.

ACR can be measured as mass of albumin per concentration of creatinine in the urine. In some embodiments, the disclosure provides methods of treating moderate albuminuria or microalbuminuria comprising an ACR of between about 2.5 and about 35 mg albumin/mmol of creatinine in a subject in need thereof. In some embodiments, the disclosure provides methods of treating severe albuminuria or macroalbuminuria comprising an ACR of above about 35 mg albumin/mmol of creatinine in a subject in need thereof.

Disease stages describing the extent of renal damage and loss of function in a subject are typically assigned to subjects with renal diseases or conditions. Albuminuria stages are typically measured in terms of an ACR. In some embodiments, the present disclosure relates to methods of treating a subject with Stage A1 albuminuria. Stage A1 albuminuria comprises normal to moderately increased levels of albumin in the urine, with an ACR of less than 30 mg albumin/g creatinine (or less than 3 mg albumin/mmol creatinine). In some embodiments, the present disclosure relates to methods of treating a subject with Stage A2 albuminuria. Stage A2 comprises moderate albuminuria or microalbuminuria, with an ACR of between about 30 and about 300 mg albumin/g creatinine(or between about 3 and about 30 mg albumin/mmol creatinine). In some embodiments, the present disclosure relates to methods of treating a subject with Stage A3 albuminuria. Stage A3 comprises severe albuminuria or macroalbuminuria, with an ACR of greater than 300 mg albumin/g creatinine (or greater than 30 mg albumin/mmol creatinine). In some embodiments, administration of therapy to a subject with a renal disease or condition will delay or prevent development of end stage renal disease. In some embodiments, administration of therapy to a subject with a renal disease or condition will lower said subject’s albuminuria stage. In some embodiments, the present disclosure relates to methods of reducing severity, occurrence and/or duration of Stage A1 albuminuria. In some embodiments, the present disclosure relates to methods of reducing severity, occurrence and/or duration of Stage A2 albuminuria. In some embodiments, the present disclosure relates to methods of reducing severity, occurrence and/or duration of Stage A3 albuminuria. In some embodiments, the present disclosure provides methods of treating a subject with Stage A1 albuminuria that delay or prevent progression to Stage A2 albuminuria. In some embodiments, the present disclosure provides methods of treating a subject with Stage A2 albuminuria that delay or prevent progression to Stage A3 albuminuria. In some embodiments, the present disclosure provides methods of delaying and/or preventing worsening of albuminuria stage progression in a subject in need thereof. In some embodiments, the present disclosure provides an improvement in renal damage and/or a downgrade in albuminuria stage classification in a subject in need thereof. In some embodiments, the present disclosure provides methods of improving albuminuria classification in a subject by one or more stages.

In some embodiments, a subject has proteinuria in the nephrotic range. In some embodiments, proteinuria in the nephrotic range comprises between about 3 and about 3.5 g of protein in the urine per 24 hours per 1.73 m² body surface area. In some embodiments, a subject with nephrotic syndrome has proteinuria of greater than 3.5 g/24 hrs/1.73 m².

In some embodiments, the disclosure relates to methods of reducing an ACR in a subject with a renal disease or condition, comprising administering to a subject in need thereof an effective amount of a variant ActRIIB polypeptide (e.g., a variant ActRIIB homomultimer protein or a variant ActRIIB heteromultimer protein). In some embodiments, the method relates to reducing the subject’s ACR by between about 0.1 and about 2.5 mg albumin/g creatinine compared to a baseline measurement. In some embodiments, the method relates to reducing the subject’s ACR by between about 2.5 and about 3.5 mg albumin/g creatinine compared to a baseline measurement. In some embodiments, the method relates to reducing the subject’s ACR by between about 3.5 and about 5.0 mg albumin/g creatinine compared to a baseline measurement. In some embodiments, the method relates to reducing the subject’s ACR by between about 5.0 and about 7.5 mg albumin/g creatinine compared to a baseline measurement.. In some embodiments, the method relates to reducing the subject’s ACR by between about 7.5 and about 10.0 mg albumin/g creatinine compared to a baseline measurement. In some embodiments, the method relates to reducing the subject’s ACR by between about 10.0 and about 15.0 mg albumin/g creatinine compared to a baseline measurement. In some embodiments, the method relates to reducing the subject’s ACR by between about 15.0 and about 20.0 mg albumin/g creatinine compared to a baseline measurement. In some embodiments, the method relates to reducing the subject’s ACR by between about 20.0 and about 25.0 mg albumin/g creatinine compared to a baseline measurement. In some embodiments, the method relates to reducing the subject’s ACR by between about 30.0 and about 35.0 mg albumin/g creatinine compared to a baseline measurement. In some embodiments, the method relates to reducing the subject’s ACR by between about 40.0 and about 45.0 mg albumin/g creatinine compared to a baseline measurement. In some embodiments, the method relates to reducing the subject’s ACR by between about 45.0 and about 50.0 mg albumin/g creatinine compared to a baseline measurement. In some embodiments, the method relates to reducing the subject’s ACR by between about 50.0 and about 60.0 mg albumin/g creatinine compared to a baseline measurement. In some embodiments, the method relates to reducing the subject’s ACR by between about 60.0 and about 70.0 mg albumin/g creatinine compared to a baseline measurement. In some embodiments, the method relates to reducing the subject’s ACR by between about 70.0 and about 80.0 mg albumin/g creatinine compared to a baseline measurement. In some embodiments, the method relates to reducing the subject’s ACR by between about 80.0 and about 90.0 mg albumin/g creatinine compared to a baseline measurement. In some embodiments, the method relates to reducing the subject’s ACR by between about 90.0 and about 100.0 mg albumin/g creatinine compared to a baseline measurement.

In some embodiments, the method relates to reducing the subject’s ACR by at least 2.5% compared to a baseline measurement. In some embodiments, the method relates to reducing the subject’s ACR by at least 5% compared to a baseline measurement. In some embodiments, the method relates to reducing the subject’s ACR by at least 10% compared to a baseline measurement. In some embodiments, the method relates to reducing the subject’s ACR by at least 15% compared to a baseline measurement. In some embodiments, the method relates to reducing the subject’s ACR by at least 20% compared to a baseline measurement. In some embodiments, the method relates to reducing the subject’s ACR by at least 25% compared to a baseline measurement. In some embodiments, the method relates to reducing the subject’s ACR by at least 30% compared to a baseline measurement. In some embodiments, the method relates to reducing the subject’s ACR by at least 40% compared to a baseline measurement. In some embodiments, the method relates to reducing the subject’s ACR by at least 50% compared to a baseline measurement. In some embodiments, the method relates to reducing the subject’s ACR by at least 60% compared to a baseline measurement. In some embodiments, the method relates to reducing the subject’s ACR by at least 70% compared to a baseline measurement. In some embodiments, the method relates to reducing the subject’s ACR by at least 80% compared to a baseline measurement. In some embodiments, the method relates to reducing the subject’s ACR by at least 90% compared to a baseline measurement. In some embodiments, the method relates to reducing the subject’s ACR by at least 95% compared to a baseline measurement. In some embodiments, the method relates to reducing the subject’s ACR by at least 99% compared to a baseline measurement.

In some embodiments, total urine protein may be measured and compared against creatinine presence in the urine (e.g., UPCR). In some embodiments, UPCR is a measurement of proteinuria. In some embodiments, proteinuria comprises a urinary protein-creatinine ratio (UPCR) of greater than 0.2 mg/mg. In some embodiments, proteinuria comprises a urinary protein excretion of greater than 4 mg/m² per hour. In some embodiments, complete remission (CR) of a renal disease or condition is defined as a consistent UPCR measurement of less than 0.2 g protein/g creatinine. In some embodiments, a partial remission (PR) of a renal disease or condition is defined as having about a 50% reduction from baseline proteinuria and a consistent UPCR of less than about 2 g protein/g creatinine.

In some embodiments, the disclosure relates to methods of reducing an UPCR in a subject with a renal disease or condition, comprising administering to a subject in need thereof an effective amount of a variant ActRIIB polypeptide (e.g., a variant ActRIIB homomultimer protein or a variant ActRIIB heteromultimer protein). In some embodiments, the method relates to reducing the subject’s UPCR by between about 0.2 and about 1 mg/mg. In some embodiments, the method relates to reducing the subject’s UPCR by less than 0.5 mg/mg. In some embodiments, the method relates to reducing the subject’s UPCR by between about by between about 0.1 and about 100.0 mg urinary protein/mg creatinine. In some embodiments, the method relates to reducing the subject’s UPCR by between about 0.1 and about 2.5 mg urinary protein/mg creatinine. In some embodiments, the method relates to reducing the subject’s UPCR by between about 2.5 and about 3.5 mg urinary protein/mg creatinine. In some embodiments, the method relates to reducing the subject’s UPCR by between about 3.5 and about 5.0 mg urinary protein/mg creatinine. In some embodiments, the method relates to reducing the subject’s UPCR by between about 5.0 and about 7.5 mg urinary protein/mg creatinine. In some embodiments, the method relates to reducing the subject’s UPCR by between about 7.5 and about 10.0 mg urinary protein/mg creatinine. In some embodiments, the method relates to reducing the subject’s UPCR by between about 10.0 and about 15.0 mg urinary protein/mg creatinine. In some embodiments, the method relates to reducing the subject’s UPCR by between about 15.0 and about 20.0 mg urinary protein/mg creatinine. In some embodiments, the method relates to reducing the subject’s UPCR by between about 20.0 and about 25.0 mg urinary protein/mg creatinine. In some embodiments, the method relates to reducing the subject’s UPCR by between about 30.0 and about 35.0 mg urinary protein/mg creatinine. In some embodiments, the method relates to reducing the subject’s UPCR by between about 40.0 and about 45.0 mg urinary protein/mg creatinine. In some embodiments, the method relates to reducing the subject’s UPCR by between about 45.0 and about 50.0 mg urinary protein/mg creatinine. In some embodiments, the method relates to reducing the subject’s UPCR by between about 50.0 and about 60.0 mg urinary protein/mg creatinine. In some embodiments, the method relates to reducing the subject’s UPCR by between about 60.0 and about 70.0 mg urinary protein/mg creatinine. In some embodiments, the method relates to reducing the subject’s UPCR by between about 70.0 and about 80.0 mg urinary protein/mg creatinine. In some embodiments, the method relates to reducing the subject’s UPCR by between about 80.0 and about 90.0 mg urinary protein/mg creatinine. In some embodiments, the method relates to reducing the subject’s UPCR by between about 90.0 and about 100.0 mg urinary protein/mg creatinine.

In some embodiments, administration of therapy decreases urinary protein excretion. In some embodiments, the method relates to reducing the subject’s UPCR by at least 2.5% compared to a baseline measurement. In some embodiments, the method relates to reducing the subject’s UPCR by at least 5% compared to a baseline measurement. In some embodiments, the method relates to reducing the subject’s UPCR by at least 10% compared to a baseline measurement. In some embodiments, the method relates to reducing the subject’s UPCR by at least 15% compared to a baseline measurement. In some embodiments, the method relates to reducing the subject’s UPCR by at least 20% compared to a baseline measurement. In some embodiments, the method relates to reducing the subject’s UPCR by at least 25% compared to a baseline measurement. In some embodiments, the method relates to reducing the subject’s UPCR by at least 30% compared to a baseline measurement. In some embodiments, the method relates to reducing the subject’s UPCR by at least 40% compared to a baseline measurement. In some embodiments, the method relates to reducing the subject’s UPCR by at least 50% compared to a baseline measurement. In some embodiments, the method relates to reducing the subject’s UPCR by at least 60% compared to a baseline measurement. In some embodiments, the method relates to reducing the subject’s UPCR by at least 70% compared to a baseline measurement. In some embodiments, the method relates to reducing the subject’s UPCR by at least 80% compared to a baseline measurement. In some embodiments, the method relates to reducing the subject’s UPCR by at least 90% compared to a baseline measurement. In some embodiments, the method relates to reducing the subject’s UPCR by at least 95% compared to a baseline measurement. In some embodiments, the method relates to reducing the subject’s UPCR by at least 99% compared to a baseline measurement.

A subject may be administered a blood test to determine presence of a kidney disease or condition (e.g., Alport syndrome, focal segmental glomerulosclerosis (FSGS), polycystic kidney disease, chronic kidney disease), by determining how well the kidney is filtering the blood. Typically, a glomerular filtration rate (GFR) is determined, which measures the flow rate of filtered fluid (e.g., blood) through the kidney into the Bowman’s capsule. GFR is equal to the clearance rate of when any solute is freely filtered and is neither reabsorbed nor secreted by the kidneys. GFR is therefore a measurement of the quantity of the substance in the urine that originated from a calculable volume of blood, and is typically recorded in units of volume per time, e.g., milliliters per minute (mL/min). A normal range of GFR, adjusted for body surface area, is between about 100 and about 130 mL/min/1.73 m² in men, with an average GFR of 125 mL/min/1.73 m² in men. A normal range of GFR, adjusted for body surface area, is between about 90 and about 120 mL/min/1.73 m² in women younger than age 40. GFR measured by inulin clearance in children under 2 years old is about 110 mL/min/1.73 m², which progressively decreases. After age 40, GFR decreases progressively with age, by between about 0.4 and about 1.2 mL/min per year. GFR may also be calculated by comparative measurements of substances in the blood and urine, estimated using a blood test result (e.g., eGFR). In some embodiments, eGFR is measured using serum creatinine, age, ethnicity, and gender variables. In some embodiments, eGFR is measured using one or more of Cockcroft-Gault formula, Modification of Diet in Renal Disease (MDRD) formula, CKD-EPI formula, Mayo quadratic formula, and Schwartz formula.

A glomerular filtration rate (GFR) ≥ 60 ml/min/1.73 m2 is considered normal in a subject without chronic kidney disease if there is no kidney damage present, which comprises signs of damage seen in blood, urine, or imaging studies which includes lab albumin/creatinine ratio (ACR) ≥ 30. Subjects with a GFR <60 ml/min/1.73 m2 for at least 3 months are diagnosed as having chronic kidney disease.

In general, protein in the urine is regarded as an independent marker for decline of kidney function and cardiovascular disease, and the stages of chronic kidney disease (often used for renal diseases and/or conditions in general) is determined by measuring a subject’s GFR. In some embodiments, the present disclosure provides methods of treating stage 1 CKD. Stage 1 CKD comprises normal kidney function, kidney damage with normal or relatively high GFR (e.g., ≥90 ml/min/1.73 m²), and lower creatinine levels. Kidney damage may be defined as pathological abnormalities or markers of damage, including abnormalities in blood or urine tests or imaging studies. In some embodiments, the present disclosure provides methods of treating stage 2 CKD. Stage 2 CKD comprises mild reduction in kidney function and GFR (e.g., between about 60 and about 89 ml/min/1.73 m²) with kidney damage. In some embodiments, the present disclosure provides methods of treating stage 3 CKD. Stage 3 CKD comprises mild to moderate reduction in kidney function and GFR (e.g., between about 30 and about 59 ml/min/1.73 m²). Stage 3 CKD may be split into stages 3a (e.g., mild to moderate reduction in kidney function and GFR between about 45 and about 59 ml/min/1.73 m² and 3b (e.g., moderate to severe reduction in kidney function and GFR between about 30 and about 44 ml/min/1.73 m². In some embodiments, the present disclosure provides methods of treating stage 3a CKD. In some embodiments, the present disclosure provides methods of treating stage 3b CKD. In some embodiments, the present disclosure provides methods of treating stage 4 CKD. Stage 4 CKD comprises severe reduction in kidney function and GFR (e.g., between about 15 and about 29 ml/min/1.73 m²). In some embodiments, the present disclosure provides methods of treating stage 5 CKD. Stage 5 CKD comprises established kidney failure (e.g., GFR about <15 ml/min/1.73 m²), permanent kidney replacement therapy, end-stage renal disease. (ESRD), and high creatinine levels. In some embodiments, the present disclosure relates to methods of reducing severity, occurrence and/or duration of stage 1 CKD. In some embodiments, the present disclosure relates to methods of reducing severity, occurrence and/or duration of stage 2 CKD. In some embodiments, the present disclosure relates to methods of reducing severity, occurrence and/or duration of stage 3 CKD. In some embodiments, the present disclosure relates to methods of reducing severity, occurrence and/or duration of stage 3a CKD. In some embodiments, the present disclosure relates to methods of reducing severity, occurrence and/or duration of stage 3b CKD. In some embodiments, the present disclosure relates to methods of reducing severity, occurrence and/or duration of stage 4 CKD. In some embodiments, the present disclosure relates to methods of reducing severity, occurrence and/or duration of stage 5 CKD. In some embodiments, the present disclosure provides methods of treating a subject with Stage 1 CKD that delay or prevent progression to Stage 2 CKD. In some embodiments, the present disclosure provides methods of treating a subject with Stage 2 CKD that delay or prevent progression to Stage 3 CKD. In some embodiments, the present disclosure provides methods of treating a subject with Stage 2 CKD that delay or prevent progression to Stage 3a CKD. In some embodiments, the present disclosure provides methods of treating a subject with Stage 2 CKD that delay or prevent progression to Stage 3b CKD. In some embodiments, the present disclosure provides methods of treating a subject with Stage 3a CKD that delay or prevent progression to Stage 3b CKD. In some embodiments, the present disclosure provides methods of treating a subject with Stage 3 CKD that delay or prevent progression to Stage 4 CKD. In some embodiments, the present disclosure provides methods of treating a subject with Stage 3a CKD that delay or prevent progression to Stage 4 CKD. In some embodiments, the present disclosure provides methods of treating a subject with Stage 3b CKD that delay or prevent progression to Stage 4 CKD. In some embodiments, the present disclosure provides methods of treating a subject with Stage 4 CKD that delay or prevent progression to Stage 5 CKD. In some embodiments, the present disclosure provides methods of delaying and/or preventing worsening of CKD stage progression in a subject in need thereof. In some embodiments, the present disclosure provides an improvement in renal damage and/or a downgrade in CKD stage classification in a subject in need thereof. In some embodiments, the present disclosure provides methods of improving CKD classification in a subject by one or more stages.

In some embodiments, the disclosure relates to methods of increasing GFR and/or eGFR in a subject with a renal disease or condition, comprising administering to a subject in need thereof an effective amount of a variant ActRIIB polypeptide (e.g., a variant ActRIIB homomultimer protein or a variant ActRIIB heteromultimer protein). In some embodiments, the method relates to increasing the subject’s GFR and/or eGFR by at least 2.5% compared to a baseline measurement. In some embodiments, the method relates to increasing the subject’s GFR and/or eGFR by at least 5% compared to a baseline measurement. In some embodiments, the method relates to increasing the subject’s GFR and/or eGFR by at least 10% compared to a baseline measurement. In some embodiments, the method relates to increasing the subject’s GFR and/or eGFR by at least 15% compared to a baseline measurement. In some embodiments, the method relates to increasing the subject’s GFR and/or eGFR by at least 20% compared to a baseline measurement. In some embodiments, the method relates to increasing the subject’s GFR and/or eGFR by at least 25% compared to a baseline measurement. In some embodiments, the method relates to increasing the subject’s GFR and/or eGFR by at least 30% compared to a baseline measurement. In some embodiments, the method relates to increasing the subject’s GFR and/or eGFR by at least 40% compared to a baseline measurement. In some embodiments, the method relates to increasing the subject’s GFR and/or eGFR by at least 50% compared to a baseline measurement. In some embodiments, the method relates to increasing the subject’s GFR and/or eGFR by at least 60% compared to a baseline measurement. In some embodiments, the method relates to increasing the subject’s GFR and/or eGFR by at least 70% compared to a baseline measurement. In some embodiments, the method relates to increasing the subject’s GFR and/or eGFR by at least 80% compared to a baseline measurement. In some embodiments, the method relates to increasing the subject’s GFR and/or eGFR by at least 90% compared to a baseline measurement. In some embodiments, the method relates to increasing the subject’s GFR and/or eGFR by at least 95% compared to a baseline measurement. In some embodiments, the method relates to increasing the subject’s GFR and/or eGFR by at least 99% compared to a baseline measurement.

In some embodiments, the method relates to increasing the subject’s GFR by about 1 mL/min/1.73 m² compared to a baseline measurement. In some embodiments, the method relates to increasing the subject’s GFR by about 3 mL/min/1.73 m² compared to a baseline measurement. In some embodiments, the method relates to increasing the subject’s GFR by about 5 mL/min/1.73 m² compared to a baseline measurement. In some embodiments, the method relates to increasing the subject’s GFR by about 7 mL/min/1.73 m² compared to a baseline measurement. In some embodiments, the method relates to increasing the subject’s GFR by about 9 mL/min/1.73 m² compared to a baseline measurement. In some embodiments, the method relates to increasing the subject’s GFR by about 10 mL/min/1.73 m² compared to a baseline measurement. In some embodiments, the method relates to increasing the subject’s GFR by about 15 mL/min/1.73 m² compared to a baseline measurement. In some embodiments, the method relates to increasing the subject’s GFR by about 20 mL/min/1.73 m² compared to a baseline measurement. In some embodiments, the method relates to increasing the subject’s GFR by about 25 mL/min/1.73 m² compared to a baseline measurement. In some embodiments, the method relates to increasing the subject’s GFR by about 30 mL/min/1.73 m² compared to a baseline measurement. In some embodiments, the method relates to increasing the subject’s GFR by about 35 mL/min/1.73 m² compared to a baseline measurement. In some embodiments, the method relates to increasing the subject’s GFR by about 40 mL/min/1.73 m² compared to a baseline measurement. In some embodiments, the method relates to increasing the subject’s GFR by about 45 mL/min/1.73 m² compared to a baseline measurement. In some embodiments, the method relates to increasing the subject’s GFR by about 50 mL/min/1.73 m² compared to a baseline measurement. In some embodiments, the method relates to increasing the subject’s GFR by about 55 mL/min/1.73 m² compared to a baseline measurement. In some embodiments, the method relates to increasing the subject’s GFR by about 60 mL/min/1.73 m² compared to a baseline measurement. In some embodiments, the method relates to increasing the subject’s GFR by about 65 mL/min/1.73 m² compared to a baseline measurement. In some embodiments, the method relates to increasing the subject’s GFR by about 70 mL/min/1.73 m² compared to a baseline measurement. In some embodiments, the method relates to increasing the subject’s GFR by about 75 mL/min/1.73 m² compared to a baseline measurement. In some embodiments, the method relates to increasing the subject’s GFR by about 80 mL/min/1.73 m² compared to a baseline measurement. In some embodiments, the method relates to increasing the subject’s GFR by about 85 mL/min/1.73 m² compared to a baseline measurement. In some embodiments, the method relates to increasing the subject’s GFR by about 90 mL/min/1.73 m² compared to a baseline measurement. In some embodiments, the method relates to increasing the subject’s GFR by about 95 mL/min/1.73 m² compared to a baseline measurement. In some embodiments, the method relates to increasing the subject’s GFR by about 100 mL/min/1.73 m² compared to a baseline measurement.

In some embodiments, GFR and/or eGFR can be determined by injecting inulin or the inulin-analog sinistrin into plasma. Since both inulin and sinistrin are neither reabsorbed nor secreted by the kidney after glomerular filtration, their rate of excretion is directly proportional to the rate of filtration of water and solutes across the glomerular filter. In some embodiments, GFR and/or eGFR is measured using radioactive substances. In some embodiments, GFR and/or eGFR is measured using chromium-51. In some embodiments, GFR and/or eGFR is measured using renal or plasma clearance of 51Cr-EDTA. In some embodiments, GFR and/or eGFR is measured using technetium-99m. In some embodiments, GFR and/or eGFR is measured using 99mTc-DTPA. A benefit of using radioactive substances is they come close to the ideal properties of inulin (undergoing only glomerular filtration) but can be measured more practically with only a few urine or blood samples. Renal and plasma clearance 51Cr-EDTA has been shown to be accurate in comparison with inulin. In some embodiments, inulin clearance slightly overestimates glomerular function. In early stage renal disease, inulin clearance may remain normal due to hyperfiltration in the remaining nephrons. Incomplete urine collection is an important source of error in inulin clearance measurement.

Creatinine clearance rate (CCr or CrCl) is the volume of blood plasma that is cleared of creatinine per unit time and is a useful measure for approximating the GFR. Creatinine clearance exceeds GFR due to creatinine secretion, which can be blocked by cimetidine. Both GFR and CCr may be accurately calculated by comparative measurements of substances in the blood and urine, or estimated by formulas using just a blood test result (eGFR and eCCr).

In some embodiments, the disclosure relates to methods of reducing total kidney volume in subject with a renal disease or condition, comprising administering to a subject in need thereof an effective amount of a variant ActRIIB polypeptide (e.g., a variant ActRIIB homomultimer protein or a variant ActRIIB heteromultimer protein). In some embodiments, total kidney volume is measured by ultrasound. In some embodiments, total kidney volume is measured by magnetic resonance imaging (MRI). In some embodiments, total kidney volume reflects a sum volume of the kidney and cysts in renal diseases or disorders (e.g., ADPKD). In some embodiments, the method relates to reducing total kidney volume in the subject by at least 2.5% compared to a baseline measurement. In some embodiments, the method relates to reducing total kidney volume in the subject by at least 5% compared to a baseline measurement. In some embodiments, the method relates to reducing total kidney volume in the subject by at least 10% compared to a baseline measurement. In some embodiments, the method relates to reducing total kidney volume in the subject by at least 15% compared to a baseline measurement. In some embodiments, the method relates to reducing total kidney volume in the subject by at least 20% compared to a baseline measurement. In some embodiments, the method relates to reducing total kidney volume in the subject by at least 25% compared to a baseline measurement. In some embodiments, the method relates to reducing total kidney volume in the subject by at least 30% compared to a baseline measurement. In some embodiments, the method relates to reducing total kidney volume in the subject by at least 40% compared to a baseline measurement. In some embodiments, the method relates to reducing total kidney volume in the subject by at least 50% compared to a baseline measurement. In some embodiments, the method relates to reducing total kidney volume in the subject by at least 60% compared to a baseline measurement. In some embodiments, the method relates to reducing total kidney volume in the subject by at least 70% compared to a baseline measurement. In some embodiments, the method relates to reducing total kidney volume in the subject by at least 80% compared to a baseline measurement. In some embodiments, the method relates to reducing total kidney volume in the subject by at least 90% compared to a baseline measurement. In some embodiments, the method relates to reducing total kidney volume in the subject by at least 95% compared to a baseline measurement. In some embodiments, the method relates to reducing total kidney volume in the subject by at least 99% compared to a baseline measurement.

In some embodiments, blood urea nitrogen (BUN) is measured. In some embodiments, a BUN test measures the amount of urea nitrogen in blood. In some embodiments, if kidneys are impaired, the amount of urea nitrogen can be higher. In some embodiments, the disclosure relates to methods of reducing BUN in a subject with a renal disease or condition, comprising administering to a subject in need thereof an effective amount of a variant ActRIIB polypeptide (e.g., a variant ActRIIB homomultimer protein or a variant ActRIIB heteromultimer protein). In some embodiments, a normal BUN level for a human is between about 7 mg/dL and about 20 mg/dL. In some embodiments, the method relates to reducing BUN in the subject by at least 2.5% compared to a baseline measurement. In some embodiments, the method relates to reducing BUN in the subject by at least 5% compared to a baseline measurement. In some embodiments, the method relates to reducing BUN in the subject by at least 10% compared to a baseline measurement. In some embodiments, the method relates to reducing BUN in the subject by at least 15% compared to a baseline measurement. In some embodiments, the method relates to reducing BUN in the subject by at least 20% compared to a baseline measurement. In some embodiments, the method relates to reducing BUN in the subject by at least 25% compared to a baseline measurement. In some embodiments, the method relates to reducing BUN in the subject by at least 30% compared to a baseline measurement. In some embodiments, the method relates to reducing BUN in the subject by at least 40% compared to a baseline measurement. In some embodiments, the method relates to reducing BUN in the subject by at least 50% compared to a baseline measurement. In some embodiments, the method relates to reducing BUN in the subject by at least 60% compared to a baseline measurement. In some embodiments, the method relates to reducing BUN in the subject by at least 70% compared to a baseline measurement. In some embodiments, the method relates to reducing BUN in the subject by at least 80% compared to a baseline measurement. In some embodiments, the method relates to reducing BUN in the subject by at least 90% compared to a baseline measurement. In some embodiments, the method relates to reducing BUN in the subject by at least 95% compared to a baseline measurement. In some embodiments, the method relates to reducing BUN in the subject by at least 99% compared to a baseline measurement.

Optionally, methods disclosed herein for treating, preventing, or reducing the progression rate and/or severity of a renal disease or condition (e.g., Alport syndrome, focal segmental glomerulosclerosis (FSGS), polycystic kidney disease, chronic kidney disease), particularly treating, preventing, or reducing the progression rate and/or severity of one or more complications of a renal disease or condition, may further comprise administering to the subject one or more additional active agents and/or supportive therapies for treating a renal disease or condition. In some embodiments, a subject is administered an additional active agent and/or supportive therapy for treating a renal disease or condition (e.g., Alport syndrome, focal segmental glomerulosclerosis (FSGS), polycystic kidney disease, chronic kidney disease). In some embodiments, ARBs and ACE inhibitors are mainstays of therapy for renal diseases and conditions (e.g., Alport syndrome, focal segmental glomerulosclerosis (FSGS), polycystic kidney disease, chronic kidney disease), with beta-blockade and calcium-channel blockers as second-line therapy. In some embodiments, as third-line therapy, thiazides are preferred in subjects with normal renal function, while loop diuretics are preferred in subjects with impaired renal function.

In some embodiments, a subject with a renal disease or condition (e.g., Alport syndrome, focal segmental glomerulosclerosis (FSGS), polycystic kidney disease, chronic kidney disease) is administered an antagonist of the Renin-angiotensin-aldosterone system (RAAS). In some embodiments, RAAS inhibitors include, but are not limited to, angiotensin antagonists (e.g., angiotensin blockade therapy, angiotensin system inhibitor, renin-angiotensin system inhibitor, angiotensin II blockade, angiotensin II type 1 receptor blocker, ARB, angiotensin II receptor antagonist, AT₁ receptor antagonist, or a sartan) and an angiotensin-converting enzyme (ACE) inhibitor. In some embodiments, RAAS antagonism and particularly, the combination of an ACE inhibitor and ARB, will lower GFR by reducing efferent arteriolar vascular tone and thus, reducing intraglomerular capillary pressure, the driving force for glomerular filtration. Thus, a modest decrease in GFR may be tolerated, providing evidence that RAAS antagonism has been achieved.

In some embodiments, a subject is administered an angiotensin antagonist (e.g., angiotensin receptor blocker, ARB), when the subject shows signs of proteinuria. In some embodiments, an ARB reduces proteinuria in subjects with a renal disease or condition. In some embodiments, an angiotensin antagonist diminishes the rate of glomerulosclerosis in subjects with a renal disease or condition. In some embodiments, administration of an ARB decreases renal disease progression. In some embodiments, a subject is administered one or more ARBs selected from the group consisting of losartan, irbesartan, olmesartan, candesartan, valsartan, fimasartan, azilsartan, salprisartan, and telmisartan. In some embodiments a subject is administered losartan. In some embodiments, a subject is administered irbesartan. In some embodiments, a subject is administered olmesartan. In some embodiments, a subject is administered candesartan. In some embodiments, a subject is administered valsartan. In some embodiments, a subject is administered fimasartan. In some embodiments, a subject is administered azilsartan. In some embodiments, a subject is administered salprisartan. In some embodiments, a subject is administered telmisartan.

In some embodiments, a subject with a renal disease or condition is administered an ACE inhibitor. In some embodiments, an ACE inhibitor is selected from the group consisting of benazepril, captopril, enalapril, lisinopril, perindopril, ramipril (e.g., ramipen), trandolapril, and zofenopril. In some embodiments, a subject is administered benazepril. In some embodiments, a subject is administered captopril. In some embodiments, a subject is administered enalapril. In some embodiments, a subject is administered lisinopril. In some embodiments, a subject is administered perindopril. In some embodiments, a subject is administered ramipril. In some embodiments, a subject is administered trandolapril. In some embodiments, a subject is administered zofenopril. In some embodiments, administration of an ACE inhibitor delays dialysis in a subject with proteinuria and normal kidney function. In some embodiments, administration of an ACE inhibitor slows decline in renal function in a subject. In some embodiments, administration of an ACE inhibitor reduces proteinuria in a subject. In some embodiments, administration of an ACE inhibitor decreases kidney damage in a subject.

In some embodiments, a subject with a renal disease or condition is administered an ARB and an ACE inhibitor. In some embodiments, a subject with a renal disease or condition comprising proteinuria and/or microalbuminuria is administered an ARB and an ACE inhibitor.

In some embodiments, an alternative approach to angiotensin antagonism is to combine an ACE inhibitor and/or ARB with an aldosterone antagonist.

In some embodiments, a subject with a renal disease or condition (e.g., primary FSGS) is administered an immunosuppressive treatment. In some embodiments, subjects with a renal disease or condition are treated with immunosuppressive medications. In some embodiments, immunosuppression is not administered to subjects with secondary FSGS. In some embodiments, immunosuppressants are not administered to subjects that do not have primary FSGS. In some embodiments, an immunosuppressant is selected from the group consisting of corticosteroids, calcineurin inhibitors, janus kinase inhibitors, mammalian target of rapamycin (mTOR) inhibitors, IMDH inhibitors, and biologics (including, but not limited to monoclonal antibodies).

In some embodiments, a subject with a renal disease or condition is administered a corticosteroid. In some embodiments, a glucocorticoid is a corticosteroid. In some embodiments, a subject with a renal disease or condition is administered one or more glucocorticoids. In some embodiments, administration of a glucocorticoid is an initial therapy. In some embodiments, a glucocorticoid is selected from the group consisting of beclomethasone, betamethasone, budesonide, cortisone, dexamethasone, hydrocortisone, methylprednisolone, prednisolone, methylprednisone, prednisone, and triamcinolone. In some embodiments, a subject with a renal disease or condition is administered prednisone. In some embodiments, a subject with a renal disease or condition is administered prednisolone.

In some embodiments, a calcineurin inhibitor is selected from the group consisting of cyclosporine (e.g., cyclosporin, ciclosporin, ciclosporine, Neoral, Sandimmune, SangCya) and tacrolimus (e.g., Astagraf XL, Envarsus XR, Prograf). In some embodiments, calcineurin inhibitors are administered to steroid-sensitive subjects who cannot tolerate continued steroid therapy, and/or to subjects with steroid-resistant renal disease (e.g. steroid-resistant FSGS). In some embodiments, a subject with a renal disease or condition is administered cyclosporine. In some embodiments, a subject with a renal disease or condition is administered tacrolimus.

In some embodiments, a subject with a renal disease or condition maybe administered a combination of one or more corticosteroids and/or calcineurin inhibitors. In some embodiments, a subject with a kidney disease or condition may be administered cyclosporine and prednisone. In some embodiments, a subject with a renal disease or condition is administered tacrolimus and prednisone. In some embodiments, cyclosporine and prednisone are administered to preserve renal function assessed as creatinine clearance.

In some embodiments, treatment with mycophenolate mofetil (MMF) combined with glucocorticoids may be beneficial in subjects who cannot take calcineurin inhibitors. In some embodiments, a subject with a renal disease or condition is administered mycophenolate mofetil (MMF) in combination with one or more glucocorticoids. In some embodiments, a subject with a renal disease or condition is administered MMF and prednisone. In some embodiments, a subject with a renal disease or condition is administered prednisolone and MMF.

In some embodiments, a subject with a renal disease or condition is administered cyclophosphamide and/or prednisone. In some embodiments, a subject with a renal disease or condition is administered prednisolone and/or chlorambucil. In some embodiments, a subject with a renal disease or condition is administered cyclophosphamide. In some embodiments, a subject with a renal disease or condition is administered chlorambucil.

In some embodiments, a janus kinase inhibitor is tofacitinib (e.g., Xeljanz).

In some embodiments, an mTOR inhibitor is selected from the group consisting of sirolimus (e.g., Rapamune) and everolimus (e.g., Afinitor, Zortress).

In some embodiments, an IMDH inhibitor is selected from the group consisting of azathioprine (e.g., Azasan, Imuran), leflunomide (e.g., Arava), and mycophenolate (e.g., CellCept, Myfortic).

In some embodiments, a biologic is selected from the group consisting of abatacept (e.g., Orencia), adalimumab (e.g., Humira), anakinra (e.g., Kineret), basiliximab (e.g., Simulect), certolizumab (e.g., Cimzia), daclizumab (e.g., Zinbryta), etanercept (e.g., Enbrel), fresolimumab, golimumab (e.g., Simponi), infliximab (e.g., Remicade), ixekizumab (e.g., Taltz), natalizumab (e.g., Tysabri), rituximab (e.g., Rituxan), secukinumab (e.g., Cosentyx), tocilizumab (e.g., Actemra), ustekinumab (e.g., Stelara), and vedolizumab (e.g., Entyvio).

In some embodiments, a subject with a renal disease or condition (e.g., Alport syndrome, focal segmental glomerulosclerosis (FSGS), polycystic kidney disease, chronic kidney disease) is administered a statin (e.g., benazepril, valsartan, Fluvastatin, pravastatin).

In some embodiments, a subject with a renal disease or condition (e.g., Alport syndrome, focal segmental glomerulosclerosis (FSGS), polycystic kidney disease, chronic kidney disease) is administered bardoxolone methyl. Bardoxolone methyl is an activator of the KEAP1-Nrf2 pathway and bardoxolone methyl also inhibits the pro-inflammatory transcription factor NF-xB.

In some embodiments, a subject with a renal disease or condition (e.g., Alport syndrome, focal segmental glomerulosclerosis (FSGS), polycystic kidney disease, chronic kidney disease) is administered Achtar gel. Achtar gel was approved in the 1950s by the US Food and Drug Administration for nephrotic syndrome under criteria that were less stringent than required today. In some embodiments, some case studies suggest limited efficacy of Acthar in some subjects with FSGS. In some embodiments, a subject with FSGS is administered Achtar gel.

In some embodiments, a subject with ADPKD is administered Tolvaptan (e.g., OPC-41061). In some embodiments, Tolvaptan has demonstrated a slower decline than placebo in the eGFR over a one year period in patients with late-stage chronic kidney disease but is associated with elevations of bilirubin and alanine aminotransferase levels.

In some embodiments, a subject a renal disease or condition (e.g., Alport syndrome, focal segmental glomerulosclerosis (FSGS), polycystic kidney disease, chronic kidney disease) is administered one or more of abatacept in combination with sparsentan, aliskiren, allopurinol, ANG-3070, atorvastatin, bleselumab, bosutinib, CCX140-B, CXA-10, D6-25-hydroxyvitamin D3, dapagliflozin, dexamethasone in combination with MMF, emodin, FG-3019, FK506, FK-506 and MMF, FT-011, galactose, GC1008, GFB-887, isotretinoin, lademirsen, lanreotide, levamisole, lixivaptan, losmapimod, metformin, mizorbine, N-acetylmannosamine, octreotide, paricalcitol, PF-06730512, pioglitazone, propagermanium, propagermanium and irbesartan, rapamune, rapamycin, RE-021 (e.g., sparsentan), RG012, rosiglitazone (e.g., Avandia), saquinivir, SAR339375, somatostatin, spironolactone, tesevatinib (KD019), tetracosactin, tripterygium wilfordii (TW), valproic acid, VAR-200, venglustat (GZ402671), verinurad, voclosporin, or VX-147.

In some embodiments, a subject with a renal disease or condition (e.g., Alport syndrome, focal segmental glomerulosclerosis (FSGS), polycystic kidney disease, chronic kidney disease) undergoes kidney dialysis. In some embodiments, a subject with a renal disease or condition (e.g., Alport syndrome, focal segmental glomerulosclerosis (FSGS), polycystic kidney disease, chronic kidney disease) undergoes a kidney transplant. In some embodiments, a subject with ESRD undergoes a kidney transplantation. In some embodiments, a subject with a kidney transplant does not experience recurrent renal disease. In some embodiments, a subject with a kidney transplant contracts anti-glomerular basement membrane antibody disease. In some embodiments, anti-glomerular basement membrane antibody disease occurs within one year after kidney transplantation. In some embodiments, a subject with anti-glomerular basement membrane antibody disease is administered methylprednisone and/or cyclophosphamide. In some embodiments, a subject with anti-glomerular basement membrane antibody disease undergoes plasmapheresis.

In some embodiments, a subject with a renal disease or condition (e.g., Alport syndrome, focal segmental glomerulosclerosis (FSGS), polycystic kidney disease, chronic kidney disease) is administered mesenchymal stem cell therapy. In some embodiments, a subject with a renal disease or condition (e.g., Alport syndrome, focal segmental glomerulosclerosis (FSGS), polycystic kidney disease, chronic kidney disease) is administered bone marrow stem cells. In some embodiments, a subject with a renal disease or condition (e.g., Alport syndrome, focal segmental glomerulosclerosis (FSGS), polycystic kidney disease, chronic kidney disease) undergoes lipoprotein removal. In some embodiments, a subject with a renal disease or condition (e.g., Alport syndrome, focal segmental glomerulosclerosis (FSGS), polycystic kidney disease, chronic kidney disease) is administered a Liposorber LA-15 device. In some embodiments, a subject with a renal disease or condition (e.g., Alport syndrome, focal segmental glomerulosclerosis (FSGS), polycystic kidney disease, chronic kidney disease) undergoes plasmapheresis. In some embodiments, a subject with a renal disease or condition (e.g., Alport syndrome, focal segmental glomerulosclerosis (FSGS), polycystic kidney disease, chronic kidney disease) undergoes plasma exchange. In some embodiments, a subject with a renal disease or condition (e.g., Alport syndrome, focal segmental glomerulosclerosis (FSGS), polycystic kidney disease, chronic kidney disease) undergoes a change in diet (e.g., dietary sodium intake).

In some embodiments, methods of the present disclosure delay clinical worsening of a renal disease or condition (e.g., Alport syndrome, focal segmental glomerulosclerosis (FSGS), polycystic kidney disease, chronic kidney disease) in a subject. In some embodiments, methods of the present disclosure reduce the risk of hospitalization for one or more complications associated with a renal disease or condition (e.g., Alport syndrome, focal segmental glomerulosclerosis (FSGS), polycystic kidney disease, chronic kidney disease).

### 7. Pharmaceutical Compositions

In certain embodiments, compounds of the present disclosure (e.g., variant ActRIIB proteins in either homomeric or heteromeric forms) are formulated with a pharmaceutically acceptable carrier. For example, a variant ActRIIB protein can be administered alone or as a component of a pharmaceutical formulation (therapeutic composition). The subject compounds may be formulated for administration in any convenient way for use in human or veterinary medicine.

In certain embodiments, the disclosure relates to pharmaceutical preparations comprising an ActRIIB polypeptide, including variant ActRIIB polypeptides as well as homomultimer and heteromultimers comprising the same, and a pharmaceutically acceptable carrier. In some embodiments, pharmaceutical preparations comprising one or more variant ActRIIB heteromulitmers comprises less than about 10%, 9%, 8%, 7%, 6%, 5%, 4%, 3%, 2%, or less than about 1% homomultimers.

In certain embodiments, the therapeutic method described herein includes administering the composition topically, systemically, or locally as an implant or device. When administered, the therapeutic composition for use in this disclosure is, of course, in a pyrogen-free, physiologically acceptable form. Further, the composition may desirably be encapsulated or injected in a viscous form for delivery to a target tissue site (e.g., renal, pulmonary, cardiac, bone, cartilage, muscle, fat or neurons), for example, a site having a tissue damage. Topical administration may be suitable for wound healing and tissue repair. Therapeutically useful agents other than the variant ActRIIB proteins which may also optionally be included in the composition as described above, may alternatively or additionally, be administered simultaneously or sequentially with the subject compounds (e.g., variant ActRIIB proteins in either homomeric or heteromeric forms) in the methods described herein.

In certain embodiments, compositions of the present disclosure may include a matrix capable of delivering one or more therapeutic compounds (e.g., variant ActRIIB proteins in either homomeric or heteromeric forms) to a target tissue site, providing a structure for the developing tissue and optimally capable of being resorbed into the body. For example, the matrix may provide slow release of the variant ActRIIB proteins. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalciumphosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

In certain embodiments, variant ActRIIB proteins described herein (e.g., variant ActRIIB proteins in either homomeric or heteromeric forms) can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more variant ActRIIB proteins of the present disclosure (e.g., variant ActRIIB proteins in either homomeric or heteromeric forms) may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Certain compositions disclosed herein may be administered topically, either to skin or to mucosal membranes. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, and patches. The active compound may be mixed under sterile conditions with a pharmaceutically acceptable carrier, and with any preservatives, buffers, or propellants which may be required. The ointments, pastes, creams and gels may contain, in addition to a subject compound described herein (e.g., variant ActRIIB proteins in either homomeric or heteromeric forms), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a subject compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

In certain embodiments, pharmaceutical compositions suitable for parenteral administration may comprise one or more variant ActRIIB proteins in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In some embodiments, a parenteral route of administration is selected from the group consisting of intramuscular, intraperitoneal, intradermal, intravitreal, epidural, intracerebral, intra-arterial, intraarticular, intra-cavernous, intra-lesional, intraosseous, intraocular, intrathecal, intravenous, transdermal, trans-mucosal, extra-amniotic administration, subcutaneous, and combinations thereof. In some embodiments, a parenteral route of administration is subcutaneous. In some embodiments, a parenteral route of administration is a subcutaneous injection. In some embodiments, compositions of the present disclosure are administered by subcutaneous injection.

The compositions described herein may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds described herein (e.g., variant ActRIIB proteins in either homomeric or heteromeric forms). The various factors will depend upon the disease to be treated. In the case of muscle disorders, factors may include, but are not limited to, amount of muscle mass desired to be formed, the muscles most affected by disease, the condition of the deteriorated muscle, the patient’s age, sex, and diet, time of administration, and other clinical factors. The addition of other known growth factors to the final composition, may also affect the dosage. Progress can be monitored by periodic assessment of muscle growth and/or repair, for example, by strength testing, MRI assessment of muscle size and analysis of muscle biopsies.

In certain embodiments, one or more variant ActRIIB proteins (e.g., variant ActRIIB proteins in either homomeric or heteromeric forms) can be administered, in combination (same formulation or in separate formulations), concomitantly, sequentially, or on different schedules. In addition, variant ActRIIB proteins can be administered with another type of therapeutic agents, for example, a cartilage-inducing agent, a bone-inducing agent, a muscle-inducing agent, a fat-reducing, a neuron-inducing agent, an agent for pulmonary disease (e.g., sildenafil), an agent for renal disease, or an agent for cardiac disease. The two types of compounds may be administered simultaneously or at different times. It is expected that the variant ActRIIB proteins described herein may act in concert with or perhaps synergistically with another therapeutic agent.

In a specific example, a variety of osteogenic, cartilage-inducing and bone-inducing factors have been described, particularly bisphosphonates. See e.g., European Patent Application Nos. 148,155 and 169,016. For example, other factors that can be combined with the subject variant ActRIIB proteins (e.g., variant ActRIIB proteins in either homomeric or heteromeric forms) include various growth factors such as epidermal growth factor (EGF), platelet derived growth factor (PDGF), transforming growth factors (TGF-α and TGF-β), and insulin-like growth factor (IGF).

In certain embodiments, the present disclosure also provides gene therapy for the in vivo production of variant ActRIIB proteins (e.g., variant ActRIIB proteins in either homomeric or heteromeric forms). Such therapy would achieve its therapeutic effect by introduction of the variant ActRIIB polynucleotide sequences into cells or tissues having the disorders as listed above. Delivery of variant ActRIIB polynucleotide sequences can be achieved using a recombinant expression vector such as a chimeric virus or a colloidal dispersion system. Preferred for therapeutic delivery of variant ActRIIB polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the variant ActRIIB polynucleotide. In one preferred embodiment, the vector is targeted to renal, pulmonary, cardiac, bone, cartilage, muscle or neuron cells/tissues.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for variant ActRIIB polynucleotides (e.g., variant ActRIIB proteins in either homomeric or heteromeric forms) is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this disclosure is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

### Exemplification

The invention now being generally described, it will be more readily understood by reference to the following examples, which are included merely for purposes of illustration of certain embodiments and embodiments of the present invention, and are not intended to limit the invention.

### Example 1. Generation of an ActRIIB-Fc Fusion Protein

Applicants constructed a soluble ActRIIB fusion protein that has the extracellular domain of human ActRIIB fused to a human GlFc domain with a linker (three glycine amino acids) in between. The construct is referred to as ActRIIB-G1Fc.

ActRIIB-G1Fc is shown below in SEQ ID NO: 5 (with the linker underlined) as purified from CHO cell lines:

GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAPTGGGTHTCPPCPAPELLGGPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
(SEQ ID NO: 5)

The ActRIIB-G1Fc protein was expressed in CHO cell lines. Three different leader sequences were considered:
(i) Honey bee mellitin (HBML): MKFLVNVALVFMVVYISYIYA (SEQ ID NO: 7)
(ii) Tissue plasminogen activator (TPA): MDAMKRGLCCVLLLCGAVFVSP (SEQ ID NO: 8)
(iii) Native: MTAPWVALALLWGSLCAG (SEQ ID NO: 9).

The selected form employs the TPA leader and has the following unprocessed amino acid sequence:

MDAMKRGLCCVLLLCGAVFVSPGASGRGEAETRECIYYNANWELERTNQS
GLERCEGEQDKRLHCYASWRNSSGTIELVKKGCWLDDFNCYDRQECVATE
ENPQVYFCCCEGNFCNERFTHLPEAGGPEVTYEPPPTAPTGGGTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK(SEQ ID NO: 6)

This polypeptide is encoded by the following nucleic acid sequence (SEQ ID NO: 10):

ATGGATGCAAT GAAGAGAGGG CTCTGCTGTG TGCTGCTGCT GTGTGGAGCA GTCTTCGTTT
CGCCCGGCGC CTCTGGGCGT GGGGAGGCTG AGACACGGGA GTGCATCTAC TACAACGCCA
ACTGGGAGCT GGAGCGCACC AACCAGAGCG GCCTGGAGCG CTGCGAAGGC GAGCAGGACA
AGCGGCTGCA CTGCTACGCC TCCTGGCGCA ACAGCTCTGG CACCATCGAG CTCGTGAAGA
AGGGCTGCTG GCTAGATGAC TTCAACTGCT ACGATAGGCA GGAGTGTGTG GCCACTGAGG
AGAACCCCCA GGTGTACTTC TGCTGCTGTG AAGGCAACTT CTGCAACGAG CGCTTCACTC
ATTTGCCAGA GGCTGGGGGC CCGGAAGTCA CGTACGAGCC ACCCCCGACA GCCCCCACCG
GTGGTGGAAC TCACACATGC CCACCGTGCC CAGCACCTGA ACTCCTGGGG GGACCGTCAG
TCTTCCTCTT CCCCCCAAAA CCCAAGGACA CCCTCATGAT CTCCCGGACC CCTGAGGTCA
CATGCGTGGT GGTGGACGTG AGCCACGAAG ACCCTGAGGT CAAGTTCAAC TGGTACGTGG
ACGGCGTGGA GGTGCATAAT GCCAAGACAA AGCCGCGGGA GGAGCAGTAC AACAGCACGT
ACCGTGTGGT CAGCGTCCTC ACCGTCCTGC ACCAGGACTG GCTGAATGGC AAGGAGTACA
AGTGCAAGGT CTCCAACAAA GCCCTCCCAG CCCCCATCGA GAAAACCATC TCCAAAGCCA
AAGGGCAGCC CCGAGAACCA CAGGTGTACA CCCTGCCCCC ATCCCGGGAG GAGATGACCA
AGAACCAGGT CAGCCTGACC TGCCTGGTCA AAGGCTTCTA TCCCAGCGAC ATCGCCGTGG
AGTGGGAGAG CAATGGGCAG CCGGAGAACA ACTACAAGAC CACGCCTCCC GTGCTGGACT
CCGACGGCTC CTTCTTCCTC TATAGCAAGC TCACCGTGGA CAAGAGCAGG TGGCAGCAGG
GGAACGTCTT CTCATGCTCC GTGATGCATG AGGCTCTGCA CAACCACTAC ACGCAGAAGA
GCCTCTCCCT GTCTCCGGGT AAATGA (SEQ ID NO: 10)

N-terminal sequencing of the CHO-cell produced material revealed a major sequence of -GRGEAE (SEQ ID NO: 11). Notably, other constructs reported in the literature begin with an -SGR... sequence.

ActRIIB-G1Fc can also be presented as shown below in SEQ ID NO: 519 (with the linker underlined) as purified from CHO cell lines:

GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEVTYEPPPTAPTGGGTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPVPIEKTISKAKGQPREPQVY
TLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
(SEQ ID NO: 519)

The selected form employs the TPA leader and has the following unprocessed amino acid sequence (SEQ ID NO: 520):

1 MDAMKRGLCC VLLLCGAVFV SPGASGRGEA ETRECIYYNA NWELERTNQS
51 GLERCEGEQD KRLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 VPIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 520)

This polypeptide is encoded by the following nucleic acid sequence (SEQ ID NO: 521):

1 ATGGATGCAA TGAAGAGAGG GCTCTGCTGT GTGCTGCTGC TGTGTGGAGC AGTCTTCGTT
61 TCGCCCGGCG CCTCTGGGCG TGGGGAGGCT GAGACACGGG AGTGCATCTA CTACAACGCC
121 AACTGGGAGC TGGAGCGCAC CAACCAGAGC GGCCTGGAGC GCTGCGAAGG CGAGCAGGAC
181 AAGCGGCTGC ACTGCTACGC CTCCTGGCGC AACAGCTCTG GCACCATCGA GCTCGTGAAG
241 AAGGGCTGCT GGCTAGATGA CTTCAACTGC TACGATAGGC AGGAGTGTGT GGCCACTGAG
301 GAGAACCCCC AGGTGTACTT CTGCTGCTGT GAAGGCAACT TCTGCAACGA GCGCTTCACT
361 CATTTGCCAG AGGCTGGGGG CCCGGAAGTC ACGTACGAGC CACCCCCGAC AGCCCCCACC
421 GGTGGTGGAA CTCACACATG CCCACCGTGC CCAGCACCTG AACTCCTGGG GGGACCGTCA
481 GTCTTCCTCT TCCCCCCAAA ACCCAAGGAC ACCCTCATGA TCTCCCGGAC CCCTGAGGTC
541 ACATGCGTGG TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA CAACAGCACG
661 TACCGTGTGG TCAGCGTCCT CACCGTCCTG CACCAGGACT GGCTGAATGG CAAGGAGTAC
721 AAGTGCAAGG TCTCCAACAA AGCCCTCCCA GTCCCCATCG AGAAAACCAT CTCCAAAGCC
781 AAAGGGCAGC CCCGAGAACC ACAGGTGTAC ACCCTGCCCC CATCCCGGGA GGAGATGACC
841 AAGAACCAGG TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC CGTGCTGGAC
961 TCCGACGGCT CCTTCTTCCT CTATAGCAAG CTCACCGTGG ACAAGAGCAG GTGGCAGCAG
1021 GGGAACGTCT TCTCATGCTC CGTGATGCAT GAGGCTCTGC ACAACCACTA CACGCAGAAG
1081 AGCCTCTCCC TGTCTCCGGG TAAATGA (SEQ ID NO: 521)

N-terminal sequencing of the CHO-cell produced material revealed a major sequence of -GRGEAE (SEQ ID NO: 11). Notably, other constructs reported in the literature begin with an -SGR... sequence.

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

The ActRIIB-Fc fusion protein was also expressed in HEK293 cells and COS cells. Although material from all cell lines and reasonable culture conditions provided protein with muscle-building activity in vivo, variability in potency was observed perhaps relating to cell line selection and/or culture conditions.

### Example 2: Computational Methods

The Activin IIB receptor (ActRIIB) binds multiple TGFβ superfamily ligands, including activin A, activin B, GDF8, and GDF11, that stimulate Smad2/3 activation, as well as bone morphogenic proteins (BMPs), such as BMP9 and BMP10, that stimulate Smadl/5/8 activation. ActRIIB-Fc fusion proteins can function as ligand traps that bind to soluble ligands and block Smad activation by preventing ligands from binding to cell surface receptors. ActRIIB-Fc antagonism of BMP9-mediated Smadl/5/8 activation has been known to result in undesired side effects, including epistaxis and telangiectasias (Campbell, C. et al. Muscle Nerve 55: 458-464, 2017). In order to design mutations in ActRIIB that diminish BMP9 binding, while retaining binding to ligands that stimulate Smad2/3 activation, we compared the crystal structures of three ActRIIB ligand complexes: (1) BMP9:ActRIIB:A1k1, PDB ID=4fao, (2) ActRIIB:Activin A, PDB ID:1s4y, and (3) GDF11:ActRIIB:Alk5, PDB ID: 6mac (available from the Protein Data Bank (PDB) https://www.rcsb.org/). Comparison of contacts between ActRIIB and the three ligands based on the crystal structures revealed residues for mutational focus based on charge, polarity, and hydrophobicity differences of the ligand residues contacted by the same corresponding ActRIIB residue. After identifying residues to target for mutation, the Schrodinger Bioluminate biologics modeling software platform (version 2017-4: Bioluminate, Schrodinger, LLC, New York, NY) was used to computationally predict mutations in ActRIIB that would diminish binding to BMP9, while maintaining other ligand-binding activities.

All residues identified from the comparison of the crystal structures were considered for mutation. Residue Scanning Calculations were performed considering both stability and affinity of the molecules in the structural complex, producing a specified list of potential mutations and energies for each molecule (ligand and receptor) and complex structure, as well as energy differences for both the wild type and the mutant form. After analyzing affinity/stability/prime energy, etc. parameters, the top 5%-10% of the single mutations were identified. This analysis was followed by potential combination of these mutations. Selected single mutations and mutation combinations were structurally analyzed in order to understand structural differences and formed/lost contacts. Ultimately, 817 single mutations were screened for each complex (ActRIIB:ligand), and top hits were selected based on Δaffinity, and also taking into selective consideration Δstability (solvated) and Δprime energy. Other properties were also considered when regarding striking of outliers.

### Example 3: Generation of Variant ActRIIB-Fc Proteins

Based on the findings described in Example 1, Applicants generated a series of mutations (sequence variations) in the extracellular domain of ActRIIB and produced these variant polypeptides as soluble homodimeric fusion proteins comprising a variant ActRIIB extracellular domain and an Fc domain joined by an optional linker. The background ActRIIB-Fc fusion used for the generation of variant ActRIIB-Fc proteins was ActRIIB-GIFc, and is shown in Example 1 above as SEQ ID NO: 5.

Various substitution mutations were introduced into the background ActRIIB-G1Fc protein. Based on the data presented in Example 1, it is expected that these constructs, if expressed with a TPA leader, will lack the N-terminal serine. Thus, the majority of mature sequences may begin with a glycine (lacking the N-terminal serine) but some species may be present with the N-terminal serine. Mutations were generated in the ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNA was isolated. For murine constructs (mFc), a murine IgG2a was substituted for the human IgG1. All mutants were sequence verified.

The amino acid sequence of unprocessed ActRIIB(F82I-N83R)-G1Fc is shown below (SEQ ID NO: 276). The signal sequence and linker sequence are indicated by solid underline, and the F82I and N83R substitutions are indicated by double underline. The amino acid sequence of SEQ ID NO: 276 may optionally be provided with the lysine removed from the C-terminus.

1 MDAMKRGLCC VLLLCGAVFV SPGASGRGEA ETRECIYYNA NWELERTNQS
51 GLERCEGEQD KRLHCYASWR NSSGTIELVK KGCWLDDIRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 276)

This ActRIIB(F82I-N83R)-G1Fc fusion polypeptide is encoded by the following nucleic acid sequence (SEQ ID NO: 277):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CATCCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 277)

A mature ActRIIB(F82I-N83R)-G1Fc fusion polypeptide (SEQ ID NO: 278) is as follows and may optionally be provided with the lysine removed from the C-terminus.

1 GRGEAETREC IYYNANWELE RTNQSGLERC EGEQDKRLHC YASWRNSSGT
51 IELVKKGCWL DDIRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 278)

The amino acid sequence of unprocessed ActRIIB(F82K-N83R)-G1Fc is shown below (SEQ ID NO: 279). The signal sequence and linker sequence are indicated by solid underline, and the F82K and N83R substitutions are indicated by double underline. The amino acid sequence of SEQ ID NO: 279 may optionally be provided with the lysine removed from the C-terminus.

1 MDAMKRGLCC VLLLCGAVFV SPGASGRGEA ETRECIYYNA NWELERTNQS
51 GLERCEGEQD KRLHCYASWR NSSGTIELVK KGCWLDDKRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 279)

This ActRIIB(F82K-N83R)-G1Fc fusion polypeptide is encoded by the following nucleic acid sequence (SEQ ID NO: 331):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CAAGCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 331)

A mature ActRIIB(F82K-N83R)-G1Fc fusion polypeptide (SEQ ID NO: 332) is as follows and may optionally be provided with the lysine removed from the C-terminus.

1 GRGEAETREC IYYNANWELE RTNQSGLERC EGEQDKRLHC YASWRNSSGT
51 IELVKKGCWL DDKRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 332)

The amino acid sequence of unprocessed ActRIIB(F82T-N83R)-G1Fc is shown below (SEQ ID NO: 333). The signal sequence and linker sequence are indicated by solid underline, and the F82T and N83R substitutions are indicated by double underline. The amino acid sequence of SEQ ID NO: 333 may optionally be provided with the lysine removed from the C-terminus.

1 MDAMKRGLCC VLLLCGAVFV SPGASGRGEA ETRECIYYNA NWELERTNQS
51 GLERCEGEQD KRLHCYASWR NSSGTIELVK KGCWLDDTRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 333)

This ActRIIB(F82T-N83R)-G1Fc fusion polypeptide is encoded by the following nucleic acid sequence (SEQ ID NO: 334):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CACCCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 334)

A mature ActRIIB(F82T-N83R)-G1Fc fusion polypeptide (SEQ ID NO: 335) is as follows and may optionally be provided with the lysine removed from the C-terminus.

1 GRGEAETREC IYYNANWELE RTNQSGLERC EGEQDKRLHC YASWRNSSGT
51 IELVKKGCWL DDTRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 335)

The amino acid sequence of unprocessed ActRIIB(F82T)-G1Fc is shown below (SEQ ID NO: 336). The signal sequence and linker sequence are indicated by solid underline, and the F82T substitution is indicated by double underline. The amino acid sequence of SEQ ID NO: 336 may optionally be provided with the lysine removed from the C-terminus.

1 MDAMKRGLCC VLLLCGAVFV SPGASGRGEA ETRECIYYNA NWELERTNQS
51 GLERCEGEQD KRLHCYASWR NSSGTIELVK KGCWLDDTNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 336)

This ActRIIB(F82T)-G1Fc fusion polypeptide is encoded by the following nucleic acid sequence (SEQ ID NO: 337):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CACCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 337)

A mature ActRIIB(F82T)-G1Fc fusion polypeptide (SEQ ID NO: 338) is as follows and may optionally be provided with the lysine removed from the C-terminus.

1 GRGEAETREC IYYNANWELE RTNQSGLERC EGEQDKRLHC YASWRNSSGT
51 IELVKKGCWL DDTNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 338)

The amino acid sequence of unprocessed ActRIIB(L79H-F82I)-GlFc is shown below (SEQ ID NO: 339). The signal sequence and linker sequence are indicated by solid underline, and the L79H and F82I substitutions are indicated by double underline. The amino acid sequence of SEQ ID NO: 339 may optionally be provided with the lysine removed from the C-terminus.

1 MDAMKRGLCC VLLLCGAVFV SPGASGRGEA ETRECIYYNA NWELERTNQS
51 GLERCEGEQD KRLHCYASWR NSSGTIELVK KGCWHDDINC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 339)

This ActRIIB(L79H-F82I)-GlFc fusion polypeptide is encoded by the following nucleic acid sequence (SEQ ID NO: 340):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCACGATGA CATCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 340)

A mature ActRIIB(L79H-F82I)-GlFc fusion polypeptide (SEQ ID NO: 341) is as follows and may optionally be provided with the lysine removed from the C-terminus.

1 GRGEAETREC IYYNANWELE RTNQSGLERC EGEQDKRLHC YASWRNSSGT
51 IELVKKGCWH DDINCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 341)

The amino acid sequence of unprocessed ActRIIB(L79H)-GlFc is shown below (SEQ ID NO: 342). The signal sequence and linker sequence are indicated by solid underline, and the L79H substitution is indicated by double underline. The amino acid sequence of SEQ ID NO: 342 may optionally be provided with the lysine removed from the C-terminus.

1 MDAMKRGLCC VLLLCGAVFV SPGASGRGEA ETRECIYYNA NWELERTNQS
51 GLERCEGEQD KRLHCYASWR NSSGTIELVK KGCWHDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 342)

This ActRIIB(L79H)-GlFc fusion polypeptide is encoded by the following nucleic acid sequence (SEQ ID NO: 343):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCACGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 343)

A mature ActRIIB(L79H)-GlFc fusion polypeptide (SEQ ID NO: 344) is as follows and may optionally be provided with the lysine removed from the C-terminus.

1 GRGEAETREC IYYNANWELE RTNQSGLERC EGEQDKRLHC YASWRNSSGT
51 IELVKKGCWH DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 344)

The amino acid sequence of unprocessed ActRIIB(L79H-F82K)-GlFc is shown below (SEQ ID NO: 345). The signal sequence and linker sequence are indicated by solid underline, and the L79H and F82K substitutions are indicated by double underline. The amino acid sequence of SEQ ID NO: 345 may optionally be provided with the lysine removed from the C-terminus.

1 MDAMKRGLCC VLLLCGAVFV SPGASGRGEA ETRECIYYNA NWELERTNQS
51 GLERCEGEQD KRLHCYASWR NSSGTIELVK KGCWHDDKNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 345)

This ActRIIB(L79H-F82K)-GlFc fusion polypeptide is encoded by the following nucleic acid sequence (SEQ ID NO: 346):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCACGATGA CAAGAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 346)

A mature ActRIIB(L79H-F82K)-GlFc fusion polypeptide (SEQ ID NO: 347) is as follows and may optionally be provided with the lysine removed from the C-terminus.

1 GRGEAETREC IYYNANWELE RTNQSGLERC EGEQDKRLHC YASWRNSSGT
51 IELVKKGCWH DDKNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 347)

The amino acid sequence of unprocessed ActRIIB(E50L)-GlFc is shown below (SEQ ID NO: 348). The signal sequence and linker sequence are indicated by solid underline, and the E50L substitution is indicated by double underline. The amino acid sequence of SEQ ID NO: 348 may optionally be provided with the lysine removed from the C-terminus.

1 MDAMKRGLCC VLLLCGAVFV SPGASGRGEA ETRECIYYNA NWELERTNQS
51 GLERCLGEQD KRLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 348)

This ActRIIB(E50L)-GlFc fusion polypeptide is encoded by the following nucleic acid sequence (codon optimized) (SEQ ID NO: 349):

1 ATGGATGCGA TGAAACGCGG CCTGTGCTGC GTGCTGCTGC TGTGCGGCGC
51 GGTGTTTGTG AGCCCGGGCG CCAGCGGCCG CGGCGAAGCG GAAACCCGCG
101 AATGCATTTA TTATAACGCG AACTGGGAAC TGGAACGCAC CAACCAGAGC
151 GGCCTGGAAC GCTGCCTGGG CGAACAGGAT AAACGCCTGC ATTGCTATGC
201 GAGCTGGCGC AACAGCAGCG GCACCATTGA ACTGGTGAAA AAAGGCTGCT
251 GGCTGGATGA TTTTAACTGC TATGATCGCC AGGAATGCGT GGCGACCGAA
301 GAAAACCCGC AGGTGTATTT TTGCTGCTGC GAAGGCAACT TTTGCAACGA
351 ACGCTTTACC CATCTGCCGG AAGCGGGCGG CCCGGAAGTG ACCTATGAAC
401 CGCCGCCGAC CGCGCCGACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 349)

A mature ActRIIB(E50L)-GlFc fusion polypeptide (SEQ ID NO: 350) is as follows and may optionally be provided with the lysine removed from the C-terminus.

1 GRGEAETREC IYYNANWELE RTNQSGLERC LGEQDKRLHC YASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 350)

The amino acid sequence of unprocessed ActRIIB(L38N-L79R)-GlFc is shown below (SEQ ID NO: 351). The signal sequence and linker sequence are indicated by solid underline, and the L38N and L79R substitutions are indicated by double underline. The amino acid sequence of SEQ ID NO: 351 may optionally be provided with the lysine removed from the C-terminus.

1 MDAMKRGLCC VLLLCGAVFV SPGASGRGEA ETRECIYYNA NWENERTNQS
51 GLERCEGEQD KRLHCYASWR NSSGTIELVK KGCWRDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 351)

This ActRIIB(L38N-L79R)-GlFc fusion polypeptide is encoded by the following nucleic acid sequence (SEQ ID NO: 352):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGA ACGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCGCGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 352)

A mature ActRIIB(L38N-L79R)-GlFc fusion polypeptide (SEQ ID NO: 353) is as follows and may optionally be provided with the lysine removed from the C-terminus.

1 GRGEAETREC IYYNANWENE RTNQSGLERC EGEQDKRLHC YASWRNSSGT
51 IELVKKGCWR DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 353)

The amino acid sequence of unprocessed ActRIIB(V99G)-GlFc is shown below (SEQ ID NO: 354). The signal sequence and linker sequence are indicated by solid underline, and the V99G substitution is indicated by double underline. The amino acid sequence of SEQ ID NO: 354 may optionally be provided with the lysine removed from the C-terminus.

1 MDAMKRGLCC VLLLCGAVFV SPGASGRGEA ETRECIYYNA NWELERTNQS
51 GLERCEGEQD KRLHCYASWR NSSGTIELVK KGCWLDDFNC YDRQECVATE
101 ENPQGYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK* (SEQ ID NO: 354)

This ActRIIB(V99G)-GlFc fusion polypeptide is encoded by the following nucleic acid sequence (codon optimized) (SEQ ID NO: 355):

1 ATGGATGCGA TGAAACGCGG CCTGTGCTGC GTGCTGCTGC TGTGCGGCGC
51 GGTGTTTGTG AGCCCGGGCG CCAGCGGCCG CGGCGAAGCG GAAACCCGCG
101 AATGCATTTA TTATAACGCG AACTGGGAAC TGGAACGCAC CAACCAGAGC
151 GGCCTGGAAC GCTGCGAAGG CGAACAGGAT AAACGCCTGC ATTGCTATGC
201 GAGCTGGCGC AACAGCAGCG GCACCATTGA ACTGGTGAAA AAAGGCTGCT
251 GGCTGGATGA TTTTAACTGC TATGATCGCC AGGAATGCGT GGCGACCGAA
301 GAAAACCCGC AGGGCTATTT TTGCTGCTGC GAAGGCAACT TTTGCAACGA
351 ACGCTTTACC CATCTGCCGG AAGCGGGCGG CCCGGAAGTG ACCTATGAAC
401 CGCCGCCGAC CGCGCCGACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAATGA (SEQ ID NO: 355)

A mature ActRIIB(V99G)-GlFc fusion polypeptide (SEQ ID NO: 356) is as follows and may optionally be provided with the lysine removed from the C-terminus.

1 GRGEAETREC IYYNANWELE RTNQSGLERC EGEQDKRLHC YASWRNSSGT
51 IELVKKGCWL DDFNCYDRQE CVATEENPQG YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK*
(SEQ ID NO: 356)

Constructs were expressed in COS or CHO cells by transient infection and purified by filtration and protein A chromatography. In some instances, assays were performed with conditioned medium rather than purified proteins. Purity of samples for reporter gene assays was evaluated by SDS-PAGE and analytical size exclusion chromatography.

Mutants were tested in binding assays and/or bioassays described below.

Alternatively, similar mutations could be introduced into an ActRIIB extracellular domain possessing an N-terminal truncation of five amino acids and a C-terminal truncation of three amino acids as shown below (SEQ ID NO: 357). This truncated ActRIIB extracellular domain is denoted ActRIIB(25-131) based on numbering in SEQ ID NO: 2.

25 ETRECIYYNA NWELERTNQS GLERCEGEQD KRLHCYASWR NSSGTIELVK
75 KGCWLDDFNC YDRQECVATE ENPQVYFCCC EGNFCNERFT HLPEAGGPEV
125 TYEPPPT (SEQ ID NO: 53)

The corresponding background fusion polypeptide, ActRIIB(25-131)-GlFc, is shown below (SEQ ID NO: 12).

1 ETRECIYYNA NWELERTNQS GLERCEGEQD KRLHCYASWR NSSGTIELVK
51 KGCWLDDFNC YDRQECVATE ENPQVYFCCC EGNFCNERFT HLPEAGGPEV
101 TYEPPPTGGG THTCPPCPAP ELLGGPSVFL FPPKPKDTLM ISRTPEVTCV
151 VVDVSHEDPE VKFNWYVDGV EVHNAKTKPR EEQYNSTYRV VSVLTVLHQD
201 WLNGKEYKCK VSNKALPAPI EKTISKAKGQ PREPQVYTLP PSREEMTKNQ
251 VSLTCLVKGF YPSDIAVEWE SNGQPENNYK TTPPVLDSDG SFFLYSKLTV
301 DKSRWQQGNV FSCSVMHEAL HNHYTQKSLS LSPGK (SEQ ID NO: 12)

### Example 4. Activity and Ligand Binding Profiles of Variant ActRIIB-Fc Proteins

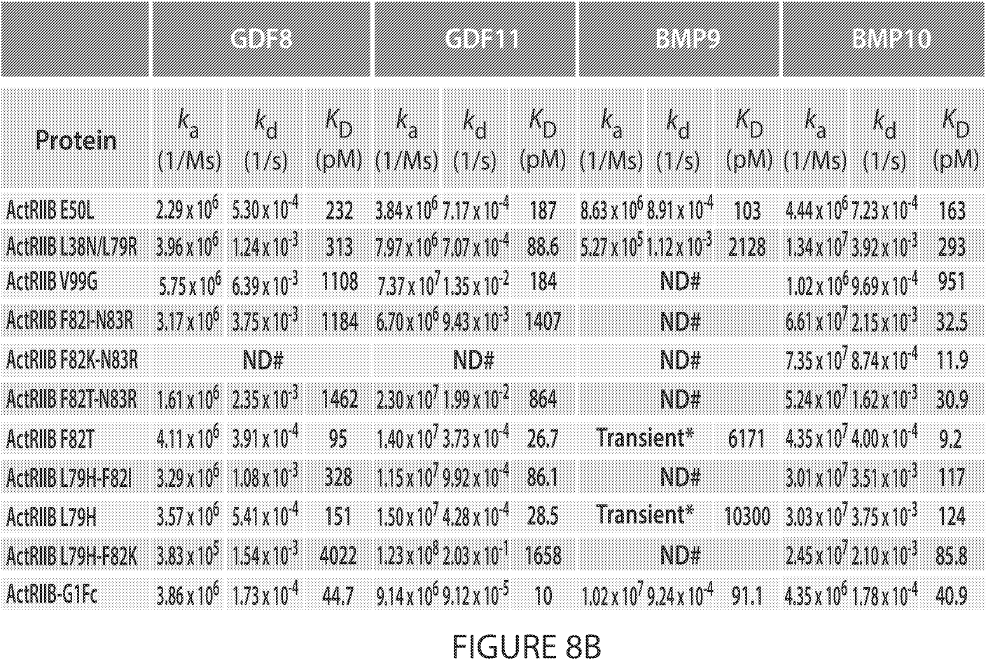

To determine ligand binding profiles of variant ActRIIB-Fc homodimers, a Biacore™-based binding assay was used to compare ligand binding kinetics of certain variant ActRIIB-Fc proteins. ActRIIB-Fc proteins to be tested were independently captured onto the system using an anti-Fc antibody. Ligands were then injected and allowed to flow over the captured receptor protein. Results of variant ActRIIB-Fc proteins analyzed at 37° C. are shown in FIGS. 8A and 8B. ActRIIB-G1Fc was used as the control protein.

To determine activity of variant ActRIIB-Fc proteins, an A204 cell-based assay was used to compare effects among variant ActRIIB-Fc proteins on signaling by activin A, activin B, GDF8, GDF11, BMP9, and BMP10, in comparison to ActRIIB-G1Fc. In brief, this assay uses a human A204 rhabdomyosarcoma cell line (ATCC®: HTB-82™) derived from muscle and the reporter vector pGL3(CAGA)12 (Dennler et al., 1998, EMBO 17: 3091-3100) as well as a Renilla reporter plasmid (pRLCMV) to control for transfection efficiency. The CAGA12 motif is present in TGF-β responsive genes (e.g., PAI-1 gene), so this vector is of general use for ligands that can signal through Smad2/3, including activin A, GDF11, and BMP9.

On day 1, A204 cells were transferred into one or more 48-well plates. On day 2, these cells were transfected with 10 µg pGL3(CAGA)12 or pGL3(CAGA)12(10 µg) + pRLCMV (1 µg) and Fugene. On day 3, ligands diluted in medium containing 0.1% BSA were preincubated with ActRIIB-Fc proteins for 1 hr before addition to cells. Approximately six hour later, the cells were rinsed with PBS and lysed. Cell lysates were analyzed in a luciferase assay to determine the extent of Smad activation.

This assay was used to screen variant ActRIIB-Fc proteins for inhibitory effects on cell signaling by activin A, activin B, GDF8, GDF11, BMP9, and BMP10. Potencies of homodimeric Fc fusion proteins incorporating amino acid substitutions in the human ActRIIB extracellular domain were compared with that of an Fc fusion protein comprising unmodified human ActRIIB extracellular domain, ActRIIB-G1Fc. For some variants tested, it was not possible to calculate an accurate IC₅₀, but signs of inhibition in the slope of the curves were detectable. For these variants, an estimate was included of the order of magnitude of the relative IC50, i.e. >10 nM or > 100 nM instead of a definite number. Such data points are indicated by a (*) in Table 1 below. For some variants tested, there was no detectable inhibition in the slope of the curves over the concentration range tested, which is indicated by “ND” in Table 1.

TABLE 1

Inhibitory Potency of Homodimeric ActRIIB-Fc Constructs
Inhibitory Potency of Homodimeric ActRIIB-Fc Constructs

| ActRIIB protein | IC₅₀ (nM) | | | | | |
|---|---|---|---|---|---|---|
| | GDF8 | GDF11 | Activin A | Activin B | BMP9 | BMP10 |
| ActRIIB-GIFc | 0.95 | 0.12 | 0.05 | 0.067 | 1.82 | 0.036 |
| F82I-N83R | ND | 9.95 | 1.67 | 0.08 | ND | 13.25 |
| F82K-N83R | ND | ND | 1.32 | 0.09 | ND | 0.53 |
| F82T-N83R | ND | 17.94 | 1.52 | 0.11 | ND | 12.57 |
| F82T | 2.17 | 0.27 | 0.10 | 0.09 | ND | 0.07 |
| L79H-F82I | >10* | 0.36 | >100* | 0.15 | ND | >100* |
| L79H | 5.76 | 0.24 | >10* | 0.07 | ND | >100* |
| L79H-F82K | ND | >100* | ND | 0.10 | ND | >100* |

ND: not detectable over concentration range tested * estimate of the order of magnitude of the IC₅₀

As shown in Table 1 above as well as in FIGS. 8A and 8B, amino acid substitutions in the ActRIIB extracellular domain can alter the balance between ActRIIB:ligand binding and downstream signaling activities in various in vitro assay. In general, applicant achieved the goal of generating variants in the ActRIIB extracellular domain that exhibited decreased or non-detectable binding to BMP9, compared to a fusion protein containing unmodified ActRIIB extracellular domain (ActRIIB-GlFc), while retaining other ligand binding properties.

Additionally, variants ActRIIB (L79H-F82I), ActRIIB (L79H), and ActRIIB (L79H-F82K), while demonstrating a decrease in binding to BMP9, also exhibited a significant decrease in in activin A binding while retaining relatively high affinity for activin B, as compared to ActRIIB-G1Fc. IC₅₀ values showing inhibitory potency in Table 1 are consistent with this ligand binding trend. Similarly, variants ActRIIB (F82K-N83R), ActRIIB (F82I-N83R), and ActRIIB (F82T-N83R) demonstrate a similar trend.

Furthermore, variants ActRIIB (F82K-N83R), ActRIIB (F82I-N83R), ActRIIB (F82T-N83R), and ActRIIB (L79H-F82K), while demonstrating a decrease in binding to BMP9 and retaining relatively high affinity for activin B, also exhibited a significant decrease in GDF8 and GDF11 binding, as compared to ActRIIB-G1Fc. IC₅₀ values showing inhibitory potency in Table 1 are consistent with this ligand binding trend.

It was further noted that, variants ActRIIB (L79H-F82I), ActRIIB (L79H), and ActRIIB (L79H-F82K), while demonstrating a decrease in binding to BMP9 and retaining relatively high affinity for activin B, also exhibited a decrease in BMP10 binding as compared to ActRIIB-G1Fc. IC₅₀ values showing inhibitory potency in Table 1 are consistent with this ligand binding trend.

Therefore, in addition to achieving the goal of producing ActRIIB variants that exhibit reduced to non-detectable binding to BMP9, Applicant has generated a diverse array of novel variant polypeptides, many of which are characterized in part by unique ligand binding/inhibition profiles. Accordingly, these variants may be more useful than ActRIIB-GlFc in certain applications where such selective antagonism is advantageous. Examples include therapeutic applications where it is desirable to retain antagonism of activin B, while reducing antagonism of BMP9 and optionally one or more of activin A, GDF8, GDF11 and BMP10.

### Example 5: Generation of Variant ActRIIB-Fc Proteins

Applicants generated a series of mutations (sequence variations) in the extracellular domain of ActRIIB and produced these variant polypeptides as soluble homodimeric fusion proteins comprising a variant ActRIIB extracellular domain and an Fc domain joined by an optional linker. The background ActRIIB-Fc fusion was ActRIIB-G1Fc as shown in SEQ ID NO: 5.

Various substitution mutations were introduced into the background ActRIIB-Fc protein. Based on the data presented in Example 1, it is expected that these constructs, if expressed with a TPA leader, will lack the N-terminal serine. Mutations were generated in the ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNA was isolated. For murine constructs (mFc), a murine IgG2a was substituted for the human IgG1. Murine constructs have similar biological characteristics to corresponding human constructs. All mutants were sequence verified.

The amino acid sequence of unprocessed ActRIIB(K55A)-GlFc is shown below (SEQ ID NO: 31). The signal sequence and linker sequence are indicated by solid underline, and the K55A substitution is indicated by double underline. The amino acid sequence of SEQ ID NO:31 may optionally be provided with the lysine removed from the C-terminus. The GIFc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD ARLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 31)

This ActRIIB(K55A)-GlFc fusion polypeptide is encoded by the following nucleic acid sequence (SEQ ID NO: 32):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC GCCCGGCTGC ACTGCTACGC
201 CTCCTGGCGC AACAGCTCTG GCACCATCGA GCTCGTGAAG AAGGGCTGCT
251 GGCTAGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCCCCGGG
1101 TAAA (SEQ ID NO: 32)

The mature ActRIIB(K55A)-GlFc fusion polypeptide (SEQ ID NO: 33) is as follows and may optionally be provided with the lysine removed from the C-terminus. The GIFc region may also comprise a mutation to an alanine at position 234 or a mutation to an alanine at position 235, or a combination thereof.

1 GRGEAETREC IYYNANWELE RTNQSGLERC EGEQDARLHC YASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 33)

The amino acid sequence of unprocessed ActRIIB(K55E)-GlFc is shown below (SEQ ID NO: 34). The signal sequence and linker sequence are indicated by solid underline, and the K55E substitution is indicated by double underline. The amino acid sequence of SEQ ID NO:34 may optionally be provided with the lysine removed from the C-terminus. The GIFc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD ERLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 34)

This ActRIIB(K55E)-GlFc fusion polypeptide is encoded by the following nucleic acid sequence (SEQ ID NO: 35):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC GAGCGGCTGC ACTGCTACGC
201 CTCCTGGCGC AACAGCTCTG GCACCATCGA GCTCGTGAAG AAGGGCTGCT
251 GGCTAGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCCCCGGG
1101 TAAA (SEQ ID NO: 35)

The mature ActRIIB(K55E)-GlFc fusion polypeptide (SEQ ID NO: 36) is as follows and may optionally be provided with the lysine removed from the C-terminus. The GIFc region may also comprise a mutation to an alanine at position 234 or a mutation to an alanine at position 235, or a combination thereof.

1 GRGEAETREC IYYNANWELE RTNQSGLERC EGEQDERLHC YASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 36)

The amino acid sequence of unprocessed ActRIIB(F82I)-GlFc is shown below (SEQ ID NO: 37). The signal sequence and linker sequence are indicated by solid underline, _and the F82I substitution is indicated by double underline. The amino acid sequence of SEQ ID NO: 37 may optionally be provided with the lysine removed from the C-terminus. The GIFc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDINC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 37)

This ActRIIB(F82I)-GlFc fusion polypeptide is encoded by the following nucleic acid sequence (SEQ ID NO: 38):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CATCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 38)

The mature ActRIIB(F82I)-GlFc fusion polypeptide (SEQ ID NO: 39) is as follows and may optionally be provided with the lysine removed from the C-terminus. The GIFc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDINCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 39)

The amino acid sequence of unprocessed ActRIIB(F82K)-GlFc is shown below (SEQ ID NO: 40). The signal sequence and linker sequence are indicated by solid underline, _and the F82K substitution is indicated by double underline. The amino acid sequence of SEQ ID NO: 40 may optionally be provided with the lysine removed from the C-terminus. The GIFc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDKNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 40)

This ActRIIB(F82K)-GlFc fusion polypeptide is encoded by the following nucleic acid sequence (SEQ ID NO: 41):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CAAGAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 41)

The mature ActRIIB(F82K)-GlFc fusion polypeptide (SEQ ID NO: 42) is as follows and may optionally be provided with the lysine removed from the C-terminus, and the GIFc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDKNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 42)

The amino acid sequence of unprocessed ActRIIB(F82K)-GlFc (LALA) is shown below (SEQ ID NO: 522). The signal sequence and linker sequence are indicated by solid underline, and the F82K substitution is indicated by double underline. The amino acid sequence of SEQ ID NO: 522 may optionally be provided with the lysine removed from the C-terminus.

1 MDAMKRGLCC VLLLCGAVFV SPGASGRGEA ETRECIYYNA NWELERTNQS
51 GLERCEGEQD KRLHCYASWR NSSGTIELVK KGCWLDDKNC YDRQECVATE
101 ENPQVYFCCC EGNFCNERFT HLPEAGGPEV TYEPPPTAPT GGGTHTCPPC
151 PAPEAAGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 522)

This ActRIIB(F82K)-GlFc (LALA) fusion polypeptide is encoded by the following nucleic acid sequence (SEQ ID NO: 523):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CAAGAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AAGCCGCTGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCCCCGGG
1101 TTGA (SEQ ID NO: 523)

The mature ActRIIB(F82K)-GlFc (LALA) fusion polypeptide (SEQ ID NO: 524) is as follows and may optionally be provided with the lysine removed from the C-terminus:

1 GRGEAETREC IYYNANWELE RTNQSGLERC EGEQDKRLHC YASWRNSSGT
51 IELVKKGCWL DDKNCYDRQE CVATEENPQV YFCCCEGNFC NERFTHLPEA
101 GGPEVTYEPP PTAPTGGGTH TCPPCPAPEA AGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 524)

Constructs were expressed in COS or CHO cells and purified by filtration and protein A chromatography. In some instances, assays were performed with conditioned medium rather than purified proteins. Purity of samples for reporter gene assays was evaluated by SDS-PAGE and Western blot analysis.

Mutants were tested in binding assays and/or bioassays described below.

Alternatively, similar mutations could be introduced into an ActRIIB extracellular domain possessing an N-terminal truncation of five amino acids and a C-terminal truncation of three amino acids as shown below (SEQ ID NO: 53). This truncated ActRIIB extracellular domain is denoted ActRIIB(25-131) based on numbering in SEQ ID NO: 2.

25 ETRECIYYNA NWELERTNQS GLERCEGEQD KRLHCYASWR NSSGTIELVK
75 KGCWLDDFNC YDRQECVATE ENPQVYFCCC EGNFCNERFT HLPEAGGPEV
125 TYEPPPT (SEQ ID NO: 53)

The corresponding background fusion polypeptide, ActRIIB(25-131)-GlFc, is shown below (SEQ ID NO: 12).

1 ETRECIYYNA NWELERTNQS GLERCEGEQD KRLHCYASWR NSSGTIELVK
51 KGCWLDDFNC YDRQECVATE ENPQVYFCCC EGNFCNERFT HLPEAGGPEV
101 TYEPPPTGGG THTCPPCPAP ELLGGPSVFL FPPKPKDTLM ISRTPEVTCV
151 VVDVSHEDPE VKFNWYVDGV EVHNAKTKPR EEQYNSTYRV VSVLTVLHQD
201 WLNGKEYKCK VSNKALPAPI EKTISKAKGQ PREPQVYTLP PSREEMTKNQ
251 VSLTCLVKGF YPSDIAVEWE SNGQPENNYK TTPPVLDSDG SFFLYSKLTV
301 DKSRWQQGNV FSCSVMHEAL HNHYTQKSLS LSPGK (SEQ ID NO: 12)

### Example 6. Ligand Binding Profiles of Variant ActRIIB-Fc Homodimers and Activity of Variant ActRIIB-Fc Proteins in a Cell-Based Assay

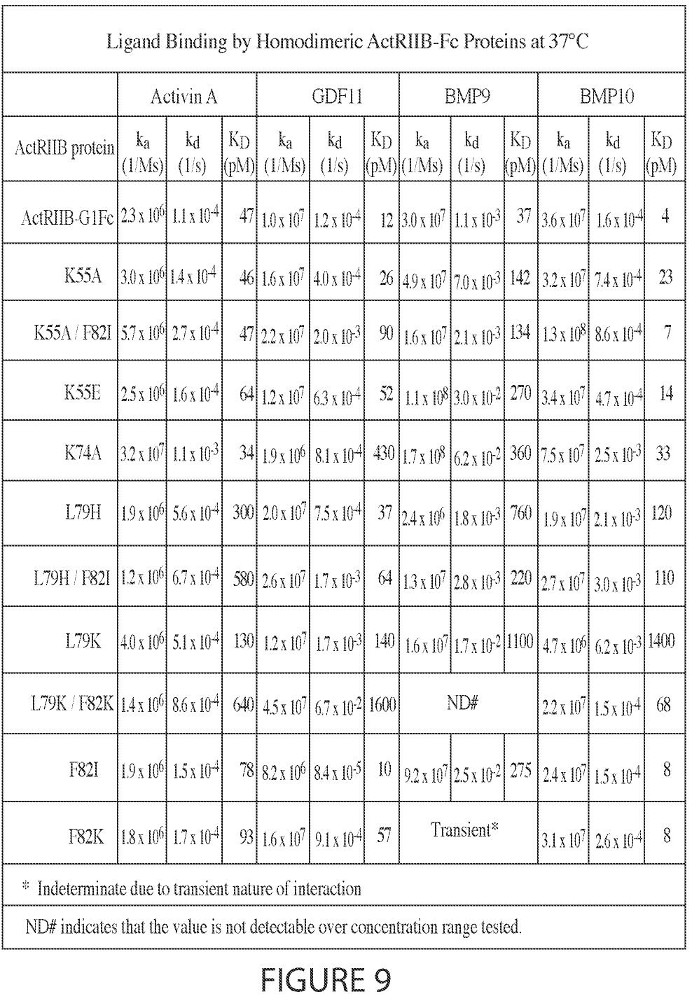

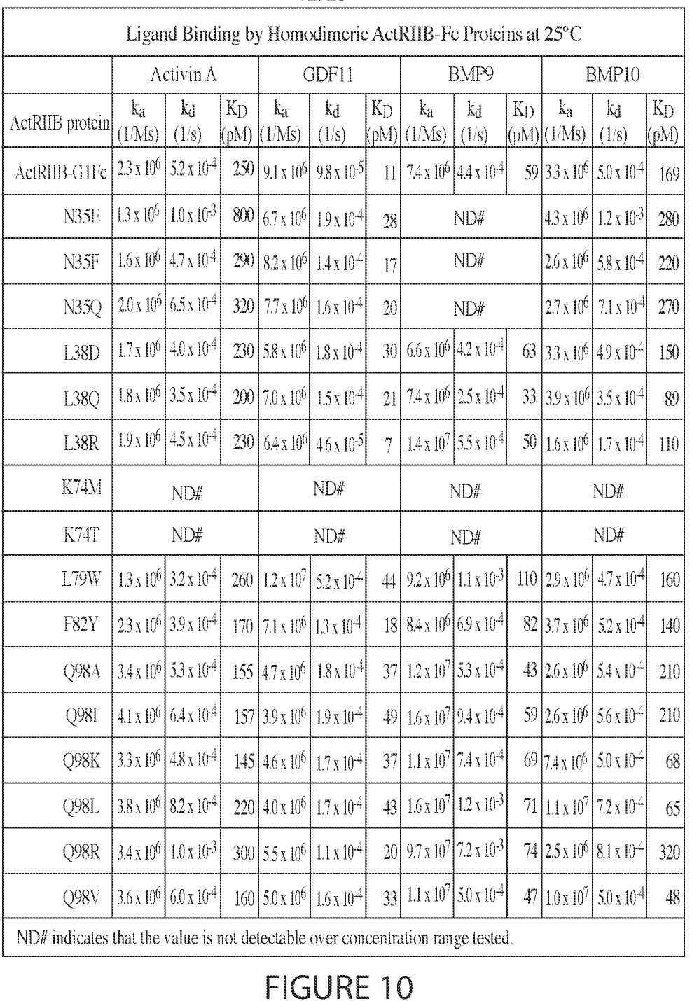

To determine ligand binding profiles of variant ActRIIB-Fc homodimers, a Biacore™-based binding assay was used to compare ligand binding kinetics of certain variant ActRIIB-Fc proteins. ActRIIB-Fc proteins to be tested were independently captured onto the system using an anti-Fc antibody. Ligands were then injected and allowed to flow over the captured receptor protein. Results of variant ActRIIB-Fc proteins analyzed at 37° C. are shown in FIG. 9. Compared to Fc-fusion protein comprising unmodified ActRIIB extracellular domain, the variant proteins ActRIIB(K55A)-Fc, ActRIIB(K55E)-Fc, ActRIIB(F82I)-Fc, and ActRIIB(F82K)-Fc exhibited greater reduction in their affinity for BMP9 than for GDF11. Results of additional variant ActRIIB-Fc proteins analyzed at 25° C. are shown in FIG. 10.

These results confirm K55A, K55E, F82I, and F82K as substitutions that reduce ActRIIB binding affinity for BMP9 more than they reduce ActRIIB affinity for activin A or GDF11. Accordingly, these variant ActRIIB-Fc proteins may be more useful than unmodified ActRIIB-Fc protein in certain applications where such selective antagonism is advantageous. Examples include therapeutic applications where it is desirable to retain antagonism of one or more of activin A, activin B, GDF8, and GDF11 while reducing antagonism of BMP9.

To determine activity of variant ActRIIB-Fc proteins, an A204 cell-based assay was used to compare effects among variant ActRIIB-Fc proteins on signaling by activin A, GDF11, and BMP9. In brief, this assay uses a human A204 rhabdomyosarcoma cell line (ATCC^{®}: HTB-82™) derived from muscle and the reporter vector pGL3(CAGA)12 (Dennler et al., 1998, EMBO 17: 3091-3100) as well as a Renilla reporter plasmid (pRLCMV) to control for transfection efficiency. The CAGA12 motif is present in TGF-β responsive genes (e.g., PAI-1 gene), so this vector is of general use for ligands that can signal through Smad2/3, including activin A, GDF11, and BMP9.

On day 1, A-204 cells were transferred into one or more 48-well plates. On day 2, these cells were transfected with 10 µg pGL3(CAGA)12 or pGL3(CAGA)12(10 µg) + pRLCMV (1 µg) and Fugene. On day 3, ligands diluted in medium containing 0.1% BSA were preincubated with ActRIIB-Fc proteins for 1 hr before addition to cells. Approximately six hour later, the cells were rinsed with PBS and lysed. Cell lysates were analyzed in a luciferase assay to determine the extent of Smad activation.

This assay was used to screen variant ActRIIB-Fc proteins for inhibitory effects on cell signaling by activin A, GDF11, and BMP9. Potencies of homodimeric Fc fusion proteins incorporating amino acid substitutions in the human ActRIIB extracellular domain were compared with that of an Fc fusion protein comprising unmodified human ActRIIB extracellular domain.

Inhibitory Potency of Homodimeric ActRIIB-Fc Constructs

| ActRIIB protein | IC₅₀ (ng/mL) | | |
|---|---|---|---|
| | Activin A | GDF11 | BMP9 |
| Wild-type | 8 | 9 | 31 |
| A24N | 128 | 99 | 409 |
| R40A | --- | 591 | 1210 |
| E50K | 132 | 180 | 721 |
| E50P | 756 | 638 | ~3000 |
| E52A | 198 | 71 | 359 |
| E52K | 762 | 296 | ∼10000 |
| K55A | 15 | 11 | 122 |
| K55D | 396 | 365 | 5500 |
| K55E | 19 | 14 | 290 |
| K55R | 206 | 318 | 777 |
| Y60K | --- | 414 | ND |
| Y60P | --- | 544 | ND |
| K74R | --- | 45 | 165 |
| K74Y | --- | ND | ND |
| K74A / L79P | --- | ND | ND |
| L79K | --- | 477 | ND |
| L79P | --- | ND | ND |
| L79R | --- | 234 | ND |
| D80A | --- | ND | ND |
| F82I | 11 | 9 | 277 |
| F82K | 10 | 15 | ~5000 |
| F82W | --- | 276 | ND |
| F82W / N83A | --- | 389 | ~40000 |
| V99E | --- | ND | ND |
| V99K | --- | ND | --- |

ND: not detectable over concentration range tested --- Not tested

As shown in the table above, single amino acid substitutions in the ActRIIB extracellular domain can alter the balance between activin A or GDF11 inhibition and BMP9 inhibition in a cell-based reporter gene assay. Compared to a fusion protein containing unmodified ActRIIB extracellular domain, the variants ActRIIB(K55A)-Fc, ActRIIB(K55E)-Fc, ActRIIB(F82I)-Fc, and ActRIIB(F82K)-Fc showed less potent inhibition of BMP9 (increased IC₅₀ values) while maintaining essentially undiminished inhibition of activin A and GDF11.

These results indicate that variant ActRIIB-Fc proteins such as ActRIIB(K55A)-Fc, ActRIIB(K55E)-Fc, ActRIIB(F82I)-Fc, and ActRIIB(F82K)-Fc are more selective antagonists of activin A and GDF11compared to an Fc fusion protein comprising unmodified ActRIIB extracellular domain. Accordingly, these variants may be more useful than ActRIIB-Fc in certain applications where such selective antagonism is advantageous. Examples include therapeutic applications where it is desirable to retain antagonism of one or more of activin A, GDF8, and GDF11while reducing antagonism of BMP9 and potentially BMP10.

### Example 7. Generation of an ActRIIB-Fc:ActRIIB(L79E)-Fc Heterodimer

Applicants envision generation of a soluble ActRIIB-Fc:ActRIIB(L79E)-Fc heteromeric complex comprising the extracellular domains of unmodified human ActRIIB and human ActRIIB with a leucine-to-glutamate substitution at position 79, which are each separately fused to an GlFc domain with a linker positioned between the extracellular domain and the GlFc domain. The individual constructs are referred to as ActRIIB-Fc fusion polypeptide and ActRIIB(L79E)-Fc fusion polypeptide, respectively, and the sequences for each are provided below.

A methodology for promoting formation of ActRIIB-Fc:ActRIIB(L79E)-Fc heteromeric complexes, as opposed to the ActRIIB-Fc or ActRIIB(L79E)-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIB(L79E)-Fc and ActRIIB-Fc polypeptide sequences of SEQ ID NOs: 43-45 and 46-48, respectively, one Fc domain can be altered to introduce cationic amino acids at the interaction face, while the other Fc domain can be altered to introduce anionic amino acids at the interaction face. The ActRIIB(L79E)-Fc fusion polypeptide and ActRIIB-Fc fusion polypeptide can each employ the TPA leader (SEQ ID NO: 8).

The ActRIIB(L79E)-Fc polypeptide sequence (SEQ ID NO: 43) is shown below:

1 MDAMKRGLCC VLLLCGAVFV SPGASGRGEA ETRECIYYNA NWELERTNQS
51 GLERCEGEQD KRLHCYASWR NSSGTIELVK KGCWEDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYDTTPPVLD SDGSFFLYSD LTVDKSRWQQ GNVFSCSVMH
351 EALHNHYTQK SLSLSPG (SEQ ID NO: 43)

The leader (signal) sequence and linker are underlined, and the L79E substitution is indicated by double underline. To promote formation of the ActRIIB-Fc:ActRIIB(L79E)-Fc heterodimer rather than either of the possible homodimeric complexes, two amino acid substitutions (replacing lysines with acidic amino acids) can be introduced into the Fc domain of the ActRIIB fusion protein as indicated by double underline above. The amino acid sequence of SEQ ID NO: 43 may optionally be provided with lysine added to the C-terminus. The GIFc region may also comprise a mutation to an alanine at position 234 or a mutation to an alanine at position 235, or a combination thereof.

This ActRIIB(L79E)-Fc fusion protein can be encoded by the following nucleic acid sequence (SEQ ID NO: 44):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGGAAGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACGACA CCACGCCTCC
951 CGTGCTGGAC TCCGACGGCT CCTTCTTCCT CTATAGCGAC CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 T (SEQ ID NO: 44)

The mature ActRIIB(L79E)-Fc fusion polypeptide (SEQ ID NO: 45) is as follows, and may optionally be provided with lysine added to the C-terminus. The GIFc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWE DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYDTT PPVLDSDGSF
301 FLYSDLTVDK SRWQQGNVFS CSVMHEALHN HYTQKSLSLS PG
(SEQ ID NO: 45)

The complementary form of ActRIIB-Fc fusion polypeptide (SEQ ID NO: 46) is as follows:

1 MDAMKRGLCC VLLLCGAVFV SPGASGRGEA ETRECIYYNA NWELERTNQS
51 GLERCEGEQD KRLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSRKEMT KNQVSLTCLV KGFYPSDIAV
301 EWESNGQPEN NYKTTPPVLK SDGSFFLYSK LTVDKSRWQQ GNVFSCSVMH
351 EALHNHYTQK SLSLSPGK (SEQ ID NO: 46)

The leader sequence and linker sequence are underlined. To guide heterodimer formation with the ActRIIB(L79E)-Fc fusion polypeptide of SEQ ID NOs: 43 and 45 above, two amino acid substitutions (replacing a glutamate and an aspartate with lysines) can be introduced into the Fc domain of the ActRIIB-Fc fusion polypeptide as indicated by double underline above. The amino acid sequence of SEQ ID NO: 46 may optionally be provided with lysine removed from the C-terminus. The GIFc region may also comprise a mutation to an alanine at position 234 or a mutation to an alanine at position 235, or a combination thereof.

This ActRIIB-Fc fusion protein can be encoded by the following nucleic acid (SEQ ID NO: 47):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGAA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGAAG TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 47)

The mature ActRIIB-Fc fusion protein sequence (SEQ ID NO: 48) is as follows and may optionally be provided with lysine removed from the C-terminus. The GIFc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 RKEMTKNQVS LTCLVKGFYP SDIAVEWESN GQPENNYKTT PPVLKSDGSF
301 FLYSKLTVDK SRWQQGNVFS CSVMHEALHN HYTQKSLSLS PGK
(SEQ ID NO: 48)

The ActRIIB(L79E)-Fc and ActRIIB-Fc polypeptides of SEQ ID NO: 45 and SEQ ID NO: 48, respectively, may be co-expressed and purified from a CHO cell line, to give rise to a heteromeric protein complex comprising ActRIIB-Fc:ActRIIB(L79E)-Fc.

In another approach to promote the formation of heteromultimer complexes using asymmetric Fc fusion proteins, the Fc domains can be altered to introduce complementary hydrophobic interactions and an additional intermolecular disulfide bond as illustrated in the ActRIIB(L79E)-Fc and ActRIIB-Fc polypeptide sequences of SEQ ID NOs: 49-50 and 51-52, respectively. The ActRIIB(L79E)-Fc fusion polypeptide and ActRIIB-Fc fusion polypeptide can each employ the TPAleader (SEQ ID NO: 8). ActRIIB(L79E)-Fc polypeptide sequence (SEQ ID NO: 49) is shown below:

1 MDAMKRGLCC VLLLCGAVFV SPGASGRGEA ETRECIYYNA NWELERTNQS
51 GLERCEGEQD KRLHCYASWR NSSGTIELVK KGCWEDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPCREEMT KNQVSLWCLV KGFYPSDIAV
301 EWESNGQPEN NYKTTPPVLD SDGSFFLYSK LTVDKSRWQQ GNVFSCSVMH
351 EALHNHYTQK SLSLSPG (SEQ ID NO: 49)

The signal sequence and linker sequence are underlined, and the L79E substitution is indicated by double underline. To promote formation of the ActRIIB-Fc:ActRIIB(L79E)-Fc heterodimer rather than either of the possible homodimeric complexes, two amino acid substitutions (replacing a serine with a cysteine and a threonine with a tryptophan) can be introduced into the Fc domain of the fusion protein as indicated by double underline above. The amino acid sequence of SEQ ID NO: 49 may optionally be provided with lysine added to the C-terminus. The GIFc region may also comprise a mutation to an alanine at position 234 or a mutation to an alanine at position 235, or a combination thereof. Mature ActRIIB(L79E)-Fc fusion polypeptide (SEQ ID NO: 50) is as follows:

1 GRGEAETREC IYYNANWELE RTNQSGLERC EGEQDKRLHC YASWRNSSGT
51 IELVKKGCWE DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPC
251 REEMTKNQVS LWCLVKGFYP SDIAVEWESN GQPENNYKTT PPVLDSDGSF
301 FLYSKLTVDK SRWQQGNVFS CSVMHEALHN HYTQKSLSLS PG
(SEQ ID NO: 50)

The complementary form of ActRIIB-Fc fusion polypeptide (SEQ ID NO: 51) is as follows and may optionally be provided with lysine removed from the C-terminus. The GIFc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVC TLPPSREEMT KNQVSLSCAV KGFYPSDIAV
301 EWESNGQPEN NYKTTPPVLD SDGSFFLVSK LTVDKSRWQQ GNVFSCSVMH
351 EALHNHYTQK SLSLSPGK (SEQ ID NO: 51)

The leader sequence and linker are underlined. To guide heterodimer formation with the ActRIIB(L79E)-Fc fusion polypeptide of SEQ ID NOs: 49-50 above, four amino acid substitutions (replacement of tyrosine with cysteine, threonine with serine, leucine with alanine, and tyrosine with valine) can be introduced into the Fc domain of the ActRIIB-Fc fusion polypeptide as indicated by double underline above. The amino acid sequence of SEQ ID NO: 51 may optionally be provided with lysine removed from the C-terminus. The GIFc region may also comprise a mutation to an alanine at position 234 or a mutation to an alanine at position 235, or a combination thereof.

The mature ActRIIB-Fc fusion protein sequence is as follows and may optionally be provided with lysine removed from the C-terminus.

1 GRGEAETREC IYYNANWELE RTNQSGLERC EGEQDKRLHC YASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVCTLPPS
251 REEMTKNQVS LSCAVKGFYP SDIAVEWESN GQPENNYKTT PPVLDSDGSF
301 FLVSKLTVDK SRWQQGNVFS CSVMHEALHN HYTQKSLSLS PGK
(SEQ ID NO: 52)

The ActRIIB(L79E)-Fc and ActRIIB-Fc polypeptides of SEQ ID NO: 50 and SEQ ID NO: 52, respectively, may be co-expressed and purified from a CHO cell line, to give rise to a heteromeric protein complex comprising ActRIIB-Fc:ActRIIB(L79E)-Fc.

Purification of various ActRIIB-Fc:ActRIIB(L79E)-Fc complexes can be achieved by a series of column chromatography steps, including, for example, three or more of the following, in any order: protein A chromatography, Q sepharose chromatography, phenylsepharose chromatography, size exclusion chromatography, cation exchange chromatography, multimodal chromatography (e.g., with resin containing both electrostatic and hydrophobic ligands), and epitope-based affinity chromatography (e.g., with an antibody or functionally equivalent ligand directed against an epitope of ActRIIB). The purification can be completed with viral filtration and buffer exchange.

### Example 8. Ligand Binding Profile of ActRIIB-Fc:ActRIIB(L79E)-Fc Heteromer

A Biacore™-based binding assay was used to compare the ligand binding kinetics of an ActRIIB-Fc:ActRIIB(L79E)-Fc heterodimer with those of unmodified ActRIIB-Fc homodimer. Fusion proteins were captured onto the system using an anti-Fc antibody. Ligands were then injected and allowed to flow over the captured receptor protein at 37° C. Results are summarized in the table below, in which ligand off-rates (k_{d}) most indicative of effective ligand traps are denoted in bold.

Ligand binding of ActRIIB-Fc:ActRIIB(L79E)-Fc heterodimer compared to ActRII-Fc homodimer at 37° C.

| Ligand | ActRIIB-Fc homodimer | | | ActRIIB-Fc:ActRIIB(L79E)-Fc heterodimer | | |
|---|---|---|---|---|---|---|
| | kₐ (1/Ms) | k_{d} (1/s) | K_{D} (pM) | kₐ (1/Ms) | k_{d} (1/s) | K_{D} (pM) |
| Activin A | 7.4 x10⁶ | 1.9 x10⁻⁴ | 25 | 8.8 x10⁶ | 1.5 x10⁻³ | 170 |
| Activin B | 8.1 x10⁶ | 6.6 x10⁻⁵ | 8 | 8.3 x10⁶ | 2.1 x10⁻⁴ | 25 |
| GDF3 | 1.4 x10⁶ | 2.2 x10⁻³ | 1500 | 5.8 x10⁵ | 5.9 x10⁻³ | 10000 |
| GDF8 | 3.8 x10⁶ | 2.6 x10⁻⁴ | 70 | 3.4 x10⁶ | 5.0 x10⁻⁴ | 150 |
| GDF11 | 4.1 x10⁷ | 1.7 x10⁻⁴ | 4 | 4.0 x10⁷ | 3.6 x10⁻⁴ | 9 |
| BMP6 | 1.3 x10⁸ | 7.4 x10⁻³ | 56 | 3.3 x10⁸ | 1.8 x10⁻² | 56 |
| BMP9 | 5.0 x10⁶ | 1.3 x10⁻³ | 250 | Transient* | | >2800 |
| BMP10 | 5.1 x10⁷ | 2.0 x10⁻⁴ | 4 | 4.8 x10⁷ | 2.0 x10⁻³ | 42 |

* Indeterminate due to transient nature of interaction

In this example, a single amino acid substitution in one of two ActRIIB polypeptide chains altered ligand binding selectivity of the Fc-fusion protein relative to unmodified ActRIIB-Fc homodimer. Compared to ActRIIB-Fc homodimer, the ActRIIB(L79E)-Fc heterodimer largely retained high-affinity binding to activin B, GDF8, GDF11, and BMP6 but exhibited approximately ten-fold faster off-rates for activin A and BMP10 and an even greater reduction in the strength of binding to BMP9. Accordingly, a variant ActRIIB-Fc heteromer may be more useful than unmodified ActRIIB-Fc homodimer in certain applications where such selective antagonism is advantageous. Examples include therapeutic applications where it is desirable to retain antagonism of one or more of activin B, GDF8, GDF11, and BMP6, while reducing antagonism of activin A, BMP9, or BMP10.9. Generation of ActRIIB mutants:

A series of mutations in the extracellular domain of ActRIIB were generated and these mutant proteins were produced as soluble fusion proteins between extracellular ActRIIB and an Fc domain. A co-crystal structure of Activin and extracellular ActRIIB did not show any role for the final (C-terminal) 15 amino acids (referred to as the “tail” herein) of the extracellular domain in ligand binding. This sequence failed to resolve on the crystal structure, suggesting that these residues are present in a flexible loop that did not pack uniformly in the crystal. ThompsonEMBO J. 2003 Apr 1;22(7): 1555-66. This sequence is also poorly conserved between ActRIIB and ActRIIA. Accordingly, these residues were omitted in the basic, or background, ActRIIB-Fc fusion construct. Additionally, in this example position 64 in the background form is occupied by an alanine. Thus, the background ActRIIB-Fc fusion in this example has the sequence (Fc portion underlined)(SEQ ID NO: 54):

SGRGEAETRECIYYNANWELERTNQSGLERCEGEQDKRLHCYAS
WANSSGTIELVKKGCWLDDFNCYDRQECVATEENPQVYFCCCEGNFCNE
RFTHLPEAGGGTHTCPPCPAPELLGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALPVPIEKTISKAKGQPREPQVYTLPPSREEMTKN
QVSLTCLVKGFYPSDIAVEWESNGQPENNYKTTPPVLDSDGSFFLYSKLT
VDKSRWQQGNVFSCSVMHEALHNHYTQKSLSLSPGK

Surprisingly, as discussed below, the C-terminal tail was found to enhance activin and GDF-11 binding, thus a preferred version of ActRIIB-Fc has a sequence (Fc portion underlined)(SEQ ID NO: 55):

SGRGEAETRECIYYNANWELERTNQSGLERCEGEQDKRLHCYAS
WANSSGTIELVKKGCWLDDFNCYDRQECVATEENPQVYFCCCEGNFCNER
FTHLPEAGGPEVTYEPPPTAPTGGGTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPVPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK

Various mutations were introduced into the background ActRIIB-Fc protein. Mutations were generated in ActRIIB extracellular domain by PCR mutagenesis. After PCR, fragments were purified thru Qiagen column, digested with SfoI and AgeI and gel purified. These fragments were ligated into expression vector pAID4 such that upon ligation it created fusion chimera with human IgG1. DNAs were isolated. All of the mutants were produced in HEK293T cells by transient transfection. In summary, in a 500 ml spinner, HEK293T cells were set up at 6x10⁵ cells/ml in Freestyle (Invitrogen) media in 250 ml volume and grown overnight. Next day, these cells were treated with DNA:PEI (1:1) complex at 0.5 ug/ml final DNA concentration. After 4 hrs, 250 ml media was added and cells were grown for 7 days. Conditioned media was harvested by spinning down the cells and concentrated.

All the mutants were purified over protein A column and eluted with low pH (3.0) glycine buffer . After neutralization, these were dialyzed against PBS.

Mutants were also produced in CHO cells by similar methodology.

Mutants were tested in binding assays and bioassays described below. Proteins expressed in CHO cells and HEK293 cells were indistinguishable in the binding assays and bioassays.

### Example 9. ActRIIB (F82K)-mFc Treatment Suppresses Kidney Fibrosis and Inflammation and Reduces Kidney Injury

The effects of variant ActRIIB F82K mFc fusion (ActRIIB (F82K)-mFc), described in Example 5 on kidney disease was assessed in a mouse unilateral ureteral obstruction model. See, e.g., Klahr and Morrissey (2002) Am J Physiol Renal Physiol 283: F861-F875.

Sixteen C57BL/6 male mice 12 weeks of age underwent left unilateral ureteral ligation twice at the level of the lower pole of kidney. After 3 days, mice were randomized into two groups: i) eight mice were injected subcutaneously with vehicle control, phosphate buffered saline (PBS), at day 3, day 7, day 10, and day 14 after surgery (“UUO/PBS”), and ii) eight mice were injected subcutaneously with ActRIIB (F82K)-mFc at a dose of 10 mg/kg at day 3, day 7, day 10, and day 14 after surgery (“UUO/F82K”). Both groups were sacrificed at day 17 in accordance with the relevant Animal Care Guidelines. Half kidneys from individual animals were collected for histology analysis (H&E, and Masson’s Trichrome stain), from both the UUO kidney and contralateral kidney, and ¼ kidneys were used for RNA extraction (RNeasy Midi Kit, Qiagen, IL).

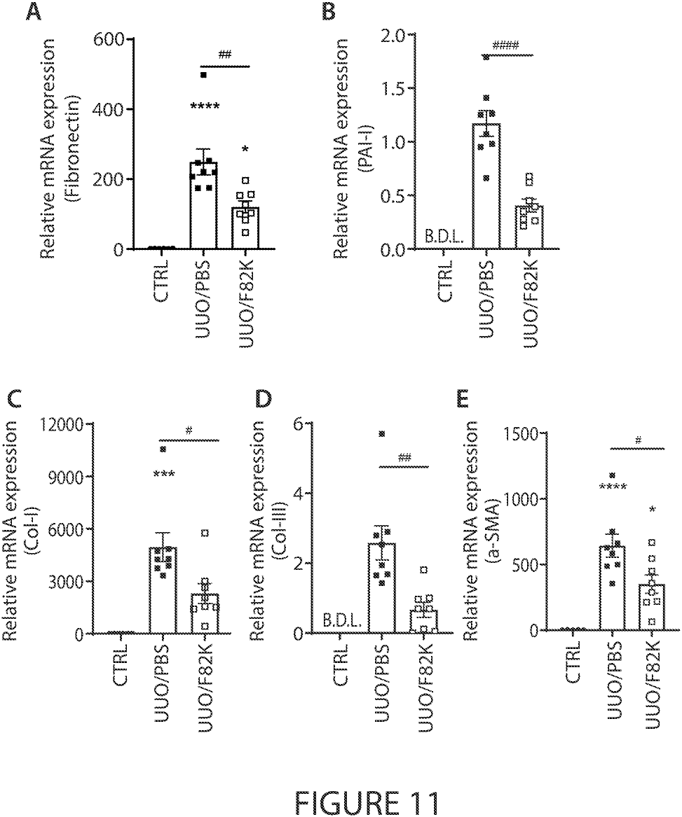

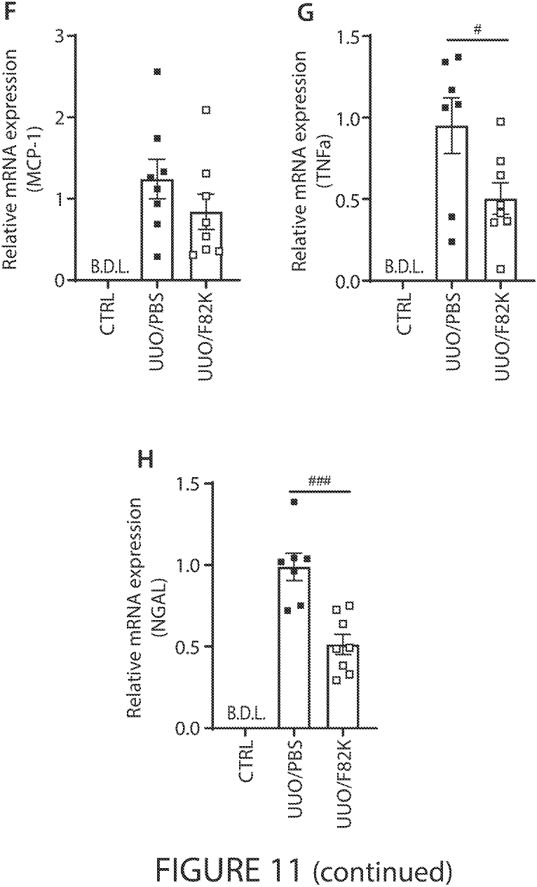

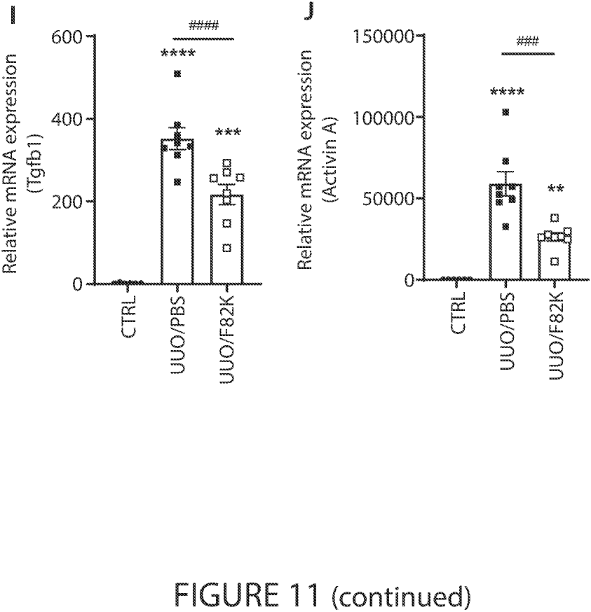

Gene expression analysis on UUO kidney samples was performed to assess levels of various genes. QRT-PCR was performed on a CFX Connect™ Real-time PCR detection system (Bio-Rad, CA) to evaluate the expression of various fibrotic genes (Col1a1, Co13al, Fibronectin, PAI-1, and a-SMA), inflammatory genes (Tnfa, and MCP1), cytokines (TGFβ1 and activin A), and kidney injury genes (NGAL). See FIGS. 11A-11J. Treatment of mice with ActRIIB (F82K)-mFc significantly suppressed the expression of fibrotic and inflammatory genes, inhibited the upregulation of TGFβ 1 and Activin A, and reduced kidney injury.

Together, these data demonstrate that ActRIIB (F82K)-mFc treatment suppresses kidney fibrosis and inflammation and reduces kidney injury. Moreover, these data indicate that other ActRIIB-Fc variants may be useful in the treatment or preventing of kidney disease including, for example, ActRIIB variants that maintain strong binding to activin A, GDF11, and BMP10, but have diminished binding to BMP9.

### Example 10. ActRIIB (F82K)-mFc Treatment Reduces Albuminuria and Improves Renal Function in Alport Mouse Model

The effects of variant ActRIIB F82K mFc fusion (“ActRIIB (F82K)-mFc”) in Example 5 on kidney disease was assessed in a mouse Alport model (Col4a3-/-). See, e.g., Cosgrove D, et al (1996) Genes Dev 10(23): 2981-92.

Fourteen Col4a3-/- mice 4 weeks of age were randomized into two groups: i) eight mice were injected subcutaneously with vehicle control, phosphate buffered saline (PBS), twice a week (“Col4a3-Veh”), and a ii) six mice were injected subcutaneously with ActRIIB (F82K)-mFc at a dose of 10 mg/kg twice a week (“Col4a3-F82K”). Urine samples were collected from normal mice (WT), Col4a3-/- mice, and Col4a3-/- mice treated with ActRIIB (F82K)-mFc on the day before treatment starts (4 weeks), day 49 (7 weeks), and day 63 (9 weeks) to measure albumin (mouse albumin ELISA kit, Molecular Innovations, MI) and creatinine (creatinine assay kit, BioAssay Systems, CA). Both groups were sacrificed at day 63 (9 weeks) in accordance with the relevant Animal Care Guidelines. One kidneys from individual animals were collected for histology analysis (H&E, and Masson’s Trichrome stain), and the other kidneys were snap-frozen at -80° C.

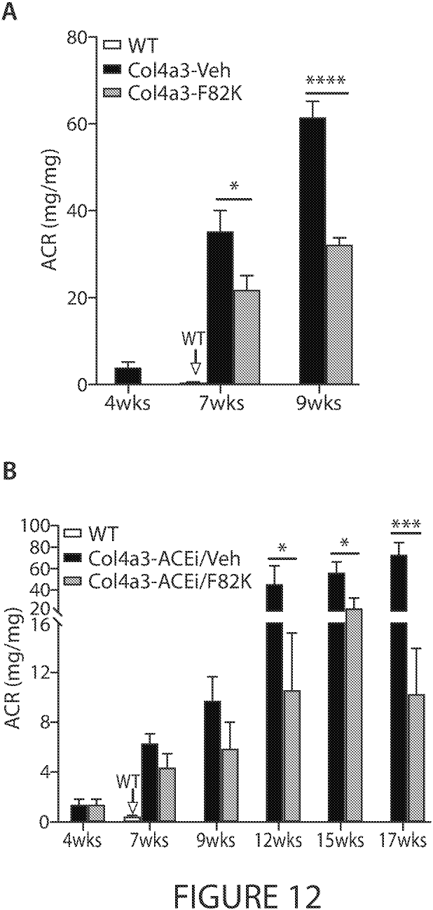

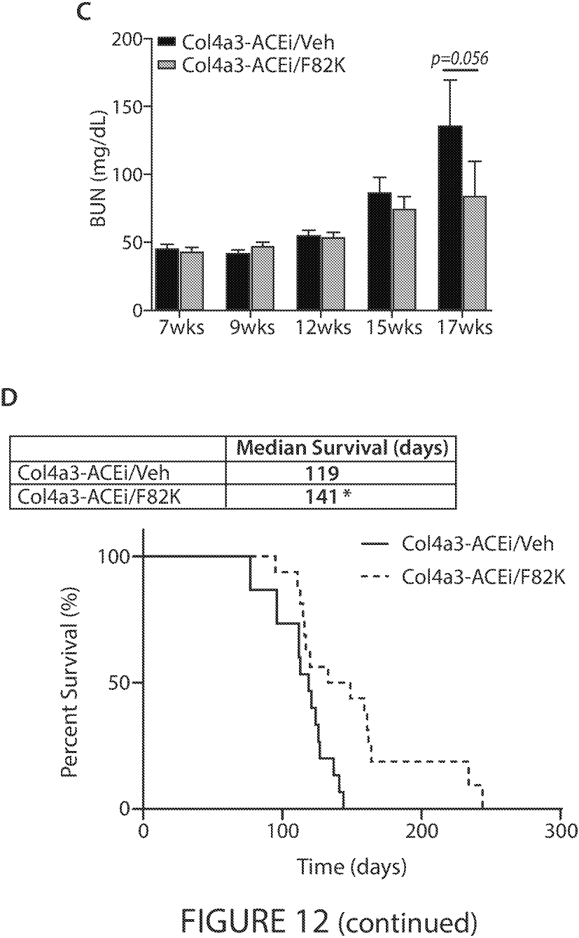

Urinary albumin to creatinine ratio (ACR) was calculated to measure albuminuria. See FIG. 12A. Albuminuria was significantly increased from 4 weeks to 9 weeks in Col4a3-/- mice. Treatment of mice with ActRIIB (F82K)-mFc significantly reduced albuminuria by 38.9% (p<0.05) at 7 weeks, and 45.1% (p<0.001) at 9 weeks in Col4a3-/-mice.

To evaluate the therapeutic benefits of ActRIIB (F82K)-mFc in the presence of angiotensin-converting enzyme inhibitor (ACEi) in Alport model, ActRIIB (F82K)-mFc was further assessed in Col4a3-/- mice treated with Ramipril. See, e.g., Gross O, et al (2003) Kidney Int. 63(2): 438-46.

Thirty-one Col4a3-/- mice 4 weeks of age were fed with Ramipril (10 mg/kg/day) in drinking water throughout the study, and randomized into two groups: i) sixteen mice were injected subcutaneously with vehicle control, phosphate buffered saline (PBS), twice a week (Col4a3-ACEi/Veh″), and ii) fifteen mice were injected subcutaneously with ActRIIB (F82K)-mFc at a dose of 10 mg/kg twice a week (“Col4a3-ACEi/F82K”). Urine samples were collected from normal mice (WT), Col4a3-/- mice, and Col4a3-/- mice treated with ActRIIB (F82K)-mFc on the day before treatment starts (4 weeks), day 49 (7 weeks), day 63 (9 weeks), day 84 (12 weeks), day 105 (15 weeks), and day 119 (17 weeks) to measure albumin (mouse albumin ELISA kit, Molecular Innovations, MI) and creatinine (creatinine assay kit, BioAssay Systems, CA). Blood samples were collected from normal mice (WT), Co14a3-/-mice, and Co14a3-/- mice treated with ActRIIB (F82K)-mFc on day 49 (7 weeks), day 63 (9 weeks), day 84 (12 weeks), day 105 (15 weeks), and day 119 (17 weeks) for blood urea nitrogen (BUN) measurement (DRI-CHEM 7000 chemistry analyzer, HESKA, CO). Mice in both groups were euthanized when body weight loss was more than 25% to calculate the survival time in accordance with the relevant Animal Care Guidelines.

Urinary albumin to creatinine ratio (ACR) was calculated to measure albuminuria. See FIG. 12B. Albuminuria was significantly increased from 4 weeks to 17 weeks in Co14a3-/- mice treated with Ramipril. Treatment of these mice with ActRIIB (F82K)-mFc significantly reduced albuminuria by 76.7%% (p<0.05) at 12 weeks, 59% (p<0.05) at 15 weeks, and 86% (p<0.001) at 17 weeks respectively in Co14a3-/- mice, which was associated with decreased BUN in Co14a3-/- mice (FIG. 12C).

Moreover, ActRIIB (F82K)-mFc significantly increased life span in Co14a3-/- mice treated with Ramipril (p<0.05), with a median survival time of 141 days in the mice treated with ActRIIB (F82K)-mFc and 119 days in the cohort treated with PBS (FIG. 12D).

Together, these data demonstrate that ActRIIB (F82K)-mFc treatment reduces albuminuria, improves renal function, and increases life span in Alport mouse model either as a monotherapy or on top of Ramipril treatment. Moreover, these data indicate that ActRIIB (F82K)-mFc and other ActRIIB-mFc variants may be useful in the treatment or preventing of kidney disease including, for example, ActRIIB variants that maintain strong binding to activin A, GDF11, and BMP10, but have diminished binding to BMP9.

### Example 11. ActRIIB (K55A)-mFc, ActRIIB (K55E)-mFc, and ActRIIB (F82I)-mFc Treatment Suppresses Kidney Fibrosis and Inflammation and Reduces Kidney Injury

The effects of the ActRIIB (K55A)-mFc, ActRIIB (K55E)-mFc, and ActRIIB (F82I)-mFc described in Example 5 on kidney disease was assessed in a mouse unilateral ureteral obstruction model. See, e.g., Klahr and Morrissey (2002) Am J Physiol Renal Physiol 283: F861-F875.

Thirty-Two C57BL/6 male mice 12 weeks of age underwent left unilateral ureteral ligation twice at the level of the lower pole of kidney. After 3 days, mice were randomized into four groups: i) eight mice were injected subcutaneously with vehicle control, phosphate buffered saline (PBS), at day 3, day 7, day 10, and day 14 after surgery (“UUO/PBS”), ii)eight mice were injected subcutaneously with ActRIIB (K55A)-mFc at a dose of 10 mg/kg at day 3, day 7, day 10, and day 14 after surgery, iii) eight mice were injected subcutaneously with ActRIIB (K55E)-mFc at a dose of 10 mg/kg at day 3, day 7, day 10, and day 14 after surgery, and iv) eight mice were injected subcutaneously with ActRIIB (F82I)-mFc at a dose of 10 mg/kg at day 3, day 7, day 10, and day 14 after surgery. All groups were sacrificed at day 17 in accordance with the relevant Animal Care Guidelines. Half kidneys from individual animals were collected for histology analysis (H&E, and Masson’s Trichrome stain), from both the UUO kidney and contralateral kidney, and ¼ kidneys were used for RNA extraction (RNeasy Midi Kit, Qiagen, IL).

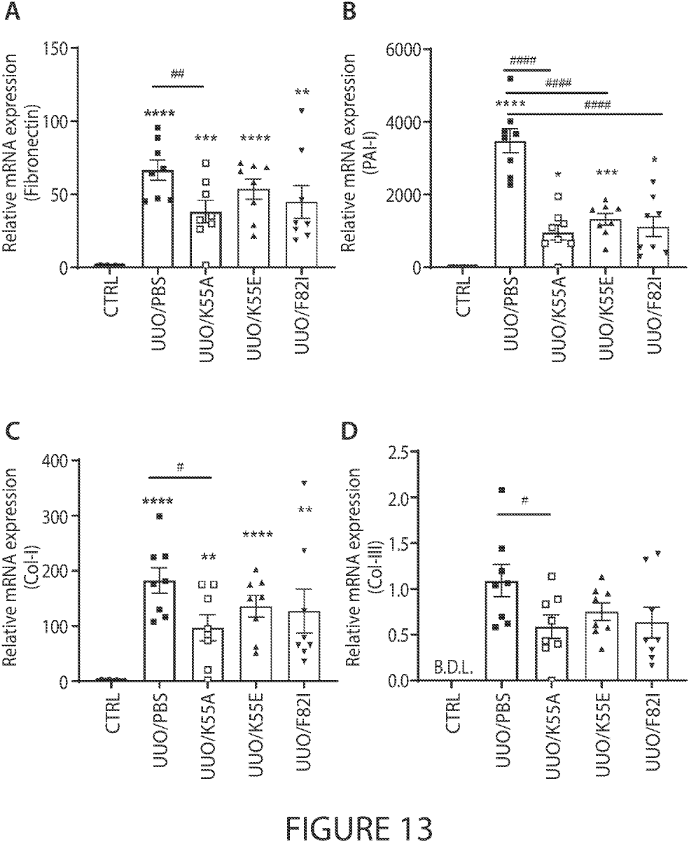

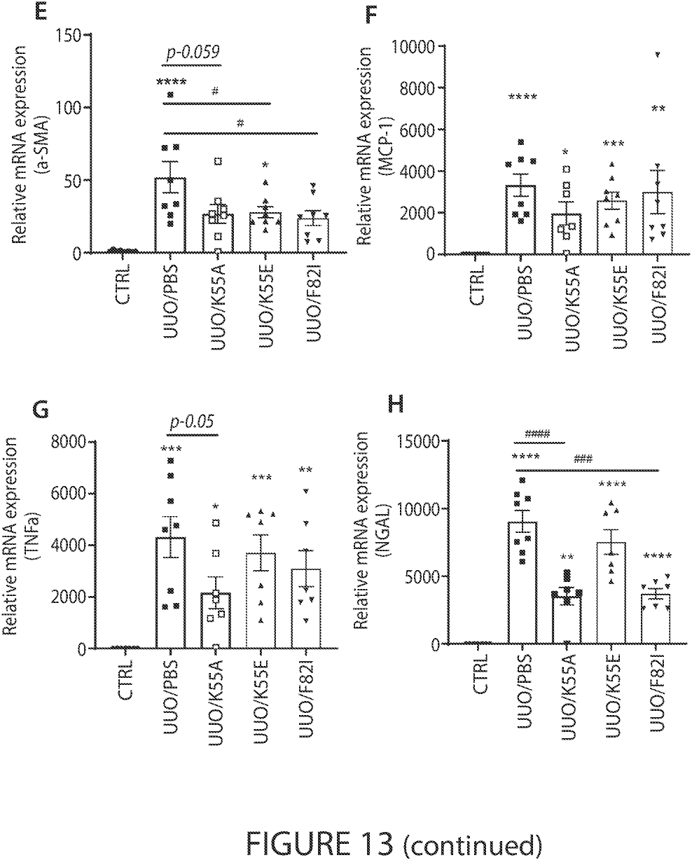

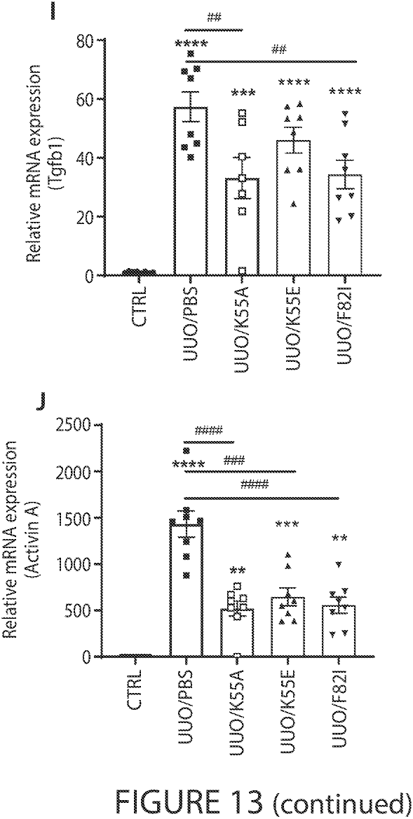

Gene expression analysis on UUO kidney samples was performed to assess levels of various genes. QRT-PCR was performed on a CFX Connect™ Real-time PCR detection system (Bio-Rad, CA) to evaluate the expression of various fibrotic genes (Col1a1, Co13a1, Fibronectin, PAI-1, and a-SMA), inflammatory genes (Tnfa, and MCP1), cytokines (TGFβ1 and activin A), and kidney injury genes (NGAL). See FIGS. 13A-J.

Treatment of mice with ActRIIB (K55A)-mFc significantly suppressed the expression of fibrotic and inflammatory genes, inhibited the upregulation of TGFβ 1 and Activin A, and reduced kidney injury. Treatment of mice with ActRIIB (K55E)-mFc significantly suppressed the expression of PAI-1 and a-SMA, but not Col1a1, Co13a1,and Fibronectin. In addition, treatment of mice with ActRIIB (K55E)-mFc did not significantly suppress inflammatory genes expression, nor reduce kidney injury. Treatment of mice with ActRIIB (F82I)-mFc significantly suppressed the expression of PAI-1 and a-SMA, but not Col1a1, Co13a1,and Fibronectin. In addition, treatment of mice with ActRIIB (F82I)-mFc significantly inhibited the upregulation of TGFβ 1 and Activin A, and reduced kidney injury. However, ActRIIB (F82I)-mFc did not significantly suppress inflammatory genes expression.

Together, these data demonstrate that ActRIIB (K55A)-mFc is more potent than ActRIIB (K55E)-mFc and ActRIIB (F82I)-mFc to suppress kidney fibrosis and inflammation and reduce kidney injury in a UUO model.

### Example 12: Generation of Variant ActRIIB-Fc Proteins

Applicants generated a series of mutations (sequence variations) in the extracellular domain of ActRIIB and produced these variant polypeptides as soluble homodimeric fusion proteins comprising a variant ActRIIB extracellular domain and an Fc domain joined by an optional linker. The background ActRIIB-Fc fusion was ActRIIB-G1Fc as shown in SEQ ID NO: 5.

Various substitution mutations were introduced into the background ActRIIB-Fc protein. Based on the data presented in Example 1, it is expected that these constructs, if expressed with a TPA leader, may lack the N-terminal serine. Mutations were generated in the ActRIIB extracellular domain by gene synthesis at Twist Bioscience or Genscript. Fragments were ligated into the NheI and AgeI sites of the . expression vector pAID4 (see WO2006/012627) such that upon ligation it created fusion chimera with human IgG1 Fc domain. Upon transformation into E. coli DH5 alpha, colonies were picked and DNA was isolated. All mutants were sequence verified.

The amino acid sequence of unprocessed ActRIIB(N35E)-G1Fc is shown below (SEQ ID NO: 366). The signal sequence and linker sequences are indicated by solid underline, and the N35E substitution is indicated by double underline. The amino acid sequence of SEQ ID NO: 366 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA EWELERTNQS
51 GLERCEGEQD KRLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 366)

This ActRIIB(N35E)-G1Fc fusion polypeptide is encoded by the following nucleic acid sequence (SEQ ID NO: 367):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC GAGTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 367)

A mature ActRIIB(N35E)-G1Fc fusion polypeptide (SEQ ID NO: 368) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNAEWELE RTNQSGLERC EGEQDKRLHC YASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 368)

The amino acid sequence of unprocessed ActRIIB(E52N)-G1Fc is shown below (SEQ ID NO: 369). The signal sequence and linker sequences are indicated by solid underline, and the E52N substitution is indicated by double underline. The amino acid sequence of SEQ ID NO: 369 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGNQD KRLHCYASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 369)

This ActRIIB(E52N)-G1Fc fusion polypeptide is encoded by the following nucleic acid sequence (SEQ ID NO: 370):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CAACCAGGAC AAGCGGCTGC ACTGCTACGC
201 CTCCTGGCGC AACAGCTCTG GCACCATCGA GCTCGTGAAG AAGGGCTGCT
251 GGCTAGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 370)

A mature ActRIIB(E52N)-G1Fc fusion polypeptide (SEQ ID NO: 371) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGNQDKRLHC YASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 371)

The amino acid sequence of unprocessed ActRIIB(Y60D)-G1Fc is shown below (SEQ ID NO: 372). The signal sequence and linker sequences are indicated by solid underline, and the Y60D substitution is indicated by double underline. The amino acid sequence of SEQ ID NO: 372 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCDASWR NSSGTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 372)

This ActRIIB(Y60D)-G1Fc fusion polypeptide is encoded by the following nucleic acid sequence (SEQ ID NO: 373):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCGACGC
201 CTCCTGGCGC AACAGCTCTG GCACCATCGA GCTCGTGAAG AAGGGCTGCT
251 GGCTAGATGA CTTTAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 373)

A mature ActRIIB(Y60D)-G1Fc fusion polypeptide (SEQ ID NO: 374) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC DASWRNSSGT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 374)

The amino acid sequence of unprocessed ActRIIB(G68R)-G1Fc is shown below (SEQ ID NO: 375). The signal sequence and linker sequences are indicated by solid underline, and the G68R substitution is indicated by double underline. The amino acid sequence of SEQ ID NO: 375 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSRTIELVK KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 375)

This ActRIIB(G68R)-G1Fc fusion polypeptide is encoded by the following nucleic acid sequence (SEQ ID NO: 376):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTA GGACCATCGA GCTCGTGAAG AAGGGCTGCT
251 GGCTAGATGA CTTTAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 376)

A mature ActRIIB(G68R)-G1Fc fusion polypeptide (SEQ ID NO: 377) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSRT
51 IELVKKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 377)

The amino acid sequence of unprocessed ActRIIB(K74E)-G1Fc is shown below (SEQ ID NO: 378). The signal sequence and linker sequences are indicated by solid underline, and the K74E substitution is indicated by double underline. The amino acid sequence of SEQ ID NO: 378 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVE KGCWLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 378)

This ActRIIB(K74E)-G1Fc fusion polypeptide is encoded by the following nucleic acid sequence (SEQ ID NO: 379):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGGAG AAGGGCTGCT
251 GGCTAGATGA CTTTAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 379)

A mature ActRIIB(K74E)-G1Fc fusion polypeptide (SEQ ID NO: 380) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVEKGCWL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 380)

The amino acid sequence of unprocessed ActRIIB(W78Y)-G1Fc is shown below (SEQ ID NO: 381). The signal sequence and linker sequences are indicated by solid underline, and the W78Y substitution is indicated by double underline. The amino acid sequence of SEQ ID NO: 381 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCYLDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 381)

This ActRIIB(W78Y)-G1Fc fusion polypeptide is encoded by the following nucleic acid sequence (SEQ ID NO: 382):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 ACCTAGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 382)

A mature ActRIIB(W78Y)-G1Fc fusion polypeptide (SEQ ID NO: 383) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCYL DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 383)

The amino acid sequence of unprocessed ActRIIB (L79A)-G1Fc is shown below (SEQ ID NO: 384). The signal sequence and linker sequences are indicated by solid underline, and the L79A substitution is indicated by double underline. The amino acid sequence of SEQ ID NO: 384 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWADDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 384)

This ActRIIB (L79A)-G1Fc fusion polypeptide is encoded by the following nucleic acid sequence (SEQ ID NO: 385):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGGCCGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCCCCGGG
1101 TAAA (SEQ ID NO: 385)

A mature ActRIIB(L79A)-G1Fc fusion polypeptide (SEQ ID NO: 386) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWA DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 386)

The amino acid sequence of unprocessed ActRIIB(L79K)-G1Fc is shown below (SEQ ID NO: 387). The signal sequence and linker sequences are indicated by solid underline, and the L79K substitution is indicated by double underline. The amino acid sequence of SEQ ID NO: 387 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWKDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 387)

This ActRIIB(L79K)-G1Fc fusion polypeptide is encoded by the following nucleic acid sequence (SEQ ID NO: 388):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGAAGGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 388)

A mature ActRIIB(L79K)-G1Fc fusion polypeptide (SEQ ID NO: 389) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWK DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 389)

The amino acid sequence of unprocessed ActRIIB(L79S)-G1Fc is shown below (SEQ ID NO: 390). The signal sequence and linker sequences are indicated by solid underline, and the L79S substitution is indicated by double underline. The amino acid sequence of SEQ ID NO: 390 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWSDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 390)

This ActRIIB(L79S)-G1Fc fusion polypeptide is encoded by the following nucleic acid sequence (SEQ ID NO: 391):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGAGCGATGA CTTCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 391)

A mature ActRIIB(L79S)-G1Fc fusion polypeptide (SEQ ID NO: 392) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWS DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 392)

The amino acid sequence of unprocessed ActRIIB(L79W)-G1Fc is shown below (SEQ ID NO: 393). The signal sequence and linker sequences are indicated by solid underline, and the L79W substitution is indicated by double underline. The amino acid sequence of SEQ ID NO: 393 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWWDDFNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 393)

This ActRIIB(L79W)-G1Fc fusion polypeptide is encoded by the following nucleic acid sequence (SEQ ID NO: 394):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGTGGGATGA CTTTAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 394)

A mature ActRIIB(L79W)-G1Fc fusion polypeptide (SEQ ID NO: 395) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWW DDFNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 395)

The amino acid sequence of unprocessed ActRIIB(F82D)-G1Fc is shown below (SEQ ID NO: 396). The signal sequence and linker sequences are indicated by solid underline, and the F82D substitution is indicated by double underline. The amino acid sequence of SEQ ID NO: 396 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDDNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 396)

This ActRIIB(F82D)-G1Fc fusion polypeptide is encoded by the following nucleic acid sequence (SEQ ID NO: 397):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CGACAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 397)

A mature ActRIIB(F82D)-G1Fc fusion polypeptide (SEQ ID NO: 398) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDDNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 398)

The amino acid sequence of unprocessed ActRIIB(F82E)-G1Fc is shown below (SEQ ID NO: 399). The signal sequence and linker sequences are indicated by solid underline, and the F82E substitution is indicated by double underline. The amino acid sequence of SEQ ID NO: 399 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDENC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 399)

This ActRIIB(F82E)-G1Fc fusion polypeptide is encoded by the following nucleic acid sequence (SEQ ID NO: 400):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CGAGAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 400)

A mature ActRIIB(F82E)-G1Fc fusion polypeptide (SEQ ID NO: 401) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDENCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 401)

The amino acid sequence of unprocessed ActRIIB(F82L)-G1Fc is shown below (SEQ ID NO: 402). The signal sequence and linker sequences are indicated by solid underline, and the F82L substitution is indicated by double underline. The amino acid sequence of SEQ ID NO: 402 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDLNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 402)

This ActRIIB(F82L)-G1Fc fusion polypeptide is encoded by the following nucleic acid sequence (SEQ ID NO: 403):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CCTGAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 403)

A mature ActRIIB(F82L)-G1Fc fusion polypeptide (SEQ ID NO: 404) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDLNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 404)

The amino acid sequence of unprocessed ActRIIB(F82S)-G1Fc is shown below (SEQ ID NO: 405). The signal sequence and linker sequences are indicated by solid underline, and the F82S substitution is indicated by double underline. The amino acid sequence of SEQ ID NO: 405 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDSNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 405)

This ActRIIB(F82S)-G1Fc fusion polypeptide is encoded by the following nucleic acid sequence (SEQ ID NO: 406):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CTCCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 406)

A mature ActRIIB(F82S)-G1Fc fusion polypeptide (SEQ ID NO: 407) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDSNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 407)

The amino acid sequence of unprocessed ActRIIB(F82Y)-G1Fc is shown below (SEQ ID NO: 408). The signal sequence and linker sequences are indicated by solid underline, and the F82Y substitution is indicated by double underline. The amino acid sequence of SEQ ID NO: 408 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDYNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 408)

This ActRIIB(F82Y)-G1Fc fusion polypeptide is encoded by the following nucleic acid sequence (SEQ ID NO: 409):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CTACAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 409)

A mature ActRIIB(F82Y)-G1Fc fusion polypeptide (SEQ ID NO: 410) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDYNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 410)

The amino acid sequence of unprocessed ActRIIB(E94K)-G1Fc is shown below (SEQ ID NO: 411). The signal sequence and linker sequences are indicated by solid underline, and the E94K substitution is indicated by double underline. The amino acid sequence of SEQ ID NO: 411 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDFNC YDRQECVATK
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 411)

This ActRIIB(E94K)-G1Fc fusion polypeptide is encoded by the following nucleic acid sequence (SEQ ID NO: 412):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CTTCAACTGC TACGATAGGC AGGAGTGTGT GGCCACTAAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 412)

A mature ActRIIB(E94K)-G1Fc fusion polypeptide (SEQ ID NO: 413) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDFNCYDRQE CVATKENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 413)

The amino acid sequence of unprocessed ActRIIB(E52D, F82D)-G1Fc is shown below (SEQ ID NO: 414). The signal sequence and linker sequences are indicated by solid underline, and the E52D, F82D substitutions are indicated by double underline. The amino acid sequence of SEQ ID NO: 414 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGDQD KRLHCYASWR NSSGTIELVK KGCWLDDDNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 414)

This ActRIIB(E52D, F82D)-G1Fc fusion polypeptide is encoded by the following nucleic acid sequence (SEQ ID NO: 415):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGACCAGGAC AAGCGGCTGC ACTGCTACGC
201 CTCCTGGCGC AACAGCTCTG GCACCATCGA GCTCGTGAAG AAGGGCTGCT
251 GGCTAGATGA CGACAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 415)

A mature ActRIIB(E52D, F82D)-G1Fc fusion polypeptide (SEQ ID NO: 416) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGDQDKRLHC YASWRNSSGT
51 IELVKKGCWL DDDNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 416)

The amino acid sequence of unprocessed ActRIIB(E52D, F82T)-G1Fc is shown below (SEQ ID NO: 417). The signal sequence and linker sequences are indicated by solid underline, and the E52D, F82T substitutions are indicated by double underline. The amino acid sequence of SEQ ID NO: 417 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGDQD KRLHCYASWR NSSGTIELVK KGCWLDDTNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 417)

This ActRIIB(E52D, F82T)-G1Fc fusion polypeptide is encoded by the following nucleic acid sequence (SEQ ID NO: 418):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGACCAGGAC AAGCGGCTGC ACTGCTACGC
201 CTCCTGGCGC AACAGCTCTG GCACCATCGA GCTCGTGAAG AAGGGCTGCT
251 GGCTAGATGA CACCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 418)

A mature ActRIIB(E52D, F82T)-G1Fc fusion polypeptide (SEQ ID NO: 419) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGDQDKRLHC YASWRNSSGT
51 IELVKKGCWL DDTNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 419)

The amino acid sequence of unprocessed ActRIIB(L57R, F82D)-G1Fc is shown below (SEQ ID NO: 420). The signal sequence and linker sequences are indicated by solid underline, and the L57R, F82D substitutions are indicated by double underline. The amino acid sequence of SEQ ID NO: 420 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRRHCYASWR NSSGTIELVK KGCWLDDDNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 420)

This ActRIIB(L57R, F82D)-G1Fc fusion polypeptide is encoded by the following nucleic acid sequence (SEQ ID NO: 421):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGAGGC ACTGCTACGC
201 CTCCTGGCGC AACAGCTCTG GCACCATCGA GCTCGTGAAG AAGGGCTGCT
251 GGCTAGATGA CGACAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 421)

A mature ActRIIB(L57R, F82D)-G1Fc fusion polypeptide (SEQ ID NO:) 422 is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRRHC YASWRNSSGT
51 IELVKKGCWL DDDNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 422)

The amino acid sequence of unprocessed ActRIIB(L57R, F82S)-G1Fc is shown below (SEQ ID NO: 423). The signal sequence and linker sequences are indicated by solid underline, and the L57R, F82S substitutions are indicated by double underline. The amino acid sequence of SEQ ID NO: 423 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRRHCYASWR NSSGTIELVK KGCWLDDSNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 423)

This ActRIIB(L57R, F82S)-G1Fc fusion polypeptide is encoded by the following nucleic acid sequence (SEQ ID NO: 424):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGAGGC ACTGCTACGC
201 CTCCTGGCGC AACAGCTCTG GCACCATCGA GCTCGTGAAG AAGGGCTGCT
251 GGCTAGATGA CTCCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 424)

A mature ActRIIB(L57R, F82S)-G1Fc fusion polypeptide (SEQ ID NO: 425) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRRHC YASWRNSSGT
51 IELVKKGCWL DDSNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 425)

The amino acid sequence of unprocessed ActRIIB(L57R, F82T)-G1Fc is shown below (SEQ ID NO: 426). The signal sequence and linker sequences are indicated by solid underline, and the L57R, F82T substitutions are indicated by double underline. The amino acid sequence of SEQ ID NO: 426 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRRHCYASWR NSSGTIELVK KGCWLDDTNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 426)

This ActRIIB(L57R, F82T)-G1Fc fusion polypeptide is encoded by the following nucleic acid sequence (SEQ ID NO: 427):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCGGC ACTGCTACGC
201 CTCCTGGCGC AACAGCTCTG GCACCATCGA GCTCGTGAAG AAGGGCTGCT
251 GGCTAGATGA CACCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 427)

A mature ActRIIB(L57R, F82T)-G1Fc fusion polypeptide (SEQ ID NO: 428) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRRHC YASWRNSSGT
51 IELVKKGCWL DDTNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 428)

The amino acid sequence of unprocessed ActRIIB(L79F, F82D)-G1Fc is shown below (SEQ ID NO: 429). The signal sequence and linker sequences are indicated by solid underline, and the L79F, F82D substitution is indicated by double underline. The amino acid sequence of SEQ ID NO: 429 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWFDDDNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 429)

This ActRIIB(L79F, F82D)-G1Fc fusion polypeptide is encoded by the following nucleic acid sequence (SEQ ID NO: 430):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGTTCGATGA CGACAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 430)

A mature ActRIIB(L79F, F82D)-G1Fc fusion polypeptide (SEQ ID NO: 431) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWF DDDNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 431)

The amino acid sequence of unprocessed ActRIIB(L79F, F82T)-G1Fc is shown below (SEQ ID NO: 432). The signal sequence and linker sequences are indicated by solid underline, and the L79F, F82T substitutions are indicated by double underline. The amino acid sequence of SEQ ID NO: 432 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWFDDTNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 432)

This ActRIIB(L79F, F82T)-G1Fc fusion polypeptide is encoded by the following nucleic acid sequence (SEQ ID NO: 433):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGTTCGATGA CACCAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 433)

A mature ActRIIB(L79F, F82T)-G1Fc fusion polypeptide (SEQ ID NO: 434) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWF DDTNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 434)

The amino acid sequence of unprocessed ActRIIB(F82D, N83R)-G1Fc is shown below (SEQ ID NO: 435). The signal sequence and linker sequences are indicated by solid underline, and the F82D, N83R substitutions are indicated by double underline. The amino acid sequence of SEQ ID NO: 435 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFGV SPASGRGEA ETRECIYYNA NWELERTNQS
51 GLERCEGEQD KRLHCYASWR NSSGTIELVK KGCWLDDDRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 435)

This ActRIIB(F82D, N83R)-G1Fc fusion polypeptide is encoded by the following nucleic acid sequence (SEQ ID NO: 436):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CGACCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 436)

A mature ActRIIB(F82D, N83R)-G1Fc fusion polypeptide (SEQ ID NO: 437) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDDRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 437)

The amino acid sequence of unprocessed ActRIIB(F82E, N83R)-G1Fc is shown below (SEQ ID NO: 438). The signal sequence and linker sequences are indicated by solid underline, and the F82E, N83R substitutions are indicated by double underline. The amino acid sequence of SEQ ID NO: 438 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDERC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 438)

This ActRIIB(F82E, N83R)-G1Fc fusion polypeptide is encoded by the following nucleic acid sequence (SEQ ID NO: 439):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CGAGCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 439)

A mature ActRIIB(F82E, N83R)-G1Fc fusion polypeptide (SEQ ID NO: 440) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDERCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 440)

The amino acid sequence of unprocessed ActRIIB(F82S, N83R)-G1Fc is shown below (SEQ ID NO: 441). The signal sequence and linker sequences are indicated by solid underline, and the F82S, N83R substitutions are indicated by double underline. The amino acid sequence of SEQ ID NO: 441 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDSRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 441)

This ActRIIB(F82S, N83R)-G1Fc fusion polypeptide is encoded by the following nucleic acid sequence (SEQ ID NO: 442):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CTCCCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 442)

A mature ActRIIB(F82S, N83R)-G1Fc fusion polypeptide (SEQ ID NO: 443) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDSRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 443)

The amino acid sequence of unprocessed ActRIIB(F82W, N83A)-G1Fc is shown below (SEQ ID NO: 444). The signal sequence and linker sequences are indicated by solid underline, and the F82W N83A substitutions are indicated by double underline. The amino acid sequence of SEQ ID NO: 444 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDWAC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 444)

This ActRIIB(F82W, N83A)-G1Fc fusion polypeptide is encoded by the following nucleic acid sequence (SEQ ID NO: 445):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CTGGGCCTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 445)

A mature ActRIIB(F82W, N83A)-G1Fc fusion polypeptide (SEQ ID NO: 446) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDWACYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 446)

The amino acid sequence of unprocessed ActRIIB(F82I, E94K)-G1Fc is shown below (SEQ ID NO: 447). The signal sequence and linker sequences are indicated by solid underline, and the F82I, E94K substitutions are indicated by double underline. The amino acid sequence of SEQ ID NO: 447 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSGTIELVK KGCWLDDINC YDRQECVATK
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 447)

This ActRIIB(F82I, E94K)-G1Fc fusion polypeptide is encoded by the following nucleic acid sequence (SEQ ID NO: 448):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CATCAACTGC TACGATAGGC AGGAGTGTGT GGCCACTAAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 448)

A mature ActRIIB(F82I, E94K)-G1Fc fusion polypeptide (SEQ ID NO: 449) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSGT
51 IELVKKGCWL DDINCYDRQE CVATKENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 449)

The amino acid sequence of unprocessed ActRIIB(E50L, F82D, N83R)-G1Fc is shown below (SEQ ID NO: 450). The signal sequence and linker sequences are indicated by solid underline, and the E50L, F82D, N83R substitutions are indicated by double underline. The amino acid sequence of SEQ ID NO: 450 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCLGEQD KRLHCYASWR NSSGTIELVK KGCWLDDDRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 450)

This ActRIIB(E50L, F82D, N83R)-G1Fc fusion polypeptide is encoded by the following nucleic acid sequence (SEQ ID NO: 451):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCCTGGG CGAGCAGGAC AAGCGGCTGC ACTGCTACGC
201 CTCCTGGCGC AACAGCTCTG GCACCATCGA GCTCGTGAAG AAGGGCTGCT
251 GGCTAGATGA CGACCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 451)

A mature ActRIIB(E50L, F82D, N83R)-G1Fc fusion polypeptide (SEQ ID NO: 452) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC LGEQDKRLHC YASWRNSSGT
51 IELVKKGCWL DDDRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 452)

The amino acid sequence of unprocessed ActRIIB(E52D, F82D, N83R)-G1Fc is shown below (SEQ ID NO: 453). The signal sequence and linker sequences are indicated by solid underline, and the E52D, F82D, N83R substitutions are indicated by double underline. The amino acid sequence of SEQ ID NO: 453 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGDQD KRLHCYASWR NSSGTIELVK KGCWLDDDRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 453)

This ActRIIB(E52D, F82D, N83R)-G1Fc fusion polypeptide is encoded by the following nucleic acid sequence (SEQ ID NO: 454):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGACCAGGAC AAGCGGCTGC ACTGCTACGC
201 CTCCTGGCGC AACAGCTCTG GCACCATCGA GCTCGTGAAG AAGGGCTGCT
251 GGCTAGATGA CGACCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 454)

A mature ActRIIB(E52D, F82D, N83R)-G1Fc fusion polypeptide (SEQ ID NO: 455) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGDQDKRLHC YASWRNSSGT
51 IELVKKGCWL DDDRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 455)

The amino acid sequence of unprocessed ActRIIB(E52D, F82E, N83R)-G1Fc is shown below (SEQ ID NO: 456). The signal sequence and linker sequences are indicated by solid underline, and the E52D, F82E, N83R substitutions are indicated by double underline. The amino acid sequence of SEQ ID NO: 456 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGDQD KRLHCYASWR NSSGTIELVK KGCWLDDERC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 456)

This ActRIIB(E52D, F82E, N83R)-G1Fc fusion polypeptide is encoded by the following nucleic acid sequence (SEQ ID NO: 457):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGACCAGGAC AAGCGGCTGC ACTGCTACGC
201 CTCCTGGCGC AACAGCTCTG GCACCATCGA GCTCGTGAAG AAGGGCTGCT
251 GGCTAGATGA CGAGCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 457)

A mature ActRIIB(E52D, F82E, N83R)-G1Fc fusion polypeptide (SEQ ID NO: 458) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGDQDKRLHC YASWRNSSGT
51 IELVKKGCWL DDERCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 458)

The amino acid sequence of unprocessed ActRIIB(E52D, F82T, N83R)-G1Fc is shown below (SEQ ID NO: 459). The signal sequence and linker sequences are indicated by solid underline, and the E52D, F82T, N83R substitutions are indicated by double underline. The amino acid sequence of SEQ ID NO: 459 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGDQD KRLHCYASWR NSSGTIELVK KGCWLDDTRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 459)

This ActRIIB(E52D, F82T, N83R)-G1Fc fusion polypeptide is encoded by the following nucleic acid sequence (SEQ ID NO: 460):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGACCAGGAC AAGCGGCTGC ACTGCTACGC
201 CTCCTGGCGC AACAGCTCTG GCACCATCGA GCTCGTGAAG AAGGGCTGCT
251 GGCTAGATGA CACCCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 460)

A mature ActRIIB(E52D, F82T, N83R)-G1Fc fusion polypeptide (SEQ ID NO: 461) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGDQDKRLHC YASWRNSSGT
51 IELVKKGCWL DDTRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 461)

The amino acid sequence of unprocessed ActRIIB(E52N, F82I, N83R)-G1Fc is shown below (SEQ ID NO: 462). The signal sequence and linker sequences are indicated by solid underline, and the E52N, F82I, N83R substitutions are indicated by double underline. The amino acid sequence of SEQ ID NO: 462 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGNQD KRLHCYASWR NSSGTIELVK KGCWLDDIRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 462)

This ActRIIB(E52N, F82I, N83R)-G1Fc fusion polypeptide is encoded by the following nucleic acid sequence (SEQ ID NO: 463):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CAACCAGGAC AAGCGGCTGC ACTGCTACGC
201 CTCCTGGCGC AACAGCTCTG GCACCATCGA GCTCGTGAAG AAGGGCTGCT
251 GGCTAGATGA CATCCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 463)

A mature ActRIIB(E52N, F82I, N83R)-G1Fc fusion polypeptide (SEQ ID NO: 464) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGNQDKRLHC YASWRNSSGT
51 IELVKKGCWL DDIRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 464)

The amino acid sequence of unprocessed ActRIIB(E52N, F82Y, N83R)-G1Fc is shown below (SEQ ID NO: 465). The signal sequence and linker sequences are indicated by solid underline, and the E52N, F82Y, N83R substitutions are indicated by double underline. The amino acid sequence of SEQ ID NO: 465 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGNQD KRLHCYASWR NSSGTIELVK KGCWLDDYRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 465)

This ActRIIB(E52N, F82Y, N83R)-G1Fc fusion polypeptide is encoded by the following nucleic acid sequence (SEQ ID NO: 466):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CAACCAGGAC AAGCGGCTGC ACTGCTACGC
201 CTCCTGGCGC AACAGCTCTG GCACCATCGA GCTCGTGAAG AAGGGCTGCT
251 GGCTAGATGA CTACCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 466)

A mature ActRIIB(E52N, F82Y, N83R)-G1Fc fusion polypeptide (SEQ ID NO: 467) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGNQDKRLHC YASWRNSSGT
51 IELVKKGCWL DDYRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 467)

The amino acid sequence of unprocessed ActRIIB(E52Y, F82D,N83R)-G1Fc is shown below (SEQ ID NO: 468). The signal sequence and linker sequences are indicated by solid underline, and the E52Y, F82D, N83R substitutions are indicated by double underline. The amino acid sequence of SEQ ID NO: 468 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGYQD KRLHCYASWR NSSGTIELVK KGCWLDDDRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 468)

This ActRIIB(E52Y, F82D,N83R)-G1Fc fusion polypeptide is encoded by the following nucleic acid sequence (SEQ ID NO: 469):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CTACCAGGAC AAGCGGCTGC ACTGCTACGC
201 CTCCTGGCGC AACAGCTCTG GCACCATCGA GCTCGTGAAG AAGGGCTGCT
251 GGCTAGATGA CGACCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 469)

A mature ActRIIB(E52Y, F82D,N83R)-G1Fc fusion polypeptide (SEQ ID NO: 470) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGYQDKRLHC YASWRNSSGT
51 IELVKKGCWL DDDRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 470)

The amino acid sequence of unprocessed ActRIIB(L57E, F82E, N83R)-G1Fc is shown below (SEQ ID NO: 471). The signal sequence and linker sequences are indicated by solid underline, and the L57E, F82E, N83R substitutions are indicated by double underline. The amino acid sequence of SEQ ID NO: 471 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KREHCYASWR NSSGTIELVK KGCWLDDERC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 471)

This ActRIIB(L57E, F82E, N83R)-G1Fc fusion polypeptide is encoded by the following nucleic acid sequence (SEQ ID NO: 472):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGGAGC ACTGCTACGC
201 CTCCTGGCGC AACAGCTCTG GCACCATCGA GCTCGTGAAG AAGGGCTGCT
251 GGCTAGATGA CGAGCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 472)

A mature ActRIIB(L57E, F82E, N83R)-G1Fc fusion polypeptide (SEQ ID NO: 473) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKREHC YASWRNSSGT
51 IELVKKGCWL DDERCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 473)

The amino acid sequence of unprocessed ActRIIB(L57I, F82D, N83R)-G1Fc is shown below (SEQ ID NO: 474). The signal sequence and linker sequences are indicated by solid underline, and the L57I, F82D, N83R substitutions are indicated by double underline. The amino acid sequence of SEQ ID NO: 474 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRIHCYASWR NSSGTIELVK KGCWLDDDRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 474)

This ActRIIB(L57I, F82D, N83R)-G1Fc fusion polypeptide is encoded by the following nucleic acid sequence (SEQ ID NO: 475):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGATCC ACTGCTACGC
201 CTCCTGGCGC AACAGCTCTG GCACCATCGA GCTCGTGAAG AAGGGCTGCT
251 GGCTAGATGA CGACCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 475)

A mature ActRIIB(L57I, F82D, N83R)-G1Fc fusion polypeptide (SEQ ID NO: 476) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRIHC YASWRNSSGT
51 IELVKKGCWL DDDRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 476)

The amino acid sequence of unprocessed ActRIIB(L57I, F82E, N83R)-G1Fc is shown below (SEQ ID NO: 477). The signal sequence and linker sequences are indicated by solid underline, and the L57I, F82E, N83R substitutions are indicated by double underline. The amino acid sequence of SEQ ID NO: 477 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRIHCYASWR NSSGTIELVK KGCWLDDERC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 477)

This ActRIIB(L57I, F82E, N83R)-G1Fc fusion polypeptide is encoded by the following nucleic acid sequence (SEQ ID NO: 478):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGATCC ACTGCTACGC
201 CTCCTGGCGC AACAGCTCTG GCACCATCGA GCTCGTGAAG AAGGGCTGCT
251 GGCTAGATGA CGAGCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 478)

A mature ActRIIB(L57I, F82E, N83R)-G1Fc fusion polypeptide (SEQ ID NO: 479) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRIHC YASWRNSSGT
51 IELVKKGCWL DDERCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 479)

The amino acid sequence of unprocessed ActRIIB(L57R, F82D, N83R)-G1Fc is shown below (SEQ ID NO: 480). The signal sequence and linker sequences are indicated by solid underline, and the L57R, F82D, N83R substitutions are indicated by double underline. The amino acid sequence of SEQ ID NO: 480 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRRHCYASWR NSSGTIELVK KGCWLDDDRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 480)

This ActRIIB(L57R, F82D, N83R)-G1Fc fusion polypeptide is encoded by the following nucleic acid sequence (SEQ ID NO: 481):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGAGGC ACTGCTACGC
201 CTCCTGGCGC AACAGCTCTG GCACCATCGA GCTCGTGAAG AAGGGCTGCT
251 GGCTAGATGA CGACCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 481)

A mature ActRIIB(L57R, F82D, N83R)-G1Fc fusion polypeptide (SEQ ID NO: 482) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRRHC YASWRNSSGT
51 IELVKKGCWL DDDRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 482)

The amino acid sequence of unprocessed ActRIIB(L57R, F82E, N83R)-G1Fc is shown below (SEQ ID NO: 483). The signal sequence and linker sequences are indicated by solid underline, and the L57R, F82E, N83R substitutions are indicated by double underline. The amino acid sequence of SEQ ID NO: 483 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRRHCYASWR NSSGTIELVK KGCWLDDERC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 483)

This ActRIIB(L57R, F82E, N83R)-G1Fc fusion polypeptide is encoded by the following nucleic acid sequence (SEQ ID NO: 484):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGAGGC ACTGCTACGC
201 CTCCTGGCGC AACAGCTCTG GCACCATCGA GCTCGTGAAG AAGGGCTGCT
251 GGCTAGATGA CGAGCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 484)

A mature ActRIIB(L57R, F82E, N83R)-G1Fc fusion polypeptide (SEQ ID NO: 485) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRRHC YASWRNSSGT
51 IELVKKGCWL DDERCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 485)

The amino acid sequence of unprocessed ActRIIB(L57R, F82L, N83R)-G1Fc is shown below (SEQ ID NO: 486). The signal sequence and linker sequences are indicated by solid underline, and the L57R, F82L, N83R substitutions are indicated by double underline. The amino acid sequence of SEQ ID NO: 486 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRRHCYASWR NSSGTIELVK KGCWLDDLRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 486)

This ActRIIB(L57R, F82L, N83R)-G1Fc fusion polypeptide is encoded by the following nucleic acid sequence (SEQ ID NO: 487):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCGGC ACTGCTACGC
201 CTCCTGGCGC AACAGCTCTG GCACCATCGA GCTCGTGAAG AAGGGCTGCT
251 GGCTAGATGA CCTGCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 487)

A mature ActRIIB(L57R, F82L, N83R)-G1Fc fusion polypeptide (SEQ ID NO: 488) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRRHC YASWRNSSGT
51 IELVKKGCWL DDLRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 488)

The amino acid sequence of unprocessed ActRIIB(L57T, F82Y, N83R)-G1Fc is shown below (SEQ ID NO: 489). The signal sequence and linker sequences are indicated by solid underline, and the L57T, F82Y, N83R substitutions are indicated by double underline. The amino acid sequence of SEQ ID NO: 489 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRTHCYASWR NSSGTIELVK KGCWLDDYRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 489)

This ActRIIB(L57T, F82Y, N83R)-G1Fc fusion polypeptide is encoded by the following nucleic acid sequence (SEQ ID NO: 490):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGACCC ACTGCTACGC
201 CTCCTGGCGC AACAGCTCTG GCACCATCGA GCTCGTGAAG AAGGGCTGCT
251 GGCTAGATGA CTACCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 490)

A mature ActRIIB(L57T, F82Y, N83R)-G1Fc fusion polypeptide (SEQ ID NO: 491) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRTHC YASWRNSSGT
51 IELVKKGCWL DDYRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 491)

The amino acid sequence of unprocessed ActRIIB(L57V, F82D, N83R)-G1Fc is shown below (SEQ ID NO: 492). The signal sequence and linker sequences are indicated by solid underline, and the L57V, F82D, N83R substitutions are indicated by double underline. The amino acid sequence of SEQ ID NO: 492 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRVHCYASWR NSSGTIELVK KGCWLDDDRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 492)

This ActRIIB(L57V, F82D, N83R)-G1Fc fusion polypeptide is encoded by the following nucleic acid sequence (SEQ ID NO: 493):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGGTGC ACTGCTACGC
201 CTCCTGGCGC AACAGCTCTG GCACCATCGA GCTCGTGAAG AAGGGCTGCT
251 GGCTAGATGA CGACCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 493)

A mature ActRIIB(L57V, F82D, N83R)-G1Fc fusion polypeptide (SEQ ID NO: 494) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRVHC YASWRNSSGT
51 IELVKKGCWL DDDRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 494)

The amino acid sequence of unprocessed ActRIIB(L57V, F82Y, N83R)-G1Fc is shown below (SEQ ID NO: 495). The signal sequence and linker sequences are indicated by solid underline, and the L57V, F82Y, N83R substitutions are indicated by double underline. The amino acid sequence of SEQ ID NO: 495 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRVHCYASWR NSSGTIELVK KGCWLDDYRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 495)

This ActRIIB(L57V, F82Y, N83R)-G1Fc fusion polypeptide is encoded by the following nucleic acid sequence (SEQ ID NO: 496):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGGTGC ACTGCTACGC
201 CTCCTGGCGC AACAGCTCTG GCACCATCGA GCTCGTGAAG AAGGGCTGCT
251 GGCTAGATGA CTACCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 496)

A mature ActRIIB(L57V, F82Y, N83R)-G1Fc fusion polypeptide (SEQ ID NO: 497) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRVHC YASWRNSSGT
51 IELVKKGCWL DDYRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 497)

The amino acid sequence of unprocessed ActRIIB(G68R, W78Y, F82Y)-G1Fc is shown below (SEQ ID NO: 498). The signal sequence and linker sequences are indicated by solid underline, and the G68R, W78Y, F82Y substitutions are indicated by double underline. The amino acid sequence of SEQ ID NO: 498 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSRTIELVK KGCYLDDYNC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 498)

This ActRIIB(G68R, W78Y, F82Y)-G1Fc fusion polypeptide is encoded by the following nucleic acid sequence (SEQ ID NO: 499):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTC GGACCATCGA GCTCGTGAAG AAGGGCTGCT
251 ACCTAGATGA CTACAACTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 499)

A mature ActRIIB(G68R, W78Y, F82Y)-G1Fc fusion polypeptide (SEQ ID NO: 500) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSRT
51 IELVKKGCYL DDYNCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 500)

The amino acid sequence of unprocessed ActRIIB(G68R, F82S, N83R)-G1Fc is shown below (SEQ ID NO: 501). The signal sequence and linker sequences are indicated by solid underline, and the G68R, F82S, N83R substitutions are indicated by double underline. The amino acid sequence of SEQ ID NO: 501 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSRTIELVK KGCWLDDSRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 501)

This ActRIIB(G68R, F82S, N83R)-G1Fc fusion polypeptide is encoded by the following nucleic acid sequence (SEQ ID NO: 502):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTA GGACCATCGA GCTCGTGAAG AAGGGCTGCT
251 GGCTAGATGA CTCCCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 502)

A mature ActRIIB(G68R, F82S, N83R)-G1Fc fusion polypeptide (SEQ ID NO: 503) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSRT
51 IELVKKGCWL DDSRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 503)

The amino acid sequence of unprocessed ActRIIB(E52N, G68R, F82Y, N83R)-G1Fc is shown below (SEQ ID NO: 504). The signal sequence and linker sequences are indicated by solid underline, and the E52N, G68R, F82Y, N83R substitutions are indicated by double underline. The amino acid sequence of SEQ ID NO: 504 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGNQD KRLHCYASWR NSSRTIELVK KGCWLDDYRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 504)

This ActRIIB(E52N, G68R, F82Y, N83R)-G1Fc fusion polypeptide is encoded by the following nucleic acid sequence (SEQ ID NO: 505):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CAACCAGGAC AAGCGGCTGC ACTGCTACGC
201 CTCCTGGCGC AACAGCTCTC GGACCATCGA GCTCGTGAAG AAGGGCTGCT
251 GGCTAGATGA CTACCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 505)

A mature ActRIIB(E52N, G68R, F82Y, N83R)-G1Fc fusion polypeptide (SEQ ID NO: 506) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGNQDKRLHC YASWRNSSRT
51 IELVKKGCWL DDYRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 506)

The amino acid sequence of unprocessed ActRIIB(G68R, L79E, F82T, N83R)-G1Fc is shown below (SEQ ID NO: 507). The signal sequence and linker sequences are indicated by solid underline, and the G68R, L79E, F82T, N83R substitutions are indicated by double underline. The amino acid sequence of SEQ ID NO: 507 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSRTIELVK KGCWEDDTRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 507)

This ActRIIB(G68R, L79E, F82T, N83R)-G1Fc fusion polypeptide is encoded by the following nucleic acid sequence (SEQ ID NO: 508):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTA GGACCATCGA GCTCGTGAAG AAGGGCTGCT
251 GGGAGGATGA CACCCGTTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 508)

A mature ActRIIB(G68R, L79E, F82T, N83R)-G1Fc fusion polypeptide (SEQ ID NO: 509) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSRT
51 IELVKKGCWE DDTRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 509)

The amino acid sequence of unprocessed ActRIIB(G68R, L79E, F82Y, N83R)-G1Fc is shown below (SEQ ID NO: 510). The signal sequence and linker sequences are indicated by solid underline, and the G68R, L79E, F82Y, N83R substitutions are indicated by double underline. The amino acid sequence of SEQ ID NO: 510 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSRTIELVK KGCWEDDYRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 510)

This ActRIIB(G68R, L79E, F82Y, N83R)-G1Fc fusion polypeptide is encoded by the following nucleic acid sequence (SEQ ID NO: 511):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTC GGACCATCGA GCTCGTGAAG AAGGGCTGCT
251 GGGAAGATGA CTACCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 511)

A mature ActRIIB(G68R, L79E, F82Y, N83R)-G1Fc fusion polypeptide (SEQ ID NO: 512) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSRT
51 IELVKKGCWE DDYRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 512)

The amino acid sequence of unprocessed ActRIIB(G68R, L79T, F82T, N83R)-G1Fc is shown below (SEQ ID NO: 513). The signal sequence and linker sequences are indicated by solid underline, and the G68R, L79T, F82T, N83R substitutions are indicated by double underline. The amino acid sequence of SEQ ID NO: 513 may optionally be provided with the lysine removed from the C-terminus. The G1Fc region may also comprise a mutation to an alanine at position 234 or a mutation to an alanine at position 235, or a combination thereof.

1 MDAMKRGLCC VLLLCGAVFV SPGASGRGEA ETRECIYYNA NWELERTNQS
51 GLERCEGEQD KRLHCYASWR NSSRTIELVK KGCWTDDTRC YDRQECVATE
101 ENPQVYFCCC EGNFCNERFT HLPEAGGPEV TYEPPPTAPT GGGTHTCPPC
151 PAPELLGGPS VFLFPPKPKD TLMISRTPEV TCVVVDVSHE DPEVKFNWYV
201 DGVEVHNAKT KPREEQYNST YRVVSVLTVL HQDWLNGKEY KCKVSNKALP
251 APIEKTISKA KGQPREPQVY TLPPSREEMT KNQVSLTCLV KGFYPSDIAV
301 EWESNGQPEN NYKTTPPVLD SDGSFFLYSK LTVDKSRWQQ GNVFSCSVMH
351 EALHNHYTQK SLSLSPGK (SEQ ID NO: 513)

This ActRIIB(G68R, L79T, F82T, N83R)-G1Fc fusion polypeptide is encoded by the following nucleic acid sequence (SEQ ID NO: 514):

1 ATGGATGCAA TGAAGAGAGG GCTCTGCTGT GTGCTGCTGC TGTGTGGAGC
51 AGTCTTCGTT TCGCCCGGCG CCTCTGGGCG TGGGGAGGCT GAGACACGGG
101 AGTGCATCTA CTACAACGCC AACTGGGAGC TGGAGCGCAC CAACCAGAGC
151 GGCCTGGAGC GCTGCGAAGG CGAGCAGGAC AAGCGGCTGC ACTGCTACGC
201 CTCCTGGCGC AACAGCTCTC GGACCATCGA GCTCGTGAAG AAGGGCTGCT
251 GGACCGATGA CACCCGGTGC TACGATAGGC AGGAGTGTGT GGCCACTGAG
301 GAGAACCCCC AGGTGTACTT CTGCTGCTGT GAAGGCAACT TCTGCAACGA
351 GCGCTTCACT CATTTGCCAG AGGCTGGGGG CCCGGAAGTC ACGTACGAGC
401 CACCCCCGAC AGCCCCCACC GGTGGTGGAA CTCACACATG CCCACCGTGC
451 CCAGCACCTG AACTCCTGGG GGGACCGTCA GTCTTCCTCT TCCCCCCAAA
501 ACCCAAGGAC ACCCTCATGA TCTCCCGGAC CCCTGAGGTC ACATGCGTGG
551 TGGTGGACGT GAGCCACGAA GACCCTGAGG TCAAGTTCAA CTGGTACGTG
601 GACGGCGTGG AGGTGCATAA TGCCAAGACA AAGCCGCGGG AGGAGCAGTA
651 CAACAGCACG TACCGTGTGG TCAGCGTCCT CACCGTCCTG CACCAGGACT
701 GGCTGAATGG CAAGGAGTAC AAGTGCAAGG TCTCCAACAA AGCCCTCCCA
751 GCCCCCATCG AGAAAACCAT CTCCAAAGCC AAAGGGCAGC CCCGAGAACC
801 ACAGGTGTAC ACCCTGCCCC CATCCCGGGA GGAGATGACC AAGAACCAGG
851 TCAGCCTGAC CTGCCTGGTC AAAGGCTTCT ATCCCAGCGA CATCGCCGTG
901 GAGTGGGAGA GCAATGGGCA GCCGGAGAAC AACTACAAGA CCACGCCTCC
951 CGTGCTGGAC TCCGACGGCT CCTTCTTCCT CTATAGCAAG CTCACCGTGG
1001 ACAAGAGCAG GTGGCAGCAG GGGAACGTCT TCTCATGCTC CGTGATGCAT
1051 GAGGCTCTGC ACAACCACTA CACGCAGAAG AGCCTCTCCC TGTCTCCGGG
1101 TAAA (SEQ ID NO: 514)

A mature ActRIIB(G68R, L79T, F82T, N83R)-G1Fc fusion polypeptide (SEQ ID NO: 515) is as follows and may optionally be provided with the lysine removed from the C-terminus. The G1Fc region may also comprise a mutation to an alanine at position 234 or a mutation to an alanine at position 235, or a combination thereof.

1 GRGEAETREC IYYNANWELE RTNQSGLERC EGEQDKRLHC YASWRNSSRT
51 IELVKKGCWT DDTRCYDRQE CVATEENPQV YFCCCEGNFC NERFTHLPEA
101 GGPEVTYEPP PTAPTGGGTH TCPPCPAPEL LGGPSVFLFP PKPKDTLMIS
151 RTPEVTCVVV DVSHEDPEVK FNWYVDGVEV HNAKTKPREE QYNSTYRVVS
201 VLTVLHQDWL NGKEYKCKVS NKALPAPIEK TISKAKGQPR EPQVYTLPPS
251 REEMTKNQVS LTCLVKGFYP SDIAVEWESN GQPENNYKTT PPVLDSDGSF
301 FLYSKLTVDK SRWQQGNVFS CSVMHEALHN HYTQKSLSLS PGK
(SEQ ID NO: 515)

### Example 13. Ligand Binding Profiles of Variant ActRIIB-Fc Homodimers and Activity of Variant ActRIIB-Fc Proteins in a Cell-Based Assay

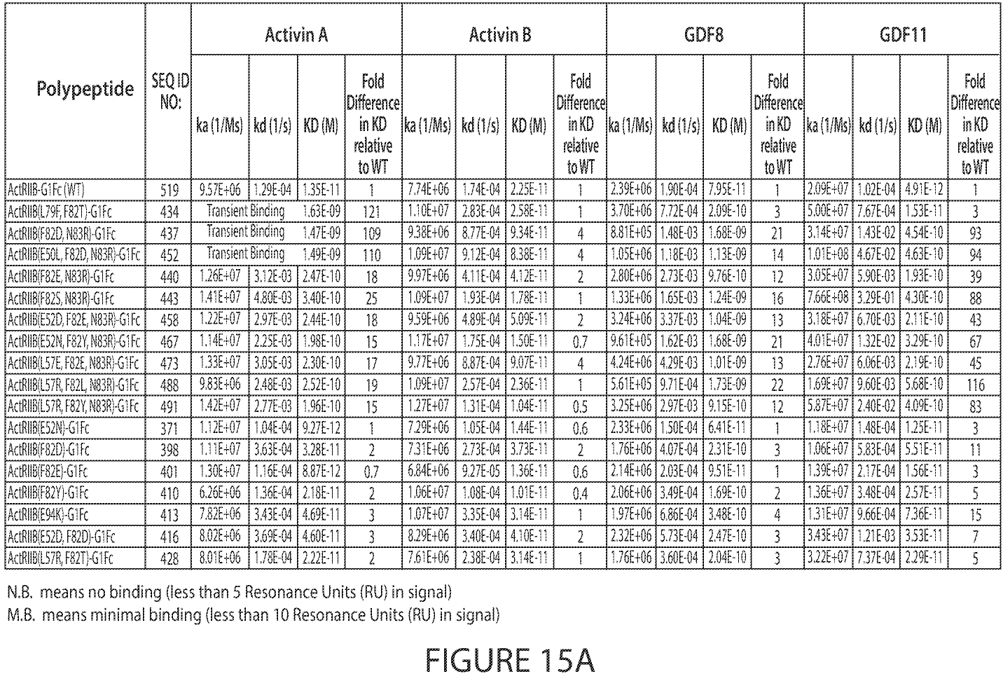

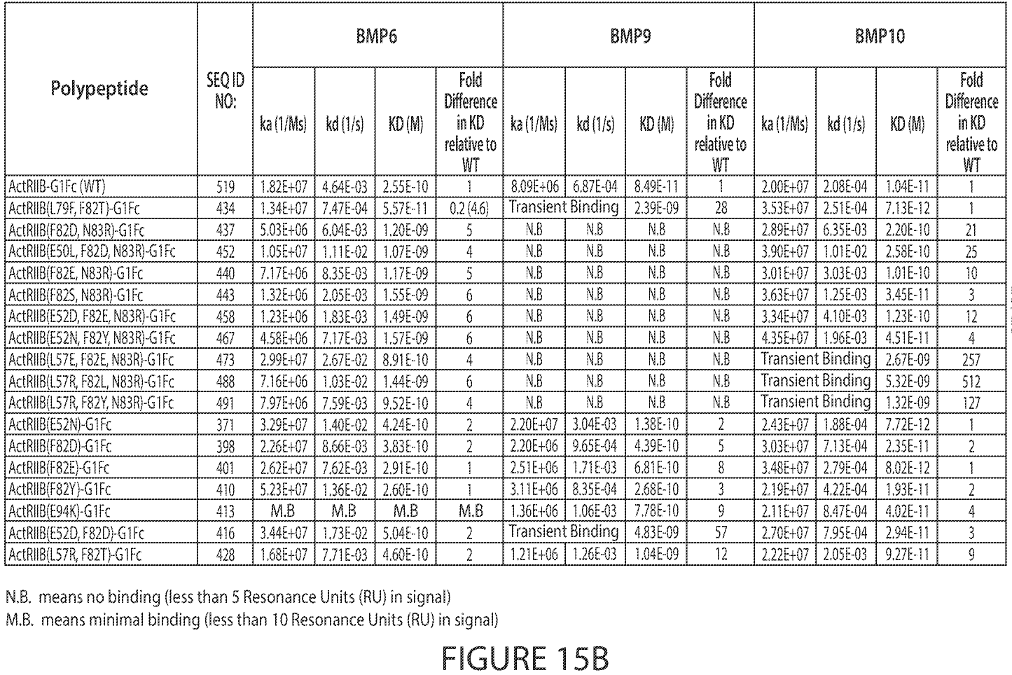

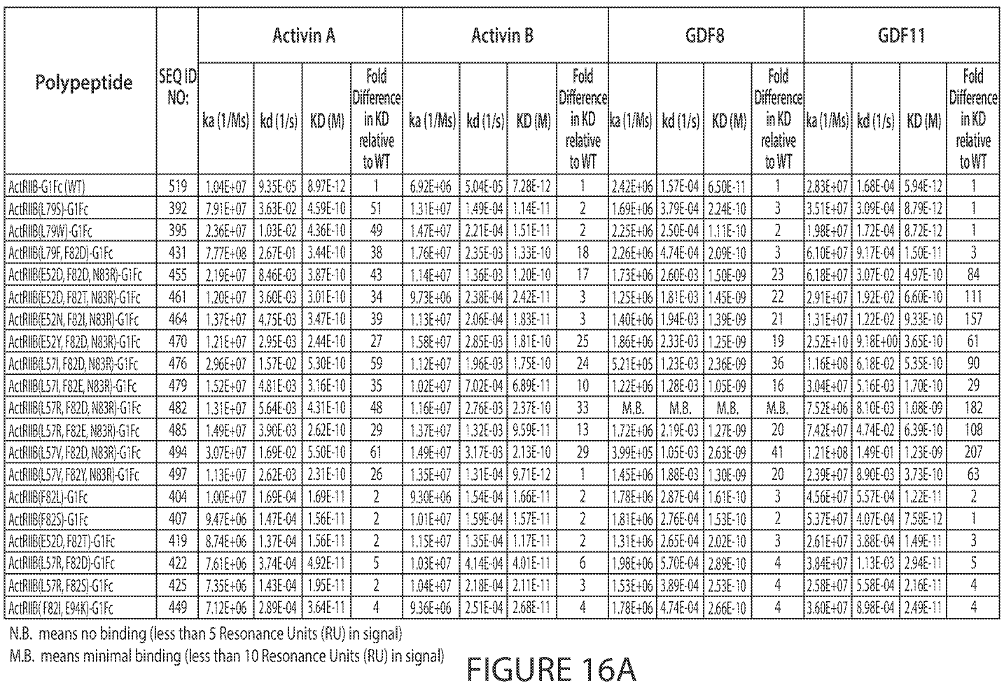

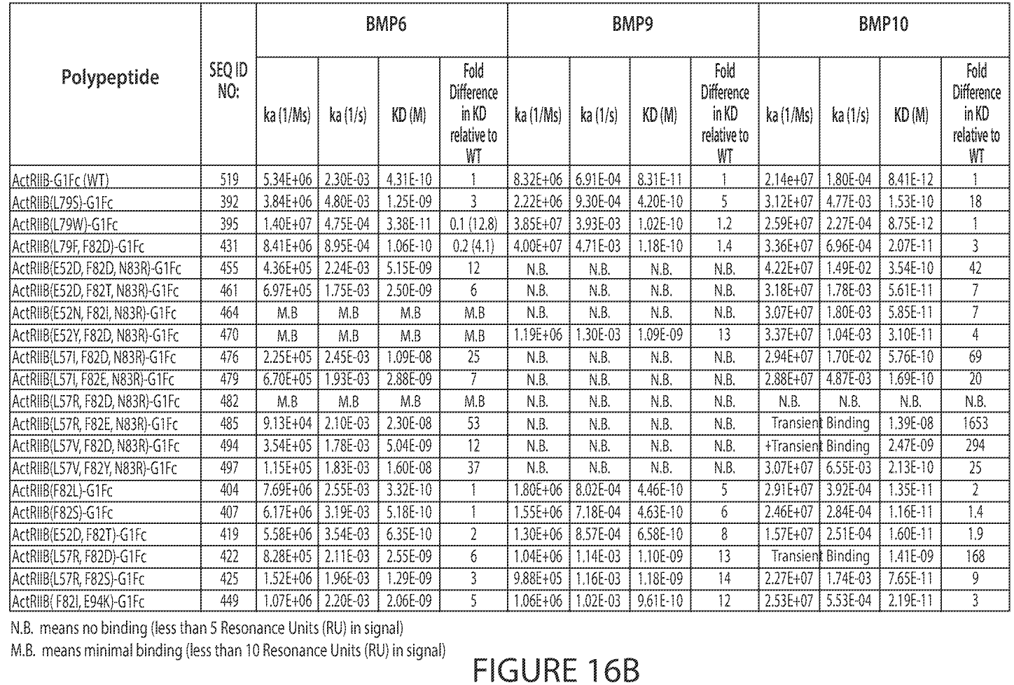

To determine ligand binding profiles of variant ActRIIB-Fc homodimers, a Biacore™-based binding assay was used to compare ligand binding kinetics of certain variant ActRIIB-Fc proteins. ActRIIB-Fc proteins to be tested were independently captured onto the system using an anti-Fc antibody. Ligands were then injected and allowed to flow over the captured receptor protein. Results of variant ActRIIB-Fc proteins analyzed at 37° C. are shown in FIGS. 15A, 15B, 16A, and 16B.

To determine activity of variant ActRIIB-Fc proteins, an A204 cell-based assay was used to compare effects among variant ActRIIB-Fc proteins on signaling by activin A, activin B, GDF8, and GDF11, whereas a HepG2 cell-based assay was used for BMP6 and a T98G-cell based assay was used for BMP10, and BMP9. In brief, the A204 assay uses a human A204 rhabdomyosarcoma cell line (ATCC^{®}: HTB-82™) derived from muscle and the reporter vector pGL3(CAGA)12 (Dennler et al., 1998, EMBO 17: 3091-3100) as well as a Renilla reporter plasmid (pRLCMV) to control for transfection efficiency. The CAGA12 motif is present in TGF-β responsive genes (e.g., PAI-1 gene), so this vector is of general use for ligands that can signal through Smad2/3, including activin A, activin B, GDF8, and GDF11. The HepG2 assay uses a human hepatocellular carcinoma cell line (ATCC^{®}: HB-8065™) and the T98G assay uses a human T98G glioblastoma cell line (ATCC^{®} CRL-1690™). Both lines were used with the reporter vector pGL3 BRE-luciferase as wells as a Renilla reporter plasmid (pRLCMV) to control for transfection efficiency. The BMP-responsive element (BRE) was identified in the Idl gene (Korchynskyi, O. and ten Dijke, P., Journal of Biological Chemistry, 2002, 277:4883-4891) so this vector is of general use for ligands that can signal through Smad1/5/8, including BMP6, BMP10, and BMP9.

On day 1, A-204 cells were transferred into one or more 48-well plates. On day 2, each 48-well plate was transfected with pGL3(CAGA)12(10 µg) + pRLCMV (100 ng) and X-tremeGENE 9 (0.1% BSA containing medium). On day 3, ligands diluted in medium containing 0.1% BSA were preincubated with ActRIIB-Fc proteins for 30 min before addition to cells. Approximately six hours later, the cells were rinsed with PBS and lysed. Cell lysates were analyzed in a luciferase assay to determine the extent of Smad activation. The HepG2 and T98G assays were performed similarly only substituting pGL3-BRE for the reporter plasmid and X-tremeGENE HP for the transfection reagent and assaying the cells after an overnight incubation with ligand/test samples.

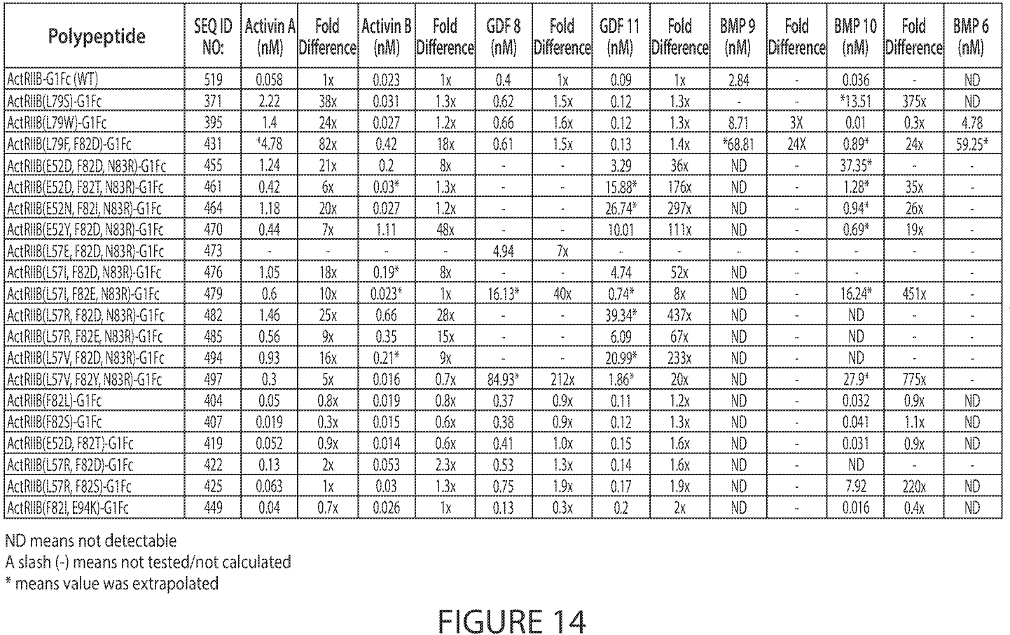

This assay was used to screen variant ActRIIB-Fc proteins for inhibitory effects on cell signaling by activin A, activin B, GDF8, GDF11, BMP6, BMP10, and BMP9 (FIG. 14). Potencies of homodimeric Fc fusion proteins incorporating amino acid substitutions in the human ActRIIB extracellular domain were compared with that of an Fc fusion protein comprising unmodified human ActRIIB extracellular domain.

As shown in FIG. 14-16B, substitutions in the ActRIIB extracellular domain can alter the balance between activin A, activin B, GDF8, GDF11, BMP6, BMP9, and BMP10 inhibition in both a Biacore™-based binding assay and a cell-based assay. Binding profiles differed significantly between sets of variants.

Variants ActRIIB(F82D, N83R)-G1Fc (e.g., SEQ ID NO: 437), and ActRIIB(E50L, F82D, N83R)-G1Fc (e.g., SEQ ID NO: 452), compared to an unmodified ActRIIB-Fc protein (e.g., “WT”), displayed a significant reduction in activin A binding, a significant reduction in GDF11 binding, no detected BMP10 binding, a minor reduction in BMP6 binding, and a significant reduction in BMP9 binding. Further, ActRIIB(F82D, N83R)-G1Fc (e.g., SEQ ID NO: 437), and ActRIIB(E50L, F82D, N83R)-G1Fc (e.g., SEQ ID NO: 452), compared to WT, displayed near-WT levels of activin B binding.

Variant ActRIIB(L79F, F82T)-G1Fc (e.g., SEQ ID NO: 434), compared to WT, displayed a significant reduction in activin A binding, near-WT levels of GDF11 binding, near-WT levels of BMP10 binding, a slight increase in BMP6 binding, and a modest reduction in BMP9 binding. Further, ActRIIB(L79F, F82T)-G1Fc (e.g., SEQ ID NO: 434), compared WT, displayed near-WT levels of activin B binding.

Variant ActRIIB(L79S)-G1Fc (e.g., SEQ ID NO: 392), compared to WT, displayed a significant reduction in activin A binding, near-WT levels of GDF11 binding, a minor reduction in BMP10 binding, near-WT levels of BMP6 binding, and a minor reduction in BMP9 binding. Further, ActRIIB(L79S)-G1Fc (e.g., SEQ ID NO: 392), compared to WT, displayed near-WT levels of activin B binding.

Variant ActRIIB(L57R, F82L, N83R)-G1Fc (e.g., SEQ ID NO: 488), compared to WT, displayed a modest reduction in activin A binding, a significant reduction in GDF11 binding, no detectable BMP10 binding, a minor reduction in BMP6 binding, and a significant reduction in BMP9 binding. Further, ActRIIB(L57R, F82L, N83R)-G1Fc (e.g., SEQ ID NO: 488), compared to WT, displayed near-WT levels of activin B binding.

Variant ActRIIB(L57V, F82D, N83R)-G1Fc (e.g., SEQ ID NO: 494), compared to WT, displayed a modest reduction in activin A binding, a significant reduction in GDF11 binding, no detectable BMP10 binding, a minor reduction in BMP6 binding, and a significant reduction in BMP9 binding. Further, ActRIIB(L57V, F82D, N83R)-G1Fc (e.g., SEQ ID NO: 494), compared to WT, displayed modestly reduced activin B binding.

Variants ActRIIB(L57R, F82D, N83R)-G1Fc (e.g., SEQ ID NO: 482), and ActRIIB(L57R, F82E, N83R)-G1Fc (e.g., SEQ ID NO: 485), compared to WT, displayed a modest reduction in activin A binding, a significant reduction in GDF11 binding, no detectable BMP10 binding, a significant reduction in BMP6 binding, and a significant reduction in BMP9 binding. Further, ActRIIB(L57R, F82D, N83R)-G1Fc (e.g., SEQ ID NO: 482), and ActRIIB(L57R, F82E, N83R)-G1Fc (e.g., SEQ ID NO: 485), compared to WT, displayed modestly reduced activin B binding.

Variants ActRIIB(F82S, N83R)-G1Fc (e.g., SEQ ID NO: 443), and ActRIIB(E52N, F82Y, N83R)-G1Fc (e.g., SEQ ID NO: 467), compared to WT, displayed a modest reduction in activin A binding, a significant reduction in GDF11 binding, no detectable BMP10 binding, a minor reduction in BMP6 binding, and a significant reduction in BMP9 binding. Further, ActRIIB(F82S, N83R)-G1Fc (e.g., SEQ ID NO: 443), and ActRIIB(E52N, F82Y, N83R)-G1Fc (e.g., SEQ ID NO: 467), compared to WT, displayed near-WT levels of activin B binding. , compared to an unmodified ActRIIB-Fc protein

Variant ActRIIB(L57T, F82Y, N83R)-G1Fc (e.g., SEQ ID NO: 491), compared to WT, displayed a modest reduction in activin A binding, a significant reduction in GDF11 binding, minimal BMP10 binding, a minor reduction in BMP6 binding, and a significant reduction in BMP9 binding. Further, ActRIIB(L57T, F82Y, N83R)-G1Fc (e.g., SEQ ID NO: 491), compared to WT, displayed near-WT levels of activin B binding.

Variant ActRIIB(E52D, F82D, N83R)-G1Fc (e.g., SEQ ID NO: 455), compared to WT, displayed a modest reduction in activin A binding, a significant reduction in GDF11 binding, minimal BMP10 binding, a minor reduction in BMP6 binding, and a significant reduction in BMP9 binding. Further, ActRIIB(E52D, F82D, N83R)-G1Fc (e.g., SEQ ID NO: 455), compared to WT, displayed a modest reduction in activin B binding.

Variant ActRIIB(L57I, F82D, N83R)-G1Fc (e.g., SEQ ID NO: 476), compared to WT, displayed a modest reduction in activin A binding, a significant reduction in GDF11 binding, minimal BMP10 binding, a modest reduction in BMP6 binding, and a significant reduction in BMP9 binding. Further, ActRIIB(L57I, F82D, N83R)-G1Fc (e.g., SEQ ID NO: 476), compared to WT, displayed a modest reduction in activin B binding.

Variant ActRIIB(E52D, F82T, N83R)-G1Fc (e.g., SEQ ID NO: 461), compared to WT, displayed a modest reduction in activin A binding, a significant reduction in GDF11 binding, a modest reduction in BMP10 binding, a minor reduction in BMP6 binding, and a significant reduction in BMP9 binding. Further, ActRIIB(E52D, F82T, N83R)-G1Fc (e.g., SEQ ID NO: 461), compared to WT, displayed near-WT levels of activin B binding.

Variant ActRIIB(E52N, F82I, N83R)-G1Fc (e.g., SEQ ID NO: 464), compared to WT, displayed a modest reduction in activin A binding, a significant reduction in GDF11 binding, a modest reduction in BMP10 binding, minimal BMP6 binding, and a significant reduction in BMP9 binding. Further, ActRIIB(E52N, F82I, N83R)-G1Fc (e.g., SEQ ID NO: 464), compared to WT, displayed near-WT levels of activin B binding.

Variant ActRIIB(E52Y, F82D, N83R)-G1Fc (e.g., SEQ ID NO: 470), compared to WT, displayed a modest reduction in activin A binding, a significant reduction in GDF11 binding, a modest reduction in BMP10 binding, minimal BMP6 binding, and a modest reduction in BMP9 binding. Further, ActRIIB(E52Y, F82D, N83R)-G1Fc (e.g., SEQ ID NO: 470), compared to WT, displayed a modest reduction in activin B binding.

Variants ActRIIB(F82E, N83R)-G1Fc (e.g., SEQ ID NO: 440), ActRIIB(E52D, F82E, N83R)-G1Fc (e.g., SEQ ID NO: 458), ActRIIB(L57I, F82E, N83R)-G1Fc (e.g., SEQ ID NO: 479), and ActRIIB(L57E, F82E, N83R)-G1Fc (e.g., SEQ ID NO: 473), compared to WT, displayed a modest reduction in activin A binding, a modest reduction in GDF11 binding, no detectable BMP10 binding, a minor reduction in BMP6 binding, and a significant reduction in BMP9 binding. Further, ActRIIB(F82E, N83R)-G1Fc (e.g., SEQ ID NO: 440), ActRIIB(E52D, F82E, N83R)-G1Fc (e.g., SEQ ID NO: 458), ActRIIB(L57I, F82E, N83R)-GlFc (e.g., SEQ ID NO: 479), and ActRIIB(L57E, F82E, N83R)-G1Fc (e.g., SEQ ID NO: 473), compared to WT, displayed a minor reduction in activin B binding.

Variant ActRIIB(L79W)-GlFc (e.g., SEQ ID NO: 395), compared to WT, displayed a modest reduction in activin A binding, near-WT levels of GDF11 binding, near-WT levels of BMP10 binding, a slight increase in BMP6 binding, and near-WT levels of BMP9 binding. Further, ActRIIB(L79W)-G1Fc (e.g., SEQ ID NO: 395), compared to WT, displayed near-WT levels of activin B binding.

Variant ActRIIB(L79F, F82D)-G1Fc (e.g., SEQ ID NO: 431), compared to WT, displayed a modest reduction in activin A binding, near-WT levels of GDF11 binding, near-WT levels of BMP10 binding, a slight increase in BMP6 binding, and near-WT levels of BMP9 binding. Further, ActRIIB(L79F, F82D)-G1Fc (e.g., SEQ ID NO: 431), compared to WT, displayed a modest reduction in activin B binding.

Variant ActRIIB(L57V, F82Y, N83R)-G1Fc (e.g., SEQ ID NO: 497), compared to WT, displayed a minor reduction in activin A binding, a modest reduction in GDF11 binding, a modest reduction in BMP10 binding, a significant reduction in BMP6 binding, and a significant reduction in BMP9 binding. Further, ActRIIB(L57V, F82Y, N83R)-G1Fc (e.g., SEQ ID NO: 497), compared to WT, displayed near-WT levels of activin B binding.

Variant ActRIIB(L57R, F82D)-G1Fc (e.g., SEQ ID NO: 422), compared to WT, displayed near-WT levels of activin A binding, near-WT levels of GDF11 binding, a modest reduction in BMP10 binding, a modest reduction in BMP6 binding, and a modest reduction in BMP9 binding. Further, ActRIIB(L57R, F82D)-G1Fc (e.g., SEQ ID NO: 422), compared to WT, displayed a minor reduction in activin B binding.

Variant ActRIIB(E94K)-G1Fc (e.g., SEQ ID NO: 413), compared to WT, displayed near-WT levels of activin A binding, near-WT levels of GDF11 binding, near-WT levels of BMP10 binding, a minimal binding to BMP6, and a minor reduction in BMP9 binding. Further, ActRIIB(E94K)-G1Fc (e.g., SEQ ID NO: 413), compared to WT, displayed near-WT levels of activin B binding.

Variants ActRIIB(F82D)-G1Fc (e.g., SEQ ID NO: 398), ActRIIB(F82E)-G1Fc (e.g., SEQ ID NO: 401), ActRIIB(F82S)-G1Fc (e.g., SEQ ID NO: 407), ActRIIB(F82L)-G1Fc (e.g., SEQ ID NO: 404), and ActRIIB(E52D, F82T)-G1Fc (e.g., SEQ ID NO: 419), compared to WT, displayed near-WT levels of activin A binding, near-WT levels of GDF11 binding, near-WT levels of BMP10 binding, near-ActRIIB levels of BMP6 binding, and a minor reduction in BMP9 binding. Further, ActRIIB(F82D)-G1Fc (e.g., SEQ ID NO: 398), ActRIIB(F82E)-G1Fc (e.g., SEQ ID NO: 401), ActRIIB(F82S)-G1Fc (e.g., SEQ ID NO: 407), ActRIIB(F82L)-G1Fc (e.g., SEQ ID NO: 404), and ActRIIB(E52D, F82T)-G1Fc (e.g., SEQ ID NO: 419), compared to WT, displayed near-WT levels of activin B binding.

Variants ActRIIB(E52N)-G1Fc (e.g., SEQ ID NO: 371) and ActRIIB(F82Y)-G1Fc (e.g., SEQ ID NO: 410), compared to WT, displayed near-WT levels of activin A binding, near-WT levels of GDF11 binding, near-WT levels of BMP10 binding, near-ActRIIB levels of BMP6 binding, and near-WT levels of BMP9 binding. Further, ActRIIB(E52N)-G1Fc (e.g., SEQ ID NO: 371), ActRIIB(F82Y)-GlFc (e.g., SEQ ID NO: 410), compared to WT, displayed near-WT levels of activin B binding.

Variant ActRIIB(E52D, F82D)-G1Fc (e.g., SEQ ID NO: 416), compared to WT, displayed near-WT levels of activin A binding, near-WT levels of GDF11 binding, near-WT levels of BMP10 binding, near-ActRIIB levels of BMP6 binding, and a modest reduction in BMP9 binding. Further, ActRIIB(E52D, F82D)-G1Fc (e.g., SEQ ID NO: 416), compared to WT, displayed a minor reduction in activin B binding.

Variant ActRIIB(L57R, F82T)-G1Fc (e.g., SEQ ID NO: 428), compared to WT, displayed near-WT levels of activin A binding, near-WT levels of GDF11 binding, a minor reduction in BMP10 binding, near-ActRIIB levels of BMP6 binding, and a modest reduction in BMP9 binding. Further, ActRIIB(L57R, F82T)-G1Fc (e.g., SEQ ID NO: 428), compared to WT, displayed a minor reduction in activin B binding.

Variant ActRIIB(L57R, F82S)-G1Fc (e.g., SEQ ID NO: 425), compared to WT, displayed near-WT levels of activin A binding, near-WT levels of GDF11 binding, a minor reduction in BMP10 binding, near-ActRIIB levels of BMP6 binding, and a modest reduction in BMP9 binding. Further, ActRIIB(L57R, F82S)-G1Fc (e.g., SEQ ID NO: 425), compared to WT, displayed near-WT levels of activin B binding.

Variant ActRIIB(F82I, E94K)-G1Fc (e.g., SEQ ID NO: 449), compared to WT, displayed near-WT levels of activin A binding, near-WT levels of GDF11 binding, near-WT levels of BMP10 binding, a minor reduction in BMP6 binding, and a modest reduction in BMP9 binding. Further, ActRIIB(F82I, E94K)-G1Fc (e.g., SEQ ID NO: 449), compared to WT, displayed a minor reduction in activin B binding.

These results indicate that variant ActRIIB-Fc proteins can display varying ligand binding profiles compared to an Fc fusion protein comprising unmodified ActRIIB extracellular domain. Accordingly, these variants may be more useful than ActRIIB-Fc in certain applications where such selective antagonism profiles are advantageous. Examples include therapeutic applications where it is desirable to retain antagonism of one or more of activin A, activin B, GDF8, GDF11, BMP6, and/or BMP10, while reducing antagonism of BMP9.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

ATGGCGGAGTCGGCCGGAGCCTCCTCCTTCTTCCCCCTTGTTGTCCTCCT
GCTCGCCGGCAGCGGCGGG TCCGGGCCCCGGGGGGTCCAGGCTCTGCTGT
GTGCGTGCACCAGCTGCCTCCAGGCCAACTACACGTGTGAGACAGATGGG
GCCTGCATGGTTTCCATTTTCAATCTGGATGGGATGGAGCACCATGTGCG
CACCTGCATCCCCAAAGTGGAGCTGGTCCCTGCCGGGAAGCCCTTCTACT
GCCTGAGCTCGGAGGACCTGCGCAACACCCACTGCTGCTACACTGACTAC
TGCAACAGGATCGACTTGAGGGTGCCCAGTGGTCACCTCAAGGAGCCTGA
GCACCCGTCCATGTGGGGCCCGGTGGAGCTGGTAGGCATCATCGCCGGCC
CGGTGTTCCTCCTGTTCCTCATCATCATCATTGTTTTCCTTGTCATTAAC
TATCATCAGCGTGTCTATCACAACCGCCAGAGACTGGACATGGAAGATCC
CTCATGTGAGATGTGTCTCTCCAAAGACAAGACGCTCCAGGATCTTGTCT
ACGATCTCTCCACCTCAGGGTCTGGCTCAGGGTTACCCCTCTTTGTCCAG
CGCACAGTGGCCCGAACCATCGTTTTACAAGAGATTATTGGCAAGGGTCG
GTTTGGGGAAGTATGGCGGGGCCGCTGGAGGGGTGGTGATGTGGCTGTGA
AAATATTCTCTTCTCGTGAAGAACGGTCTTGGTTCAGGGAAGCAGAGATA
TACCAGACGGTCATGCTGCGCCATGAAAACATCCTTGGATTTATTGCTGC
TGACAATAAAGATAATGGCACCTGGACACAGCTGTGGCTTGTTTCTGACT
ATCATGAGCACGGGTCCCTGTTTGATTATCTGAACCGGTACACAGTGACA
ATTGAGGGGATGATTAAGCTGGCCTTGTCTGCTGCTAGTGGGCTGGCACA
CCTGCACATGGAGATCGTGGGCACCCAAGGGAAGCCTGGAATTGCTCATC
GAGACTTAAAGTCAAAGAACATTCTGGTGAAGAAAAATGGCATGTGTGCC
ATAGCAGACCTGGGCCTGGCTGTCCGTCATGATGCAGTCACTGACACCAT
TGACATTGCCCCGAATCAGAGGGTGGGGACCAAACGATACATGGCCCCTG
AAGTACTTGATGAAACCATTAATATGAAACACTTTGACTCCTTTAAATGT
GCTGATATTTATGCCCTCGGGCTTGTATATTGGGAGATTGCTCGAAGATG
CAATTCTGGAGGAGTCCATGAAGAATATCAGCTGCCATATTACGACTTAG
TGCCCTCTGACCCTTCCATTGAGGAAATGCGAAAGGTTGTATGTGATCAG
AAGCTGCGTCCCAACATCCCCAACTGGTGGCAGAGTTATGAGGCACTGCG
GGTGATGGGGAAGATGATGCGAGAGTGTTGGTATGCCAACGGCGCAGCCC
GCCTGACGGCCCTGCGCATCAAGAAGACCCTCTCCCAGCTCAGCGTGCAG
GAAGACGTGAAGATC (SEQ ID NO: 221)

TCCGGGCCCCGGGGGGTCCAGGCTCTGCTGTGTGCGTGCACCAGCTGCCT
CCAGGCCAACTACACGTGTGAGACAGATGGGGCCTGCATGGTTTCCATTT
TCAATCTGGATGGGATGGAGCACCATGTGCGCACCTGCATCCCCAAAGTG
GAGCTGGTCCCTGCCGGGAAGCCCTTCTACTGCCTGAGCTCGGAGGACCT
GCGCAACACCCACTGCTGCTACACTGACTACTGCAACAGGATCGACTTGA
GGGTGCCCAGTGGTCACCTCAAGGAGCCTGAGCACCCGTCCATGTGGGGC
CCGGTGGAG (SEQ ID NO: 222)

ATGGCGGAGTCGGCCGGAGCCTCCTCCTTCTTCCCCCTTGTTGTCCTCCT
GCTCGCCGGCAGCGGCGGG TCCGGGCCCCGGGGGGTCCAGGCTCTGCTGT
GTGCGTGCACCAGCTGCCTCCAGGCCAACTACACGTGTGAGACAGATGGG
GCCTGCATGGTTTCCATTTTCAATCTGGATGGGATGGAGCACCATGTGCG
CACCTGCATCCCCAAAGTGGAGCTGGTCCCTGCCGGGAAGCCCTTCTACT
GCCTGAGCTCGGAGGACCTGCGCAACACCCACTGCTGCTACACTGACTAC
TGCAACAGGATCGACTTGAGGGTGCCCAGTGGTCACCTCAAGGAGCCTGA
GCACCCGTCCATGTGGGGCCCGGTGGAGCTGGTAGGCATCATCGCCGGCC
CGGTGTTCCTCCTGTTCCTCATCATCATCATTGTTTTCCTTGTCATTAAC
TATCATCAGCGTGTCTATCACAACCGCCAGAGACTGGACATGGAAGATCC
CTCATGTGAGATGTGTCTCTCCAAAGACAAGACGCTCCAGGATCTTGTCT
ACGATCTCTCCACCTCAGGGTCTGGCTCAGGGTTACCCCTCTTTGTCCAG
CGCACAGTGGCCCGAACCATCGTTTTACAAGAGATTATTGGCAAGGGTCG
GTTTGGGGAAGTATGGCGGGGCCGCTGGAGGGGTGGTGATGTGGCTGTGA
AAATATTCTCTTCTCGTGAAGAACGGTCTTGGTTCAGGGAAGCAGAGATA
TACCAGACGGTCATGCTGCGCCATGAAAACATCCTTGGATTTATTGCTGC
TGACAATAAAGCAGACTGCTCATTCCTCACATTGCCATGGGAAGTTGTAA
TGGTCTCTGCTGCCCCCAAGCTGAGGAGCCTTAGACTCCAATACAAGGGA
GGAAGGGGAAGAGCAAGATTTTTATTCCCACTGAATAATGGCACCTGGAC
ACAGCTGTGGCTTGTTTCTGACTATCATGAGCACGGGTCCCTGTTTGATT
ATCTGAACCGGTACACAGTGACAATTGAGGGGATGATTAAGCTGGCCTTG
TCTGCTGCTAGTGGGCTGGCACACCTGCACATGGAGATCGTGGGCACCCA
AGGGAAGCCTGGAATTGCTCATCGAGACTTAAAGTCAAAGAACATTCTGG
TGAAGAAAAATGGCATGTGTGCCATAGCAGACCTGGGCCTGGCTGTCCGT
CATGATGCAGTCACTGACACCATTGACATTGCCCCGAATCAGAGGGTGGG
GACCAAACGATACATGGCCCCTGAAGTACTTGATGAAACCATTAATATGA
AACACTTTGACTCCTTTAAATGTGCTGATATTTATGCCCTCGGGCTTGTA
TATTGGGAGATTGCTCGAAGATGCAATTCTGGAGGAGTCCATGAAGAATA
TCAGCTGCCATATTACGACTTAGTGCCCTCTGACCCTTCCATTGAGGAAA
TGCGAAAGGTTGTATGTGATCAGAAGCTGCGTCCCAACATCCCCAACTGG
TGGCAGAGTTATGAGGCACTGCGGGTGATGGGGAAGATGATGCGAGAGTG
TTGGTATGCCAACGGCGCAGCCCGCCTGACGGCCCTGCGCATCAAGAAGA
CCCTCTCCCAGCTCAGCGTGCAGGAAGACGTGAAGATC
(SEQ ID NO:223)

TCCGGGCCCCGGGGGGTCCAGGCTCTGCTGTGTGCGTGCACCAGCTGCCT
CCAGGCCAACTACACGTGTGAGACAGATGGGGCCTGCATGGTTTCCATTT
TCAATCTGGATGGGATGGAGCACCATGTGCGCACCTGCATCCCCAAAGTG
GAGCTGGTCCCTGCCGGGAAGCCCTTCTACTGCCTGAGCTCGGAGGACCT
GCGCAACACCCACTGCTGCTACACTGACTACTGCAACAGGATCGACTTGA
GGGTGCCCAGTGGTCACCTCAAGGAGCCTGAGCACCCGTCCATGTGGGGC
CCGGTGGAG
(SEQ ID NO: 224)

ATGACCCGGGCGCTCTGCTCAGCGCTCCGCCAGGCTCTCCTGCTGCTCGC
AGCGGCCGCC GAGCTCTCGCCAGGACTGAAGTGTGTATGTCTTTTGTGTG
ATTCTTCAAACTTTACCTGCCAAACAGAAGGAGCATGTTGGGCATCAGTC
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGCATCACCAAATGCCCCAAAACTTGGACCCATGGAGCTGGCCATCAT
TATTACTGTGCCTGTTTGCCTCCTGTCCATAGCTGCGATGCTGACAGTAT
GGGCATGCCAGGGTCGACAGTGCTCCTACAGGAAGAAAAAGAGACCAAAT
GTGGAGGAACCACTCTCTGAGTGCAATCTGGTAAATGCTGGAAAAACTCT
GAAAGATCTGATTTATGATGTGACCGCCTCTGGATCTGGCTCTGGTCTAC
CTCTGTTGGTTCAAAGGACAATTGCAAGGACGATTGTGCTTCAGGAAATA
GTAGGAAAAGGTAGATTTGGTGAGGTGTGGCATGGAAGATGGTGTGGGGA
AGATGTGGCTGTGAAAATATTCTCCTCCAGAGATGAAAGATCTTGGTTTC
GTGAGGCAGAAATTTACCAGACGGTCATGCTGCGACATGAAAACATCCTT
GGTTTCATTGCTGCTGACAACAAAGATAATGGAACTTGGACTCAACTTTG
GCTGGTATCTGAATATCATGAACAGGGCTCCTTATATGACTATTTGAATA
GAAATATAGTGACCGTGGCTGGAATGATCAAGCTGGCGCTCTCAATTGCT
AGTGGTCTGGCACACCTTCATATGGAGATTGTTGGTACACAAGGTAAACC
TGCTATTGCTCATCGAGACATAAAATCAAAGAATATCTTAGTGAAAAAGT
GTGAAACTTGTGCCATAGCGGACTTAGGGTTGGCTGTGAAGCATGATTCA
ATACTGAACACTATCGACATACCTCAGAATCCTAAAGTGGGAACCAAGAG
GTATATGGCTCCTGAAATGCTTGATGATACAATGAATGTGAATATCTTTG
AGTCCTTCAAACGAGCTGACATCTATTCTGTTGGTCTGGTTTACTGGGAA
ATAGCCCGGAGGTGTTCAGTCGGAGGAATTGTTGAGGAGTACCAATTGCC
TTATTATGACATGGTGCCTTCAGATCCCTCGATAGAGGAAATGAGAAAGG
TTGTTTGTGACCAGAAGTTTCGACCAAGTATCCCAAACCAGTGGCAAAGT
TGTGAAGCACTCCGAGTCATGGGGAGAATAATGCGTGAGTGTTGGTATGC
CAACGGAGCGGCCCGCCTAACTGCTCTTCGTATTAAGAAGACTATATCTC
AACTTTGTGTCAAAGAAGACTGCAAAGCC (SEQ ID NO: 233)

GAGCTCTCGCCAGGACTGAAGTGTGTATGTCTTTTGTGTGATTCTTCAAA
CTTTACCTGCCAAACAGAAGGAGCATGTTGGGCATCAGTCATGCTAACCA
ATGGAAAAGAGCAGGTGATCAAATCCTGTGTCTCCCTTCCAGAACTGAAT
GCTCAAGTCTTCTGTCATAGTTCCAACAATGTTACCAAAACCGAATGCTG
CTTCACAGATTTTTGCAACAACATAACACTGCACCTTCCAACAGCATCAC
CAAATGCCCCAAAACTTGGACCCATGGAG (SEQ ID NO: 234)

ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGCATCACCAAATGCCCCAAAACTTGGACCCATGGAGCTGGCCATCAT
TATTACTGTGCCTGTTTGCCTCCTGTCCATAGCTGCGATGCTGACAGTAT
GGGCATGCCAGGGTCGACAGTGCTCCTACAGGAAGAAAAAGAGACCAAAT
GTGGAGGAACCACTCTCTGAGTGCAATCTGGTAAATGCTGGAAAAACTCT
GAAAGATCTGATTTATGATGTGACCGCCTCTGGATCTGGCTCTGGTCTAC
CTCTGTTGGTTCAAAGGACAATTGCAAGGACGATTGTGCTTCAGGAAATA
GTAGGAAAAGGTAGATTTGGTGAGGTGTGGCATGGAAGATGGTGTGGGGA
AGATGTGGCTGTGAAAATATTCTCCTCCAGAGATGAAAGATCTTGGTTTC
GTGAGGCAGAAATTTACCAGACGGTCATGCTGCGACATGAAAACATCCTT
GGTTTCATTGCTGCTGACAACAAAGATAATGGAACTTGGACTCAACTTTG
GCTGGTATCTGAATATCATGAACAGGGCTCCTTATATGACTATTTGAATA
GAAATATAGTGACCGTGGCTGGAATGATCAAGCTGGCGCTCTCAATTGCT
AGTGGTCTGGCACACCTTCATATGGAGATTGTTGGTACACAAGGTAAACC
TGCTATTGCTCATCGAGACATAAAATCAAAGAATATCTTAGTGAAAAAGT
GTGAAACTTGTGCCATAGCGGACTTAGGGTTGGCTGTGAAGCATGATTCA
ATACTGAACACTATCGACATACCTCAGAATCCTAAAGTGGGAACCAAGAG
GTATATGGCTCCTGAAATGCTTGATGATACAATGAATGTGAATATCTTTG
AGTCCTTCAAACGAGCTGACATCTATTCTGTTGGTCTGGTTTACTGGGAA
ATAGCCCGGAGGTGTTCAGTCGGAGGAATTGTTGAGGAGTACCAATTGCC
TTATTATGACATGGTGCCTTCAGATCCCTCGATAGAGGAAATGAGAAAGG
TTGTTTGTGACCAGAAGTTTCGACCAAGTATCCCAAACCAGTGGCAAAGT
TGTGAAGCACTCCGAGTCATGGGGAGAATAATGCGTGAGTGTTGGTATGC
CAACGGAGCGGCCCGCCTAACTGCTCTTCGTATTAAGAAGACTATATCTC
AACTTTGTGTCAAAGAAGACTGCAAAGCC (SEQ ID NO: 235)

ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGCATCACCAAATGCCCCAAAACTTGGACCCATGGAG
(SEQ ID NO: 236)

ATGACCCGGGCGCTCTGCTCAGCGCTCCGCCAGGCTCTCCTGCTGCTCGC
AGCGGCCGCCGAGCTCTCGCCAGGACTGAAGTGTGTATGTCTTTTGTGTG
ATTCTTCAAACTTTACCTGCCAAACAGAAGGAGCATGTTGGGCATCAGTC
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGGTCTACCTCTGTTGGTTCAAAGGACAATTGCAAGGACGATTGTGCT
TCAGGAAATAGTAGGAAAAGGTAGATTTGGTGAGGTGTGGCATGGAAGAT
GGTGTGGGGAAGATGTGGCTGTGAAAATATTCTCCTCCAGAGATGAAAGA
TCTTGGTTTCGTGAGGCAGAAATTTACCAGACGGTCATGCTGCGACATGA
AAACATCCTTGGTTTCATTGCTGCTGACAACAAAGATAATGGAACTTGGA
CTCAACTTTGGCTGGTATCTGAATATCATGAACAGGGCTCCTTATATGAC
TATTTGAATAGAAATATAGTGACCGTGGCTGGAATGATCAAGCTGGCGCT
CTCAATTGCTAGTGGTCTGGCACACCTTCATATGGAGATTGTTGGTACAC
AAGGTAAACCTGCTATTGCTCATCGAGACATAAAATCAAAGAATATCTTA
GTGAAAAAGTGTGAAACTTGTGCCATAGCGGACTTAGGGTTGGCTGTGAA
GCATGATTCAATACTGAACACTATCGACATACCTCAGAATCCTAAAGTGG
GAACCAAGAGGTATATGGCTCCTGAAATGCTTGATGATACAATGAATGTG
AATATCTTTGAGTCCTTCAAACGAGCTGACATCTATTCTGTTGGTCTGGT
TTACTGGGAAATAGCCCGGAGGTGTTCAGTCGGAGGAATTGTTGAGGAGT
ACCAATTGCCTTATTATGACATGGTGCCTTCAGATCCCTCGATAGAGGAA
ATGAGAAAGGTTGTTTGTGACCAGAAGTTTCGACCAAGTATCCCAAACCA
GTGGCAAAGTTGTGAAGCACTCCGAGTCATGGGGAGAATAATGCGTGAGT
GTTGGTATGCCAACGGAGCGGCCCGCCTAACTGCTCTTCGTATTAAGAAG
ACTATATCTCAACTTTGTGTCAAAGAAGACTGCAAAGCC
(SEQ ID NO: 237)

GAGCTCTCGCCAGGACTGAAGTGTGTATGTCTTTTGTGTGATTCTTCAAA
CTTTACCTGCCAAACAGAAGGAGCATGTTGGGCATCAGTCATGCTAACCA
ATGGAAAAGAGCAGGTGATCAAATCCTGTGTCTCCCTTCCAGAACTGAAT
GCTCAAGTCTTCTGTCATAGTTCCAACAATGTTACCAAAACCGAATGCTG
CTTCACAGATTTTTGCAACAACATAACACTGCACCTTCCAACAGGTCTAC
CTCTGTTGGTTCAAAGGACAATTGCAAGGACGATTGTGCTTCAGGAAATA
GTAGGAAAAGGTAGATTTGGTGAGGTGTGGCATGGAAGATGGTGTGGGGA
AGATGTGGCTGTGAAAATATTCTCCTCCAGAGATGAAAGATCTTGGTTTC
GTGAGGCAGAAATTTACCAGACGGTCATGCTGCGACATGAAAACATCCTT
GGTTTCATTGCTGCTGACAACAAAGATAATGGAACTTGGACTCAACTTTG
GCTGGTATCTGAATATCATGAACAGGGCTCCTTATATGACTATTTGAATA
GAAATATAGTGACCGTGGCTGGAATGATCAAGCTGGCGCTCTCAATTGCT
AGTGGTCTGGCACACCTTCATATGGAGATTGTTGGTACACAAGGTAAACC
TGCTATTGCTCATCGAGACATAAAATCAAAGAATATCTTAGTGAAAAAGT
GTGAAACTTGTGCCATAGCGGACTTAGGGTTGGCTGTGAAGCATGATTCA
ATACTGAACACTATCGACATACCTCAGAATCCTAAAGTGGGAACCAAGAG
GTATATGGCTCCTGAAATGCTTGATGATACAATGAATGTGAATATCTTTG
AGTCCTTCAAACGAGCTGACATCTATTCTGTTGGTCTGGTTTACTGGGAA
ATAGCCCGGAGGTGTTCAGTCGGAGGAATTGTTGAGGAGTACCAATTGCC
TTATTATGACATGGTGCCTTCAGATCCCTCGATAGAGGAAATGAGAAAGG
TTGTTTGTGACCAGAAGTTTCGACCAAGTATCCCAAACCAGTGGCAAAGT
TGTGAAGCACTCCGAGTCATGGGGAGAATAATGCGTGAGTGTTGGTATGC
CAACGGAGCGGCCCGCCTAACTGCTCTTCGTATTAAGAAGACTATATCTC
AACTTTGTGTCAAAGAAGACTGCAAAGCC (SEQ ID NO:238)

ATGACCCGGGCGCTCTGCTCAGCGCTCCGCCAGGCTCTCCTGCTGCTCGC
AGCGGCCGCCGAGCTCTCGCCAGGACTGAAGTGTGTATGTCTTTTGTGTG
ATTCTTCAAACTTTACCTGCCAAACAGAAGGAGCATGTTGGGCATCAGTC
ATGCTAACCAATGGAAAAGAGCAGGTGATCAAATCCTGTGTCTCCCTTCC
AGAACTGAATGCTCAAGTCTTCTGTCATAGTTCCAACAATGTTACCAAAA
CCGAATGCTGCTTCACAGATTTTTGCAACAACATAACACTGCACCTTCCA
ACAGATAATGGAACTTGGACTCAACTTTGGCTGGTATCTGAATATCATGA
ACAGGGCTCCTTATATGACTATTTGAATAGAAATATAGTGACCGTGGCTG
GAATGATCAAGCTGGCGCTCTCAATTGCTAGTGGTCTGGCACACCTTCAT
ATGGAGATTGTTGGTACACAAGGTAAACCTGCTATTGCTCATCGAGACAT
AAAATCAAAGAATATCTTAGTGAAAAAGTGTGAAACTTGTGCCATAGCGG
ACTTAGGGTTGGCTGTGAAGCATGATTCAATACTGAACACTATCGACATA
CCTCAGAATCCTAAAGTGGGAACCAAGAGGTATATGGCTCCTGAAATGCT
TGATGATACAATGAATGTGAATATCTTTGAGTCCTTCAAACGAGCTGACA
TCTATTCTGTTGGTCTGGTTTACTGGGAAATAGCCCGGAGGTGTTCAGTC
GGAGGAATTGTTGAGGAGTACCAATTGCCTTATTATGACATGGTGCCTTC
AGATCCCTCGATAGAGGAAATGAGAAAGGTTGTTTGTGACCAGAAGTTTC
GACCAAGTATCCCAAACCAGTGGCAAAGTTGTGAAGCACTCCGAGTCATG
GGGAGAATAATGCGTGAGTGTTGGTATGCCAACGGAGCGGCCCGCCTAAC
TGCTCTTCGTATTAAGAAGACTATATCTCAACTTTGTGTCAAAGAAGACT
GCAAAGCCTAA (SEQ ID NO: 239)

GAGCTCTCGCCAGGACTGAAGTGTGTATGTCTTTTGTGTGATTCTTCAAA
CTTTACCTGCCAAACAGAAGGAGCATGTTGGGCATCAGTCATGCTAACCA
ATGGAAAAGAGCAGGTGATCAAATCCTGTGTCTCCCTTCCAGAACTGAAT
GCTCAAGTCTTCTGTCATAGTTCCAACAATGTTACCAAAACCGAATGCTG
CTTCACAGATTTTTGCAACAACATAACACTGCACCTTCCAACAGATAATG
GAACTTGGACTCAACTTTGGCTGGTATCTGAATATCATGAACAGGGCTCC
TTATATGACTATTTGAATAGAAATATAGTGACCGTGGCTGGAATGATCAA
GCTGGCGCTCTCAATTGCTAGTGGTCTGGCACACCTTCATATGGAGATTG
TTGGTACACAAGGTAAACCTGCTATTGCTCATCGAGACATAAAATCAAAG
AATATCTTAGTGAAAAAGTGTGAAACTTGTGCCATAGCGGACTTAGGGTT
GGCTGTGAAGCATGATTCAATACTGAACACTATCGACATACCTCAGAATC
CTAAAGTGGGAACCAAGAGGTATATGGCTCCTGAAATGCTTGATGATACA
ATGAATGTGAATATCTTTGAGTCCTTCAAACGAGCTGACATCTATTCTGT
TGGTCTGGTTTACTGGGAAATAGCCCGGAGGTGTTCAGTCGGAGGAATTG
TTGAGGAGTACCAATTGCCTTATTATGACATGGTGCCTTCAGATCCCTCG
ATAGAGGAAATGAGAAAGGTTGTTTGTGACCAGAAGTTTCGACCAAGTAT
CCCAAACCAGTGGCAAAGTTGTGAAGCACTCCGAGTCATGGGGAGAATAA
TGCGTGAGTGTTGGTATGCCAACGGAGCGGCCCGCCTAACTGCTCTTCGT
ATTAAGAAGACTATATCTCAACTTTGTGTCAAAGAAGACTGCAAAGCCTA
A (SEQ ID NO: 240)

1-488. (canceled)

489. A protein comprising a variant ActRIIB amino acid sequence that is at least 90% identical to an amino acid sequence that begins at any one of amino acids 20-29 of SEQ ID NO: 2 and ends at any one of amino acids 109-134 of SEQ ID NO: 2, wherein the variant ActRIIB amino acid sequence comprises one or more amino acid substitutions with respect to SEQ ID NO: 2 at a position selected from A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, E106, F108, E111, R112, A119, G120, E123, P129, P130, and A132.

490. The protein of claim 489, wherein the variant ActRIIB amino acid sequence is at least 96% identical to amino acids 29-109 of SEQ ID NO: 2.

491. The protein of claim 489, wherein the variant ActRIIB amino acid sequence is at least 96% identical to amino acids 20-134 of SEQ ID NO: 2.

492. The protein of claim 489, wherein the variant ActRIIB amino acid sequence comprises one or more amino acid substitutions with respect to SEQ ID NO: 2 selected from A24N, S26T, N35E, E37A, E37D, L38N, R40A, R40K, S44T, L46V, L46I, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F108I, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.

493. The protein of claim 489, wherein the variant ActRIIB amino acid sequence comprises F82K substitution with respect to SEQ ID NO: 2.

494. The protein of claim 489, wherein the protein is a fusion protein that further comprises an Fc polypeptide amino acid sequence that is at least 94% identical to any one of SEQ ID NO: 13 to 30.

495. The protein of claim 494, further comprising a linker amino acid sequence between the variant ActRIIB amino acid sequence and the Fc polypeptide amino acid sequence, wherein the linker amino acid sequence is GGG or the amino acid sequence of any one of SEQ ID NOs: 262-267.

496. The protein of claim 489, wherein the protein comprises an amino acid sequence that is at least 94% identical to the amino acid sequence of SEQ ID NO: 522.

497. The protein of claim 489, wherein the protein comprises an amino acid sequence that is at least 94% identical to the amino acid sequence of SEQ ID NO: 524.

498. The protein of claim 489, wherein the protein comprises an amino acid sequence that is at least 98% identical to the amino acid sequence of SEQ ID NO: 524.

499. The protein of claim 489, wherein the protein is a homodimer.

500. The protein of claim 489, wherein the protein is a heteromultimer.

501. The protein of claim 500, wherein the protein further comprises an ALK4 polypeptide or an ALK7 polypeptide.

502. The protein of claim 501, wherein the heteromultimer is a heterodimer.

503. A recombinant nucleic acid comprising a coding sequence for the protein of claim 489.

504. A vector comprising the nucleic acid of claim 503.

505. A method of increasing red blood cell levels or hemoglobin levels in a subject, comprising administering to the subject in need thereof the protein of claim 489.

506. A method of increasing muscle mass or muscle strength in a subject, comprising administering to the subject in need thereof the protein of claim 489.

507. A method of treating a disorder in a subject, comprising administering to the subject in need thereof the protein of claim 489, wherein the disorder is selected from anemia, MDS, thalassemia, myelofibrosis, Duchenne muscular dystrophy (DMD), Becker muscular dystrophy (BMD), Charcot-Marie-Tooth disease (CMT), facioscapulohumeral muscular dystrophy (FSH or FSHD), Amyotrophic Lateral Sclerosis (ALS), spinal muscular atrophy (SMA), pulmonary arterial hypertension, interstitial lung disease, Alport syndrome, focal segmental glomerulosclerosis, polycystic kidney disease, chronic kidney disease, osteoporosis, hyperparathyroidism, Cushing’s disease, thyrotoxicosis, chronic diarrheal state or malabsorption, renal tubular acidosis, anorexia nervosa, and fibrodysplasia ossificans progressiva (FOP).

508. The method of claim 507, further comprising administering to the subject losartan, irbesartan, olmesartan, candesartan, valsartan, fimasartan, azilsartan, salprisartan, telmisartan, benazepril, captopril, enalapril, lisinopril, perindopril, ramipril, trandolapril, zofenopril, beclomethasone, betamethasone, budesonide, cortisone, dexamethasone, hydrocortisone, methylprednisolone, prednisolone, methylprednisone, prednisone, triamcinolone, cyclosporine, tacrolimus, cyclophosphamide, chlorambucil, tofacitinib, sirolimus, everolimus, azathioprine, leflunomide, mycophenolate, abatacept, adalimumab, anakinra, basiliximab, certolizumab, daclizumab, etanercept, fresolimumab, golimumab, infliximab, ixekizumab, natalizumab, rituximab, secukinumab, tocilizumab, ustekinumab, vedolizumab, benazepril, valsartan, fluvastatin, pravastatin, bardoxolone methyl, Achtar gel, tolvaptan, abatacept in combination with sparsentan, aliskiren, allopurinol, ANG-3070, atorvastatin, bleselumab, bosutinib, CCX140-B, CXA-10, D6-25-hydroxyvitamin D3, dapagliflozin, dexamethasone in combination with MMF, emodin, FG-3019, FK506, FK-506 and MMF, FT-011, galactose, GC1008, GFB-887, isotretinoin, lademirsen, lanreotide, levamisole, lixivaptan, losmapimod, metformin, mizorbine, N-acetylmannosamine, octreotide, paricalcitol, PF-06730512, pioglitazone, propagermanium, propagermanium and irbesartan, rapamune, rapamycin, RE-021, sparsentan, RG012, rosiglitazone, saquinivir, SAR339375, somatostatin, spironolactone, tesevatinib (KD019), tetracosactin, tripterygium wilfordii (TW), valproic acid, VAR-200, venglustat (GZ402671), verinurad, voclosporin, VX-147, kidney dialysis, kidney transplant, mesenchymal stem cell therapy, bone marrow stem cells, lipoprotein removal, a Liposorber LA-15 device, plasmapheresis, plasma exchange, or a change in dietary sodium intake.
